SEQUENCE LISTING (32 sequences)

SEQ ID NO: 1 - DNA, 1620 bp, Absidia coerulea
atgggtatttttttgatgaagttgttgtttttgttggtttcttctattattactttggtttcttgtttgcaattggaaccaacttttagatctactccaaagagaattgttttttgttctttgttgccaggttcttctcatgttaactgggttttgccatggttggatgaattggctttgagaggtcatgatgttacttttattactatggatatgaacgttaagtttgctgttccatttccaagaattgaaactattccagtttctcatgctaagattgatgttaagtctatttttagagatttgggttctcatccagatccattgaagatgatttctaagatttttactattcataagtctggttggagaactggttttgattctgtttctaactacattgaatctaaccaagttgatgttgttatttgtgatcatatgtctgatccatgtattgatgctgttttggctcatgatttgccattggttgttacttctactttggctttttttccagatgctaagactggttacattaacaacgatccattgactatgactgaatttactactcaacatcaatctatttctagaagattttacaacaagtacattttgccattgattttgttggctaagattaagccacaattgaaccaattggatcaattgaagagacaagctggttttattccaccaccacatgatcaaagaacttctaacgctttgaagattgtttcttctttgtacggtttggaaccagctagaccaatgggtccattggttgaattggttggtccagttttgtctccaaactacgctgatttggatttgacttttaagtcttacttggatactcatcataaggttgcttacgttgcttttggtcaacatgctgctccagatagacaagatattgctttgattttgacttctttgttggctcaagttgaaaagggtcatttggatggtattatttggtcttctagatctgcttctgttgatacttttccaccatctattgtttcttcttactctagaaagacttgggatttgactggtgtttttcatcaagaacaaaagggtccattgtttattacttcttggtctccacaattggctgttttgttgcatccatctgtttctgtttttttgtctcatggtggttctaactctttggttgaatctttgtacgctggtaagagattgttgttttacccattttttggtgatcaatggggtactgctagacaaatgactttgtgtggtttgggtgaatactttggtccaactactccacaagctcaagttgatgaaagagttgaagctgttgttgttgatcaacaaggtagataccaagctaacgttggtagataccaagctatggttcaaattcattctcaaactgctccaagaagagctgctgatttggttgaagaagttgcttttgcttctactcaatctactttgccacatagacatgatgttggtagatctatgtctatgattaagagaaacaactgggatattcatactgctgctgttttgattgttgttactttgattggtttggtttacagaggtgtttcttctagaagaattaagcaaaagattttgtaa

SEQ ID NO: 2 - protein, 539 aa, Absidia coerulea
MGIFLMKLLFLLVSSIITLVSCLQLEPTFRSTPKRIVFCSLLPGSSHVNWVLPWLDELALRGHDVTFITMDMNVKFAVPFPRIETIPVSHAKIDVKSIFRDLGSHPDPLKMISKIFTIHKSGWRTGFDSVSNYIESNQVDVVICDHMSDPCIDAVLAHDLPLVVTSTLAFFPDAKTGYINNDPLTMTEFTTQHQSISRRFYNKYILPLILLAKIKPQLNQLDQLKRQAGFIPPPHDQRTSNALKIVSSLYGLEPARPMGPLVELVGPVLSPNYADLDLTFKSYLDTHHKVAYVAFGQHAAPDRQDIALILTSLLAQVEKGHLDGIIWSSRSASVDTFPPSIVSSYSRKTWDLTGVFHQEQKGPLFITSWSPQLAVLLHPSVSVFLSHGGSNSLVESLYAGKRLLFYPFFGDQWGTARQMTLCGLGEYFGPTTPQAQVDERVEAVVVDQQGRYQANVGRYQAMVQIHSQTAPRRAADLVEEVAFASTQSTLPHRHDVGRSMSMIKRNNWDIHTAAVLIVVTLIGLVYRGVSSRRIKQKIL

SEQ ID NO: 3 - DNA, 972 bp, Mucor hiemalis
atggttggtgttcatatggctgaaaagttgcaagttccatactttagatctatgccatttccattttctagaactactaagtttccacatccatttgctattgctcaaacttctgctggtggtagaatttacaacgatatgacttacgttatgattgatatggctttgtgggctggtacttctaagtacgttaacagatttagaagaaacgttttgaacttgccatctactactttggatagattgcatttgtggaaggttccacatatttactctttttctccaactgttgttcatccaccaaaggattggccagattacattcattgtactggttactggtttttggataacccagatagagtttggcaaccagatccagctttggtttcttttttggaaaacaaggaaaagccaattgtttacattggttttggttctattattgttccagatgctgaagaaactactagaactattgttcaagctgttttgcaatctggtgttaaggctattatttgtaagggttggtctactagaggtactgaaaaggataagaaggaagatggtgaattgttggatcaatacccaggtattattcatcatttggattctgttccacatgattggttgtttccacaaattcaaggtgttgttcatcatggtggtgctggtactactgctgctggtttgagagctggtttgccaactattattaagccattttttggtgatcaaagattttggggtcaaagagttgaagaattgcaagttggtatttgtttgaacaagttgactaagtcttctttggctgaagctttgattgctattactcaaaacaagactatgattgctaaggctaacattattggtgaaactattagaaaggaaaacggtccaagacaagctgttgaaaacatttacagagaatttgattacgctagagaacaaagaactaaccaaggttcttaa

SEQ ID NO: 4 - protein, 323 aa, Mucor hiemalis
MVGVHMAEKLQVPYFRSMPFPFSRTTKFPHPFAIAQTSAGGRIYNDMTYVMIDMALWAGTSKYVNRFRRNVLNLPSTTLDRLHLWKVPHIYSFSPTVVHPPKDWPDYIHCTGYWFLDNPDRVWQPDPALVSFLENKEKPIVYIGFGSIIVPDAEETTRTIVQAVLQSGVKAIICKGWSTRGTEKDKKEDGELLDQYPGIIHHLDSVPHDWLFPQIQGVVHHGGAGTTAAGLRAGLPTIIKPFFGDQRFWGQRVEELQVGICLNKLTKSSLAEALIAITQNKTMIAKANIIGETIRKENGPRQAVENIYREFDYAREQRTNQGS

SEQ ID NO: 5 - DNA, 1330 bp, Camellia sinensis
atgggatccagaaaacagcctcacgccgtttgtgtaccttttcctgctcagggacacattaaccctatgatgcagcttgccaagctactgcattcaagaggcttctacataacgttcgtcaacaccgagtttaaccataggagactgctgcaatccaagggtcccgaatttttgaaaggttgtgcagacttccagtttgagtccattccagatggtttacctccctcagacagagacgccacgcaagacccacctactctgtgtattgcaatgagggataattgtcttgacccattcagggtgttattgaagaagttaaataataacaacaactccattgcttccaggcaagtcccaggagtgacatgtgtcgttagtgacggagctatgaattttgccatgaaagccgctgaagaagctggaatacccgaggtccagttctggaccgcttccgcctgcggttttatgggctatttgcattatccacagctagtgcagaggggcatcttccccttcaaggatgaaagtttccagagtgacggttcattagacactactatcgattggatccctggaatgcgtaacattagactaaaggacatgccttccttcatccgtaccacagatccaaacgatatactgtttaattaccttagtgaggaagttcagaactgtttaaaagccagtgcaatcatatttaacacattcgatacacttgagcaccaggtcctgcaggctatcgcctccaaatttcacaatatatacacgataggtcctttgagtctgctatctaaacaggttatagatggcgagtttaagtctctgaacagttctttatggaaagaagatactaaatgcctacagtggctagacacgaaggaacccaactctgttgtatacgttaattatggctcaataacagtaatgacagaccagcatcttaaggagttcgcctggggtcttgccaattctaaacaccccttcctttggatcgtgagacccgacattgtcatgggagatagtgctatcttacccgaacacttcgttgaggaaaccaaagacaggggactattagtatcatggtgtcctcaagaacaagtcttatcacatccttctattggtgtttttctaacccattgcggttggaactccaccttggagtccatatgtggtggtgtgcccattatatgctggccattcttcgccgaacaacagaccaactgcaggtacgcctgcacggaatggggtataggcatggaagtgaaccatgacgtaaagcgtaacgaaattgtggccttaataaatgagatgttagagggcgacaagggaa

SEQ ID NO: 6 - protein, 485 aa, Camellia sinensis
MGSRKQPHAVCVPFPAQGHINPMMQLAKLLHSRGFYITFVNTEFNHRRLLQSKGPEFLKGCADFQFESIPDGLPPSDRDATQDPPTLCIAMRDNCLDPFRVLLKKLNNNNNSIASRQVPGVTCVVSDGAMNFAMKAAEEAGIPEVQFWTASACGFMGYLHYPQLVQRGIFPFKDESFQSDGSLDTTIDWIPGMRNIRLKDMPSFIRTTDPNDILFNYLSEEVQNCLKASAIIFNTFDTLEHQVLQAIASKFHNIYTIGPLSLLSKQVIDGEFKSLNSSLWKEDTKCLQWLDTKEPNSVVYVNYGSITVMTDQHLKEFAWGLANSKHPFLWIVRPDIVMGDSAILPEHFVEETKDRGLLVSWCPQEQVLSHPSIGVFLTHCGWNSTLESICGGVPIICWPFFAEQQTNCRYACTEWGIGMEVNHDVKRNEIVALINEMLEGDKGKQMRKKALKLKKEAEEATDVGGLSYNNFDRLIKEALHYCEQY

SEQ ID NO: 7 - DNA, 1362 bp, Camellia sinensis
atggattcaaaaaagagcaaaatgaatgtcctaatgctaccatggttagcccaaggtcacatcactcccttcctagagcttgccaagaaactcacaaacaaaaacttccacatatatttctgttctacaccaatcaatctcatatccatcaagaaaagaatcacagacaagtactctctctccattgaacttgtagaaatccaccttccatccttgcctgagctccctcctcactaccacaccaccaatggcctccccatccatctcaattccactctcaaaacagcctttgagatggcaagcacaagcttctccaccatcctcaacacactaagcccagatcttgtaatttatgacgtcagcccctcatgggcacaatccactgccttgtcgtttgatattcccgcagtccagctcatgattaccggagccacggtggtttcttttggccaacatatgattaagcactgtggcagtgttgaattcccatttccggcaattaagctgcagggattccatgaaacgcagtttcgccactttgttgagactgttgtaaaagaatacaatgacaaacaagttgcctctgtaaatgatcaaccatcttgcaactttatgttgtacaacacttttagagagcttgaaggtaaatacattgattatctaccggttataggtgagaagaaggttgtccctgttggtccacttgttcaaggcattgatgatgatgaaaatgagcactctgagatcatacaatggcttgacaacaagggtgagtactccactttgtttgtttcttttgggagtgagtattttatgtccaaggaagagattgaagagatagctcatgggttagagcttagcatggttaatttcatatgggttgttaggtttcctgaggtagagaaagttgagcttgaagaagcacttcctaaggggtttattgacagggtaggagagagaggactggttgtggagggttgggctccacaggcaagaattctaacccattcaagcactggtggatttgtgagtcactgtgaatggaattcagtattggaaagcctaaagtttggggttccaatggtagccatacccatgcaatatgagcagccgttgaatgctaaactagtggaggaggttggtgtggctgcggaggtcaacagagacatcaatggaagactcaacagagaagagatagcacaggtaataaggaaggttgtggtggagaaaagtggagaggacataaggattaaagcgagaatttttggggataaaataagaatgaaaggagatgaagagatagacgaggcagtagaagtgttgctgcagctttgtaaggatgtgaagttgctcaaaaattag

SEQ ID NO: 8 - protein, 453 aa, Camellia sinensis
MDSKKSKMNVLMLPWLAQGHITPFLELAKKLTNKNFHIYFCSTPINLISIKKRITDKYSLSIELVEIHLPSLPELPPHYHTTNGLPIHLNSTLKTAFEMASTSFSTILNTLSPDLVIYDVSPSWAQSTALSFDIPAVQLMITGATVVSFGQHMIKHCGSVEFPFPAIKLQGFHETQFRHFVETVVKEYNDKQVASVNDQPSCNFMLYNTFRELEGKYIDYLPVIGEKKVVPVGPLVQGIDDDENEHSEIIQWLDNKGEYSTLFVSFGSEYFMSKEEIEEIAHGLELSMVNFIWVVRFPEVEKVELEEALPKGFIDRVGERGLVVEGWAPQARILTHSSTGGFVSHCEWNSVLESLKFGVPMVAIPMQYEQPLNAKLVEEVGVAAEVNRDINGRLNREEIAQVIRKVVVEKSGEDIRIKARIFGDKIRMKGDEEIDEAVEVLLQLCKDVKLLKN

SEQ ID NO: 9 - DNA, 1440 bp, Vitis vinifera
atggaatctgttgttttgtacccatctccaggtatgggtcatttgatttctatggttgaattgggtaagttgattttgaagcatcatccatctttttctattactatttttattgttactccaccatacaacactggttctactgctccatacttggctagagtttcttctactattccatctattacttttcatcatttgccaactatttctttgccattggattctttttcttctccaaaccatgaaactttggcttttgaattgttgagattgaacaacccaaacattcatcaagctttggtttctatttctaacaactcttctgttagagctttgattgttgattgtttttgtactgctgctttgtctgttgctgctcaattgaacattccattttactacttttttacttctggtgcttgttgtttggcttcttttttgtacttgccatttattcatcaacaaactactaagtcttttaaggatttgaacactcatttgcatattccaggtttgccaccagttccagcttctgatatggctaagccaattttggatagagaagataaggcttacgaattgtttgttaacatgtctattcatttgccaagatctgctggtattattgttaacacttttgaagctttggaaccaagagctgttaagactattttggatggtttgtgtgttttggatggtccaacttctccaattttttgtattggtccattgattgctgctgatgatagatctggtggtggtggtggtggtggtggtggttctggtattccagaatgtttgacttggttggaatctcaaccaaagagatctgttttgtttttgtgttttggttctttgggtttgttttctgaagaacaattgaaggaaattgctgttggtttggaaagatctggtcaaagatttttgtgggttgttagatctccaccatctaaggatccatctagaagatttttggctccaccagaaccagatttgaactctttgttgccagatggttttttggatagaactaaggaaagaggtttgatggttaagtcttgggctccacaagttgctgttttgaaccatgcttctgttggtggttttgttactcattgtggttggaactctgttttggaagctgtttgtgctggtgttccaatggttgcttggccattgtacgctgaacaaagatttaacagagttgttttggttgaagaaatgaagttggcttttccaatggaagaatctgaagaaggttttgttactgctactgaagttgaaaagagagttagagaattgatggaatctgaagaaggtaacactttgagattgagaattatggctatgaaggaagctgctgaaactgctatgtctgatggtggttcttctagaactgctttgactaagttggttaagtcttggagaccaggttaa

SEQ ID NO: 10 - protein, 479 aa, Vitis vinifera
MESVVLYPSPGMGHLISMVELGKLILKHHPSFSITIFIVTPPYNTGSTAPYLARVSSTIPSITFHHLPTISLPLDSFSSPNHETLAFELLRLNNPNIHQALVSISNNSSVRALIVDCFCTAALSVAAQLNIPFYYFFTSGACCLASFLYLPFIHQQTTKSFKDLNTHLHIPGLPPVPASDMAKPILDREDKAYELFVNMSIHLPRSAGIIVNTFEALEPRAVKTILDGLCVLDGPTSPIFCIGPLIAADDRSGGGGGGGGGSGIPECLTWLESQPKRSVLFLCFGSLGLFSEEQLKEIAVGLERSGQRFLWVVRSPPSKDPSRRFLAPPEPDLNSLLPDGFLDRTKERGLMVKSWAPQVAVLNHASVGGFVTHCGWNSVLEAVCAGVPMVAWPLYAEQRFNRVVLVEEMKLAFPMEESEEGFVTATEVEKRVRELMESEEGNTLRLRIMAMKEAAETAMSDGGSSRTALTKLVKSWRPG

SEQ ID NO: 11 - DNA, 1449 bp, Nicotiana tabacum
atgaaagagactaaaaaaattgagttagtttttatccccagtcctggtataggacacttagtctcaactgtggagatggccaaactgttgatagcccgtgaagagcaactttctattactgtcctgattatacaatggcctaatgataaaaagctagacagttatatccagtccgtcgcaaactttagttctagactgaagtttatacgtctgccccaagatgactcaatcatgcaacttttgaaatcaaacattttcacgacattcatcgcctctcacaagccagctgtaagagacgccgttgctgacatactaaagagtgaaagtaataacacattggcaggcattgtaatcgatcttttctgcacatccatgatcgatgtagccaatgagtttgagctgcctacttatgtgttttacactagtggcgcagccacgttgggtctgcactaccatattcaaaatctgcgtgatgagtttaataaagacattaccaaatataaggatgagccagaagaaaaattaagtatagccacgtaccttaacccattccctgctaagtgtctaccctccgtggcattggataaggaaggaggatcaacgatgttcctagacttagctaagaggttcagggagaccaaaggcataatgattaacacttttcttgagctggaatcatacgctctaaactcattgtctagagataaaaacttgccccctatataccctgtaggccctgttttgaacttgaacaacgttgagggtgataacttgggctctagtgatcaaaataccatgaaatggctggacgaccagccagcttcttccgttgtgttcctatgttttggctcaggaggaagtttcgaaaaacaccaagtcaaagaaatagcttatgccttagaatcttccggatgcaggttcttgtggagtttgcgtagaccccccacggaagatgctaggttcccttctaattacgaaaacttagaggaaattttaccagagggatttctggaaagaacgaaaggcattggtaaggtcattggatgggccccacagttagcaatcttgtctcacaagtccacaggaggattcgtgtctcattgcggatggaactctacccttgaaagtacctatttcggcgttcctattgctacttggccaatgtatgctgaacaacaggccaacgcttttcaacttgttaaagatttgaggatgggtgttgagatcaaaatggattataggaaggatatgaaggtaatgggcaaggaggttatcgttaaggcagaagaaattgaaaaggccataagggaaatcatggactcagaatcagaaatcagggtcaaggtcaaagagatgaaggagaaaagtcgtgcagcccaaatggaaggaggatcatcatatacctctatcggcggcttcattcaaataatcatggagaactcacagtaa

SEQ ID NO: 12 - protein, 482 aa, Nicotiana tabacum
MKETKKIELVFIPSPGIGHLVSTVEMAKLLIAREEQLSITVLIIQWPNDKKLDSYIQSVANFSSRLKFIRLPQDDSIMQLLKSNIFTTFIASHKPAVRDAVADILKSESNNTLAGIVIDLFCTSMIDVANEFELPTYVFYTSGAATLGLHYHIQNLRDEFNKDITKYKDEPEEKLSIATYLNPFPAKCLPSVALDKEGGSTMFLDLAKRFRETKGIMINTFLELESYALNSLSRDKNLPPIYPVGPVLNLNNVEGDNLGSSDQNTMKWLDDQPASSVVFLCFGSGGSFEKHQVKEIAYALESSGCRFLWSLRRPPTEDARFPSNYENLEEILPEGFLERTKGIGKVIGWAPQLAILSHKSTGGFVSHCGWNSTLESTYFGVPIATWPMYAEQQANAFQLVKDLRMGVEIKMDYRKDMKVMGKEVIVKAEEIEKAIREIMDSESEIRVKVKEMKEKSRAAQMEGGSSYTSIGGFIQIIMENSQ

SEQ ID NO: 13 - DNA, 1491 bp, Nicotiana tabacum
atggctactcaggtgcataaattgcatttcattctgttcccactgatggctcccggtcacatgatccctatgatagacatcgcaaaactattggctaaccgtggcgtgataactaccataataactacgcccgttaacgccaatcgtttttcctctacgatcactagggccattaaatcaggcctaagaatccagattttaaccttaaaattcccatcagttgaggtaggcctgcctgaaggatgtgaaaacatcgacatgttgccatctttggacttagcctctaaattctttgctgctatttctatgcttaaacaacaagtggagaacttgctagagggtattaaccctagtccctcatgcgttatttctgacatgggcttcccatggacgacacagatcgctcaaaatttcaatattcctcgtatcgtatttcatggcacgtgttgcttttctcttctttgttcttacaaaatcctgtcatccaatatcttagagaacattactagtgactcagagtattttgtcgtgccagatctgccagaccgtgtcgagctaactaaggcccaagtctctggatctacaaagaatactacatcagtaagtagttcagtactgaaggaggttacagagcagatcaggcttgcagaggaatcatcctacggtgtgatagttaattccttcgaagaactggaacaggtgtatgaaaaagagtacagaaaagccaggggcaaaaaggtctggtgcgtgggtcctgtctctttgtgcaacaaggagattgaagatcttgttactagaggaaacaaaaccgctatagacaatcaggattgtcttaagtggttagacaacttcgagactgaatccgtcgtctatgcaagtttaggctcactaagtaggcttacgttactgcaaatggttgagctgggattgggactggaggagagtaataggccatttgtatgggttctgggaggaggagacaaactaaatgatcttgagaaatggatattggagaatggctttgaacagcgtataaaggagagaggtgtcctgatacgtggctgggcacctcaagtattgattttaagtcaccccgcaattggaggagttttaacgcattgtggatggaactctacattagagggcatttcagccggactacccatggtcacctggccactatttgccgaacagttctgtaacgaaaaattagtagtgcaggttcttaaaatcggtgtctcacttggagtgaaggtccctgttaagtggggtgacgaagagaacgtaggtgtcttagtgaaaaaggatgacgttaaaaaagcactggataagctaatggatgagggtgaggagggccaggttaggaggaccaaagccaaagagcttggtgagttagctaaaaaagcctttggagagggcggatcatcctacgtgaacctaacgtccctaattgaagatataatcgagcagcagaaccataaggagaagtag

SEQ ID NO: 14 - protein, 496 aa, Nicotiana tabacum
MATQVHKLHFILFPLMAPGHMIPMIDIAKLLANRGVITTIITTPVNANRFSSTITRAIKSGLRIQILTLKFPSVEVGLPEGCENIDMLPSLDLASKFFAAISMLKQQVENLLEGINPSPSCVISDMGFPWTTQIAQNFNIPRIVFHGTCCFSLLCSYKILSSNILENITSDSEYFVVPDLPDRVELTKAQVSGSTKNTTSVSSSVLKEVTEQIRLAEESSYGVIVNSFEELEQVYEKEYRKARGKKVWCVGPVSLCNKEIEDLVTRGNKTAIDNQDCLKWLDNFETESVVYASLGSLSRLTLLQMVELGLGLEESNRPFVWVLGGGDKLNDLEKWILENGFEQRIKERGVLIRGWAPQVLILSHPAIGGVLTHCGWNSTLEGISAGLPMVTWPLFAEQFCNEKLVVQVLKIGVSLGVKVPVKWGDEENVGVLVKKDDVKKALDKLMDEGEEGQVRRTKAKELGELAKKAFGEGGSSYVNLTSLIEDIIEQQNHKEK

SEQ ID NO: 15 - DNA, 23 bp, Artificial Sequence
  UGT58A1 Forward primer
ccaccaccacatgatcaaagaac

SEQ ID NO: 16 - DNA, 23 bp, Artificial Sequence
  UGT58A1 Reverse primer
gtcatttgtctagcagtacccca

SEQ ID NO: 17 - DNA, 23 bp, Artificial Sequence
  MhGT1 Forward Primer
gattgatatggctttgtgggctg

SEQ ID NO: 18 - DNA, 23 bp, Artificial Sequence
  MhGT1 Reverse primer
ctctcaaaccagcagcagtagta

SEQ ID NO: 19 - DNA, 25 bp, Artificial Sequence
  NtGT3 Forward primer
atgaaagagactaaaaaaattgagt

SEQ ID NO: 20 - DNA, 23 bp, Artificial Sequence
  NtGT3 Reverse primer
catcacgcagattttgaatatgg

SEQ ID NO: 21 - DNA, 25 bp, Artificial Sequence
  NtGT4 Forward primer
atggctactcaggtgcataaattgc

SEQ ID NO: 22 - DNA, 22 bp, Artificial Sequence
  NtGT4 Reverse primer
ggccttagttagctcgacacgg

SEQ ID NO: 23 - DNA, 23 bp, Artificial Sequence
  VvGT7 Forward primer
tacaacactggttctactgctcc

SEQ ID NO: 24 - DNA, 23 bp, Artificial Sequence
  VvGT7 Reverse primer
gcccaagacttaaccatcaaacc

SEQ ID NO: 25 - DNA, 23 bp, Artificial Sequence
  UGT85K11 Forward primer
ccagagtgacggttcattagaca

SEQ ID NO: 26 - DNA, 23 bp, Artificial Sequence
  UGT85K11 Reverse primer
tcaacgaagtgttcgggtaagat

SEQ ID NO: 27 - DNA, 23 bp, Artificial Sequence
  UGT94P1 Forward primer
ctccaccatcctcaacacactaa

SEQ ID NO: 28 - DNA, 23 bp, Artificial Sequence
  UGT94P1 Reverse primer
ccatgctaagctctaacccatga

SEQ ID NO: 29 - DNA, 1179 bp, Bacillus subtilis
atgaaaaagtaccatatttctatgatcaatatcccagcttacggacatgtcaatcctacgcttgctttagtagagaagctttgtgagaaagggcaccgtgtcacgtacgctacgactgaggagtttgctcccgctgttcagcaagccggtggagaagcattgatctatcatacatccttgaatattgatcctaagcaaatcagggagatgatggaaaagaatgacgctcccttgtctcttttgaaagaatcattgtctattctgccacagcttgaggagttatataaggatgatcagcctgatctgatcatctatgactttgttgctctggctggtaaattgtttgctgaaaagcttaatgttccagtcattaagttgtgttcttcatatgcccaaaatgaatcctttcagttaggaaatgaagacatgctgaaaaaaataagagaagcagaggctgaatttaaagcctacttggagcaagagaagttgccagctgtttcatttgaacagttagctgtgccagaagcattaaatattgtctttatgccaaagtcttttcagattcagcatgagacgttcgatgaccgtttctgttttgtcggcccctctttgggagaaagaaaggaaaaagaatctctgttgattgacaaggatgatagaccacttatgctgatttctttgggtacggcttttaacgcatggccagaattttacaagatgtgcatcaaggcatttagagattcttcatggcaagtgatcatgtctgttgggaaaacgattgatccagaatctttggaggatattcctgctaactttaccattagacaaagtgtgccacagcttgaggtgttagagaaagctgatttgttcatctctcatggcgggatgaacagtacgatggaagctatgaacgcaggtgtgccacttgtcgtcattccacaaatgtatgagcaggagttgactgcaaatagagttgatgaattaggccttggcgtttatttgccaaaagaggaagtgactgtttcctctctgcaggaagctgttcaggctgtatccagtgatcaagagctgttgtctagagtcaagaatatgcaaaaggatgtaaaagaagctggcggagctgagcgtgctgcagctgagattgaagcttttatgaaaaaatccgctgtcccacagtaa

SEQ ID NO: 30 - protein, 392 aa, Bacillus subtilis
MKKYHISMINIPAYGHVNPTLALVEKLCEKGHRVTYATTEEFAPAVQQAGGEALIYHTSLNIDPKQIREMMEKNDAPLSLLKESLSILPQLEELYKDDQPDLIIYDFVALAGKLFAEKLNVPVIKLCSSYAQNESFQLGNEDMLKKIREAEAEFKAYLEQEKLPAVSFEQLAVPEALNIVFMPKSFQIQHETFDDRFCFVGPSLGERKEKESLLIDKDDRPLMLISLGTAFNAWPEFYKMCIKAFRDSSWQVIMSVGKTIDPESLEDIPANFTIRQSVPQLEVLEKADLFISHGGMNSTMEAMNAGVPLVVIPQMYEQELTANRVDELGLGVYLPKEEVTVSSLQEAVQAVSSDQELLSRVKNMQKDVKEAGGAERAAAEIEAFMKKSAVPQ

SEQ ID NO: 31 - DNA, 1422 bp, Vitis vinifera
atgggttctatggaaaagccacatgctgtttgtattccatacccagctcaaggtcatattaacccaatgttgaaggttgctaagttgttgcattttagaggttttagaattacttttgttaacactgaatttaaccatactagattgttgaaggctcaaggtccaaactctttgaacggtttgccaacttttcaatttgaaactattccagatggtttgccaccatctaacgttgatgctactcaagatattccatctttgtgtgcttctactaagaagaactgtttggctccatttagaagattgttggctaagttgaacgatagaggtccaccagttacttgtattttttctgatgctgttatgtcttttactttggatgctgctcaagaattgggtattccagatttgttgttgtggactgcttctgcttgtggttttatggcttacgttcaatacagatctttgattgataagggttttactccattgaaggatgaatcttacttgactaacggttacttggatactgttgttgattggattccaggtatgaagggtattagattgaaggatttgccatcttttattagaactactgatccagatgatattatgttggattttgctatgggtgaattggaaagagctagaaaggcttctgctattatttttaacacttttgatgctttggaacaagaagttttggatgctattgctccaatgtacccaccaatttacactattggtccattgcaattgttgccagatcaaattcatgattctgaattgaagttgattggttctaacttgtggaaggaagaaccagaatgtttgaagtggttggattctaaggaaccaaactctgttgtttacgttaactacggttctattactgttatgactccacaacaattgattgaatttgcttggggtttggctaactctaaccaatcttttttgtggattttgagaccagatttggtttctggtgaatctgctattttgccaccagaatttgttgctgaaactgaagatagaggtttgttggctggttggtgtccacaagaacaagttttgactcatcaagctattggtggttttttgactcataacggttggaactctactattgaaggtttgtgtgctggtgttccaatgatttgttggccattttttgctgaacaacaaactaactgtagatactgttgtactgaatggggtgttggtatggaaattgattctgatgttaagagagatgaagttgctaagttggttagagaattgatggttggtgaaaagggtaaggttatgaagaagaagactatggaatggaagcatagagctgaagttgctactactggtccagatggttcttcttacttgaacttggaaaagatttttgaacaagttttgttgtaa

SEQ ID NO: 32 - protein, 473 aa, Vitis vinifera
MGSMEKPHAVCIPYPAQGHINPMLKVAKLLHFRGFRITFVNTEFNHTRLLKAQGPNSLNGLPTFQFETIPDGLPPSNVDATQDIPSLCASTKKNCLAPFRRLLAKLNDRGPPVTCIFSDAVMSFTLDAAQELGIPDLLLWTASACGFMAYVQYRSLIDKGFTPLKDESYLTNGYLDTVVDWIPGMKGIRLKDLPSFIRTTDPDDIMLDFAMGELERARKASAIIFNTFDALEQEVLDAIAPMYPPIYTIGPLQLLPDQIHDSELKLIGSNLWKEEPECLKWLDSKEPNSVVYVNYGSITVMTPQQLIEFAWGLANSNQSFLWILRPDLVSGESAILPPEFVAETEDRGLLAGWCPQEQVLTHQAIGGFLTHNGWNSTIEGLCAGVPMICWPFFAEQQTNCRYCCTEWGVGMEIDSDVKRDEVAKLVRELMVGEKGKVMKKKTMEWKHRAEVATTGPDGSSYLNLEKIFEQVLL

(19) United States
(12) Patent Application Publication
(10) Pub. No.: US 2021/0348204 A1
(43) Pub. Date: Nov. 11, 2021

(54) Systems, Methods, And Compositions For The Production of Water-Soluble Terpenes Derived From Cannabis Plants

(71) Applicant: Trait Biosciences, Inc., Los Alamos, NM (US)

(72) Inventors: Richard T. Sayre, Los Alamos, NM (US); Elton Carvalho Goncalves, Los Alamos, NM (US); Jennifer Stamps, Los Alamos, NM (US); Tawanda Zidenga, White Rock, NM (US); Stephanie Willette, Santa Fe, NM (US)

(21) Appl. No.: 17/285,800

(22) PCT Filed: Oct. 16, 2019
(86) PCT No.: PCT/US19/56613

§ 371 Date: Apr. 15, 2021

Related U.S. Application Data
(60) Provisional application No. 62/746,053, filed on Oct. 16, 2018.

(51) Int. Cl. C12P 19/44; C12N 9/10; C12N 1/18; C07H 15/10

(52) U.S. Cl. CPC C12P 19/44; C12N 9/1048; C12R 2001/84; C07H 15/10; C12N 1/185

(57) ABSTRACT

The current inventive technology includes novel systems, methods and compositions for the in vivo production, modification and isolation of terpene compounds from Cannabis or other plants. In particular, the invention provides systems and methods for high level in vivo biosynthesis of water-soluble terpene compounds. In one preferred embodiment, the present invention generally relates to the conversion of Cannabis sativa or hemp derived terpenes into water-soluble terpene compounds, such as terpene glycosides, glycosylated-xylosylated terpenes, and an acetylated glycoside terpenes. In one preferred aspect, the invention may include system for producing water-soluble terpene compounds in yeast and tobacco cell suspension cultures, as well as an in planta system using transgenic Cannabis or hemp plants.

### CROSS-REFERENCE TO RELATED APPLICATIONS

This application claims the benefit of and priority to U.S. Provisional Patent Application No. 62/746,053, filed Oct. 16, 2018, the entirety of which is hereby incorporated by reference.

### TECHNICAL FIELD

The current inventive technology relates to the chemical modification of plant derived terpenes, and in particular the in vivo bioconversion of Cannabis-derived terpenes into water-soluble terpene glycosides.

### BACKGROUND OF THE INVENTION

Terpenes, which may be used interchangeably with the term terpenoids, are a large and diverse class of naturally occurring organic chemicals. Terpenes are essential for plant metabolism, influencing general development, herbivory defense, pollination and stress response. These compounds have been extensively used as flavoring and scenting agents in cosmetics, detergents, food and pharmaceutical products. They also display multiple biological activities in humans, such as anti-inflammatory, anti-microbial, antifungal and antiviral.

Cannabis terpenes profiles define the aroma of each plant and share the same precursor (geranyl pyrophosphate) and the same synthesis location (glandular trichomes) as phytocannabinoids. While over 400 terpenoids have been identified in Cannabis, a few of the most common include: limonene, myrcene, alpha-pinene, linalool, beta-caryophyllene, caryophyllene oxide, nerol and alpha-terpineol.

As shown in FIG. 27, terpenes, and/or terpenoids are mainly synthesized in two metabolic pathways: mevalonic acid pathway (a.k.a. HMG-CoA reductase pathway, which takes place in the cytosol) and MEP/DOXP pathway (a.k.a. The 2-C-methyl-D-erythritol 4-phosphate/l-deoxy-D-xylulose 5-phosphate pathway, non-mevalonate pathway, or mevalonic acid-independent pathway, which takes place in plastids). Geranyl pyrophosphate (GPP), which is used by Cannabis plants to produce cannabinoids, is formed by condensation of dimethylallyl pyrophosphate (DMAPP) and isopentenyl pyrophosphate (IPP) via the catalysis of GPP synthase. Alternatively, DMAPP and IPP are ligated by FPP synthase to produce farnesyl pyrophosphate (FPP), which can be used to produce sesquiterpenoids. Geranyl pyrophospliate (GPP) can also be converted into monoterpenoids by limonene synthase.

As highlighted in table 6, a survey of the terpenoid profiles of several Cannabis varieties. Additional studies indicate that these terpenoids express at high enough levels to potentially have their own pharmacological effects and also to act in synergy with cannabinoids. The biochemical and phenomenological differences between different varieties of Cannabis, which are attributed to their unique relative cannabinoid and terpene ratios is known as the “entourage effect,” and is generally considered to result in plants providing advantages over only using the natural products that are isolated from them. These advantages include synergy with THC, the primary active ingredient, and also mitigation of side effects from THC.

However, terpenes are generally insoluble in water making them difficult to use in various consumer products and formulations that require water-solubility. On strategy to solubilize terpenes is through glycosylation which involves chemically modifying metabolites with glucose molecules. Glycosylation and is generally carried out by the activity of UDP-glycosyltransferase (UGTs) enzymes. glycosyltransferases are UGTs which display high activity against monoterpenes, leading to the accumulation of monoterpene glycosides. Some UGTs display a large range of acceptors, such as the grape VvGT14 and VvGT7, which glycosylate geraniol, citronellol, and nerol, among other compounds. These promiscuous terpene UGTs could be incorporated into systems for the enzymatic conversion of Cannabis terpenes into terpene glycosides either in planta by means of integrating these genes into the plant genome or expressing them into cell cultures (yeast, tobacco, hops) that could be used for post-harvest processing of full-spectrum Cannabis extracts.

The glycosylation of terpenes in an in vivo system may enable enhanced accumulation, storage and transport of hydrophilic substances. Moreover, the glycosylation of terpenes may lead to higher solubility in water, reduced volatility, and lesser reactivity, which could improve pharmacokinetic parameters and therefore, could be used as a method for pro-drug preparations. Importantly, the glycosyl moieties can be subsequently cleaved by naturally occurring glycosidases, regenerating the parent terpene compound without generation of harmful metabolites to humans.

The glycosylation of terpenes would also enable the simultaneous water-based extraction of terpene glycosides and/or cannabinoid glycosides. This co-glycosylation may result in an enhanced reconstitution of the compounds found in Cannabis extracts, known to produce the entourage effect. In fact, many animal studies indicate that the pharmacological potential of cannabinoids combined with certain terpenes is superior to cannabinoids alone. Alternatively, the glycosylation of terpenes would also enable the generation of a number of compositions that might incorporate one or more glycosylated terpene, in some instances with one or more cannabinoids or glycosylated cannabinoids.

As detailed below, the present inventors have developed novel systems, methods, and compositions for glycosylating Cannabis derived terpenes in yeast and tobacco cell suspension cultures (BY2), as well as an in planta system using transgenic Cannabis or hemp plants. In certain preferred embodiment, cell suspension cultures are contained, sterile and can be scaled up in large-fermenters. Based on the reduced retention time on an HPLC gradient from 85% water to 85% acetonitrile, the present inventors show that the terpene glycosides produced in these novel systems elute earlier than their non-modified forms and are therefore more water-soluble. The present inventors have identified multiple glycosyltransferases with activity against diverse terpenes naturally present in different Cannabis cultivars.

In one preferred embodiment discussed below, a Cannabis extract, which preferably may include a “full spectrum” Cannabis extract containing both cannabinoids and terpenes may be introduced into one of the systems described herein for the production of water-soluble terpenes/terpene along with cannabinoids, preserving the “entourage effect.” Moreover, some of the enzymes with activity against terpenes can also glycosylate cannabinoids (such as NtGT3 and NtGT4), making the heterologous expression in transgenic hemp a novel strategy to generate a water-soluble extract that better represents the full spectrum of compounds observed in Cannabis plants, which would encompass hemp plants. Consumer product incorporating water-soluble cannabinoids and terpenes could have a more predictable onset time and could incorporate full, or approximately full Cannabis extracts, as opposed to purified cannabinoids, preserving the desired “entourage effect.”

### SUMMARY OF THE INVENTION

One aspect of the current inventive technology may encompass systems, methods and compositions for the in vivo production, modification and isolation of terpene compounds from Cannabis or other plants. In particular, the invention provides systems and methods for high level in vivo biosynthesis of water-soluble terpene compounds. In one preferred embodiment, the present invention generally relates to the conversion of Cannabis sativa or hemp terpenes into water-soluble compounds (terpene glycosides). In one preferred aspect, the invention may include system for glycosylating Cannabis derived terpenes in yeast and tobacco cell suspension cultures (BY2), as well as an in planta system using transgenic Cannabis or hemp plants.

Another aspect of the invention may include the production of terpene glycosides in multiple platforms, such as in planta or in plant cell suspension cultures. In this embodiment, such terpene glycosides may be generated in vivo by over-expressing certain glycosyltransferases (UGTs) displaying terpene glycosyltransferase activity. In this aspect, such water-soluble terpenes a may be generated in vivo by over-expressing certain exogenous and/or endogenous genes that may chemically modify said terpenes, preferably by glycosylation. In a preferred embodiment, such glycosylated terpene and cannabinoids compounds may be isolated and further included in one or more compositions as generally described herein.

Another aspect of the invention may include the production of terpene glycosides in multiple platforms, such as cultures of microorganism. In this embodiment, such terpene glycosides may be generated in vivo by over-expressing certain glycosyltransferases (UGTs) displaying terpene glycosyltransferase activity in yeast and adding one or more terpenes, or Cannabis or hemp extracts to the yeast culture wherein one or more terpenes may be bio-converted into a water-soluble terpene glycoside. Another embodiment is removal of the glycosyl moiety from the glycosylated terpenes using glycosidases to regenerate the parent compound.

Another aspect of the current inventive technology includes systems and methods for enhanced production and/or accumulation of terpene glycosides. In one embodiment, the invention may include systems and methods for enhanced production and/or accumulation of terpene glycosides in an in vivo system, such as a plant, or plant cell culture. Another aspect of the current inventive technology includes systems and methods for enhanced production of terpene glycosides in a yeast culture system. Another aspect of the current inventive technology includes systems and methods for enhanced production of terpene glycosides in a bacterial culture system. Another aspect of the current inventive technology includes systems and methods for enhanced production of terpene glycosides in a fungal culture system.

Another aspect of the current invention may include the generation of genetically modified plants overexpressing certain endogenous/exogenous genes that result in the production of terpene glycosides. Another aspect of the current inventive technology includes the generation of genetically modified yeast, or other eukaryotic/prokaryotic cells that overexpress certain endogenous/exogenous genes that result in the production of terpene glycosides. Additional aspects of this invention may include polynucleotide constructs that may be used to transform plant, bacterial, and/or yeast cell to express one or more endogenous/exogenous genes that result in the production of terpene glycosides. In certain systems, substrate terpenes may be endogenously produced, or added to the in vivo system.

Another aspect of the current invention may include the application of one or more glycosidases or other enzymes that may remove the sugar group from a terpene glycoside reconstituting it back to the parent terpene compound.

Another aim of the current inventive technology may include the generation of one or more of the above referenced genetically modified plant or plant cell cultures utilizing Agrobacterium Ti-plasmid mediated transformation. Another aim of the current inventive technology may include the generation of one or more of the above referenced genetically modified cells, such as a plant cell, a yeast cell, a bacterial cell, or a fungi cell, for example through techniques such as viral, electroporation, biolistic, and glass bead transformation techniques among others.

Additional aspects of the invention may include delivery systems and compositions that include water-soluble terpenes, preferably water-soluble glycosylated terpenes. Additional embodiments may further include methods and systems for the production of compositions that include water-soluble terpenes, preferably water-soluble glycosylated terpenes. Additional embodiments may include the isolation of these water-soluble terpenes followed by enzymatic conversion or reconstitution to their original or parent compound.

Another aspect of the current invention may include systems, methods and compositions for the delivery of water-soluble terpenes, preferably glycosylated and/or acetylated glycoside terpenes as a prodrug. Included in this invention may include novel prodrug compositions. Additional aspects may include one or more compositions of matter that may include water-soluble terpenes and cannabinoids, preferably glycosylated and/or acetylated glycoside terpenes and cannabinoids. In certain aspects, such compositions may include beverages and/or food additives that may impart an “entourage effect” on the user through the synergistic action of said terpenes and cannabinoids profiles present in the composition(s).

Additional aspects include isolated nucleotides sequences encoding one or more UGTs operably linked to a promoter including: UGT58A1 (SEQ ID NO. 1); MhGt1 (SEQ ID NO. 3); VvGt7 (SEQ ID NO. 9); UGT85K11 (SEQ ID NO. 5); UGT94P1 (SEQ ID NO. 7); NtGt3 (SEQ ID NO. 11); NtGt4 (SEQ ID NO. 13); Bs-YjiC (SEQ ID NO. 29); and VvGT14 (SEQ ID NO. 31), and homologous sequences thereof. Additional aspects include isolated nucleotides sequences encoding one or more UGTs operably linked to a promoter including amino acid sequences: UGT58A1 (SEQ ID NO. 2); MhGt1 (SEQ ID NO. 4); VvGt7 (SEQ ID NO. 10); UGT85K11 (SEQ ID NO. 6); UGT94P1 (SEQ ID NO. 8); NtGt3 (SEQ ID NO. 12); NtGt4 (SEQ ID NO. 14); Bs-YjiC (SEQ ID NO. 30); and VvGT14 (SEQ ID NO. 32), and homologs thereof.

Another aspect of the invention includes systems, methods and compositions for the in vivo formulation of terpene glycosides and/or cannabinoid glycosides. In this preferred aspect, UGTs may be selectively expressed in one or more of the in vivo systems described herein, such as an in planta, yeast, or cell culture system. Such selectively expressed UGTs may have glycosylation activity towards some terpenes or cannabinoids, and not others. By selectively co-expressing combinations of UGTs in an in vivo system, the water-solubilized glycosylated compounds may be selectively removed allowing for isolation of selective formulations of water-soluble terpenes and/or cannabinoid or a mixture of the two. Such selective formulations of water-soluble terpenes and/or cannabinoid or a mixture of the two may be selected for flavor, texture, and taste among other attributes and may further be incorporated into one or more consumer products as described herein.

Another aspect of the invention may include the composition according to Formula I:

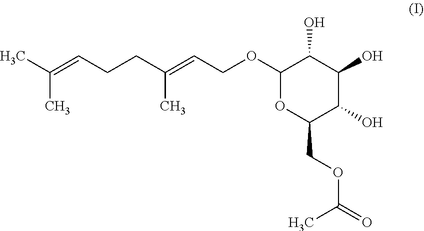 [SMILES: CC(=O)OC[C@H]1OC(OC/C=C(\C)CCC=C(C)C)[C@H](O)[C@@H](O)[C@@H]1O]

Another aspect of the invention may include the composition according to Formula II:

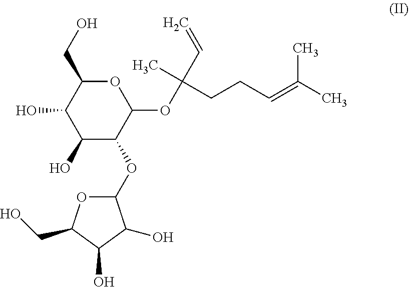 [SMILES: C=CC(C)(CCC=C(C)C)OC1O[C@H](CO)[C@@H](O)[C@H](O)[C@H]1OC1O[C@H](CO)[C@H](O)C1O]

Additional embodiments may include the following preferred embodiments:

1. An in vivo method for producing water-soluble terpenes comprising the steps:
genetically modified a yeast cell to express a nucleotide sequence encoding at least one heterologous glycosyltransferase, operably linked to a promotor, having glycosylation activity towards one or more terpenes;
establishing a suspension cell culture of genetically modified yeast cells;
introducing one or more terpenes to said suspension cell culture of genetically modified yeast cells; and
converting said one or more terpenes into one or more water-soluble glycosylated terpenes through the activity of said at least one heterologous glycosyltransferase.
   2. The method of embodiment 1, wherein said genetically modified yeast cells comprise genetically modified yeast cells selected from the group consisting of: genetically modified Pichia pastoris cells, genetically modified Saccharomyces cerevisiae cells, and/or genetically modified Kluyveromyces marxianus cells.
   3. The method of embodiment 1, wherein said nucleotide sequence encoding a heterologous glycosyltransferase having glycosylation activity towards one or more terpenes comprises a nucleotide sequence selected from the group consisting of: SEQ ID NO. 1; SEQ ID NO. 3; SEQ ID NO. 9; SEQ ID NO. 5; SEQ ID NO. 7; SEQ ID NO. 11; SEQ ID NO. 13; SEQ ID NO. 29; and SEQ ID NO. 31.
   4. The method of embodiment 1, wherein said heterologous glycosyltransferase having glycosylation activity towards one or more terpenes comprises a heterologous glycosyltransferase selected from the group consisting of: UGT58A1; MhGt1; VvGt7; UGT94P1; Bs-YjiC; VvGT14; and a homolog protein having glycosylation activity towards one or more terpenes.
   5. The method of embodiment 1, wherein said heterologous glycosyltransferase having glycosylation activity towards one or more terpenes comprises a heterologous glycosyltransferase selected from the group consisting of: SEQ ID NO. 2; SEQ ID NO. 4; SEQ ID NO. 10; SEQ ID NO. 6; SEQ ID NO. 8; (SEQ ID NO. 30; SEQ ID NO. 32; and a heterologous homolog protein having glycosylation activity towards one or more terpenes.
   6. The method of embodiments 1-5, wherein said one or more water-soluble glycosylated terpenes comprises at least one water-soluble glycosylated terpene selected from the group consisting of: a terpene glycoside, a glycosylated-xylosylated terpene, and an acetylated glycoside terpene.
   7. The method of embodiments 1-5 wherein said one or more water-soluble glycosylated terpenes comprises at least one water-soluble glycosylated terpene selected from the group consisting of: alpha-Bisabolyl monoglucoside, alpha-Bisabolyl O-acetyl glucoside, alpha-Bisabolyl oxidized glucoside, alpha-Bisabolyl diglucoside, Citronellyl monoglucoside, Geranyl O-acetyl glucoside, Geranyl monoglucoside, Linalyl monoglucoside, Linalyl glucoside-xyloside, Linalyl diglucoside, Neryl monoglucoside, Neryl diglucoside and Terpineol diglucoside.
   8. The method of embodiment 1, wherein said step of introducing one or more terpenes to said suspension cell culture of genetically modified yeast cells comprises the step of introducing one or more isolated terpenes to the yeast suspension cell culture.
   9. The method of embodiment 1, wherein said step of introducing one or more terpenes to said suspension cell culture of genetically modified yeast cells comprises the step of introducing a Cannabis extract having one or more terpenes to the yeast suspension cell culture.
   10. The method of embodiment 1, wherein said step of introducing one or more terpenes to said suspension cell culture of genetically modified yeast cells comprises the step of introducing a full spectrum Cannabis extract having one or more terpenes and one or more cannabinoid to the yeast suspension cell culture.
   11. The method of embodiments 9-10, wherein said heterologous glycosyltransferase having glycosylation activity towards one or more terpenes comprises a heterologous glycosyltransferase protein having glycosylation activity towards one or more terpenes and one or more cannabinoids.
   12. The method of embodiment 11, wherein said heterologous glycosyltransferase protein having glycosylation activity towards one or more terpenes and one or more cannabinoids comprises a heterologous glycosyltransferase protein selected from the group consisting of: NtGT3; NtGT4; and a homolog NtGT3 or NtGT4 having glycosylation activity towards one or more terpenes and one or more cannabinoids.
   13. The method of embodiment 11, wherein said heterologous glycosyltransferase protein having glycosylation activity towards one or more terpenes and one or more cannabinoids comprises a glycosyltransferase protein selected from the group consisting of: SEQ ID NO. 12, and SEQ ID NO. 14, or an amino acid sequence having at least 90% sequence identity with SEQ ID NO. 12, and SEQ ID NO. 14, and that encodes a protein having glycosylation activity towards one or more terpenes and one or more cannabinoids.
   14. The method of embodiment 1, and further comprising the step of recovering said one or more water-soluble glycosylated terpenes from said suspension cell culture of genetically modified yeast cells.
   15. The method of embodiment 14, wherein said step of recovering said one or more water-soluble glycosylated terpenes from said suspension cell culture of genetically modified yeast cells comprises the step of recovering said one or more water-soluble glycosylated terpenes from the media of said suspension cell culture of genetically modified yeast cells.
   16. The method of embodiment 1, and further comprising the step of introducing at least one glycosidase inhibitor to said suspension cell culture of genetically modified yeast cells wherein said glycosidase inhibitor is further selected from the group consisting of: D,L-1,2-Anhydro-myo-inositol (Conduritol B Epoxide (CBE); 6-Epicastanospermine (Castanospermine); 6-bromocyclohex-4-ene-1,2,3-triol (Bromoconduritol); (+)-1-Deoxynojirimycin (Deoxynojirimycin); 1,5-Dideoxy-1,5-imino-D-sorbitol hydrochloride (1-Deoxynojirimycin Hydrochloride); 1R,2S,3S,4R)-rel-5-Cyclohexene-1,2,3,4-tetrol (Conduritol B); (3R,4R,5R)-5-(Hydroxymethyl)-3,4-piperidinediol (2S,3S)-2,3-Dihydroxybutanedioate (Isofagomine D-Tartrate); O-(D-Glucopyranosylidene)amino N-Phenylcarbamate; and (3S,4S,5R,6R)-3,4,5-Trihydroxy-6-(hydroxymethyl)-2-piperidinone (D-Manno-γ-lactam).
   17. An in vivo method for producing water-soluble terpenes comprising the steps:
genetically modified a bacterial cell to express a nucleotide sequence encoding at least one heterologous glycosyltransferase, operably linked to a promotor, having glycosylation activity towards one or more terpenes;
establishing a suspension cell culture of genetically modified bacterial cells;
introducing one or more terpenes to said suspension cell culture of genetically modified bacterial cells; and
converting said one or more terpenes into one or more water-soluble glycosylated terpenes through the activity of said at least one heterologous glycosyltransferase.
   18. The method of embodiment 17, wherein said genetically modified yeast cells comprise genetically modified yeast cells selected from the group consisting of: E. coli, and Bacillus subtilis.
   19. The method of embodiment 17, wherein said nucleotide sequence encoding a heterologous glycosyltransferase having glycosylation activity towards one or more terpenes comprises a nucleotide sequence selected from the group consisting of: SEQ ID NO. 1; SEQ ID NO. 3; SEQ ID NO. 9; SEQ ID NO. 5; SEQ ID NO. 7; SEQ ID NO. 11; SEQ ID NO. 13; SEQ ID NO. 29; and SEQ ID NO. 31.
   20. The method of embodiment 17, wherein said heterologous glycosyltransferase having glycosylation activity towards one or more terpenes comprises a heterologous glycosyltransferase selected from the group consisting of: UGT58A1; MhGt1; VvGt7; UGT94P1; Bs-YjiC; VvGT14; and a homolog protein having glycosylation activity towards one or more terpenes.
   21. The method of embodiment 17, wherein said heterologous glycosyltransferase having glycosylation activity towards one or more terpenes comprises a heterologous glycosyltransferase selected from the group consisting of: SEQ ID NO. 2; SEQ ID NO. 4; SEQ ID NO. 10; SEQ ID NO. 6; SEQ ID NO. 8; (SEQ ID NO. 30; SEQ ID NO. 32; and a heterologous homolog protein having glycosylation activity towards one or more terpenes.
   22. The method of embodiments 17-21, wherein said one or more water-soluble glycosylated terpenes comprises at least one water-soluble glycosylated terpene selected from the group consisting of: a terpene glycoside, a glycosylated-xylosylated terpene, and an acetylated glycoside terpene.
   23. The method of embodiments 17-21 wherein said one or more water-soluble glycosylated terpenes comprises at least one water-soluble glycosylated terpene selected from the group consisting of: alpha-Bisabolyl monoglucoside, alpha-Bisabolyl O-acetyl glucoside, alpha-Bisabolyl oxidized glucoside, alpha-Bisabolyl diglucoside, Citronellyl monoglucoside, Geranyl O-acetyl glucoside, Geranyl monoglucoside, Linalyl monoglucoside, Linalyl glucoside-xyloside, Linalyl diglucoside, Neryl monoglucoside, Neryl diglucoside and Terpineol diglucoside.
   24. The method of embodiment 17, wherein said step of introducing one or more terpenes to said suspension cell culture of genetically modified bacterial cells comprises the step of introducing one or more isolated terpenes to the bacterial suspension cell culture.
   25. The method of embodiment 17, wherein said step of introducing one or more terpenes to said suspension cell culture of genetically modified bacterial cells comprises the step of introducing a Cannabis extract having one or more terpenes to the bacterial suspension cell culture.
   26. The method of embodiment 17, wherein said step of introducing one or more terpenes to said suspension cell culture of genetically modified bacterial cells comprises the step of introducing a full spectrum Cannabis extract having one or more terpenes and one or more cannabinoid to the bacterial suspension cell culture.
   27. The method of embodiment 25-26, wherein said heterologous glycosyltransferase having glycosylation activity towards one or more terpenes comprises a heterologous glycosyltransferase protein having glycosylation activity towards one or more terpenes and one or more cannabinoids.
   28. The method of embodiment 27, wherein said heterologous glycosyltransferase protein having glycosylation activity towards one or more terpenes and one or more cannabinoids comprises a heterologous glycosyltransferase protein selected from the group consisting of: NtGT3; NtGT4; and a homolog NtGT3 or NtGT4 having glycosylation activity towards one or more terpenes and one or more cannabinoids.
   29. The method of embodiment 27, wherein said heterologous glycosyltransferase protein having glycosylation activity towards one or more terpenes and one or more cannabinoids comprises a glycosyltransferase protein selected from the group consisting of: SEQ ID NO. 12, and SEQ ID NO. 14, or an amino acid sequence having at least 90% sequence identity with SEQ ID NO. 12, and SEQ ID NO. 14, and that encodes a protein having glycosylation activity towards one or more terpenes and one or more cannabinoids.
   30. The method of embodiment 17, and further comprising the step of recovering said one or more water-soluble glycosylated terpenes from said suspension cell culture of genetically modified bacterial cells.
   31. The method of embodiment 30, wherein said step of recovering said one or more water-soluble glycosylated terpenes from said suspension cell culture of genetically modified bacterial cells comprises the step of recovering said one or more water-soluble glycosylated terpenes from the media of said suspension cell culture of genetically modified bacterial cells.
   32. The method of embodiment 17, and further comprising the step of introducing at least one glycosidase inhibitor to said suspension cell culture of genetically modified bacterial cells wherein said glycosidase inhibitor is further selected from the group consisting of: D,L-1,2-Anhydro-myo-inositol (Conduritol B Epoxide (CBE)); 6-Epicastanospermine (Castanospermine); 6-bromocyclohex-4-ene-1,2,3-triol (Bromoconduritol); (+)-1-Deoxynojirimycin (Deoxynojirimycin); 1,5-Dideoxy-1,5-imino-D-sorbitol hydrochloride (1-Deoxynojirimycin Hydrochloride); 1R,2S,3S,4R)-rel-5-Cyclohexene-1,2,3,4-tetrol (Conduritol B); (3R,4R,5R)-5-(Hydroxymethyl)-3,4-piperidinediol (2S,3S)-2,3-Dihydroxybutanedioate (Isofagomine D-Tartrate); O-(D-Glucopyranosylidene)amino N-Phenylcarbamate; and (3S,4S,5R,6R)-3,4,5-Trihydroxy-6-(hydroxymethyl)-2-piperidinone (D-Manno-γ-lactam).
   33. A method for producing water-soluble cannabinoids comprising the steps of:
genetically modifying terpene-producing plant to express a nucleotide sequence encoding at least one heterologous glycosyltransferase, operably linked to a promotor, having glycosylation activity towards one or more terpenes;
converting said one or more terpenes into one or more water-soluble glycosylated terpenes through the activity of said at least one heterologous glycosyltransferase in planta; and
recovering said water-soluble glycosylated terpenes from the water-soluble fraction of said genetically modifying terpene-producing plant.
   34. The method of embodiment 33 wherein said terpene-producing plant is a Cannabis plant.
   35. The method of embodiments 33, and 34 wherein said nucleotide sequence encoding a heterologous glycosyltransferase glycosylation activity towards one or more terpenes comprises a nucleotide sequence selected from the group consisting of: SEQ ID NO. 1; SEQ ID NO. 3; SEQ ID NO. 9; SEQ ID NO. 5; SEQ ID NO. 7; SEQ ID NO. 11; SEQ ID NO. 13; SEQ ID NO. 29; and SEQ ID NO. 31.
   36. The method of embodiments 33, and 34, wherein said heterologous glycosyltransferase having glycosylation activity towards one or more terpenes comprises a heterologous glycosyltransferase selected from the group consisting of: UGT58A1; MhGt1; VvGt7; UGT94P1; Bs-YjiC; VvGT14; and a homolog protein having glycosylation activity towards one or more terpenes.
   37. The method of embodiments 33, and 34, wherein said heterologous glycosyltransferase having glycosylation activity towards one or more terpenes comprises a heterologous glycosyltransferase selected from the group consisting of: SEQ ID NO. 2; SEQ ID NO. 4; SEQ ID NO. 10; SEQ ID NO. 6; SEQ ID NO. 8; (SEQ ID NO. 30; SEQ ID NO. 32; and a heterologous homolog protein having glycosylation activity towards one or more terpenes.
   38. The method of embodiments 33-37, wherein said one or more water-soluble glycosylated terpenes comprises at least one water-soluble glycosylated terpene selected from the group consisting of: a terpene glycoside, a glycosylated-xylosylated terpene, and an acetylated glycoside terpene.
   39. The method of embodiment 33, wherein said one or more water-soluble glycosylated terpenes comprises at least one water-soluble glycosylated terpene selected from the group consisting of: alpha-Bisabolyl monoglucoside, alpha-Bisabolyl O-acetyl glucoside, alpha-Bisabolyl oxidized glucoside, alpha-Bisabolyl diglucoside, Citronellyl monoglucoside, Geranyl O-acetyl glucoside, Geranyl monoglucoside, Linalyl monoglucoside, Linalyl glucoside-xyloside, Linalyl diglucoside, Neryl monoglucoside, Neryl diglucoside and Terpineol diglucoside.
   40. The method of embodiment 34, wherein said heterologous glycosyltransferase having glycosylation activity towards one or more terpenes comprises a heterologous glycosyltransferase protein having glycosylation activity towards one or more terpenes and one or more cannabinoids
   41. The method of embodiment 40, wherein said heterologous glycosyltransferase protein having glycosylation activity towards one or more terpenes and one or more cannabinoids comprises a heterologous glycosyltransferase protein selected from the group consisting of: NtGT3; NtGT4; and a homolog NtGT3 or NtGT4 having glycosylation activity towards one or more terpenes and one or more cannabinoids.
   42. The method of embodiment 40, wherein said heterologous glycosyltransferase protein having glycosylation activity towards one or more terpenes and one or more cannabinoids comprises a glycosyltransferase protein selected from the group consisting of: SEQ ID NO. 12, and SEQ ID NO. 14, or an amino acid sequence having at least 90% sequence identity with SEQ ID NO. 12, and SEQ ID NO. 14, and that encodes a protein having glycosylation activity towards one or more terpenes and one or more cannabinoids
   43. The method of embodiment 33 wherein said step of recovering said water-soluble glycosylated terpenes from the water-soluble fraction of said genetically modifying terpene-producing plant comprises recovering a greater than wild-type amount of water-soluble glycosylated terpenes from the water-soluble fraction of said genetically modifying terpene-producing plant.
   44. The method of embodiments 34 and 40 wherein said step of recovering said water-soluble glycosylated terpenes from the water-soluble fraction of said genetically modifying terpene-producing plant comprises the step of pressing said Cannabis plant to remove the water-soluble fraction containing said water-soluble terpenes and said water-soluble cannabinoids.
   45. The genetically modified Cannabis plant or part thereof and its progeny of embodiment 34 wherein the plant produces greater than wild-type levels of glycosylated terpenes compared to a wild-type Cannabis plant.
   46. The genetically modified Cannabis plant or part thereof and its progeny of embodiment 34 wherein the plant produces:
a compound having the chemical structure of Formula (I);

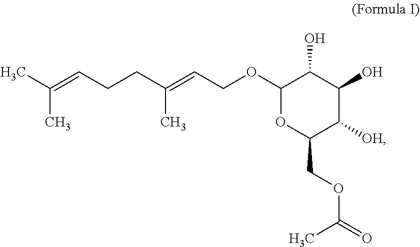 [SMILES: CC(=O)OC[C@H]1OC(OC/C=C(\C)CCC=C(C)C)[C@H](O)[C@@H](O)[C@@H]1O]

or
a compound having the chemical structure of Formula (II);

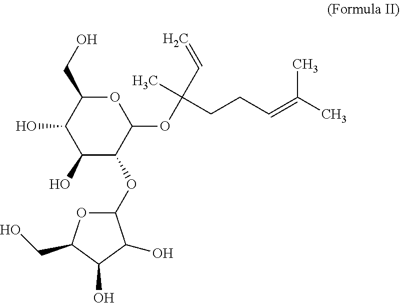 [SMILES: C=CC(C)(CCC=C(C)C)OC1O[C@H](CO)[C@@H](O)[C@H](O)[C@H]1OC1O[C@H](CO)[C@H](O)C1O]

47. The method of embodiment 34 wherein the nucleotide sequence selected from the group consisting of: SEQ ID NO. 1; SEQ ID NO. 3; SEQ ID NO. 9; SEQ ID NO. 5; SEQ ID NO. 7; SEQ ID NO. 11; SEQ ID NO. 13; SEQ ID NO. 29; and SEQ ID NO. 31, is operably linked to a promoter to produce an expression vector and wherein said expression vector is configured to be introduced via transformation to a Cannabis sativa or hemp plant.
48. A genetically modified Cannabis sativa or hemp plant or part thereof produced by the method of embodiment 47.
49. A genetically modified Cannabis sativa or hemp cell or part thereof produced by the method of embodiment 47, wherein said Cannabis sativa or hemp cell is further used to generate a cell suspension culture.
50. An in vivo method for producing water-soluble terpenes comprising the steps of:
establishing a plant suspension cell culture that expresses at least one endogenous glycosyltransferase protein having glycosylation activity towards one or more terpenes;
introducing one or more terpenes to said plant suspension cell culture; and
converting said one or more terpenes into one or more water-soluble glycosylated terpenes through the activity of said at least one endogenous glycosyltransferase.
   51. The method of embodiment 50, wherein said step of establishing a plant suspension cell culture comprises the step of establishing a tobacco suspension cell culture.
   52. The method of embodiment 51, wherein said endogenous glycosyltransferase protein comprises an endogenous glycosyltransferase protein selected from the group consisting of: NtGT3; NtGT4; and a homolog NtGT3 or NtGT4 having glycosylation activity towards one or more terpenes.
   53. The method of embodiment 51, wherein said endogenous glycosyltransferase protein comprises an endogenous glycosyltransferase protein selected from the group of amino acid sequences consisting of: SEQ ID NO. 12, and SEQ ID NO. 14, or an amino acid sequence having at least 90% sequence identity with SEQ ID NO. 12, and SEQ ID NO. 14, and that encodes a protein having glycosylation activity towards one or more terpenes.
   54. The method of embodiments 50-53, wherein said one or more water-soluble glycosylated terpenes comprises at least one water-soluble glycosylated terpene selected from the group consisting of: a terpene glycoside, a glycosylated-xylosylated terpene, and an acetylated glycoside terpene.
   55. The method of embodiments 50-53, wherein said one or more water-soluble glycosylated terpenes comprises at least one water-soluble glycosylated terpene selected from the group consisting of: alpha-Bisabolyl monoglucoside, alpha-Bisabolyl O-acetyl glucoside, alpha-Bisabolyl oxidized glucoside, alpha-Bisabolyl diglucoside, Citronellyl monoglucoside, Geranyl O-acetyl glucoside, Geranyl monoglucoside, Linalyl monoglucoside, Linalyl glucoside-xyloside, Linalyl diglucoside, Neryl monoglucoside, Neryl diglucoside and Terpineol diglucoside.
   56. The method of embodiment 50, wherein said step of introducing one or more terpenes to said plant suspension cell culture comprises the step of introducing one or more isolated terpenes to said plant suspension cell culture.
   57. The method of embodiment 50, wherein said step of introducing one or more terpenes to said plant suspension cell culture comprises the step of introducing a Cannabis extract having one or more terpenes to said plant suspension cell culture.
   58. The method of embodiment 50, wherein said step of introducing one or more terpenes to said plant suspension cell culture comprises the step of introducing a full spectrum Cannabis extract having one or more terpenes and one or more cannabinoid to said plant suspension cell culture.
   59. The method of embodiments 57-58, wherein said endogenous glycosyltransferase protein having glycosylation activity towards one or more terpenes comprises an endogenous glycosyltransferase protein having glycosylation activity towards one or more terpenes and one or more cannabinoids.
   60. The method of embodiment 59, wherein said endogenous glycosyltransferase protein having glycosylation activity towards one or more terpenes and one or more cannabinoids comprises an endogenous glycosyltransferase protein selected from the group consisting of: NtGT3; NtGT4; and a homolog NtGT3 or NtGT4 having glycosylation activity towards one or more terpenes and one or more cannabinoids.
   61. The method of embodiment 59, wherein said endogenous glycosyltransferase protein having glycosylation activity towards one or more terpenes and one or more cannabinoids comprises an endogenous glycosyltransferase protein selected from the group consisting of: SEQ ID NO. 12, and SEQ ID NO. 14, or an amino acid sequence having at least 90% sequence identity with SEQ ID NO. 12, and SEQ ID NO. 14, and that encodes a protein having glycosylation activity towards one or more terpenes and one or more cannabinoids.
   62. The method of embodiment 50, wherein said step of establishing a plant suspension cell culture comprises the step of establishing a suspension cell culture selected from the group consisting of: a Nicotiana tabacum plant suspension cell culture that expresses at least one endogenous glycosyltransferase protein having glycosylation activity towards one or more terpenes; a BY2 plant suspension cell culture that expresses at least one endogenous glycosyltransferase protein having glycosylation activity towards one or more terpenes; a Nicotiana benthamiana plant suspension cell culture that expresses at least one endogenous glycosyltransferase protein having glycosylation activity towards one or more terpenes; a Humulus lupulus plant suspension cell culture that expresses at least one endogenous glycosyltransferase protein having glycosylation activity towards one or more terpenes; a Eucalyptus perriniana; plant suspension cell culture that expresses at least one endogenous glycosyltransferase protein having glycosylation activity towards one or more terpenes; a Mentha piperita plant suspension cell culture that expresses at least one endogenous glycosyltransferase protein having glycosylation activity towards one or more terpenes; an Averrhoa carambola plant suspension cell culture that expresses at least one endogenous glycosyltransferase protein having glycosylation activity towards one or more terpenes; an Ocimum basilicum plant suspension cell culture that expresses at least one endogenous glycosyltransferase protein having glycosylation activity towards one or more terpenes; an Arabidopsis thaliana plant suspension cell culture that expresses at least one endogenous glycosyltransferase protein having glycosylation activity towards one or more terpenes; a Camellia sinensis plant suspension cell culture that expresses at least one endogenous glycosyltransferase protein having glycosylation activity towards one or more terpenes; and a Vitis vinifera plant suspension cell culture that expresses at least one endogenous glycosyltransferase protein having glycosylation activity towards one or more terpenes.
   63. The method of embodiment 50, and further comprising the step of establishing a plant suspension cell culture that expresses at least one endogenous glycosyltransferase protein having glycosylation activity towards one or more terpenes and at least one heterologous glycosyltransferase protein having glycosylation activity towards one or more terpenes.
   64. The method of embodiment 63, wherein said and at least one heterologous glycosyltransferase protein having glycosylation activity towards one or more terpenes comprises at least one heterologous glycosyltransferase protein having glycosylation activity towards one or more terpenes selected from the group consisting of: UGT58A1; MhGt1; VvGt7; UGT94P1; Bs-YjiC; VvGT14; and a homolog protein having glycosylation activity towards one or more terpenes.
   65. The method of embodiment 64, wherein said and at least one heterologous glycosyltransferase protein having glycosylation activity towards one or more terpenes comprises at least one heterologous glycosyltransferase protein having glycosylation activity towards one or more terpenes selected from the group consisting of: SEQ ID NO. 2; SEQ ID NO. 4; SEQ ID NO. 10; SEQ ID NO. 6; SEQ ID NO. 8; (SEQ ID NO. 30; SEQ ID NO. 32; and a homolog protein having glycosylation activity towards one or more terpenes.
   66. A method for producing water-soluble terpenes comprising the steps:
introducing one or more terpenes extracted from a Cannabis plant to at least one glycosyltransferase protein having glycosylation activity towards one or more terpenes; and
converting said one or more terpenes into one or more water-soluble glycosylated terpenes through the activity of said at least one glycosyltransferase protein.
   67. The method of embodiment 66, wherein said step of introducing comprises introducing one or more terpenes extracted from a Cannabis plant to at least one glycosyltransferase protein having glycosylation activity towards one or more terpenes in vitro.
   68. The method of embodiment 66, wherein said step of introducing comprises introducing one or more terpenes extracted from a Cannabis plant to at least one glycosyltransferase protein having glycosylation activity towards one or more terpenes ex vivo.
   69. The method of embodiment 66, wherein said step of introducing comprises introducing one or more terpenes extracted from a Cannabis plant to at least one glycosyltransferase protein having glycosylation activity towards one or more terpenes in a bioreactor.
   70. The method of embodiment 66, wherein said heterologous glycosyltransferase having glycosylation activity towards one or more terpenes comprises a heterologous glycosyltransferase selected from the group consisting of: UGT58A1; MhGt1; VvGt7; UGT94P1; Bs-YjiC; VvGT14; and a homolog protein having glycosylation activity towards one or more terpenes.
   71. The method of embodiment 66, wherein said heterologous glycosyltransferase having glycosylation activity towards one or more terpenes comprises a heterologous glycosyltransferase selected from the group consisting of: SEQ ID NO. 2; SEQ ID NO. 4; SEQ ID NO. 10; SEQ ID NO. 6; SEQ ID NO. 8; (SEQ ID NO. 30; SEQ ID NO. 32; and a heterologous homolog protein having glycosylation activity towards one or more terpenes.
   72. The method of embodiments 66, and 70-71, wherein said one or more water-soluble glycosylated terpenes comprises at least one water-soluble glycosylated terpene selected from the group consisting of: a terpene glycoside, a glycosylated-xylosylated terpene, and an acetylated glycoside terpene.
   73. The method of embodiments 66, 70-71, wherein said one or more water-soluble glycosylated terpenes comprises at least one water-soluble glycosylated terpene selected from the group consisting of: alpha-Bisabolyl monoglucoside, alpha-Bisabolyl O-acetyl glucoside, alpha-Bisabolyl oxidized glucoside, alpha-Bisabolyl diglucoside, Citronellyl monoglucoside, Geranyl O-acetyl glucoside, Geranyl monoglucoside, Linalyl monoglucoside, Linalyl glucoside-xyloside, Linalyl diglucoside, Neryl monoglucoside, Neryl diglucoside and Terpineol diglucoside.
   74. The method of embodiment 66, wherein said heterologous glycosyltransferase having glycosylation activity towards one or more terpenes comprises a heterologous glycosyltransferase protein having glycosylation activity towards one or more terpenes and one or more cannabinoids.
   75. The method of embodiment 74, wherein said heterologous glycosyltransferase protein having glycosylation activity towards one or more terpenes and one or more cannabinoids comprises a heterologous glycosyltransferase protein selected from the group consisting of: NtGT3; NtGT4; and a homolog NtGT3 or NtGT4 having glycosylation activity towards one or more terpenes and one or more cannabinoids.
   76. The method of embodiment 66, and further comprising the step of introducing at least one glycosidase inhibitor wherein said glycosidase inhibitor is further selected from the group consisting of: D,L-1,2-Anhydro-myo-inositol (Conduritol B Epoxide (CBE)); 6-Epicastanospermine (Castanospermine); 6-bromocyclohex-4-ene-1,2,3-triol (Bromoconduritol); (+)-1-Deoxynojirimycin (Deoxynojirimycin); 1,5-Dideoxy-1,5-imino-D-sorbitol hydrochloride (1-Deoxynojirimycin Hydrochloride); 1R,2S,3S,4R)-rel-5-Cyclohexene-1,2,3,4-tetrol (Conduritol B); (3R,4R,5R)-5-(Hydroxymethyl)-3,4-piperidinediol (2S,3S)-2,3-Dihydroxybutanedioate (Isofagomine D-Tartrate); O-(D-Glucopyranosylidene)amino N-Phenylcarbamate; and (3S,4S,5R,6R)-3,4,5-Trihydroxy-6-(hydroxymethyl)-2-piperidinone (D-Manno-γ-lactam).
   77. A compound having the chemical structure of Formula (I) or a prodrug, therapeutically active metabolite, hydrate, solvate, or pharmaceutically acceptable salt thereof:

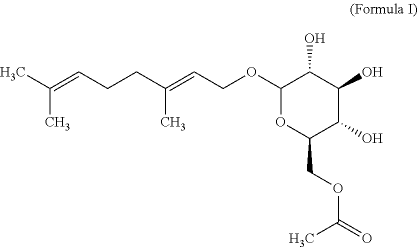 [SMILES: CC(=O)OC[C@H]1OC(OC/C=C(\C)CCC=C(C)C)[C@H](O)[C@@H](O)[C@@H]1O]

78. A compound having the chemical structure of Formula (II) or a prodrug, therapeutically active metabolite, hydrate, solvate, or pharmaceutically acceptable salt thereof:

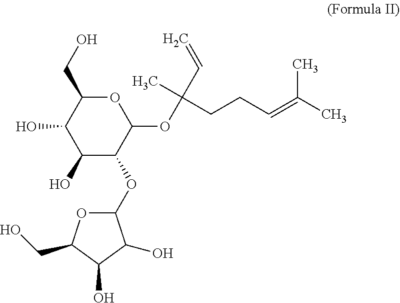 [SMILES: C=CC(C)(CCC=C(C)C)OC1O[C@H](CO)[C@@H](O)[C@H](O)[C@H]1OC1O[C@H](CO)[C@H](O)C1O]

79. A pharmaceutical composition comprising a compound of embodiment 77, or embodiment 78 and at least one pharmaceutically acceptable additive.
80. A pharmaceutical kit containing a pharmaceutical composition of embodiment 79, prescribing information for the composition, and a container.
81. A composition comprising a compound of embodiment 77, or embodiment 78, wherein the compound is co-administered to the subject with at least one cannabinoid.
82. A composition of embodiment 81 wherein said at least one cannabinoid comprises at least one glycosylated cannabinoid.
83. The compositions of embodiments 77, or embodiment 78, wherein said compositions are administered to a subject as pro-drugs.
84. At composition of embodiments 77, 78, and, 82, wherein said compositions are administered to the subject as pro-drugs.

Additional aspect will become apparent from the figures and discussion below.

### BRIEF DESCRIPTION OF THE DRAWINGS

FIG. 1. Part of the pPINK-HC vector showing the ADE2 gene (PpADE2) which produces phosphoribosylaminoimidazole carboxylase in Pichia pastoris, essential for adenine biosynthesis (Jones & Fink, 1982), and the multiple cloning site (MSC) for cloning genes of interest. All the genes were cloned in the MCS.

FIG. 2. Part of the pRI201-AN vector showing the CaMV35S strong promoter the 5′ UTR AtADH translational enhancer, the multiple cloning site (MSC) for cloning genes of interest, and the heat shock protein terminator. All the genes were cloned in the MCS.

FIG. 3. Known and predicted structures and physiochemical properties, including log 1-octanol:water partition coefficient values, molecular formulae and average masses for A) α-Bisabolol, B) α-Bisabolyl monoglucoside, C) α-Bisabolyl diglucoside, D) α-Bisabolyl oxidized glucoside and E) α-Bisabolyl O-acetyl glucoside.

FIG. 4. Known and predicted structures and physiochemical properties for A) Citronellol and B) Citronellyl monoglucoside.

FIG. 5. Known and predicted structures and physiochemical properties for A) Geraniol, B) Geranyl monoglucoside and C) Geranyl O-acetyl glucoside.

FIG. 6. Known and predicted structures and physiochemical properties for A) Linalool, B) Linalyl monoglucoside, C) Linalyl glucoside-xyloside and D) Linalyl diglucoside.

FIG. 7. Base peak intensity chromatogram (BPI) and extracted ion chromatogram (EIC) for α-bisabolyl glucoside (383.2432 m/z) observed in transgenic yeast (UGT94P1) fed α-bisabolol.

FIG. 8. BPI and EIC for α-bisabolyl glucoside+O—H₂ (397.2225 m/z) observed in transgenic yeast (VvGt7) fed α-bisabolol.

FIG. 9. BPI and EIC for α-bisabolyl diglucoside (545.2961 m/z) observed in transgenic hemp.

FIG. 10. BPI and EIC for citronellyl glucoside (317.2225 m/z) observed in transgenic yeast (NtGt3) fed citronellol.

FIG. 11. BPI and EIC for geranyl glucoside (315.1806 m/z) observed in BY2 fed geraniol.

FIG. 12. BPI and EIC for geranyl o-acetyl glucoside (357.1912 m/z) observed in transgenic hemp.

FIG. 13. BPI and EIC for linalyl monoglucoside (315.1806 m/z) observed in transgenic yeast (UGT85K11+UGT94P1) fed linalool.

FIG. 14. BPI and EIC for linalyl diglucoside (477.2335 m/z) observed in transgenic hemp leaves.

FIG. 15. BPI and EIC for linalyl glucoside-xyloside (447.2229 m/z) observed in transgenic hemp leaves.

FIG. 16: a) Detection of linalyl monoglucoside in the pellets of methanol-induced transgenic Pichia lines fed with linalool and incubated for 48 h. b) Proposed molecular structure of linalyl monoglucoside.

FIG. 17: a) Detection of alpha-bisabolyl monoglucoside in the pellets of methanol-induced transgenic Pichia lines fed with alpha-bisabolol and incubated for 48 h. b) Proposed molecular structure of alpha-bisabolyl monoglucoside.

FIG. 18. a) a) Detection of neryl glucosides in cell pellet extracts from methanol-induced transgenic Pichia lines fed with nerol and incubated for 48 h. Values are presented as signal intensity (arbitrary units) standardized to dry cell weight. b) Proposed molecular structure of neryl monoglucoside.

FIG. 19. a) Detection of citronellyl monoglucoside in the pellets of methanol-induced transgenic Pichia lines fed with citronellol and incubated for 48 h. b) Proposed molecular structure of citronellyl monoglucoside.

FIG. 20. a) Detection of oxidized alpha-bisabolyl glycoside species in the pellets of methanol-induced transgenic Pichia lines fed with alpha-bisabolol and incubated for 48 h.

FIG. 21. Gene expression confirmation of MhGT1 in transgenic Pichia induced with methanol. 1-3: Transgenic independent colonies.

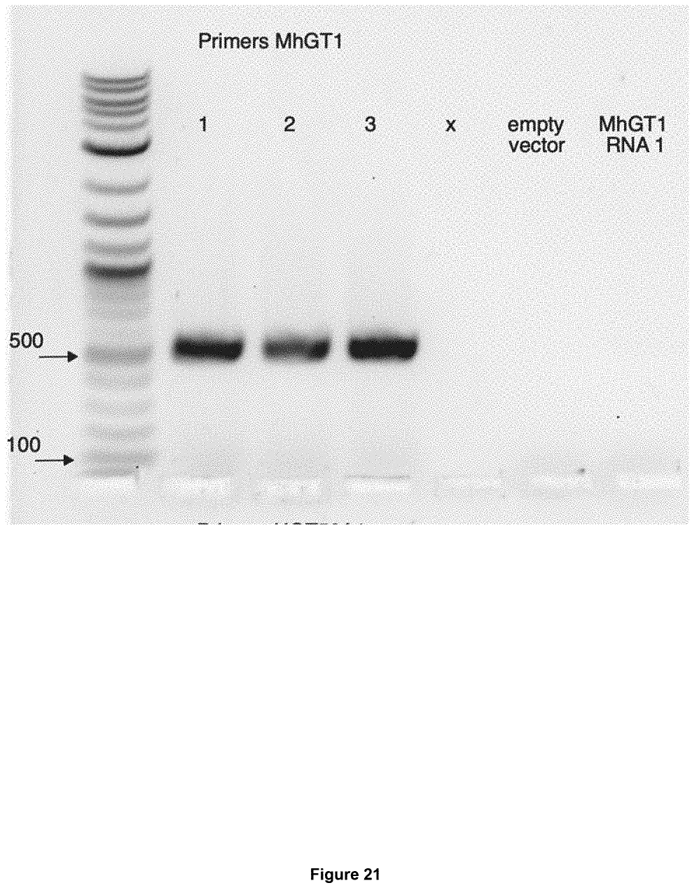

FIG. 22. Gene expression confirmation of UGT58A1 in transgenic Pichia induced with methanol. 1 and 2: Transgenic colonies.

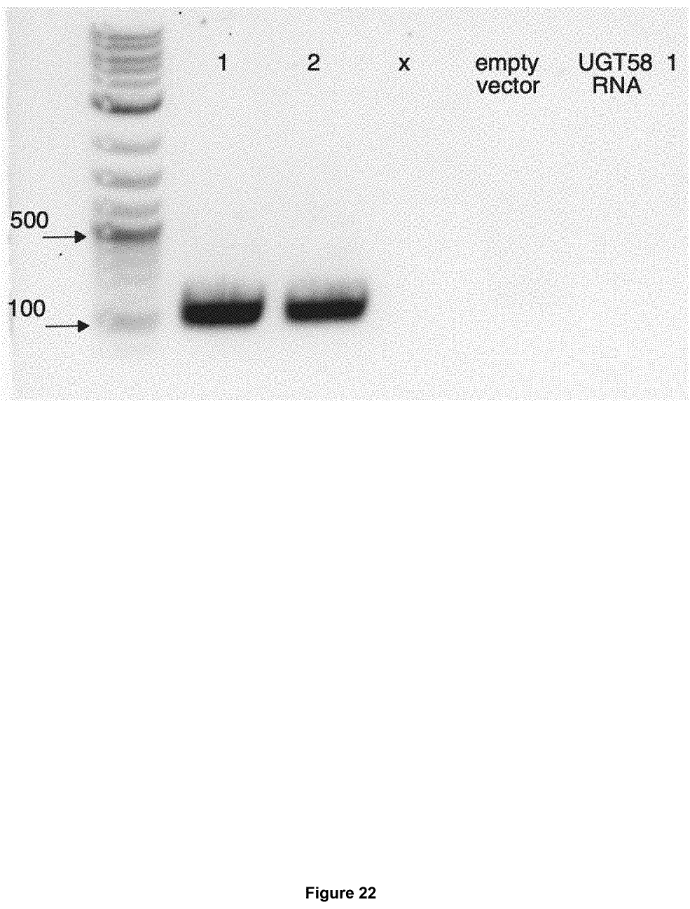

FIG. 23. Gene expression confirmation of a) VvGT7 in transgenic Pichia induced with methanol. 1-4: Transgenic colonies. 5: empty lane. 6: Empty vector control

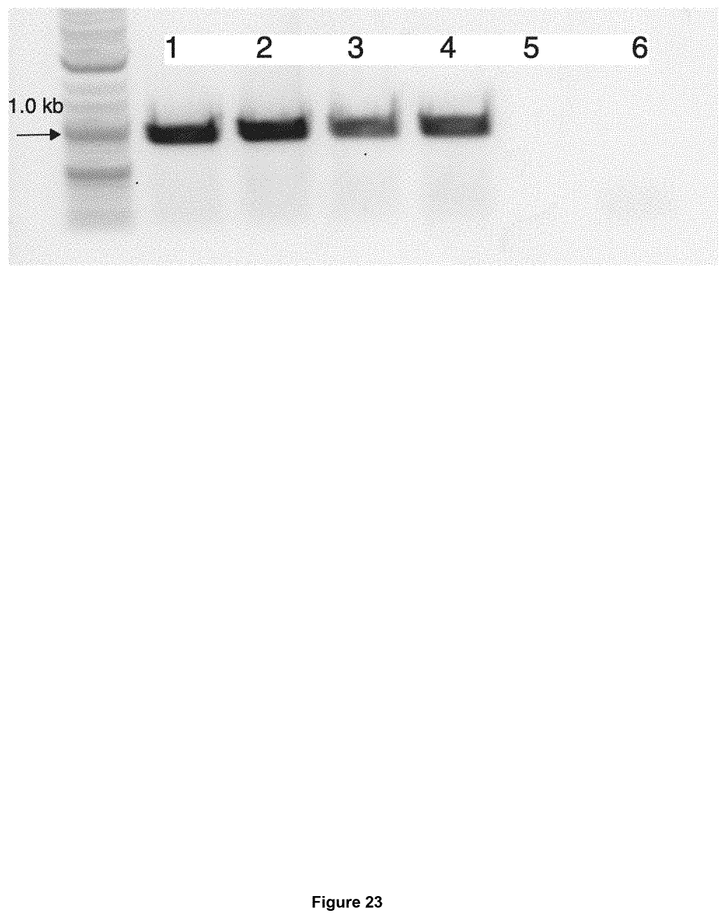

FIG. 24. Gene expression confirmation of UGT85K11 (1-5) and UGT95P1 (6-7) in transgenic Pichia induced with methanol.

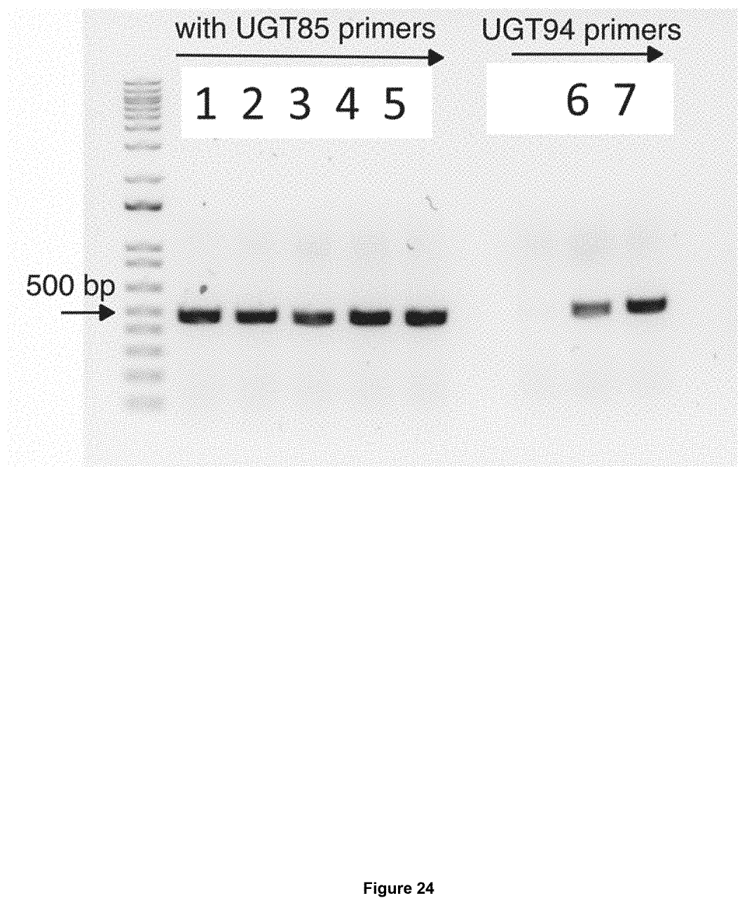

FIG. 25. Gene expression confirmation of NtGT3 and NtGT4 in transgenic Pichia induced with methanol. 1: NtGT3-expressing Pichia. 2: NtGT4-expressing Pichia. 3: empty lane. 4: Empty vector control.

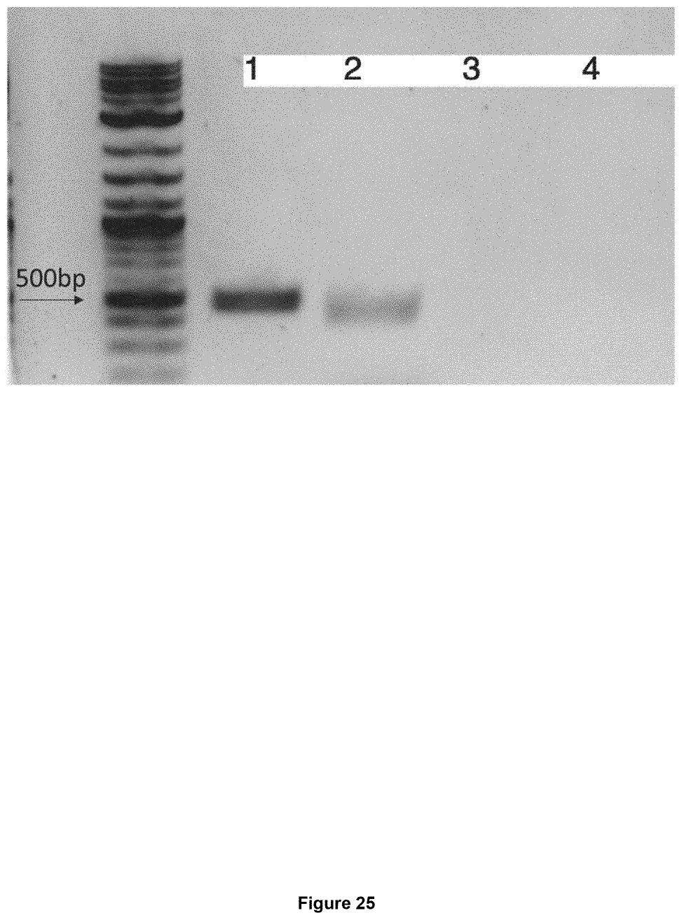

FIG. 26: Gene expression confirmation of UGT94P1 (left gel) and UGT85K11 (right gel) in hemp leaves 2 DPI. Lanes 1-3) empty vector biological replicates. Lanes 4-6) Co-infiltration of UGT94P1+UGT85K11 biological replicates. 7) RNA control. 8) water control. 9) positive control.

FIG. 27: General schematic of phytocannabinoid and Cannabis terpenoid biosynthesis.

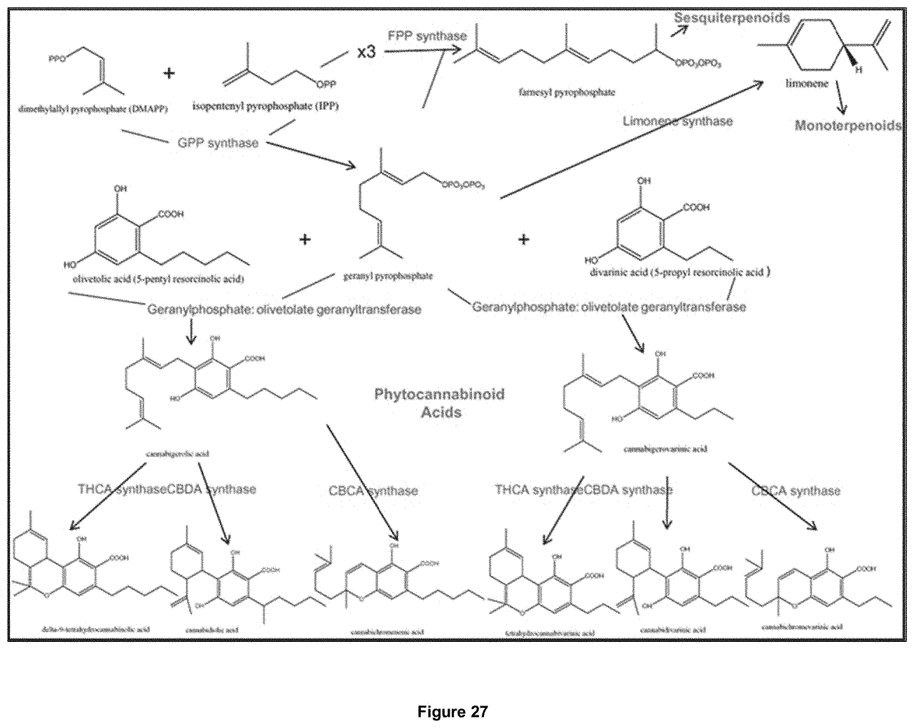

FIG. 28: Detection of alpha-bisabolyl diglucoside in hemp leaves infiltrated with Agrobacterium co-expressing UGT85K11 and UGT94P1.

FIG. 29: Detection of geranyl o-acetyl glucoside in hemp leaves infiltrated with Agrobacterium co-expressing UGT85K11 and UGT94P1 (B2).

FIG. 30: Detection of geranyl monoglucoside in the pellet extracts of BY2 cultures supplied with geraniol.

FIG. 31: Gene expression confirmation of NtGT4 in hemp leaves 4 DPI. 1-3) NtGT4-expressing plants. 4) empty lane. 5-7) Empty vector-infiltrated plants. 8) empty lane. 9) water control

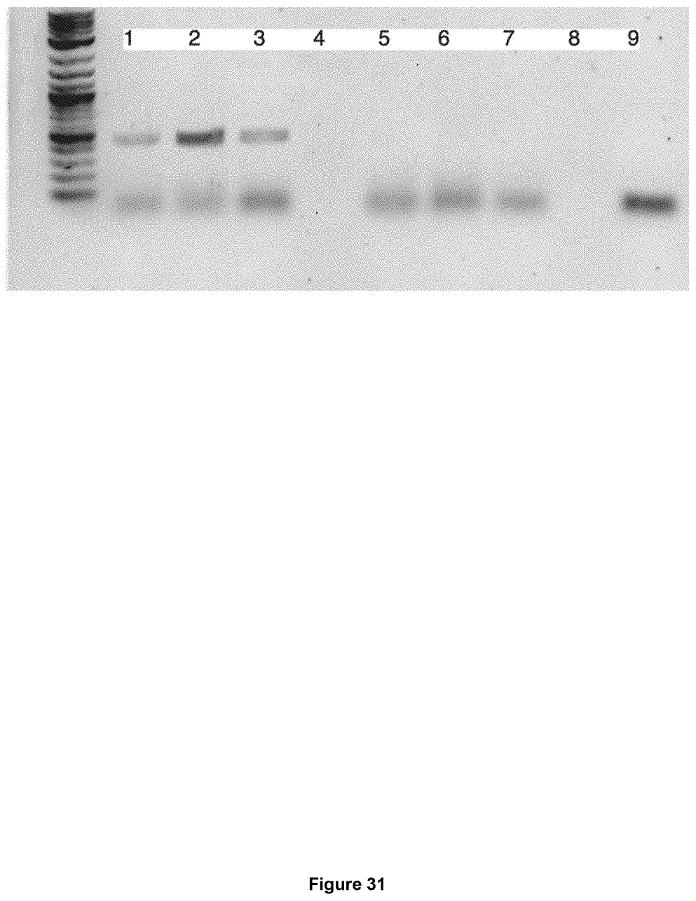

FIG. 32: Detection of α-Bisabolyl o-acetyl glucoside in hemp leaves infiltrated with Agrobacterium expressing either empty vector (EV) or NtGT4. Values are presented as signal intensity (arbitrary units) standardized to dry cell weight.

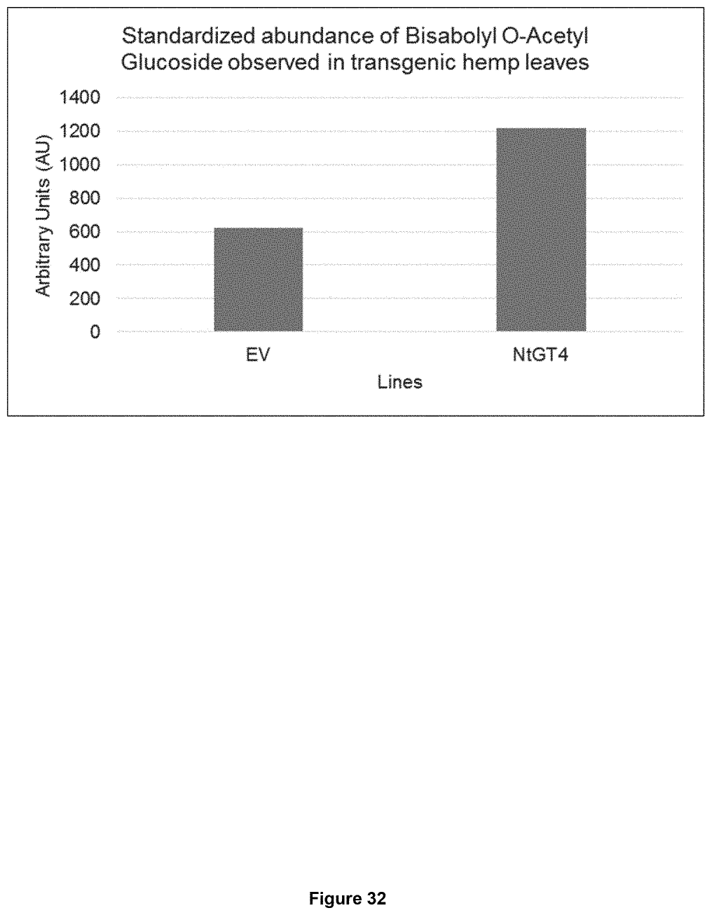

FIG. 33: Detection of Geranyl o-acetyl glucoside in hemp leaves infiltrated with Agrobacterium expressing either empty vector (EV) or NtGT4. Values are presented as signal intensity (arbitrary units) standardized to dry cell weight.

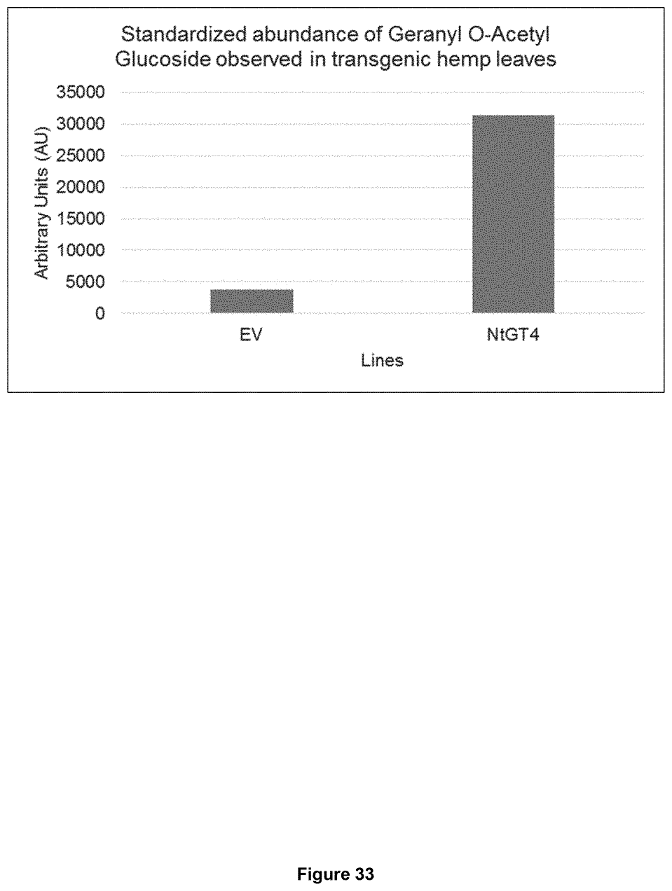

FIG. 34: Detection of Linalyl monoglucoside in hemp leaves infiltrated with Agrobacterium expressing either empty vector (EV) or NtGT4. Values are presented as signal intensity (arbitrary units) standardized to dry cell weight.

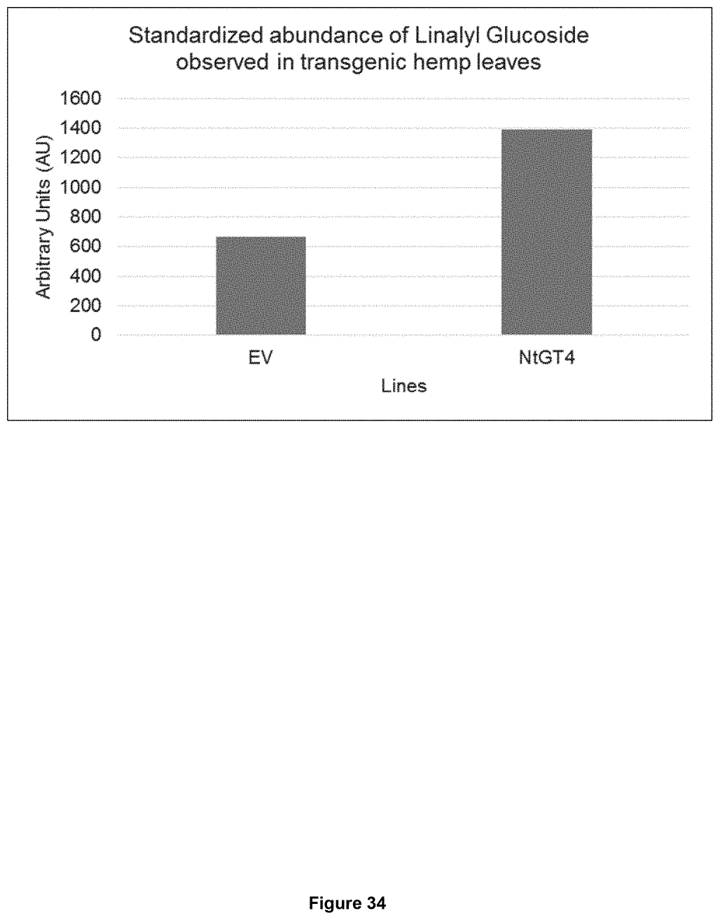

FIG. 35: Detection of Linalyl glucoside-xyloside in hemp leaves infiltrated with Agrobacterium expressing either empty vector (EV) or NtGT4. Values are presented as signal intensity (arbitrary units) standardized to dry cell weight.

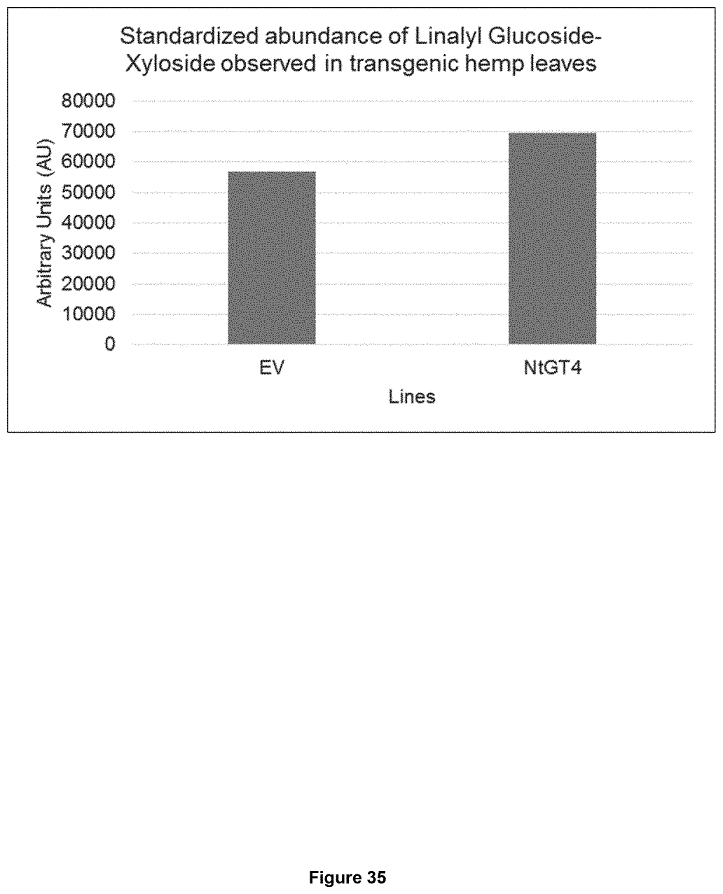

FIG. 36: a) Detection of terpineol diglucoside in cell pellet extracts from methanol-induced transgenic Pichia lines fed with terpineol and incubated for 48 h. Values are presented as signal intensity (arbitrary units) standardized to dry cell weight. b) Proposed molecular structure of neryl monoglucoside.

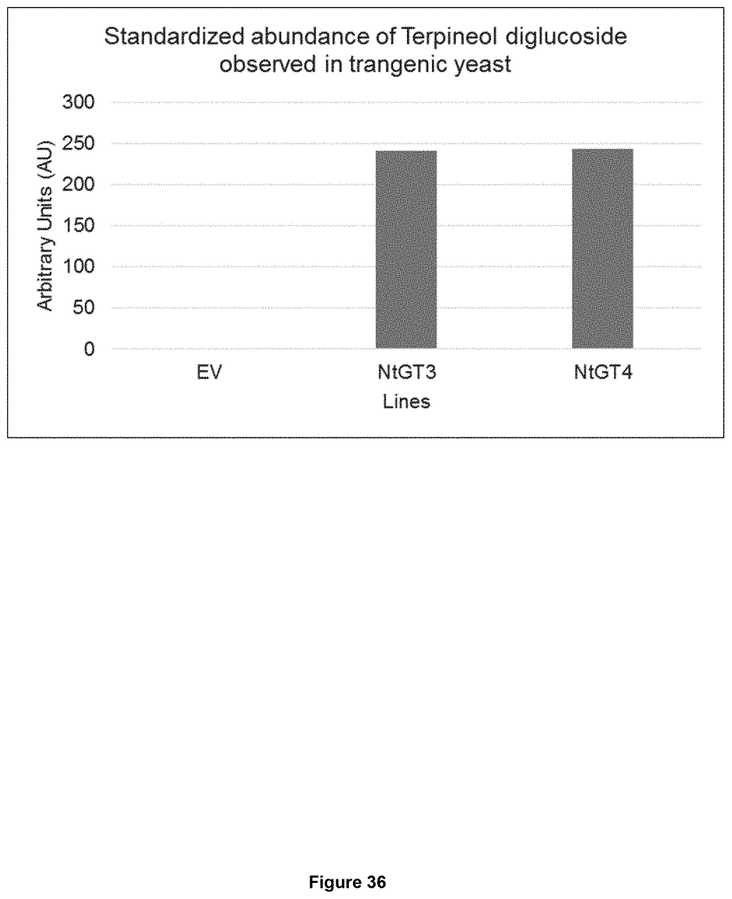

FIG. 37: Known and predicted structures and physiochemical properties, including log 1-octanol:water partition coefficient values, molecular formulae and average masses for A) Nerol, B) Neryl monoglucoside and C) Neryl diglucoside.

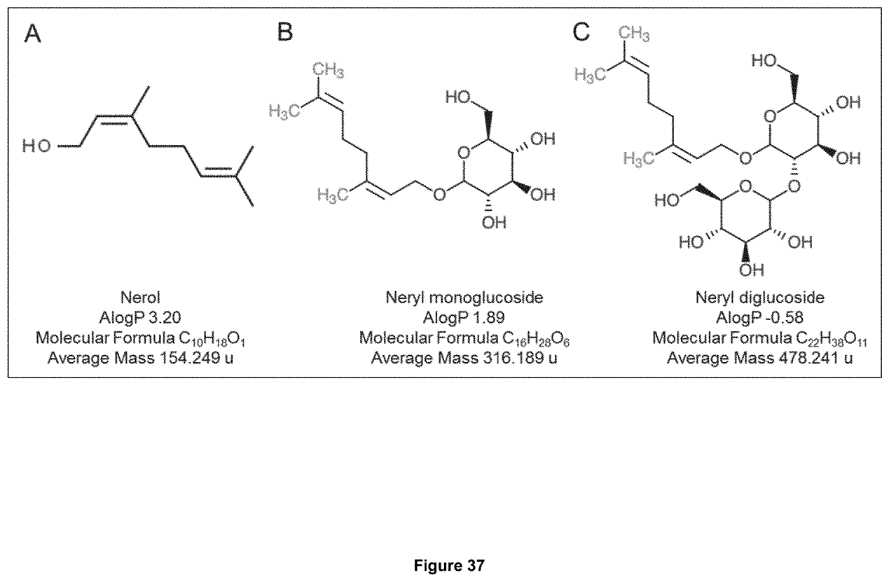

FIG. 38: Known and predicted structures and physiochemical properties, including log 1-octanol:water partition coefficient values, molecular formulae and average masses for A) Terpineol and B) Terpineol diglucoside.

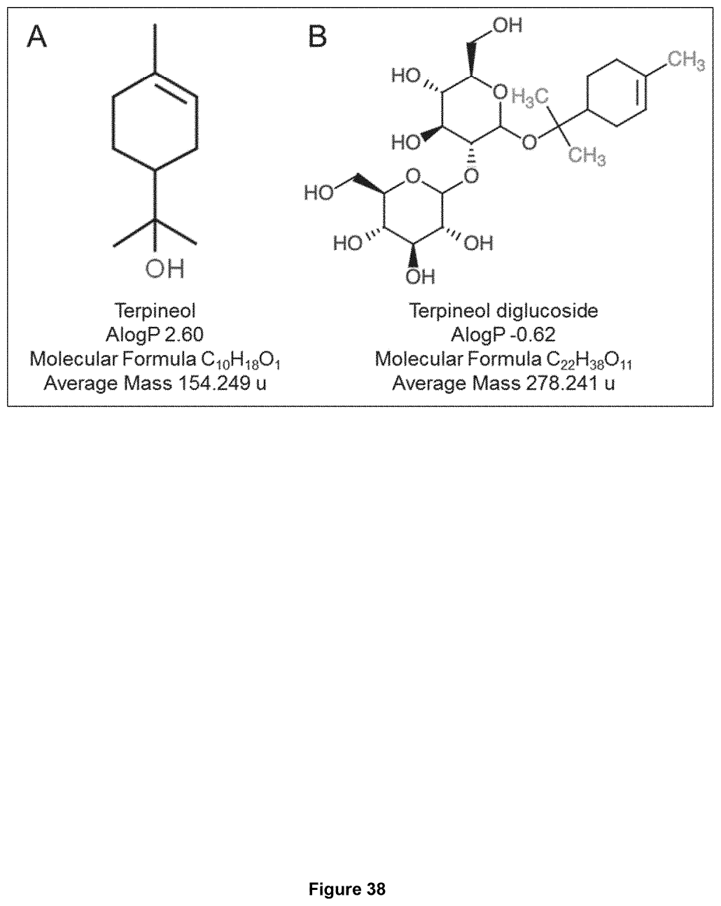

FIG. 39: BPI and EIC for neryl monoglucoside (315.1806 m/z) observed in transgenic yeast (UGT85K11) at approximately 11.15 min.

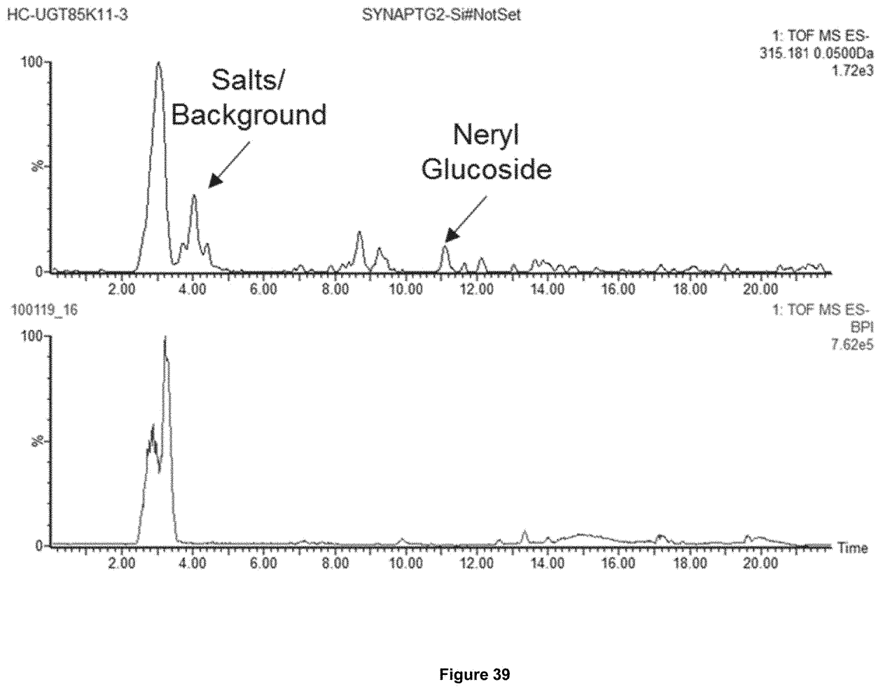

FIG. 40: BPI and EIC for neryl diglucoside (477.2335 m/z) observed in transgenic yeast (NtGT4) at approximately 7.77 min.

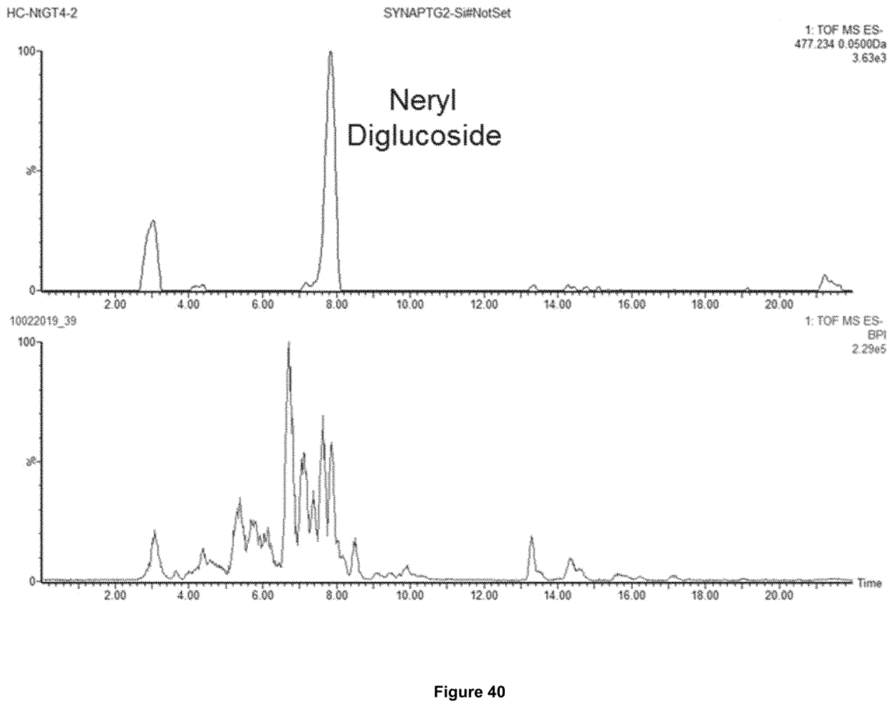

FIG. 41: BPI and EIC for terpineol diglucoside (477.2335 m/z) observed in transgenic yeast (NtGT4) at approximately 7.66 min.

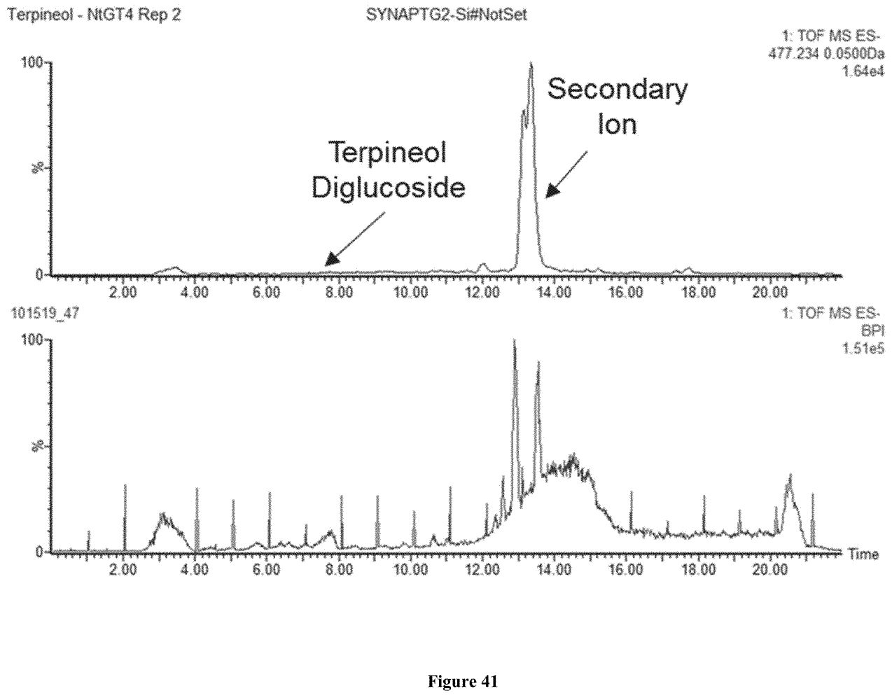

FIG. 42: BPI and EIC for α-Bisabolyl O-acetyl glucoside (425.2538 m/z) observed in transgenic hemp (NtGT4) at approximately 15.45 min.

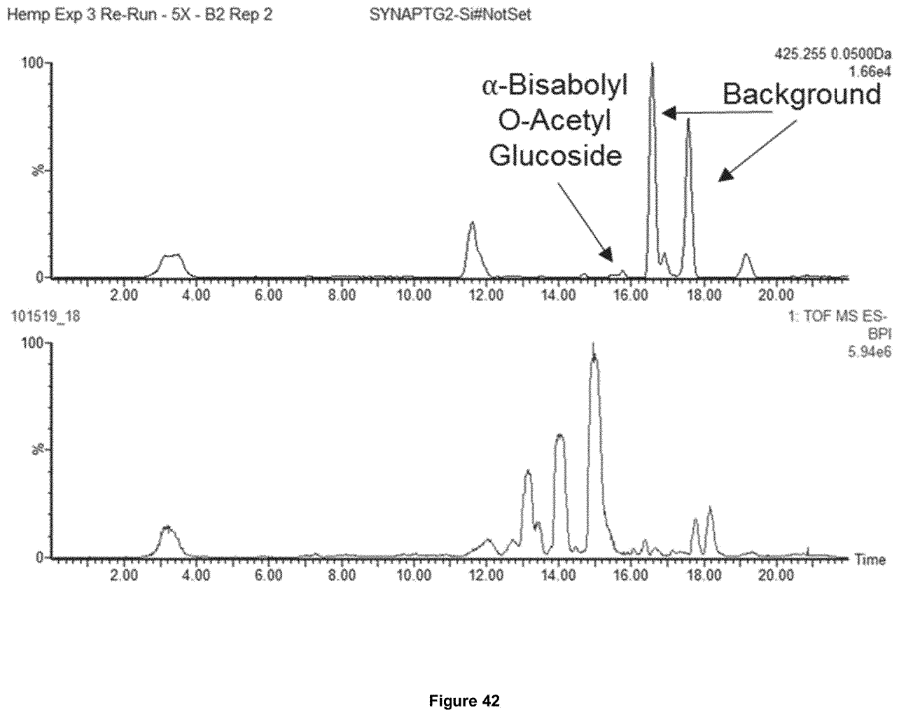

### DETAILED DESCRIPTION OF THE INVENTION

Generally, the inventive technology relates to the field of chemical modification and isolation of Cannabis terpenes in planta, as well as in plant and yeast suspension cultures. The present inventive technology further relates to improved systems and methods for the modification and isolation of pharmaceutically active terpenes from plant materials. In one embodiment, the inventive technology may encompass a novel system for the generation of chemically modified terpene compounds in planta, as well as in plant and yeast suspension cultures. The inventive technology may include systems and methods for high-efficiency chemical modification and isolation of terpene compounds in planta, as well as in plant and yeast suspension cultures. In this embodiment, various select terpene compounds may be chemically modified into soluble configurations. In other embodiments, the invention may also include the coupled chemical modification of Cannabis derived cannabinoids in planta, as well as in plant and yeast suspension cultures as generally described in U.S. application Ser. Nos. 16/110,728, and 16/110,954. (Both of which are hereby incorporated in their entity by reference).

In one embodiment the current inventive technology includes improved systems and methods for the modification of terpenes in planta, or in a sterile yeast and/or plant culture system (such systems being generally referred to as in vivo systems). In other embodiments the current inventive technology includes improved systems and methods for the modification of terpenes in vitro. Such methods including, for example incubating one or more terpenes with a sugar substrate and a glycosyltransferase or other sufficiently promiscuous UGT that may catalyze the glycosylation of the subject terpene in vitro.

In one embodiment, the inventive technology may include the production of genetically modified plant, or a sterile yeast and/or plant cell suspension culture. The inventive technology may allow for certain transgenes to be introduced into these yeast and/or plant strains to transiently modify the chemical structure of the terpene compounds. This transient modification may render the terpene soluble in water. Such modifications may also alter the rate at which the terpenes are metabolized generating a modified terpene with enhanced kinetics that may be used in certain therapeutic applications or as a prodrug. These modified terpenes, aided by their modified chemical structure, may be allowed to accumulate at higher than native levels without having a deleterious effect on the cultured yeast and/or plant cells. Being soluble, such Cannabis derived terpenes may also be secreted through endogenous and/or exogenous ABC or other trans-membrane protein transporters into the culture medium for later harvesting and isolation. These transiently modified terpenes may further be harvested and isolated from the aforementioned in vivo systems, and then enzymatically restored to their original chemical structure. Other embodiments may allow for the regulation of terpene modification and isolation. In such embodiments, discreet and known amounts of terpenes may be introduced into a yeast and/or plant suspension culture and modified into a water-soluble form. Later, such modified terpenes may be extracted from the cell culture and isolated such that the quantity and relative ratios of the various terpenes is known and quantifiable. In this manner the isolated terpene extract may be chemically consistent and as such, easily dosable for both pharmaceutical and/or other commercial applications.

In more specific embodiments, the inventive technology may include systems and methods for the in vitro and more preferably in vivo chemical modification of terpene compounds. In one preferred embodiment, a suspension or hairy root or cell suspension culture may be established in a fermenter or other similar apparatus. It should be noted that the use of suspension culture may be broadly interpreted to include both Cannabis plant strains, such C. sativa as well as non-Cannabis plants such as tobacco plants among others. For example, in certain other embodiments, various Cannabis strains, mixes of strains, hybrids of different strains or clones, as well as different varieties may be used to generate a suspension or hairy root culture. In certain embodiments, cell cultures of terpene producing as well as non-terpene producing cell cultures may be established in a fermenter. Such fermenters may include large industrial-scale fermenters allowing for a large quantity of terpene producing C. sativa cells to be cultured. In this embodiment, it may be possible to culture a large quantity of unadulterated cells from a single-strain of, for example, tobacco or C. sativa, which may establish a cell culture having a consistent production and/or modification of terpene compounds in both quantity and type. Such cultured growth may be continuously sustained with the supplementation of nutrient and other growth factors to the culture. Such features may be automated or accomplished manually.

Another embodiment of the inventive technology may include systems and methods for the production of modified terpene compounds in non-Cannabis plants. In one embodiment, a suspension or hairy root culture of one or more tobacco plant strains may be established. It should be noted that the term strain may refer to a plant strain, as well as a cell culture, or cell line derived from a tobacco plant. In one preferred embodiment, a suspension or hairy root culture of Nicotiana benthamiana plant may be established in a fermenter or other similar apparatus. It should be noted that the use of N. benthamiana in this embodiment is exemplary only. For example, in certain other embodiments, various Nicotiana strains, mixes of strains, hybrids of different strains or clones, as well as different varieties may be used to generate a cell suspension or hairy root culture.

In one embodiment, a suspension culture of one or more yeast strains may be established. In one preferred embodiment, culture, and more preferably a suspension culture of Saccharomyces cerevisiae and/or Pichia pastoris or other suitable yeast species may be established in a fermenter or other similar apparatus. It should be noted that the use of the above identified example in this embodiment is exemplary only, as various yeast strains, mixes of strains, hybrids of different strains or clones may be used to generate a suspension culture. For example, Pichia pastoris or any other appropriate yeast strain, including but not limited to all strains of yeast deposited with the ATCC. (The yeast strain deposit database(s) being incorporated by reference in its entirety.) In certain cases, such fermenters may include large industrial-scale fermenters allowing for a large quantity of yeast cells to be grown. In this embodiment, it may be possible to culture a large quantity of cells from a single-strain of, for example, P. pastoris or K. marxianus, which may establish a cell culture having a consistent or predictable rate of terpene modification. Such cultured growth may be continuously sustained with the continual addition of nutrient and other growth factors being added to the culture. Such features may be automated or accomplished manually. As noted above, terpene producing strains of Cannabis, as well as other plants may be utilized with the inventive technology. In certain preferred embodiments, Cannabis plant material containing one or more terpenes of interest may be harvested and undergo extraction through one or more of the methods generally known in the art. These extracted terpenes may be introduced into genetically modified yeast or plant suspension cell culture to be further modified as described below.

The inventive technology may include the generation of transgenic plant and yeast strains having artificial genetic constructs that may express/overexpress one or more glycosyltransferases, or other enzymes capable of glycosylating terpene compounds. In one preferred embodiment, artificial genetic constructs having genes encoding one or more UDP- and/or ADP-glycosyltransferases, including non-human analogues of those described above, as well as other isoforms, may be expressed in a genetically modified plant/plant cell and/or yeast cells and may be grown in suspension or other cell cultures. Additional embodiments may include genetic control elements such as promotors and/or enhancers as well as post-transcriptional regulatory control elements that may also be expressed in a transgenic plant or cells such that the presence, quantity and activity of any glycosyltransferases present in the suspension culture may be regulated. Additional embodiments may include artificial genetic constructs having one or more genes encoding one or more UDP- and/or ADP-glycosyltransferases having tags that may assist in the movement of the gene product to a certain portion of the cell, such as the cellular locations were terpenes and/or glycosylated terpenes may be stored, and/or excreted from the cell.

In one specific preferred embodiment, the invention may include the generation of transgenic or genetically modified strains of Cannabis, or other plants such as tobacco, having artificial genetic constructs that may express one or more genes that may glycosylate terpenes. For example, the inventive technology may include the generation of transgenic plant strains or cell lines having artificial genetic constructs that may express one or more endogenous/or exogenous glycosyltransferases or other enzymes capable of glycosylating terpene compounds. For example, in one embodiment one or more glycosyltransferases from N. benthamiana, or other non-Cannabis plants may be introduced into a Cannabis plant or cell culture and configured to glycosylate terpenes in vivo. In other embodiments, endogenous glycosyltransferases from N. benthamiana may be over-expressed so to as to increase in vivo terpene glycosylation.

An additional embodiment of the invention may include artificial genetic constructs having one or more genes encoding one or more UDP- and/or ADP-glycosyltransferases being co-expressed with one or more exogenous genes that may assist in the movement of the protein to a certain portion of the cell, such as the cellular locations were terpenes and/or glycosylated terpenes may be stored, and/or excreted from the cell.

In yet another separate embodiment, the now soluble transiently modified terpenes may be passively and/or actively excreted from a plant or yeast cell. In one exemplary model, an ATP-binding cassette transporter (ABC transporters) or other similar molecular structure may recognize the functional group (conjugate) on the glycosylated terpenes, for example and actively transport it into the surrounding media. In this embodiment, an in-vivo cell culture may be allowed to grow until an output parameter is reached. In one example, an output parameter may include allowing the cell culture to grow until a desired cell/optical density is reached, or a desired level of modified terpenes is reached. In this embodiment, the culture media containing the modified, and preferably water-soluble glycosylated terpenes, may be harvested for later extraction and/or isolation. Additionally, the modified terpenes present in the raw and/or treated media may be isolated and purified, for example, through affinity chromatography in a manner similar to that described above. The term “recovering” may generally encompass extracting and/or isolating one or more compounds of interest, and in particular one or more glycosylated compounds of interest.

In certain embodiments, this purified isolate may contain a mixture of primary and secondary glycosylated terpenes. As noted above, such purified glycosylated terpenes may be water-soluble and metabolized slower than unmodified terpenes providing a slow-release capability that may be desirable in certain pharmaceutical applications, such as for use in tissue-specific applications or as a prodrug. In this embodiment, purified glycosylated terpenes may be incorporated into a variety of pharmaceutical and/or nutraceutical applications as described in greater detail below. For example, the purified glycosylated terpenes may be incorporated into various solid and/or liquid delivery vectors for use in pharmaceutical applications. Additional therapeutic applications may include the administration of a therapeutic dose of an “entourage” of isolated and purified glycosylated terpenes and glycosylated cannabinoids.

The inventive technology may also include a system to convert or reconstitute glycosylated terpenes. In one preferred embodiment, glycosylated terpenes may be converted into non-glycosylated terpenes through their treatment with one or more generalized or specific glycosidases. In this embodiment, these glycosidase enzymes may remove a sugar moiety. Specifically, these glycosidases may remove the glucuronic acid moiety reconstituting the terpene compound. In one embodiment, glycosylated terpenes may be isolated to generate a highly purified mixture post-glycosylation, which may further include, or be combined with a highly purified mixture of glycosylated cannabinoids. These reconstituted terpene compounds may also be incorporated into various solid and/or liquid delivery vectors for use in a variety of pharmaceutical and other commercial applications. In certain embodiments, modified terpenes may be reconstituted through incubation with one or more generalized or specific glycosidases in an in vitro system.

In one embodiment, a polynucleotide may be generated that expresses one or more of the SEQ ID NOs identified herein, or any SEQ ID NOs. incorporated specifically by reference from priority document U.S. Provisional Patent Application No. 62/746,053, filed Oct. 16, 2018. In certain preferred embodiments, the proteins of the invention may be expressed using any of a number of systems to obtain the desired quantities of the protein. Typically, the polynucleotide that encodes the protein or component thereof is placed under the control of a promoter that is functional in the desired host cell. An extremely wide variety of promoters may be available, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes” or “constructs.” Accordingly, the nucleic acids that encode the joined polypeptides are incorporated for high level expression in a desired host cell. Exemplary promotors may include, but not be limited to: a non-constitutive promotor; an inducible promotor, a tissue-preferred promotor; a tissue-specific promotor, a plant-specific promotor, or a constitutive promotor. In a preferred embodiment, one or more select genes may be operably linked to a leaf-specific gene promotor, such as Cab1. Additional promoters and operable configurations for expression, as well as co-expression of one or more of the selected genes are generally known in the art.

Another embodiment comprises a tissue culture comprising a plurality of the genetically altered plant cells.

Another embodiment provides for a method for constructing a genetically altered plant or part thereof having increased cannabinoid/terpene production compared to a non-genetically altered plant or part thereof, the method comprising the steps of: introducing a polynucleotide encoding a protein into a plant or part thereof to provide a genetically altered plant or part thereof, wherein said protein comprising at least one UGT having glycosylation activity towards one or more terpenes.

In one embodiment, the invention may include an in vivo production, accumulation and modification system that may generate water-soluble terpene. In one preferred embodiment, a plant, such as Cannabis or tobacco, may be genetically modified to express one or more heterologous glycosyltransferase genes, such as UDP glycosyltransferase. In this preferred embodiment, UDP glycosyltransferase (76G1) from Stevia rebaudiana may be expressed in terpene producing plant or cell suspension culture. In a preferred embodiment, the terpene producing plant or cell suspension culture may be Cannabis. In another embodiment, one or more glycosyltransferase from Nicotiana tabacum and/or a homologous glycosyltransferase from Nicotiana benthamiana, may be expressed in a terpene-producing plant, such as Cannabis, or may be over-expressed in an endogenous plant and/or plant cell culture system. In a preferred embodiment, a glycosyltransferase gene and/or protein may be selected from the exemplary plant, such as Nicotiana tabacum. Such glycosyltransferase gene and/or protein may include, but not limited to: Glycosyltransferase (NtGT5a) Nicotiana tabacum; Glycosyltransferase (NtGT5a) Nicotiana tabacum; Glycosyltransferase (NtGT5b) Nicotiana tabacum; Glycosyltransferase (NtGT5b) Nicotiana tabacum; UDP-glycosyltransferase 73C3 (NtGT4) Nicotiana tabacum; UDP-glycosyltransferase 73C3 (NtGT4) Nicotiana tabacum; Glycosyltransferase (NtGT1b) Nicotiana; Glycosyltransferase (NtGT1b) Nicotiana tabacum; Glycosyltransferase (NtGT1a) Nicotiana tabacum (Glycosyltransferase (NtGT1a) Nicotiana tabacum; Glycosyltransferase (NtGT3) Nicotiana tabacum; Glycosyltransferase (NtGT3) Nicotiana tabacum; Glycosyltransferase (NtGT2) Nicotiana tabacum; and/or Glycosyltransferase (NtGT2) Nicotiana tabacum. The sequences from Nicotiana tabacum are exemplary only as other tobacco and non-tobacco glycosyltransferase may be used. Such glycosyltransferase may be expressed in an in planta system, as well as an in vivo yeast or bacterial system as generally described herein. As noted above, such glycosyltransferases may glycosylate the terpene in a plant or cell culture system as generally described here. Naturally, other glycosyltransferase genes from alternative sources may be included in the current invention.

It should be noted that a number of combinations and permutations of the genes/proteins described herein may be co-expressed and thereby accomplish one or more of the goals of the current invention. Such combinations are exemplary of preferred embodiments only, and not limiting in any way.

In one preferred embodiment, the present inventors demonstrate the generation of a genetically modified plant configured to produce water-soluble, and preferably glycosylated terpenes. In one preferred embodiment a cannabinoid-producing plant, such as a Cannabis sativa plant, may be transformed to over-express one or more select UGTs. In one preferred embodiment, a Cannabis plant or cell may be genetically modified to express a heterologous nucleotide sequence, operably linked to a promoter, encoding one or more UGTs having glycosylation activity towards one or more terpenes selected from the group consisting of: alpha-Pinene, Camphene, beta-Myrcene, beta-Pinene, delta-3-Carene, alpha-Terpine, Ocimene 1, d-Limonene, p-Cymene, Ocimene 2, Eucalyptol, gamma-Terpinene, Terpinoline, Linalool, Isolpulegol, Geraniol, beta-Caryophyllene, alpha-Humulene, Nerolidol 1, Nerolidol 2, Guaiol, Caryophyllene oxide, and alpha-Bisabolol.

In another preferred embodiment, a Cannabis plant or cell may be genetically modified to express a heterologous nucleotide sequence, operably linked to a promoter, encoding one or more UGTs having glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); UGT94P1 (SEQ ID NO. 7); NtGt3 (SEQ ID NO. 11); NtGt4 (SEQ ID NO. 13); Bs-YjiC (SEQ ID NO. 29); and VvGT14 (SEQ ID NO. 31). In this embodiment, one or more of the above UGTs may glycosylate at higher then wild-type levels, in vivo, one or more terpenes selected from the group consisting of: alpha-Pinene, Camphene, beta-Myrcene, beta-Pinene, delta-3-Carene, alpha-Terpine, Ocimene 1, d-Limonene, p-Cymene, Ocimene 2, Eucalyptol, gamma-Terpinene, Terpinoline, Linalool, Isolpulegol, Geraniol, beta-Caryophyllene, alpha-Humulene, Nerolidol 1, Nerolidol 2, Guaiol, Caryophyllene oxide, and alpha-Bisabolol.

In another preferred embodiment, a Cannabis plant or cell may be genetically modified to express a heterologous nucleotide sequence, operably linked to a promoter, encoding one or more UGTs having glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); UGT94P1 (SEQ ID NO. 7); NtGt3 (SEQ ID NO. 11); NtGt4 (SEQ ID NO. 13); Bs-YjiC (SEQ ID NO. 29); and VvGT14 (SEQ ID NO. 31), wherein at least one glycosylated terpene is produced, and preferably produced at higher than wild-type levels, selected from the group consisting of: alpha-Bisabolyl monoglucoside, alpha-Bisabolyl O-acetyl glucoside, alpha-Bisabolyl oxidized glucoside, alpha-Bisabolyl diglucoside, Citronellyl monoglucoside, Geranyl O-acetyl glucoside, Geranyl monoglucoside, Linalyl monoglucoside, Linalyl glucoside-xyloside, Linalyl diglucoside, Neryl monoglucoside, Neryl diglucoside and Terpineol diglucoside.

In another preferred embodiment, expression of one or more UGTs may further be in conjunction with the expression of a heterologous glycosyltransferase that has glycosylation activity directed to cannabinoids as well as terpenes. In this preferred embodiment, a Cannabis plant may be genetically modified to express a heterologous nucleotide sequence, operably linked to a promoter, encoding at least one the following UGTs having glycosylation activity directed to cannabinoids as well as terpenes selected from the group consisting of: NtGt3 (SEQ ID NO. 11); and NtGt4 (SEQ ID NO. 13), in addition to the heterologous expression of one or more UGTs having glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); and UGT94P1 (SEQ ID NO. 7). Additional UGTs that have glycosylation activity towards cannabinoids are incorporated by reference from Sayre et al. in PCT/US18/24409.

In additional embodiments, multiple UGTs having activity toward different terpenes and/or cannabinoids may be co-expressed in one or more expression cassettes operably linked to a promoter generating an expression vector. Genes encoding one or more polynucleotides and/or a homologue thereof of the invention may be introduced into a plant, and/or plant cell, and preferably a Cannabis plant or cell, using several types of transformation approaches developed for the generation of transgenic plants. Standard transformation techniques, such as Ti-plasmid Agrobacterium-mediated transformation, particle bombardment, microinjection, and electroporation may be utilized to construct stably transformed transgenic plants.

In one preferred embodiment, the present inventors demonstrate the generation of a genetically modified yeast cell configured to produce water-soluble, and preferably glycosylated terpene compounds. In one preferred embodiment a genetically modified yeast cell, such as Saccharomyces cerevisiae, K. marxianus, and Pichia pastoris or other suitable yeast species may be transformed to express one or more heterologous UGTs having activity towards at least one terpene. Select, or full spectrum terpenes, as well as cannabinoid compounds generated by the Cannabis sativa plant may be harvested, for example as an oil or other extract and may fed to such a genetically modified yeast cell culture in a fermenter. Such terpenes, as well as cannabinoid compounds may be efficiently glycosylated through interaction with the overexpressed endogenous and/or heterologous glycosyltransferase enzymes. In this embodiment, the synthesis of water-soluble cannabinoids, terpenes and/or terpenes in the yeast cells may be harvested and isolated separately or together as a “full-spectrum” water-based extract. The post-harvest conversion of terpenes into terpene glycosides may allow their inclusion in one or more of the compositions described herein, or reconstituted to their original structure through the action of one or more glycosidases. Additional embodiments include the addition of one or more “glycosidase inhibitor” to a yeast culture which may inhibit glycosidase enzymes which catalyze the hydrolysis of glyosidic bonds.

In one preferred embodiment, a yeast cell may be genetically modified to express a heterologous nucleotide sequence, operably linked to a promoter, encoding one or more UGTs having glycosylation activity towards one or more terpenes selected from the group consisting of: alpha-Pinene, Camphene, beta-Myrcene, beta-Pinene, delta-3-Carene, alpha-Terpine, Ocimene 1, d-Limonene, p-Cymene, Ocimene 2, Eucalyptol, gamma-Terpinene, Terpinoline, Linalool, Isolpulegol, Geraniol, beta-Caryophyllene, alpha-Humulene, Nerolidol 1, Nerolidol 2, Guaiol, Caryophyllene oxide, and alpha-Bisabolol.

In another preferred embodiment, a yeast cell may be genetically modified to express a heterologous nucleotide sequence, operably linked to a promoter, encoding one or more UGTs having glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); UGT94P1 (SEQ ID NO. 7); NtGt3 (SEQ ID NO. 11); NtGt4 (SEQ ID NO. 13); Bs-YjiC (SEQ ID NO. 29); and VvGT14 (SEQ ID NO. 31). In this embodiment, one or more of the above UGTs may glycosylate one or more terpenes selected from the group consisting of: alpha-Pinene, Camphene, beta-Myrcene, beta-Pinene, delta-3-Carene, alpha-Terpine, Ocimene 1, d-Limonene, p-Cymene, Ocimene 2, Eucalyptol, gamma-Terpinene, Terpinoline, Linalool, Isolpulegol, Geraniol, beta-Caryophyllene, alpha-Humulene, Nerolidol 1, Nerolidol 2, Guaiol, Caryophyllene oxide, and alpha-Bisabolol.

In another preferred embodiment, a yeast cell may be genetically modified to express a heterologous nucleotide sequence, operably linked to a promoter, encoding one or more UGTs having glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); UGT94P1 (SEQ ID NO. 7); NtGt3 (SEQ ID NO. 11); NtGt4 (SEQ ID NO. 13); Bs-YjiC (SEQ ID NO. 29); and VvGT14 (SEQ ID NO. 31), wherein at least one glycosylated terpene is selected from the group consisting of: alpha-Bisabolyl monoglucoside, alpha-Bisabolyl O-acetyl glucoside, alpha-Bisabolyl oxidized glucoside, alpha-Bisabolyl diglucoside, Citronellyl monoglucoside, Geranyl O-acetyl glucoside, Geranyl monoglucoside, Linalyl monoglucoside, Linalyl glucoside-xyloside, Linalyl diglucoside, Neryl monoglucoside, Neryl diglucoside and Terpineol diglucoside.

In another preferred embodiment, expression of one or more UGTs may further be in conjunction with the expression of a heterologous glycosyltransferase that has glycosylation activity directed to cannabinoids as well as terpenes. In this preferred embodiment, a yeast cell may be genetically modified to express a heterologous nucleotide sequence, operably linked to a promoter, encoding at least one the following UGTs having glycosylation activity directed to cannabinoids as well as terpenes selected from the group consisting of: NtGt3 (SEQ ID NO. 11); and NtGt4 (SEQ ID NO. 13), in addition to the heterologous expression of one or more UGTs having glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); and UGT94P1 (SEQ ID NO. 7). Additional UGTs that have glycosylation activity towards cannabinoids are incorporated by reference from Sayre et al. in PCT/US18/24409.

In one preferred embodiment, the present inventors demonstrate the generation of a genetically modified non-cannabinoid producing plant cell culture configured to produce water-soluble, and preferably glycosylated terpenes. Examples of such non-cannabinoid producing plant cells may include, but not be limited to tobacco plant cells, Humulus lupulus (hops) plant cells, Eucalyptus perriniana, Mentha piperita, Averrhoa carambola, Ocimum basilicum, and Arabidopsis thaliana.

In one preferred embodiment a tobacco cell may be transformed to over-express one or more select UGTs that have activity towards one or more terpenes which may further be in conjunction with the endogenous glycosyltransferase from a tobacco plant that exhibits activity toward both cannabinoids and terpene compounds. In one preferred embodiments, a tobacco cell may be transformed to over-express one or more select UGTs that have activity towards one or more terpenes which may further be in conjunction with the endogenous glycosyltransferases NtGt3 (SEQ ID NO. 12); and NtGt4 (SEQ ID NO. 14) from a tobacco plant that exhibits activity toward both cannabinoids and terpene compounds. Select, or full spectrum terpenes, as well as cannabinoid compounds generated by the Cannabis sativa plant may be harvested, for example as an oil or other extract and may fed to such a genetically modified non-cannabinoid producing plant suspension cell culture, and preferably a tobacco suspension cell culture. Such terpenes, as well as cannabinoid compounds may be efficiently glycosylated through interaction with the endogenous and/or heterologous glycosyltransferase enzymes. In this embodiment, the synthesis of water-soluble cannabinoids and terpenes in the tobacco cell suspension cell culture may be harvested and isolated separately or together as a “full-spectrum” water-based extract. The post-harvest conversion of terpenes into terpene glycosides may allow their inclusion in one or more of the compositions described herein, or reconstituted to their original structure through the action of one or more glycosidases. Additional embodiments include the addition of one or more “glycosidase inhibitor” to a cell culture which may inhibit glycosidase enzymes which catalyze the hydrolysis of glyosidic bonds.

In one preferred embodiment, a tobacco cell may be genetically modified to express a heterologous nucleotide sequence, operably linked to a promoter, encoding one or more UGTs having glycosylation activity towards one or more terpenes selected from the group consisting of: alpha-Pinene, Camphene, beta-Myrcene, beta-Pinene, delta-3-Carene, alpha-Terpine, Ocimene 1, d-Limonene, p-Cymene, Ocimene 2, Eucalyptol, gamma-Terpinene, Terpinoline, Linalool, Isolpulegol, Geraniol, beta-Caryophyllene, alpha-Humulene, Nerolidol 1, Nerolidol 2, Guaiol, Caryophyllene oxide, and alpha-Bisabolol.

In another preferred embodiment, a tobacco cell may be genetically modified to express a heterologous nucleotide sequence, operably linked to a promoter, encoding one or more UGTs having glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); UGT94P1 (SEQ ID NO. 7). In this embodiment, one or more of the above UGTs may glycosylate one or more terpenes selected from the group consisting of: alpha-Pinene, Camphene, beta-Myrcene, beta-Pinene, delta-3-Carene, alpha-Terpine, Ocimene 1, d-Limonene, p-Cymene, Ocimene 2, Eucalyptol, gamma-Terpinene, Terpinoline, Linalool, Isolpulegol, Geraniol, beta-Caryophyllene, alpha-Humulene, Nerolidol 1, Nerolidol 2, Guaiol, Caryophyllene oxide, and alpha-Bisabolol.

In another preferred embodiment, a tobacco cell may be genetically modified to express a heterologous nucleotide sequence, operably linked to a promoter, encoding one or more UGTs having glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); UGT94P1 (SEQ ID NO. 7). 13), wherein at least one glycosylated terpene is selected from the group consisting of: alpha-Bisabolyl monoglucoside, alpha-Bisabolyl O-acetyl glucoside, alpha-Bisabolyl oxidized glucoside, alpha-Bisabolyl diglucoside, Citronellyl monoglucoside, Geranyl O-acetyl glucoside, Geranyl monoglucoside, Linalyl monoglucoside, Linalyl glucoside-xyloside, Linalyl diglucoside, Neryl monoglucoside, Neryl diglucoside and Terpineol diglucoside.

In another preferred embodiment, expression in a tobacco cell, and preferably BY2 tobacco cells, of one or more UGTs may further be in conjunction with the expression of an endogenous glycosyltransferase that has glycosylation activity directed to cannabinoids as well as terpenes. In this preferred embodiment, a tobacco cell may express, or overexpress an endogenous nucleotide sequence, operably linked to a promoter, encoding at least one the following UGTs having glycosylation activity directed to cannabinoids as well as terpenes selected from the group consisting of: NtGt3 (SEQ ID NO. 11); and NtGt4 (SEQ ID NO. 13), in addition to the heterologous expression of one or more UGTs having glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); and UGT94P1 (SEQ ID NO. 7). Additional UGTs that have glycosylation activity towards cannabinoids are incorporated by reference from Sayre et al. in PCT/US18/24409.

In another preferred embodiment, in a Cannabis, or alternatively a non-cannabinoid producing cell, may express one or more endogenous glycosyltransferases that have activity toward one or more terpenes, and in some embodiments one or more terpenes and cannabinoids. In a preferred embodiment, cells expressing one or more endogenous glycosyltransferases having activity toward one or more terpenes may be established in a suspension cell culture. Select, or full spectrum terpenes, as well as cannabinoid compounds generated by the Cannabis sativa plant may be harvested, for example as an oil or other extract and may fed to such a genetically modified cell culture in a fermenter. Such terpenes, as well as cannabinoid compounds may be efficiently glycosylated through interaction with the endogenous glycosyltransferase enzymes. In this embodiment, the synthesis of water-soluble cannabinoids, terpenes and/or terpenes in the non-genetically modified cells may be harvested and isolated separately or together as a “full-spectrum” water-based extract. The post-harvest conversion of terpenes into terpene glycosides may allow their inclusion in one or more of the compositions described herein, or reconstituted to their original structure through the action of one or more glycosidases. Additional embodiments include the addition of one or more “glycosidase inhibitor” to a cell culture which may inhibit glycosidase enzymes which catalyze the hydrolysis of glyosidic bonds.

In another preferred embodiment, a non-genetically modified tobacco cell and preferably BY2 tobacco cells, expressing one or more endogenous glycosyltransferases such as NtGt3 (SEQ ID NO. 11), and/or NtGt4 (SEQ ID NO. 13) or a homolog thereof, may be established in a suspension cell culture. Select, or full spectrum terpenes, as well as cannabinoid compounds generated by the Cannabis sativa plant may be harvested, for example as an oil or other extract and may fed to such a genetically modified cell culture in a fermenter. Such terpenes, as well as cannabinoid compounds may be efficiently glycosylated through interaction with the endogenous glycosyltransferase enzymes NtGt3 (SEQ ID NO. 14), and/or NtGt4 (SEQ ID NO. 14). In this embodiment, the synthesis of water-soluble cannabinoids, terpenes and/or terpenes in the non-genetically modified tobacco cells may be harvested and isolated separately or together as a “full-spectrum” water-based extract. The post-harvest conversion of terpenes into terpene glycosides may allow their inclusion in one or more of the compositions described herein, or reconstituted to their original structure through the action of one or more glycosidases. Additional embodiments include the addition of one or more “glycosidase inhibitor” to a yeast culture which may inhibit glycosidase enzymes which catalyze the hydrolysis of glyosidic bonds.

The present inventors may demonstrate the generation of a mom-GMO plant and/or cell culture configured to produce water-soluble, and preferably converted to a water-soluble terpene glycosides utilizing endogenous UGTs in wild-type yeast, such as Pichia pastoris. In this embodiment, a wild-type cell suspension culture of yeast cells may be generated. Select, or full spectrum terpenes, as well as cannabinoid compounds generated by the Cannabis sativa plant may be harvested, for example as an oil or other extract and may fed to such a genetically modified cell culture in a fermenter. Such terpenes, as well as cannabinoid compounds may be efficiently glycosylated through interaction with the endogenous glycosyltransferase enzymes. In this embodiment, the synthesis of water-soluble cannabinoids, terpenes and/or terpenes in the non-genetically modified cells may be harvested and isolated separately or together as a “full-spectrum” water-based extract. The post-harvest conversion of terpenes into terpene glycosides may allow their inclusion in one or more of the compositions described herein, or reconstituted to their original structure through the action of one or more glycosidases. Additional embodiments include the addition of one or more “glycosidase inhibitor” to a yeast culture which may inhibit glycosidase enzymes which catalyze the hydrolysis of glyosidic bonds.

In one preferred embodiment, the present inventors demonstrate the generation of a genetically modified prokaryotic organism configured to produce water-soluble, and preferably glycosylated terpene compounds. In one preferred embodiment a genetically modified bacterial cell, or other suitable bacterial species may be transformed to express one or more heterologous UGTs having activity towards at least one terpene. Select, or full spectrum terpenes, as well as cannabinoid compounds generated by the Cannabis sativa plant may be harvested, for example as an oil or other extract and may fed to such a genetically modified bacterial cell culture in a fermenter. Such terpenes, as well as cannabinoid compounds may be efficiently glycosylated through interaction with the overexpressed endogenous and/or heterologous glycosyltransferase enzymes. In this embodiment, the synthesis of water-soluble cannabinoids, terpenes and/or terpenes in the bacterial cells may be harvested and isolated separately or together as a “full-spectrum” water-based extract. The post-harvest conversion of terpenes into terpene glycosides may allow their inclusion in one or more of the compositions described herein, or reconstituted to their original structure through the action of one or more glycosidases. Additional embodiments include the addition of one or more “glycosidase inhibitor” to a cell culture which may inhibit glycosidase enzymes which catalyze the hydrolysis of glyosidic bonds.

In one preferred embodiment, a bacterial cell may be genetically modified to express a heterologous nucleotide sequence, operably linked to a promoter, encoding one or more UGTs having glycosylation activity towards one or more terpenes selected from the group consisting of: alpha-Pinene, Camphene, beta-Myrcene, beta-Pinene, delta-3-Carene, alpha-Terpine, Ocimene 1, d-Limonene, p-Cymene, Ocimene 2, Eucalyptol, gamma-Terpinene, Terpinoline, Linalool, Isolpulegol, Geraniol, beta-Caryophyllene, alpha-Humulene, Nerolidol 1, Nerolidol 2, Guaiol, Caryophyllene oxide, and alpha-Bisabolol.

In another preferred embodiment, a bacterial cell, such as a high-protein production species of E. coli, may be genetically modified to express a heterologous nucleotide sequence, operably linked to a promoter, encoding one or more UGTs having glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); UGT94P1 (SEQ ID NO. 7); NtGt3 (SEQ ID NO. 11); NtGt4 (SEQ ID NO. 13); Bs-YjiC (SEQ ID NO. 29); and VvGT14 (SEQ ID NO. 31). In this embodiment, one or more of the above UGTs may glycosylate one or more terpenes selected from the group consisting of: alpha-Pinene, Camphene, beta-Myrcene, beta-Pinene, delta-3-Carene, alpha-Terpine, Ocimene 1, d-Limonene, p-Cymene, Ocimene 2, Eucalyptol, gamma-Terpinene, Terpinoline, Linalool, Isolpulegol, Geraniol, beta-Caryophyllene, alpha-Humulene, Nerolidol 1, Nerolidol 2, Guaiol, Caryophyllene oxide, and alpha-Bisabolol.

In another preferred embodiment, a bacterial cell may be genetically modified to express a heterologous nucleotide sequence, operably linked to a promoter, encoding one or more UGTs having glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); UGT94P1 (SEQ ID NO. 7); NtGt3 (SEQ ID NO. 11); NtGt4 (SEQ ID NO. 13); Bs-YjiC (SEQ ID NO. 29); and VvGT14 (SEQ ID NO. 31), wherein at least one glycosylated terpene is selected from the group consisting of: alpha-Bisabolyl monoglucoside, alpha-Bisabolyl O-acetyl glucoside, alpha-Bisabolyl oxidized glucoside, alpha-Bisabolyl diglucoside, Citronellyl monoglucoside, Geranyl O-acetyl glucoside, Geranyl monoglucoside, Linalyl monoglucoside, Linalyl glucoside-xyloside, Linalyl diglucoside, Neryl monoglucoside, Neryl diglucoside and Terpineol diglucoside.

In another preferred embodiment, expression of one or more UGTs may further be in conjunction with the expression of a heterologous glycosyltransferase that has glycosylation activity directed to cannabinoids as well as terpenes. In this preferred embodiment, a bacterial cell may be genetically modified to express a heterologous nucleotide sequence, operably linked to a promoter, encoding at least one the following UGTs having glycosylation activity directed to cannabinoids as well as terpenes selected from the group consisting of: NtGt3 (SEQ ID NO. 11); and NtGt4 (SEQ ID NO. 13), in addition to the heterologous expression of one or more UGTs having glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); and UGT94P1 (SEQ ID NO. 7). Additional UGTs that have glycosylation activity towards cannabinoids are incorporated by reference from Sayre et al. in PCT/US18/24409.

In one preferred embodiment, the present inventors demonstrate the generation of water-soluble, and preferably glycosylated terpene compounds in an in vitro or ex vivo system, such as a bioreactor. In one preferred embodiment, one or more UGTs having activity towards at least one terpene may be introduced in vitro, or ex vivo, for example in a cell-free bioreactor, to a select, or full spectrum terpenes, as well as cannabinoid compounds generated by the Cannabis sativa or hemp plant. Such terpenes, as well as cannabinoid compounds may be efficiently glycosylated through interaction with the glycosyltransferase enzymes. In this embodiment, the in vitro or ex vivo synthesis of water-soluble cannabinoids, terpenes and/or terpenes may be harvested and isolated separately or together as a “full-spectrum” water-based extract. In certain embodiments, such in vitro, and ex vivo system may be supplemented with an appropriate reaction buffer, as well as UDP-glucose or other appropriate glycosylation substrate. The post-harvest conversion of terpenes into terpene glycosides may allow their inclusion in one or more of the compositions described herein, or reconstituted to their original structure through the action of one or more glycosidases.

In one preferred embodiment, one or more UGTs having in vitro or ex vivo glycosylation activity towards one or more terpenes may be selected from the group consisting of: alpha-Pinene, Camphene, beta-Myrcene, beta-Pinene, delta-3-Carene, alpha-Terpine, Ocimene 1, d-Limonene, p-Cymene, Ocimene 2, Eucalyptol, gamma-Terpinene, Terpinoline, Linalool, Isolpulegol, Geraniol, beta-Caryophyllene, alpha-Humulene, Nerolidol 1, Nerolidol 2, Guaiol, Caryophyllene oxide, and alpha-Bisabolol.

In another preferred embodiment, one or more UGTs having glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); UGT94P1 (SEQ ID NO. 7); NtGt3 (SEQ ID NO. 11); NtGt4 (SEQ ID NO. 13); Bs-YjiC (SEQ ID NO. 29); and VvGT14 (SEQ ID NO. 31). In this embodiment, one or more of the above UGTs may glycosylate one or more terpenes—in vitro or ex vivo—selected from the group consisting of: alpha-Pinene, Camphene, beta-Myrcene, beta-Pinene, delta-3-Carene, alpha-Terpine, Ocimene 1, d-Limonene, p-Cymene, Ocimene 2, Eucalyptol, gamma-Terpinene, Terpinoline, Linalool, Isolpulegol, Geraniol, beta-Caryophyllene, alpha-Humulene, Nerolidol 1, Nerolidol 2, Guaiol, Caryophyllene oxide, and alpha-Bisabolol.

In another preferred embodiment, one or more UGTs having in vitro or ex vivo glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); UGT94P1 (SEQ ID NO. 7); NtGt3 (SEQ ID NO. 11); NtGt4 (SEQ ID NO. 13); Bs-YjiC (SEQ ID NO. 29); and VvGT14 (SEQ ID NO. 31), wherein at least one glycosylated terpene is selected from the group consisting of: alpha-Bisabolyl monoglucoside, alpha-Bisabolyl O-acetyl glucoside, alpha-Bisabolyl oxidized glucoside, alpha-Bisabolyl diglucoside, Citronellyl monoglucoside, Geranyl O-acetyl glucoside, Geranyl monoglucoside, Linalyl monoglucoside, Linalyl glucoside-xyloside, Linalyl diglucoside, Neryl monoglucoside, Neryl diglucoside and Terpineol diglucoside.

In another preferred embodiment, one or more UGTs may further include glycosyltransferase that has in vitro or ex vivo glycosylation activity directed to cannabinoids as well as terpenes. Such UGTs having in vitro or ex vivo glycosylation activity directed to cannabinoids as well as terpenes selected from the group consisting of: NtGt3 (SEQ ID NO. 11); and NtGt4 (SEQ ID NO. 13), in addition to one or more UGTs having in vitro or ex vivo glycosylation activity towards one or more terpenes selected from the group consisting of: UGT58A1 (SEQ ID NO. 1); MhGt1 (SEQ ID NO. 3); VvGt7 (SEQ ID NO. 9); UGT85K11 (SEQ ID NO. 5); UGT94P1 (SEQ ID NO. 7), Bs-YjiC (SEQ ID NO. 29); and VvGT14 (SEQ ID NO. 31). Additional UGTs that have glycosylation activity towards cannabinoids are incorporated by reference from Sayre et al. in PCT/US18/24409.

On one embodiment, the invention may include the biosynthesis of a novel terpene compound according to Formula I:

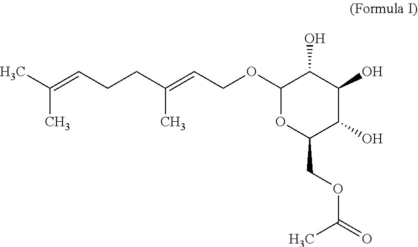 [SMILES: CC(=O)OC[C@H]1OC(OC/C=C(\C)CCC=C(C)C)[C@H](O)[C@@H](O)[C@@H]1O]

In one preferred embodiment, the compound of Formula I may be generated in vivo, and preferably in planta. In this embodiment, a Cannabis plant or cell may transformed to with an expression vector co-expressing nucleotide sequences encoding UGTs enzymes according to nucleotide sequences SEQ ID NO. 5, and SEQ ID NO. 7. In this embodiment, the co-expressed nucleotide sequences may be operably linked to a promoter, and encode UGTs enzymes according to amino acid sequences SEQ ID NO. 6, and SEQ ID NO. 8 that may convert endogenous geraniol into geranyl O-acetyl glucoside as shown in Formula I.

In another preferred embodiment, the compound of Formula I may be generated in vivo, and preferably in a yeast or cell suspension culture system as generally described herein. In this preferred embodiment, a yeast, bacterial or cell of suspension cell cultures, such as a tobacco cell suspension culture, may transformed to with an expression vector co-expressing nucleotide sequences encoding UGTs enzymes according to nucleotide sequences SEQ ID NO. 5, and SEQ ID NO. 7. In this embodiment, the co-expressed nucleotide sequences may be operably linked to a promoter, and encode UGTs enzymes according to amino acid sequences SEQ ID NO. 6, and SEQ ID NO. 8 that may convert endogenous geraniol into geranyl O-acetyl glucoside as shown in Formula I.

In another preferred embodiment, the compound of Formula I may be generated in vitro, or ex vivo such as a bioreactor. In this preferred embodiment, a quantity of geraniol may be introduced to UGTs enzymes according to amino acid sequences SEQ ID NO. 6, and SEQ ID NO. 8 that may convert the geraniol into geranyl O-acetyl glucoside as shown in Formula I. In certain embodiments, such in vitro, and ex vivo system may be supplemented with an appropriate reaction buffer, as well as UDP-glucose or other appropriate glycosylation substrate.

On one embodiment, the invention may include the biosynthesis of a novel terpene compound according to Formula III:

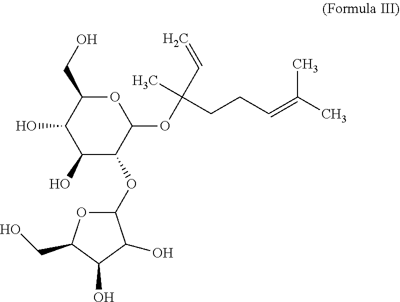 [SMILES: C=CC(C)(CCC=C(C)C)OC1O[C@H](CO)[C@@H](O)[C@H](O)[C@H]1OC1O[C@H](CO)[C@H](O)C1O]

In one preferred embodiment, the compound of Formula I may be generated in vivo, and preferably in planta. In this embodiment, a Cannabis plant or cell may transformed to with an expression vector co-expressing nucleotide sequences encoding UGTs enzymes according to nucleotide sequences SEQ ID NO. 5, and SEQ ID NO. 7. In this embodiment, the co-expressed nucleotide sequences may be operably linked to a promoter, and encode UGTs enzymes according to amino acid sequences SEQ ID NO. 6, and SEQ ID NO. 8 that may convert endogenous geraniol into geranyl O-acetyl glucoside as shown in Formula II.

In another preferred embodiment, the compound of Formula II may be generated in vivo, and preferably in a yeast or cell suspension culture system as generally described herein. In this preferred embodiment, a yeast, bacterial or cell of suspension cell cultures, such as a tobacco cell suspension culture, may transformed to with an expression vector co-expressing nucleotide sequences encoding UGTs enzymes according to nucleotide sequences SEQ ID NO. 5, and SEQ ID NO. 7. In this embodiment, the co-expressed nucleotide sequences may be operably linked to a promoter, and encode UGTs enzymes according to amino acid sequences SEQ ID NO. 6, and SEQ ID NO. 8 that may convert endogenous Linalool into Linalyl glucoside-xyloside as shown in Formula II.

In another preferred embodiment, the compound of Formula II may be generated in vitro, or ex vivo such as a bioreactor. In this preferred embodiment, a quantity of geraniol may be introduced to UGTs enzymes according to amino acid sequences SEQ ID NO. 6, and SEQ ID NO. 8 that may convert the Linalool into Linalyl glucoside-xyloside as shown in Formula II. In certain embodiments, such in vitro, and ex vivo system may be supplemented with an appropriate reaction buffer, as well as UDP-glucose or other appropriate glycosylation substrate.

The present inventors may utilize one or more of the following exemplary UGT genes/proteins to glycosylate one or more terpenes. Such UGTs may be endogenously or heterologously expressed in plants, such as a Cannabis plant, as well as and in vivo cultured systems such as plant, yeast or bacterial cell cultures. Polynucleotide as well as amino acid sequences of the following may be ascertained without undue experimentation by one of ordinary skill in the art. As such, all sequences identified herein are specifically incorporated by reference. Codon-optimized versions of each of the aforementioned genes are also included in the current invention. Such codon-optimized genes may be configured for optimized expression in heterologous systems, such as yeast or bacterial systems. Sequences identified herein as “codon-optimized” may be codon optimized for expression in yeast.

Examples may include: Vitis vinifera: VvGT7 (XM_002276510.3), VvGT14 (XM_002285734.4), VvGT15 (XM_002281477.2), VvGT16 (XM_002263122.1), VvGT17 (XM_002285743.2), VvGT18 (XM_002285372.1), VvGT19 (XM_002285744.1), VvGT20 (XM_002266592.2). Nicotiana tobaccum: NtGT1, NtGT2, NtGT3, NtGT4, and NtGT5. (Nicotiana sequences found in Sayre et al. in PCT/US18/24409 are hereby incorporated by reference.) Mucor hiemalis: a phenolic glycosyltransferase. Pichia etchellsii: beta-glucosidase II (XM_020688260.1). Prunus dulcis: almond beta-glucosidase (S17766). Actinidia deliciosa: kiwi fruit AdGT1 (KF954941.1), AdGT2 (KF954942.1), AdGT3 (KF954943.1), AdGT4 (KF954944.1). Arabidopsis thaliana: UGT76E2 (NM_125351.3), UGT76E11 (NM_114534.3), UGT76E12 (NM 114533.2), UGT76D1 (NM_128205.3), UGT84A3 (NM_117639.3), UGT84A4 (NM_117640.4), UGT84B2 (NM_127889.1), UGT84B1 (NM_127890.3), UGT75B2 (NM_100432.2), UGT75B1 (NM_001331554.1), UGT75D1, UGT74E2 (NM_100448.4), UGT74F1 (NM_129946.3), UGT74F2 (NM_129944.3), UGT74D1 (NM_128733.5), UGT74B1 (NM_102256.3), UGT85A1 (NM_102089.5), UGT85A5 (NM 202156.2), UGT85A4 (NM_106476.3), UGT85A2 (NM_102086.3), UGT85A7 (NM_102085.3), UGT73C3, UGT73C6 (NM_129234.2), UGT73C1 (NM_129230.3), UGT71C2 (NM_128528.4), UGT88A1 (NM_112524.4). (Arabidopsis thaliana sequences found in Sayre et al. in PCT/US18/24409 are hereby incorporated by reference.)

Another aspect of the invention includes systems, methods and compositions for the in vivo formulation of terpene glycosides and/or cannabinoid glycosides. In one preferred embodiment, UGTs may be selectively expressed in one or more of the in vivo systems described herein, such as an in planta, yeast, or cell culture system. Such selectively expressed UGTs may have glycosylation activity towards some terpenes or cannabinoids, and not others. By selectively co-expressing combinations of UGTs in an in vivo system, the water-solubilized glycosylated compounds may be selectively removed allowing for isolation of selective formulations of water-soluble terpenes and/or cannabinoid or a mixture of the two. Such selective formulations of water-soluble terpenes and/or cannabinoid or a mixture of the two may be selected for flavor, texture, and taste among other attributes and may further be incorporated into one or more consumer products as described herein.

For example, in one embodiment, the expression of the UGT UGT85K11 (SEQ ID NO. 6) in a yeast or plant system may selectively glycosylate nerol to neryl monoglucoside. The glycosylated form, being water-soluble may not be selectively removed and isolated in a water-fraction. In another preferred embodiment, additional UTGs may be identified, for example through bioinformatic methods known by those of ordinary skill in the art, as having homology with other identified UTGs that preferably that have glycosylation activity towards one or more terpenes and/or cannabinoids. Such newly identified UGTs may be tested in one or more of the in vivo yeast, plant or cells systems discussed herein to determine if they exhibit selective glycosylation activity towards one or more terpenes and/or cannabinoids. Such combinations of selective UGTs may be co-expressed to generate a specific profile of terpenes and/or cannabinoid or mixtures of both that may be selectively solubilized and isolated from a water fraction.

In one embodiment, one of the aforementioned compositions may act as a prodrug. The term “prodrug” is taken to mean compounds according to the invention which have been modified by means of, for example, sugars and which are cleaved in the organism to form the effective compounds according to the invention. The terms “therapeutically effective amount” or “effective dose” or “dose” are interchangeably used herein and denote an amount of the pharmaceutical compound having a prophylactically or therapeutically relevant effect on a disease or pathological conditions, i.e. which causes in a tissue, system, animal or human a biological or medical response which is sought or desired, for example, by a researcher or physician. Pharmaceutical formulations can be administered in the form of dosage units which comprise a predetermined amount of active ingredient per dosage unit. The concentration of the prophylactically or therapeutically active ingredient in the formulation may vary from about 0.1 to 100 wt %. Preferably, the compound of formula (I) or the pharmaceutically acceptable salts thereof are administered in doses of approximately 0.5 to 1000 mg, more preferably between 1 and 700 mg, most preferably 5 and 100 mg per dose unit. Generally, such a dose range is appropriate for total daily incorporation. In other terms, the daily dose is preferably between approximately 0.02 and 100 mg/kg of body weight. The specific dose for each patient depends, however, on a wide variety of factors as already described in the present specification (e.g. depending on the condition treated, the method of administration and the age, weight and condition of the patient). Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

In the meaning of the present invention, the compound is further defined to include pharmaceutically usable derivatives, solvates, prodrugs, tautomers, enantiomers, racemates and stereoisomers thereof, including mixtures thereof in all ratios.

As noted above, the present invention allows the scaled production of water-soluble terpenes. Because of this enhanced solubility, the invention allows for the addition of such water-soluble terpene to a variety of compositions without requiring oils and/or emulsions that are generally required to maintain the non-modified terpenes in suspension. As a result, the present invention may allow for the production of a variety of compositions for both the food and beverage industry, as well as pharmaceutical applications that do not required oils and emulsion suspensions and the like.

In one embodiment the invention may include aqueous compositions containing one or more water-soluble terpenes that may be introduced to a food or beverage. In a preferred embodiment, the invention may include an aqueous solution containing one or more dissolved water-soluble terpenes. In this embodiment, such water-soluble terpenes may include a glycosylated terpene, and/or an acetylated glycoside terpene (such as Geranyl O-acetyl glucoside), and/or a mixture of both. Here, the glycosylated terpene, and/or an acetylated glycoside terpene, and/or glycosylated-xylosylated terpene were generated in vivo as generally described herein, or in vitro. Moreover, in this embodiment, the aqueous may contain one or more of the following: saline, purified water, propylene glycol, deionized water, and/or an alcohol such as ethanol as well as a pH buffer that may allow the aqueous solution to be maintained at a pH below 7.4. Additional embodiments may include the addition of an acid of base, such as formic acid, or ammonium hydroxide.

In another embodiment, the invention may include a consumable food additive having at least one water-soluble terpene, such as a glycosylated and/or an acetylated glycoside terpene, and/or a mixture of both, where such water-soluble terpenes may be generated in vivo and/or in vitro. This consumable food additive may further include one or more a food additive polysaccharides, such as dextrin and/or maltodextrin, as well as an emulsifier. Example emulisifiers may include, but not be limited to: gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated glycoside tartaric acid esters of mono- and di-glycerides, citric acid esters of monoglycerides, stearoyl-2-lactylates, polysorbates, succinylated monoglycerides, acetylated glycoside monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.

The consumable food additive of the invention may be a homogenous composition and may further comprise a flavoring agent. Exemplary flavoring agents may include: sucrose (sugar), glucose, fructose, sorbitol, mannitol, corn syrup, high fructose corn syrup, saccharin, aspartame, sucralose, acesulfame potassium (acesulfame-K), neotame. The consumable food additive of the invention may also contain one or more coloring agents. Exemplary coloring agents may include: FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, saffron, Monosodium glutamate (MSG), hydrolyzed soy protein, autolyzed yeast extract, disodium guanylate or inosinate.

The consumable food additive of the invention may also contain one or more surfactants, such as glycerol monostearate and polysorbate 80. The consumable food additive of the invention may also contain one or more preservatives. Exemplary preservatives may include ascorbic acid, citric acid, sodium benzoate, calcium propionate, sodium erythorbate, sodium nitrite, calcium sorbate, potassium sorbate, BHA, BHT, EDTA, tocopherols. The consumable food additive of the invention may also contain one or more nutrient supplements, such as: thiamine hydrochloride, riboflavin, niacin, niacinamide, folate or folic acid, beta carotene, potassium iodide, iron or ferrous sulfate, alpha tocopherols, ascorbic acid, Vitamin D, amino acids, multi-vitamin, fish oil, co-enzyme Q-10, and calcium.

In one embodiment, the invention may include a consumable fluid containing at least one dissolved water-soluble terpene. In one preferred embodiment, this consumable fluid may be added to a drink or beverage to infuse it with the dissolved water-soluble terpene generated in an in vivo system as generally herein described, or through an in vitro process. As noted above, such water-soluble terpene may include a water-soluble glycosylated terpene and/or a water-soluble acetylated glycoside terpene (such as Geranyl O-acetyl glucoside) and/or a mixture of both. The consumable fluid may include a food additive polysaccharide such as maltodextrin and/or dextrin, which may further be in an aqueous form and/or solution. For example, in one embodiment, and aqueous maltodextrin solution may include a quantity of sorbic acid and an acidifying agent to provide a food grade aqueous solution of maltodextrin having a pH of 2-4 and a sorbic acid content of 0.02-0.1% by weight.

In certain embodiments, the consumable fluid may include water, as well as an alcoholic beverage; a non-alcoholic beverage, a noncarbonated beverage, a carbonated beverage, a cola, a root beer, a fruit-flavored beverage, a citrus-flavored beverage, a fruit juice, a fruit-containing beverage, a vegetable juice, a vegetable containing beverage, a tea, a coffee, a dairy beverage, a protein containing beverage, a shake, a sports drink, an energy drink, and a flavored water.

The consumable fluid may further include at least one additional ingredients, including but not limited to: xanthan gum, cellulose gum, whey protein hydrolysate, ascorbic acid, citric acid, malic acid, sodium benzoate, sodium citrate, sugar, phosphoric acid, and water.

In one embodiment, the invention may include a consumable gel having at least one water-soluble terpene and gelatin in an aqueous solution. In a preferred embodiment, the consumable gel may include a water-soluble glycosylated terpene and/or a water-soluble acetylated glycoside terpene, or a mixture of both, generated in an in vivo system, such as a whole plant or cell suspension culture system as generally herein described.

Additional embodiments may include a liquid composition having at least one water-soluble terpene solubilized in a first quantity of water; and at least one of: xanthan gum, cellulose gum, whey protein hydrolysate, ascorbic acid, citric acid, malic acid, sodium benzoate, sodium citrate, sugar, phosphoric acid, and/or a sugar alcohol. In this embodiment, a water-soluble terpene may include a glycosylated water-soluble terpene, an acetylated glycoside water-soluble terpene, or a mixture of both. In one preferred embodiment, the composition may further include a quantity of ethanol. Here, the amount of water-soluble terpene may include: less than 10 mass % water; more than 95 mass % water; about 0.1 mg to about 1000 mg of the water-soluble terpene; about 0.1 mg to about 500 mg of the water-soluble terpenes; about 0.1 mg to about 200 mg of the water-soluble terpene; about 0.1 mg to about 100 mg of the water-soluble terpene; about 0.1 mg to about 100 mg of the water-soluble terpene; about 0.1 mg to about 10 mg of the water-soluble terpene; about 0.5 mg to about 5 mg of the water-soluble terpene; about 1 mg/kg to 5 mg/kg (body weight) in a human of the water-soluble terpene.

In alternative embodiments, the composition may include at least one water-soluble terpene in the range of 50 mg/L to 300 mg/L; at least one water-soluble terpene in the range of 50 mg/L to 100 mg/L; at least one water-soluble terpene in the range of 50 mg/L to 500 mg/L; at least one water-soluble terpene over 500 mg/L; at least one water-soluble terpene under 50 mg/L. Additional embodiments may include one or more of the following additional components: a flavoring agent; a coloring agent; a coloring agent; and/or caffeine.

In one embodiment, the invention may include a liquid composition having at least one water-soluble terpene solubilized in said first quantity of water and a first quantity of ethanol in a liquid state. In a preferred embodiment, a first quantity of ethanol in a liquid state may be between 1% to 20% weight by volume of the liquid composition. In this embodiment, a water-soluble terpene may include a glycosylated water-soluble terpene, an acetylated glycoside water-soluble terpene, or a mixture of both. Such water-soluble terpenes may be generated in an in vivo and/or in vitro system as herein identified. In a preferred embodiment, the ethanol, or ethyl alcohol component may be up to about ninety-nine point nine-five percent (99.95%) by weight and the water-soluble terpene about zero point zero five percent (0.05%) by weight.

Examples of the preferred embodiment may include liquid ethyl alcohol compositions having one or more water-soluble terpenes wherein said ethyl alcohol has a proof greater than 100, and/or less than 100. Additional examples of a liquid composition containing ethyl alcohol and at least one water-soluble terpene may include, beer, wine and/or distilled spirit.

Additional embodiments of the invention may include a chewing gum composition having a first quantity of at least one water-soluble terpene. In a preferred embodiment, a chewing gum composition may further include a gum base comprising a buffering agent selected from the group consisting of acetates, glycinates, phosphates, carbonates, glycerophosphates, citrates, borates, and mixtures thereof. Additional components may include at least one sweetening agent; and at least one flavoring agent. As noted above, in a preferred embodiment,

In one embodiment, the chewing gum composition described above may include:
0.01 to 1% by weight of at least one water-soluble terpene;
25 to 85% by weight of a gum base;
10 to 35% by weight of at least one sweetening agent; and
1 to 10% by weight of a flavoring agent.

Here, such flavoring agents may include: menthol flavor, Eucalyptus, mint flavor and/or L-menthol. Sweetening agents may include one or more of the following: xylitol, sorbitol, isomalt, aspartame, sucralose, acesulfame potassium, and saccharin. Additional preferred embodiments may include a chewing gum having a pharmaceutically acceptable excipient selected from the group consisting of: fillers, disintegrants, binders, lubricants, and antioxidants. The chewing gum composition may further be non-disintegrating and also include one or more coloring and/or flavoring agents.

The invention may further include a composition for a water-soluble terpene infused solution comprising essentially of: water and/or purified water, at least one water-soluble terpene, and at least one flavoring agent. A water-soluble terpene infused solution of the invention may further include a sweetener selected from the group consisting of: glucose, sucrose, invert sugar, corn syrup, Stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same. Additional components of the water-soluble terpene infused solution may include, but not be limited to: sodium chloride, sodium chloride solution, glycerin, a coloring agent, and a demulcent. As to this last potential component, in certain embodiments, a demulcent may include: pectin, glycerin, honey, methylcellulose, and/or propylene glycol. As noted above, in a preferred embodiment, a water-soluble terpene may include at least one water-soluble acetylated glycoside terpene, and/or at least one water-soluble glycosylated terpene, and/or at least one glycosylated-xylosylated terpene or a mixture thereof. In this embodiment, such water soluble glycosylated terpene, and/or an acetylated glycoside terpene, and/or glycosylated-xylosylated terpene may have been glycosylated and/or acetylated glycoside in vivo respectively.

The invention may further include a composition for a water-soluble terpene infused anesthetic solution having water, or purified water, at least one water-soluble terpene, and at least one oral anesthetic. In a preferred embodiment, an anesthetic may include benzocaine, and/or phenol in a quantity of between 0.1% to 15% volume by weight.

Additional embodiments may include a water-soluble terpene infused anesthetic solution having a sweetener which may be selected from the group consisting of: glucose, sucrose, invert sugar, corn syrup, Stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same. Additional components of the water-soluble terpene infused solution may include, but not be limited to: sodium chloride, sodium chloride solution, glycerin, a coloring agent a demulcent. In a preferred embodiment, a demulcent may selected from the group consisting of: pectin, glycerin, honey, methylcellulose, and propylene glycol. As noted above, in a preferred embodiment, a water-soluble terpene may include at least one water-soluble acetylated glycoside terpene, and/or at least one water-soluble glycosylated terpene, and/or at least one glycosylated-xylosylated terpene or a mixture thereof. In this embodiment, such water soluble glycosylated terpene, and/or an acetylated glycoside terpene, and/or glycosylated-xylosylated terpene may have been glycosylated and/or acetylated glycoside in vivo respectively.

The invention may further include a composition for a hard lozenge for rapid delivery of water-soluble terpenes through the oral mucosa. In this embodiment, such a hard lozenge composition may include: a crystalized sugar base, and at least one water-soluble terpene, wherein the hard lozenge has a moisture content between 0.1 to 2%. In this embodiment, the water-soluble terpene may be added to the sugar base when it is in a liquefied form and prior to the evaporation of the majority of water content. Such a hard lozenge may further be referred to as a candy.

In a preferred embodiment, a crystalized sugar base may be formed from one or more of the following: sucrose, invert sugar, corn syrup, and isomalt or a combination of the same. Additional components may include at least one acidulant. Examples of acidulants may include, but not be limited to: citric acid, tartaric acid, fumaric acid, and malic acid. Additional components may include at least one pH adjustor. Examples of pH adjustors may include, but not be limited to: calcium carbonate, sodium bicarbonate, and magnesium trisilicate.

In another preferred embodiment, the composition may include at least one anesthetic. Example of such anesthetics may include benzocaine, and phenol. In this embodiment, first quantity of anesthetic may be between 1 mg to 15 mg per lozenge. Additional embodiments may include a quantity of menthol. In this embodiment, such a quantity of menthol may be between 1 mg to 20 mg. The hard lozenge composition may also include a demulcent, for example: pectin, glycerin, honey, methylcellulose, propylene glycol, and glycerin. In this embodiment, a demulcent may be in a quantity between 1 mg to 10 mg. As noted above, in a preferred embodiment, a water-soluble terpene may include at least one water-soluble acetylated glycoside terpene, and/or at least one water-soluble glycosylated terpene, and/or at least one glycosylated-xylosylated terpene or a mixture thereof. In this embodiment, such water soluble glycosylated terpene, and/or an acetylated glycoside terpene, and/or glycosylated-xylosylated terpene may have been glycosylated and/or acetylated glycoside in vivo respectively.

The invention may include a chewable lozenge for rapid delivery of water-soluble terpenes through the oral mucosa. In a preferred embodiment, the compositions may include: a glycerinated gelatin base, at least one sweetener; and at least one water-soluble terpene dissolved in a first quantity of water. In this embodiment, a sweetener may include sweetener selected from the group consisting of: glucose, sucrose, invert sugar, corn syrup, Stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same.

Additional components may include at least one acidulant. Examples of acidulants may include, but not be limited to: citric acid, tartaric acid, fumaric acid, and malic acid. Additional components may include at least one pH adjustor. Examples of pH adjustors may include, but not be limited to: calcium carbonate, sodium bicarbonate, and magnesium trisilicate.

In another preferred embodiment, the composition may include at least one anesthetic. Example of such anesthetics may include benzocaine, and phenol. In this embodiment, first quantity of anesthetic may be between 1 mg to 15 mg per lozenge. Additional embodiments may include a quantity of menthol. In this embodiment, such a quantity of menthol may be between 1 mg to 20 mg. The chewable lozenge composition may also include a demulcent, for example: pectin, glycerin, honey, methylcellulose, propylene glycol, and glycerin. In this embodiment, a demulcent may be in a quantity between 1 mg to 10 mg. As noted above, in a preferred embodiment, a water-soluble terpene may include at least one water-soluble acetylated glycoside terpene, and/or at least one water-soluble glycosylated terpene, and/or at least one glycosylated-xylosylated terpene or a mixture thereof. In this embodiment, such water soluble glycosylated terpene, and/or an acetylated glycoside terpene, and/or glycosylated-xylosylated terpene may have been glycosylated and/or acetylated glycoside in vivo respectively.

The invention may include a soft lozenge for rapid delivery of water-soluble terpenes through the oral mucosa. In a preferred embodiment, the compositions may include: polyethylene glycol base, at least one sweetener; and at least one water-soluble terpene dissolved in a first quantity of water. In this embodiment, a sweetener may include sweetener selected from the group consisting of: glucose, sucrose, invert sugar, corn syrup, Stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same. Additional components may include at least one acidulant. Examples of acidulants may include, but not be limited to: citric acid, tartaric acid, fumaric acid, and malic acid. Additional components may include at least one pH adjustor. Examples of pH adjustors may include, but not be limited to: calcium carbonate, sodium bicarbonate, and magnesium trisilicate.

In another preferred embodiment, the composition may include at least one anesthetic. Example of such anesthetics may include benzocaine, and phenol. In this embodiment, first quantity of anesthetic may be between 1 mg to 15 mg per lozenge. Additional embodiments may include a quantity of menthol. In this embodiment, such a quantity of menthol may be between 1 mg to 20 mg. The soft lozenge composition may also include a demulcent, for example: pectin, glycerin, honey, methylcellulose, propylene glycol, and glycerin. In this embodiment, a demulcent may be in a quantity between 1 mg to 10 mg. As noted above, in a preferred embodiment, a water-soluble terpene may include at least one water-soluble acetylated glycoside terpene, and/or at least one water-soluble glycosylated terpene, and/or at least one glycosylated-xylosylated terpene or a mixture thereof. In this embodiment, such water soluble glycosylated terpene, and/or an acetylated glycoside terpene, and/or glycosylated-xylosylated terpene may have been glycosylated and/or acetylated glycoside in vivo respectively.

In another embodiment, the invention may include a tablet or capsule consisting essentially of a water-soluble glycosylated terpene and a pharmaceutically acceptable excipient. Example may include solid, semi-solid and aqueous excipients such as: maltodextrin, whey protein isolate, xanthan gum, guar gum, diglycerides, monoglycerides, carboxymethyl cellulose, glycerin, gelatin, polyethylene glycol and water-based excipients.

In a preferred embodiment, a water-soluble terpene may include at least one water-soluble acetylated glycoside terpene, and/or at least one water-soluble glycosylated terpene, and/or at least one glycosylated-xylosylated terpene or a mixture thereof. In this embodiment, such water soluble glycosylated terpene, and/or an acetylated glycoside terpene, and/or glycosylated-xylosylated terpene may have been glycosylated and/or acetylated glycoside in vivo respectively. Examples of such in vivo systems being generally described herein, including in plant, as well as cell culture systems including Cannabis cell culture, tobacco cell culture and yeast cell culture systems. In one embodiment, a tablet or capsule may include an amount of water-soluble terpene of 5 milligrams or less. Alternative embodiments may include an amount of water-soluble terpene between 5 milligrams and 200 milligrams. Still other embodiments may include a tablet or capsule having amount of water-soluble terpene that is more than 200 milligrams.

The invention may further include a method of manufacturing and packaging a terpene dosage, consisting of the following steps: 1) preparing a fill solution with a desired concentration of a water-soluble terpene in a liquid carrier wherein said terpene solubilized in said liquid carrier; 2) encapsulating said fill solution in capsules; 3) packaging said capsules in a closed packaging system; and 4) removing atmospheric air from the capsules. In one embodiment, the step of removing of atmospheric air consists of purging the packaging system with an inert gas, such as, for example, nitrogen gas, such that said packaging system provides a room temperature stable product. In one preferred embodiment, the packaging system may include a plaster package, which may be constructed of material that minimizes exposure to moisture and air.

In one embodiment a preferred liquid carrier may include a water-based carrier, such as for example an aqueous sodium chloride solution. In a preferred embodiment, a water-soluble terpene may include at least one water-soluble acetylated glycoside terpene, and/or at least one water-soluble glycosylated terpene, and/or at least one glycosylated-xylosylated terpene or a mixture thereof. In this embodiment, such water soluble glycosylated terpene, and/or an acetylated glycoside terpene, and/or glycosylated-xylosylated terpene may have been glycosylated and/or acetylated glycoside in vivo respectively. Examples of such in vivo systems being generally described herein, including in plant, as well as cell culture systems including Cannabis cell culture, tobacco cell culture and yeast cell culture systems. In one embodiment, a desired terpene concentration may be about 1-10% w/w, while in other embodiments it may be about 1.5-6.5% w/w. Alternative embodiments may include an amount of water-soluble terpene between 5 milligrams and 200 milligrams. Still other embodiments may include a tablet or capsule having amount of water-soluble terpene that is more than 200 milligrams.

The invention may include an oral pharmaceutical solution, such as a sub-lingual spray, consisting essentially of a water-soluble terpene, 30-33% w/w water, about 50% w/w alcohol, 0.01% w/w butylated hydroxylanisole (BHA) or 0.1% w/w ethylenediaminetetraacetic acid (EDTA) and 5-21% w/w co-solvent, having a combined total of 100%, wherein said co-solvent is selected from the group consisting of propylene glycol, polyethylene glycol and combinations thereof, and wherein said water-soluble terpene is a glycosylated terpene, an acetylated glycoside terpene or a mixture of the two. In an alternative embodiment, such a oral pharmaceutical solution may consist essentially of 0.1 to 5% w/w of said water-soluble terpene, about 50% w/w alcohol, 5.5% w/w propylene glycol, 12% w/w polyethylene glycol and 30-33% w/w water. In a preferred composition, the alcohol component may be ethanol.

The invention may include an oral pharmaceutical solution, such as a sublingual spray, consisting essentially of about 0.1% to 1% w/w water-soluble terpene, about 50% w/w alcohol, 5.5% w/w propylene glycol, 12% w/w polyethylene glycol, 30-33% w/w water, 0.01% w/w butylated hydroxyanisole, having a combined total of 100%, and wherein said water-soluble terpene is a glycosylated terpene, an acetylated glycoside terpene or a mixture of the two wherein that were generated in vivo. In an alternative embodiment, such a oral pharmaceutical solution may consist essentially of 0.54% w/w water-soluble terpene, 31.9% w/w water, 12% w/w polyethylene glycol 400, 5.5% w/w propylene glycol, 0.01% w/w butylated hydroxyanisole, 0.05% w/w sucralose, and 50% w/w alcohol, wherein the a the alcohol components may be ethanol.

The invention may include a solution for nasal and/or sublingual administration of a terpene including: 1) an excipient of propylene glycol, ethanol anhydrous, or a mixture of both; and 2) a water-soluble terpene which may include glycosylated terpene an acetylated glycoside terpene or a mixture of the two generated in vivo and/or in vitro. In a preferred embodiment, the composition may further include a topical decongestant, which may include phenylephrine hydrochloride, Oxymetazoline hydrochloride, and Xylometazoline in certain preferred embodiments. The composition may further include an antihistamine, and/or a steroid. Preferably, the steroid component is a corticosteroid selected from the group consisting of: neclomethasone dipropionate, budesonide, ciclesonide, flunisolide, fluticasone furoate, fluticasone propionate, mometasone, triamcinolone acetonide. In alternative embodiments, the solution for nasal and/or sublingual administration of a terpene may further comprise at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.

The invention may further include an aqueous solution for nasal and/or sublingual administration of a terpene comprising: a water and/or saline solution; and a water-soluble terpene which may include a glycosylated terpene, an acetylated glycoside terpene or a mixture of the two generated in vivo and/or in vitro. In a preferred embodiment, the composition may further include a topical decongestant, which may include phenylephrine hydrochloride, Oxymetazoline hydrochloride, and Xylometazoline in certain preferred embodiments. The composition may further include an antihistamine, and/or a steroid. Preferably, the steroid component is a corticosteroid selected from the group consisting of: neclomethasone dipropionate, budesonide, ciclesonide, flunisolide, fluticasone furoate, fluticasone propionate, mometasone, triamcinolone acetonide. In alternative embodiments, the aqueous solution may further comprise at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.

The invention may include a topical formulation for the transdermal delivery of water-soluble terpene. In a preferred embodiment, a topical formulation for the transdermal delivery of water-soluble terpene may include a water-soluble glycosylated terpene, and/or water-soluble acetylated glycoside terpene, or a mixture of both, and a pharmaceutically acceptable excipient. Here, a glycosylated terpene and/or acetylated glycoside terpene may be generated in vivo and/or in vitro. Preferably a pharmaceutically acceptable excipient may include one or more: gels, ointments, cataplasms, poultices, pastes, creams, lotions, plasters and jellies or even polyethylene glycol. Additional embodiments may further include one or more of the following components: a quantity of capsaicin; a quantity of benzocaine; a quantity of lidocaine; a quantity of camphor; a quantity of benzoin resin; a quantity of methylsalicilate; a quantity of triethanolamine salicylate; a quantity of hydrocortisone; a quantity of salicylic acid.

The invention may include a gel for transdermal administration of a water soluble-terpene which may be generated in vitro and/or in vivo. In this embodiment, the mixture preferably contains from 15% to about 90% ethanol, about 10% to about 60% buffered aqueous solution or water, about 0.1 to about 25% propylene glycol, from about 0.1 to about 20% of a gelling agent, from about 0.1 to about 20% of a base, from about 0.1 to about 20% of an absorption enhancer and from about 1% to about 25% polyethylene glycol and a water-soluble terpene such as a glycosylated terpene, and/or acetylated glycoside terpene, and/or a mixture of the two.

In another embodiment, the invention may further include a transdermal composition having a pharmaceutically effective amount of a water-soluble terpene for delivery of the terpene to the bloodstream of a user. This transdermal composition may include a pharmaceutically acceptable excipient and at least one water-soluble terpene, such as a glycosylated terpene, an acetylated glycoside terpene, and a mixture of both, wherein the terpene is capable of diffusing from the composition into the bloodstream of the user. In a preferred embodiment, a pharmaceutically acceptable excipient to create a transdermal dosage form selected from the group consisting of: gels, ointments, cataplasms, poultices, pastes, creams, lotions, plasters and jellies. The transdermal composition may further include one or more surfactants. In one preferred embodiment, the surfactant may include a surfactant-lecithin organogel, which may further be present in an amount of between about between about 95% and about 98% w/w.

In an alternative embodiment, a surfactant-lecithin organogel comprises lecithin and PPG-2 myristyl ether propionate and/or high molecular weight polyacrylic acid polymers. The transdermal composition may further include a quantity of isopropyl myristate.

The invention may further include a transdermal composition having one or more permeation enhancers to facilitate transfer of the water-soluble terpene across a dermal layer. In a preferred embodiment, a permeation enhancer may include one or more of the following: propylene glycol monolaurate, diethylene glycol monoethyl ether, an oleoyl macrogolglyceride, a caprylocaproyl macrogolglyceride, and an oleyl alcohol. The invention may also include a liquid terpene liniment composition consisting of water, isopropyl alcohol solution and a water-soluble terpene, such as glycosylated terpene, and/or an acetylated glycoside terpene, and/or glycosylated-xylosylated terpene which may further have been generated in vivo. This liquid terpene liniment composition may further include approximately 97.5% to about 99.5% by weight of 70% isopropyl alcohol solution and from about 0.5% to about 2.5% by weight of a water-soluble terpene mixture.

Based on the improved solubility and other physical properties, as well as cost advantage and scalability of the invention's in vivo water-soluble production platform, the invention may include one or more commercial infusions. For example, commercially available products, such as lip balm, soap, shampoos, lotions, creams and cosmetics may be infused with one or more water-soluble terpenes.

As generally described herein, the invention may include one or more plants, such as a tobacco plant and/or cell culture that may be genetically modified to produce, for example water-soluble glycosylated terpenes in vivo. As such, in one preferred embodiment, the invention may include a tobacco plant and/or cell that may contain at least one water-soluble terpene. In a preferred embodiment, a tobacco plant containing a quantity of water-soluble terpenes may be used to generate a water-soluble terpene infused tobacco product such as a cigarette, pipe tobacco, chewing tobacco, cigar, and smokeless tobacco. In one embodiment, the tobacco plant may be treated with one or more glycosidase inhibitors. In a preferred embodiment, since the terpene being introduced to the tobacco plant may be controlled, the inventive tobacco plant may generate one or more selected water-terpenes. For example, in one embodiment, the genetically modified tobacco plant may be introduced to a single terpene, while in other embodiments the genetically modified tobacco plant may be introduced to a terpene extract containing a full and/or partial entourage of terpene compounds. The invention may further include a novel composition that may be used to supplement a cigarette, or other tobacco-based product. In this embodiment, the composition may include at least one water-soluble terpene dissolved in an aqueous solution. This aqueous solution may be wherein said composition may be introduced to a tobacco product, such as a cigarette and/or a tobacco leaf such that the aqueous solution may evaporate generating a cigarette and/or a tobacco leaf that contains the aforementioned water-soluble terpene(s), which may further have been generated in vivo as generally described herein.

In one embodiment the invention may include one or more methods of treating a medical condition in a mammal. In this embodiment, the novel method may include administering a therapeutically effective amount of a water-soluble terpene, such as an in vivo generated glycosylated terpene, and/or an acetylated glycoside terpene, and/or glycosylated-xylosylated terpene, and/or a mixture thereof or a pharmaceutically acceptable salt thereof, wherein the medical condition is selected from the group consisting of: obesity, post-traumatic stress syndrome, anorexia, nausea, emesis, pain, wasting syndrome, HIV-wasting, chemotherapy induced nausea and vomiting, alcohol use disorders, anti-tumor, amyotrophic lateral sclerosis, glioblastoma multiforme, glioma, increased intraocular pressure, glaucoma, Cannabis use disorders, Tourette's syndrome, dystonia, multiple sclerosis, inflammatory bowel disorders, arthritis, dermatitis, Rheumatoid arthritis, systemic lupus erythematosus, anti-inflammatory, anti-convulsant, anti-psychotic, anti-oxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, synovitis, and juvenile rheumatoid arthritis. In a preferred embodiment, the pharmaceutical composition may be administered by a route selected from the group consisting of: transdermal, topical, oral, buccal, sublingual, intra-venous, intra-muscular, vaginal, rectal, ocular, nasal and follicular. The amount of water-soluble terpenes may be a therapeutically effective amount, which may be determined by the patient's age, weight, medical condition terpene-delivered, route of delivery and the like. In one embodiment, a therapeutically effective amount may be 50 mg or less of a water-soluble terpene. In another embodiment, a therapeutically effective amount may be 50 mg or more of a water-soluble terpene. It should be noted that for any of the above composition, unless otherwise stated, an effective amount of water-soluble terpenes may include amounts between: 0.01 mg to 0.1 mg; 0.01 mg to 0.5 mg; 0.01 mg to 1 mg; 0.01 mg to 5 mg; 0.01 mg to 10 mg; 0.01 mg to 25 mg; 0.01 mg to 50 mg; 01 mg to 75 mg; 0.01 mg to 100 mg; 0.01 mg to 125 mg; 0.01 mg to 150 mg; 0.01 mg to 175 mg; 0.01 mg to 200 mg; 0.01 mg to 225 mg; 0.01 mg to 250 mg; 0.01 mg to 275 mg; 0.01 mg to 300 mg; 0.01 mg to 225 mg; 0.01 mg to 350 mg; 0.01 mg to 375 mg; 0.01 mg to 400 mg; 0.01 mg to 425 mg; 0.01 mg to 450 mg; 0.01 mg to 475 mg; 0.01 mg to 500 mg; 0.01 mg to 525 mg; 0.01 mg to 550 mg; 0.01 mg to 575 mg; 0.01 mg to 600 mg; 0.01 mg to 625 mg; 0.01 mg to 650 mg; 0.01 mg to 675 mg; 0.01 mg to 700 mg; 0.01 mg to 725 mg; 0.01 mg to 750 mg; 0.01 mg to 775 mg; 0.01 mg to 800 mg; 0.01 mg to 825 mg; 0.01 mg to 950 mg; 0.01 mg to 875 mg; 0.01 mg to 900 mg; 0.01 mg to 925 mg; 0.01 mg to 950 mg; 0.01 mg to 975 mg; 0.01 mg to 1000 mg; 0.01 mg to 2000 mg; 0.01 mg to 3000 mg; 0.01 mg to 4000 mg; 01 mg to 5000 mg; 0.01 mg to 0.1 mg/kg.; 0.01 mg to 0.5 mg/kg; 01 mg to 1 mg/kg; 0.01 mg to 5 mg/kg; 0.01 mg to 10 mg/kg; 0.01 mg to 25 mg/kg; 0.01 mg to 50 mg/kg; 0.01 mg to 75 mg/kg; and 0.01 mg to 100 mg/kg.

The modified terpene compounds of the present invention are useful for a variety of therapeutic applications. For example, the compounds are useful for treating or alleviating symptoms of diseases and disorders involving CB1 and CB2 receptors, as well as xanthine dehydrogenase and xanthine oxidase, including appetite loss, nausea and vomiting, pain, multiple sclerosis and epilepsy. For example, they may be used to treat pain (i.e. as analgesics) in a variety of applications including but not limited to pain management. In additional embodiments, such modified terpene compounds may be used as an appetite suppressant. Additional embodiments may include administering the modified terpene compounds.

By “treating” the present inventors mean that the compound is administered in order to alleviate symptoms of the disease or disorder being treated. Those of skill in the art will recognize that the symptoms of the disease or disorder that is treated may be completely eliminated, or may simply be lessened. Further, the compounds may be administered in combination with other drugs or treatment modalities, such as with chemotherapy or other cancer-fighting drugs. Implementation may generally involve identifying patients suffering from the indicated disorders and administering the compounds of the present invention in an acceptable form by an appropriate route. The exact dosage to be administered may vary depending on the age, gender, weight and overall health status of the individual patient, as well as the precise etiology of the disease. However, in general, for administration in mammals (e.g. humans), dosages in the range of from about 0.01 to about 300 mg of compound per kg of body weight per 24 hr., and more preferably about 0.01 to about 100 mg of compound per kg of body weight per 24 hr., are effective. Administration may be oral or parenteral, including intravenously, intramuscularly, subcutaneously, intradermal injection, intraperitoneal injection, etc., or by other routes (e.g. transdermal, sublingual, oral, rectal and buccal delivery, inhalation of an aerosol, etc.). In a preferred embodiment of the invention, the water-soluble terpene analogs are provided orally or intravenously. In particular, the phenolic esters of the invention are preferentially administered systemically in order to afford an opportunity for metabolic activation via in vivo cleavage of the ester. In addition, the water soluble compounds with azole moieties at the pentyl side chain do not require in vivo activation and may be suitable for direct administration (e.g. site specific injection).

The compounds may be administered in a pure form or in a pharmaceutically acceptable formulation including suitable elixirs, binders, and the like (generally referred to a “carriers”) or as pharmaceutically acceptable salts (e.g. alkali metal salts such as sodium, potassium, calcium or lithium salts, ammonium, etc.) or other complexes. It should be understood that the pharmaceutically acceptable formulations include liquid and solid materials conventionally utilized to prepare both injectable dosage forms and solid dosage forms such as tablets and capsules and aerosolized dosage forms. In addition, the compounds may be formulated with aqueous or oil based vehicles. Water may be used as the carrier for the preparation of compositions (e.g. injectable compositions), which may also include conventional buffers and agents to render the composition isotonic. Other potential additives and other materials (preferably those which are generally regarded as safe [GRAS]) include: colorants; flavorings; surfactants (TWEEN, oleic acid, etc.); solvents, stabilizers, elixirs, and binders or encapsulants (lactose, liposomes, etc). Solid diluents and excipients include lactose, starch, conventional disintergrating agents, coatings and the like. Preservatives such as methyl paraben or benzalkium chloride may also be used. Depending on the formulation, it is expected that the active composition will consist of about 1% to about 99% of the composition and the vehicular “carrier” will constitute about 1% to about 99% of the composition. The pharmaceutical compositions of the present invention may include any suitable pharmaceutically acceptable additives or adjuncts to the extent that they do not hinder or interfere with the therapeutic effect of the active compound.

The administration of the compounds of the present invention may be intermittent, bolus dose, or at a gradual or continuous, constant or controlled rate to a patient. In addition, the time of day and the number of times per day that the pharmaceutical formulation is administered may vary are and best determined by a skilled practitioner such as a physician. Further, the effective dose can vary depending upon factors such as the mode of delivery, gender, age, and other conditions of the patient, as well as the extent or progression of the disease. The compounds may be provided alone, in a mixture containing two or more of the compounds, or in combination with other medications or treatment modalities. The compounds may also be added to blood ex vivo and then be provided to the patient.

The term “glycosyltransferase” or “UGT” as generally used herein means an enzyme that has glycosylation and/or glycosylation and acetylation activity toward one or more terpenes, cannabinoids or both.

The term “terpene” refers to the large and diverse class of organic compounds produced by a variety of plants, including Cannabis plants. When terpenes are modified chemically, such as by oxidation or rearrangement of the carbon skeleton, the resulting compounds are generally referred to as “terpenoids.” The structure of terpenes are built with isoprenes, which are 5 carbon structures. Flavonoids are generally considered to be 15 carbon structures with two phenyl rings and a heterocyclic ring. So, there could be an overlap in which a flavonoid could be considered a terpene. However, not all terpenes could be considered flavonoids.

As used herein, the terms “terpene” and “terpenoid” are used interchangeably.

Within the context of the inventive technology, the term terpene includes: Flemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, or their derivatives. Derivatives of terpenes include Terpenoids in their forms of hemiterpenoids, monoterpenoids, sesquiterpenoids, sesterterpenoid, sesquarterpenoids, tetraterpenoids, Triterpenoids, tetraterpenoids, Polyterpenoids, isoprenoids, and steroids. They may be forms: α-, β-, γ-, oχo-, isomers, or combinations thereof.

Examples of terpenes within the context of the inventive technology include: 7,8-dihydroionone, Acetanisole, Acetic Acid, Acetyl Cedrene, Anethole, Anisole, Benzaldehyde, Bergamotene (α-cis-Bergamotene) (α-trans-Bergamotene), Bisabolol (β-Bisabolol), Borneol, Bornyl Acetate, Butanoic/Butyric Acid, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (a-Cedrene Epoxide), Cedrol, Cembrene, Chlorogenic Acid, Cinnamaldehyde (α-amyl-Cinnamaldehyde) (a-hexyl-Cinnamaldehyde), Cinnamic Acid, Cinnamyl Alcohol, Citronellal, Citronellol, Cryptone, Curcumene (α-Curcumene) (γ-Curcumene), Decanal, Dehydrovomifoliol, Diallyl Disulfide, Dihydroactinidiolide, Dimethyl Disulfide, Eicosane/lcosane, Elemene (β-Elemene), Estragole, Ethyl acetate, Ethyl Cinnamate, Ethyl maltol, Eucalyptol/1,8-Cineole, Eudesmol (a-Eudesmol) (β-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1 (10),11-diene, Guaiacol, Guaiene (a-Guaiene), Gurjunene (α-Gurjunene), Herniarin, Flexanaldehyde, Flexanoic Acid, Humulene (a-Humulene) (β-Humulene), lonol (3-oxo-a-ionol) (β-IoηoI), lonone (a-lonone) (β-lonone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, Pinene, β-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, α-Selinene, a-Sinensal, β-Sinensal, β-Sitosterol, Squalene, Taxadiene, Terpin hydrate, Terpineol, Terpine-4-ol, α-Terpinene, y-Terpinene, Terpinolene, Thiophenol, Thujone, Thymol, a-Tocopherol, Tonka Undecanone, Undecanal, Valeraldehyde/Pentanal, Verdoxan, a-Ylangene, Umbelliferone, or Vanillin. Diterpenes: oridonin, phytol, and isophytol. Triterpenes: ursolic acid, oleanolic acid, among those list elsewhere.

To “xylosylated” means to attach a xylosyl moiety to a molecule.

The term “glycosidase inhibitor” and as used in the present invention is used to mean a compound, which can inhibit glycosidase enzymes which catalyze the hydrolysis of glycosidic bonds.

As used herein, the term “homologous” with regard to a contiguous nucleic acid sequence, refers to contiguous nucleotide sequences that hybridize under appropriate conditions to the reference nucleic acid sequence. For example, homologous sequences may have from about 70%-100, or more generally 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid nonspecific binding of the nucleic acid to non-target sequences under conditions where specific binding is desired, for example, under stringent hybridization conditions. The term “homolog” as used herein means a homologous protein having a similar enzymatic action. For example, a UTG homolog would be any similar homologous enzyme having glycosylation activity towards one or more terpenes or cannabinoids.

The term, “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell.

As used herein, a “cannabinoid” is a chemical compound (such as cannabinol, THC or cannabidiol) that is found in the plant species Cannabis among others like Echinacea; Acmella oleracea; Helichrysum umbraculigerum; Radula marginata (Liverwort) and Theobroma cacao, and metabolites and synthetic analogues thereof that may or may not have psychoactive properties. Cannabinoids therefore include (without limitation) compounds (such as THC) that have high affinity for the cannabinoid receptor (for example Ki<250 nM), and compounds that do not have significant affinity for the cannabinoid receptor (such as cannabidiol, CBD). Cannabinoids also include compounds that have a characteristic dibenzopyran ring structure (of the type seen in THC) and cannabinoids which do not possess a pyran ring (such as cannabidiol). Hence a partial list of cannabinoids includes THC, CBD, dimethyl heptylpentyl cannabidiol (DMHP-CBD), 6, l2-dihydro-6-hydroxy-cannabidiol (described in U.S. Pat. No. 5,227,537, incorporated by reference); (3S,4R)-7-hydroxy-A6-tetrahydrocannabinol homologs and derivatives described in U.S. Pat. No. 4,876,276, incorporated by reference; (+)-4-[4-DMH-2,6-diacetoxy-phenyl]-2-carboxy-6,6-dimethylbicyclo[3. l. l]hept-2-en, and other 4-phenylpinene derivatives disclosed in U.S. Pat. No. 5,434,295, which is incorporated by reference; and cannabidiol (−)(CBD) analogs such as (−)CBD-monomethylether, (−)CBD dimethyl ether; (−)CBD diacetate; (−)3′-acetyl-CBD monoacetate; and ±AFl l, all of which are disclosed in Consroe et al., J. Clin. Phannacol. 2l:428S-436S, 1981, which is also incorporated by reference. Many other cannabinoids are similarly disclosed in Agurell et al., Pharmacol. Rev. 38:31-43, 1986, which is also incorporated by reference.

As claimed herein, the term “cannabinoid” may also include different modified forms of a cannabinoid such as a hydroxylated cannabinoid or cannabinoid carboxylic acid, an acetylated glycoside cannabinoid, a methylated cannabinoid. For example, if a glycosyltransferase were to be capable of glycosylating a cannabinoid, a resulting cannabinoid glycoside would be included in the term cannabinoid as defined elsewhere, as well as the aforementioned modified forms. It may further include multiple glycosylation moieties.

Examples of cannabinoids are tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid, delta-9-tetrahydrocannabivarin, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-l-benzoxocin-5-methanol-cannabiripsol, trihydroxy-delta-9-tetrahydrocannabinol, and cannabinol. Examples of cannabinoids within the context of this disclosure include tetrahydrocannabinol and cannabidiol.

A “Cannabis extract” may include one or more compounds extracted from a Cannabis plant. In one embodiment, a “Cannabis extract” may include one or more terpenes extracted from a Cannabis plant. In one embodiment, a “Cannabis extract” may include one or more cannabinoids extracted from a Cannabis plant. In one embodiment, a “Cannabis extract” may include one or more terpenes and one or more cannabinoids extracted from a Cannabis plant. As also used herein, the term Cannabis include hemp.

As used herein, the term “transformation” or “genetically modified” refers to the transfer of one or more nucleic acid molecule(s) into a cell. A plant is “transformed” or “genetically modified” by a nucleic acid molecule transduced into the plant when the nucleic acid molecule becomes stably replicated by the plant. As used herein, the term “transformation” or “genetically modified” encompasses all techniques by which a nucleic acid molecule can be introduced into, such as a plant, or yeast cell and include both stable and transient transformations. Genes encoding by a combination polynucleotide and/or a homologue thereof, may be introduced into a plant, and/or plant cell using several types of transformation approaches developed for the generation of transgenic plants. Standard transformation techniques, such as Ti-plasmid Agrobacterium-mediated transformation, particle bombardment, microinjection, and electroporation may be utilized to construct stably transformed transgenic plants.

An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.”

A polynucleotide sequence is operably linked to an expression control sequence(s) (e.g., a promoter and, optionally, an enhancer) when the expression control sequence controls and regulates the transcription and/or translation of that polynucleotide sequence.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. The Table below, contains information about which nucleic acid codons encode which amino acids.

### Amino Acid Nucleic Acid Codons

| Amino Acid | Nucleic Acid Codons |
|---|---|
| Ala/A | GCT, GCC, GCA, GCG |
| Arg/R | CGT, CGC, CGA, CGG, AGA, |
| | AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |

The term “plant” or “plant system” includes whole plants, plant organs, progeny of whole plants or plant organs, embryos, somatic embryos, embryo-like structures, protocorms, protocorm-like bodies (PLBs), and culture and/or suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The invention may also include Cannabaceae and other Cannabis strains, such as C. sativa generally.

The term “expression,” as used herein, or “expression of a coding sequence” (for example, a gene or a transgene) refers to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide. The term “sequence identity” or “identity,” as used herein in the context of two nucleic acid or polypeptide sequences, refers to the residues in the two sequences that are the same when aligned for maximum correspondence over a specified comparison window.

The terms “approximately” and “about” refer to a quantity, level, value or amount that varies by as much as 30%, or in another embodiment by as much as 20%, and in a third embodiment by as much as 10% to a reference quantity, level, value or amount. As used herein, the singular form “a,” “an,” and “the” include plural references unless the context clearly dictates otherwise.

As used herein, “heterologous” or “exogenous” in reference to a nucleic acid is a nucleic acid that originates from a foreign species, or is synthetically designed, or, if from the same species, is substantially modified from its native form in composition and/or genomic locus by deliberate human intervention. A heterologous protein may originate from a foreign species or, if from the same species, is substantially modified from its original form by deliberate human intervention. As used herein, “endogenous” in reference to a nucleic acid is a nucleic acid that originates from a host the same species, or is substantially un-modified from its native form in composition and/or genomic locus by deliberate human intervention.

The term “prodrug” refers to a precursor of a biologically active pharmaceutical agent (drug). Prodrugs must undergo a chemical or a metabolic conversion to become a biologically active pharmaceutical agent. A prodrug can be converted ex vivo to the biologically active pharmaceutical agent by chemical transformative processes. In vivo, a prodrug is converted to the biologically active pharmaceutical agent by the action of a metabolic process, an enzymatic process or a degradative process that removes the prodrug moiety to form the biologically active pharmaceutical agent.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Design of Novel Yeast-Expression System for Terpene Bioconversion

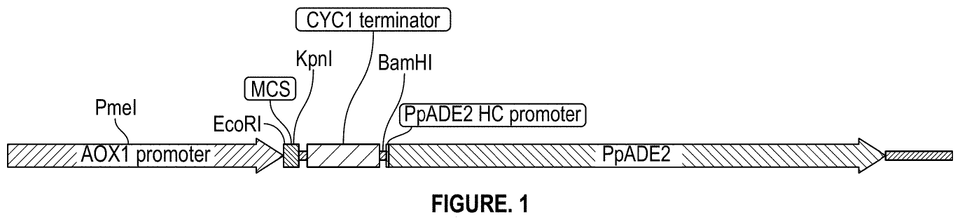

The present inventors generated an in vivo expression system for yeast-based terpene bioconversion, which in one preferred embodiment utilizes Invitrogen Pichiapink™ system. This model system is a eukaryotic expression system based on the methylotrophic yeast Pichia pastoris (Komagataella phaffii). The Pichiapink system includes protease-deficient host strains and allows both intracellular as well as secreted protein production. In addition, the use of the inducible promoter alcohol oxidase (AOX1) uncouples growth from production of desired proteins, so that cells are not stressed by the accumulation of recombinant protein during growth phase. PichiaPink strain 4 (herein referred to as wild-type, WT), a double knockout for proteases prb1, pep4 (to avoid degradation of desired protein) was utilized by the present inventors as the background strain in exemplary yeast transformations. The vector pPINK-HC was used as the backbone for expression of exemplary terpene glycosyltransferases (UGTs) in the yeast system. As shown in FIG. 1, this vector contains the ADE2 marker for selection on minimal media lacking adenine. Transformation and selection of transformants was conducted according to standard protocols known by those of ordinary skill in the art. All terpene isolates were obtained from True terpenes.

Expression analysis for introduced transgenes was carried out by RT-PCR. For yeast, 2 mL of a 2-day old culture induced by methanol overnight was centrifuged in a microfuge tube. The pellet was ground in a TissueLyser (QIAGEN Inc, USA). RNA was extracted following the EZNA plant RNA extraction kit (Omega Bio-tek Inc, USA). Up to a microgram of total RNA was used to synthesize cDNA using the superscript III cDNA synthesis kit (Thermo Fisher Scientific, USA). The cDNA was used to check for the expression of transgenes by RT-PCR.

### Example 2: Bioconversion of Terpene Glycosides in a Yeast-Based Expression System

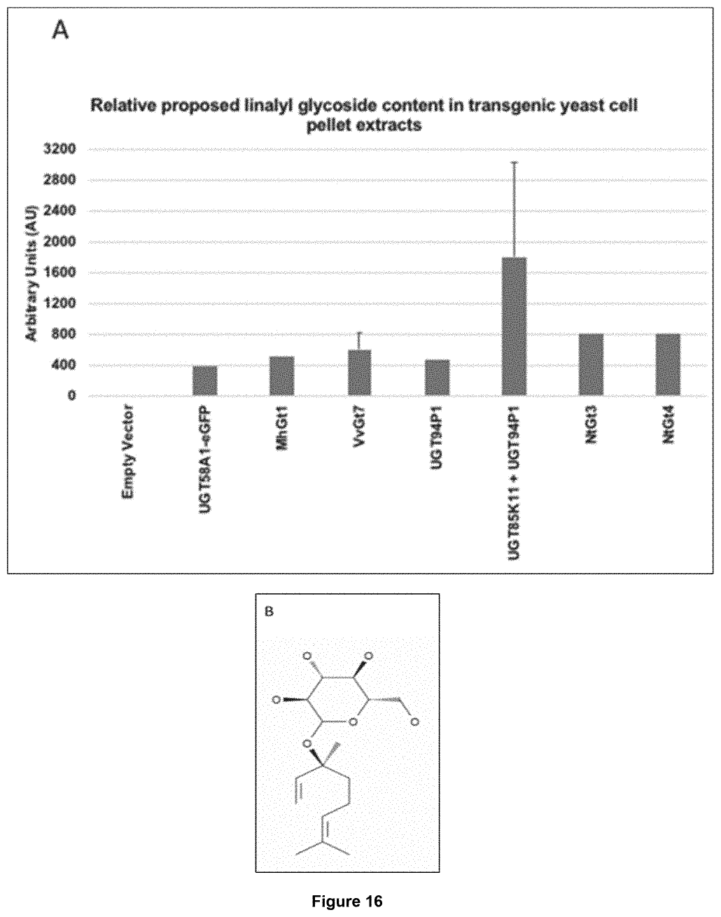

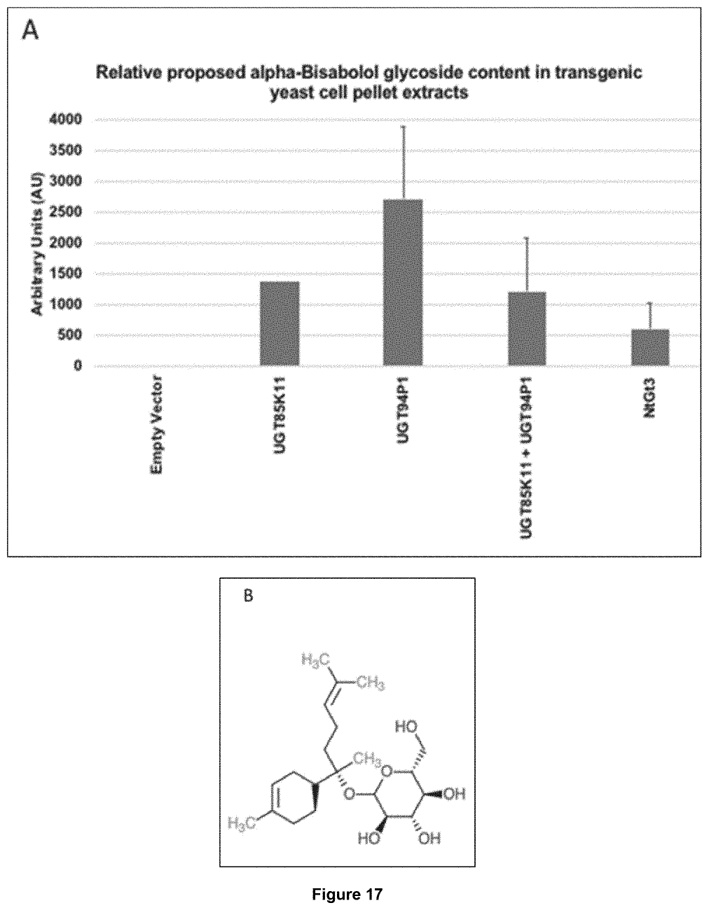

The present inventors conduct a 96-well screening procedure to identify the production of terpene glycosides in a novel yeast-based expression system. Cultures listed in Table 2 below were fed 100 μg/mL of linalool or alpha-bisabolol after overnight gene expression induction with 2% methanol. Cultures were harvested 48 h post terpene feeding. As shown in FIG. 16 and Table 3 below, results from the incubation of Pichia transgenic lines with individual terpenes indicate linalool is being glycosylated into linalyl glycoside by at least six enzymes tested including UGT58A1 (according to nucleotide sequence SEQ ID NO. 1, according to amino acid sequence SEQ ID NO. 2), MhGt1 (according to nucleotide sequence SEQ ID NO. 3, according to amino acid SEQ ID NO. 4), VvGt7 (according to nucleotide sequence SEQ ID NO. 9, according to amino acid SEQ ID NO. 10), UGT85K11 (according to nucleotide sequence SEQ ID NO. 5, according to amino acid SEQ ID NO. 6); UGT94P1 (according to nucleotide sequence SEQ ID NO. 7, according to amino acid SEQ ID NO. 8), NtGt3 (according to nucleotide sequence SEQ ID NO. 11, according to amino acid SEQ ID NO. 12), and NtGt4 (according to nucleotide sequence SEQ ID NO. 13, according to amino acid SEQ ID NO. 14), but not by empty vector. As shown in FIG. 17, at least three tested enzymes (UGT85K11, UGT94P1, and NtG3) successfully glycosylated alpha-bisabolol.

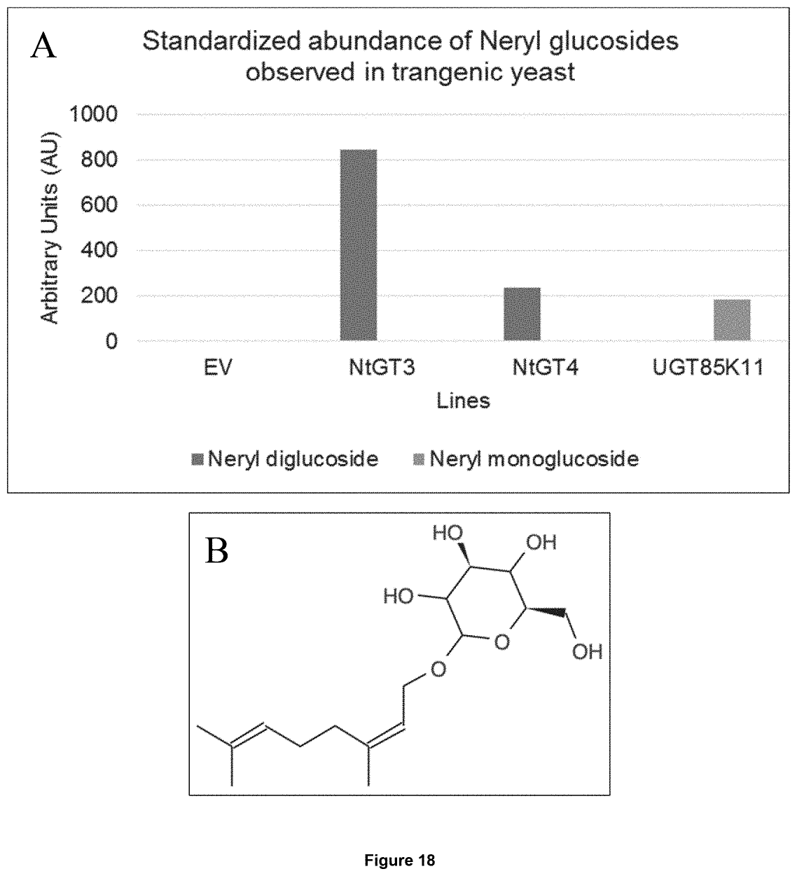

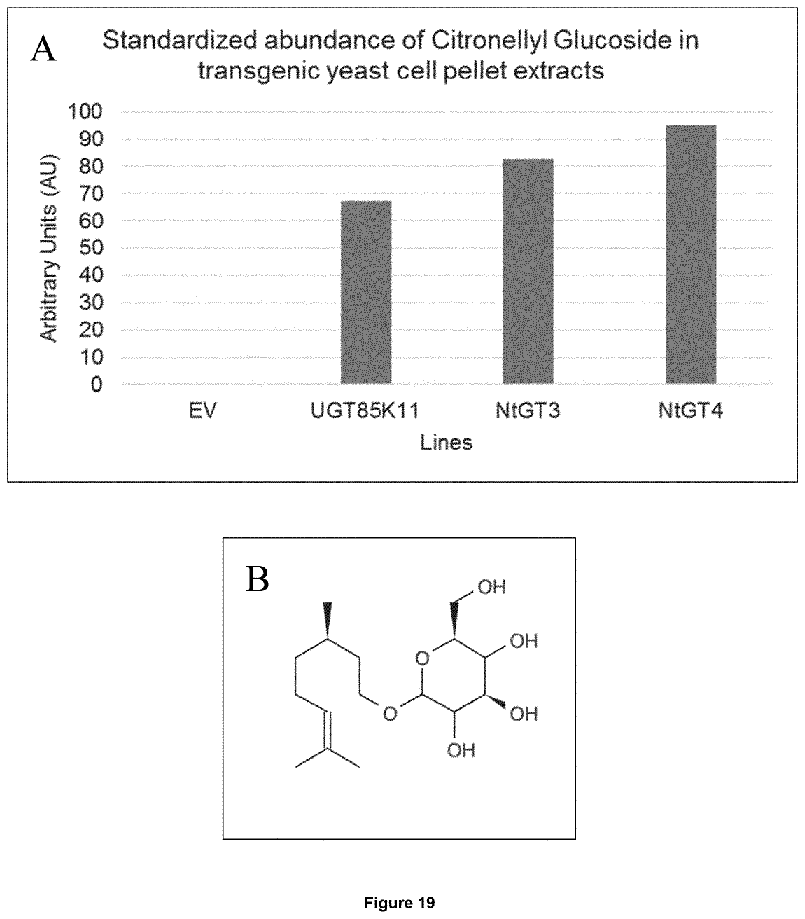

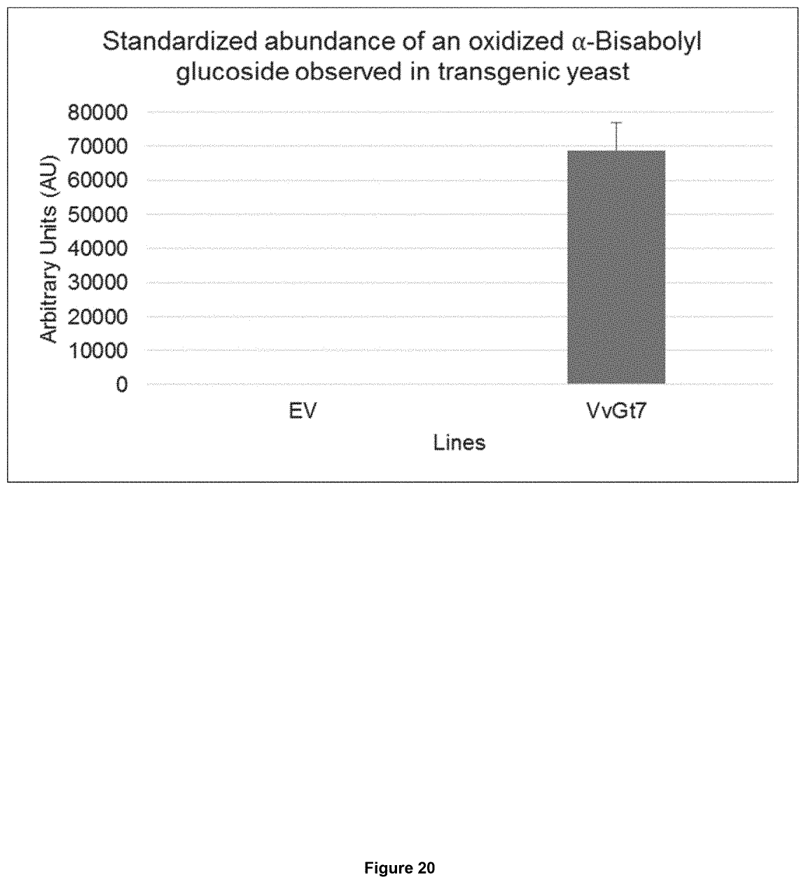

In another embodiment, the same yeast cultures as identified in Table 2 below were induced with 2% methanol overnight, and then fed with individual terpenes (alpha-bisabolol, nerol, citronellol) at 100 μg/mL final concentration. The cultures were harvested 2 days post-feeding with terpenes. There was no observed negative effect on yeast cell growth rates after treatment with terpenes at this concentration. As summarized in Table 3 below, Nerol was successfully bio-converted to neryl monoglucoside by the tea plant UGT85K11, but not by other enzymes or empty vector control (FIG. 18). Citronellol was converted into citronellyl monoglucoside by UGT85K11, NtGT3, and NtGT4, but not empty vector (FIG. 19). Alpha-bisabolol was converted into an oxidized alpha-bisabolyl monoglucoside by VvGT7 (FIG. 20).

### Example 3: Design of Cannabis Plant System for in Planta Production of Terpene Glycosides

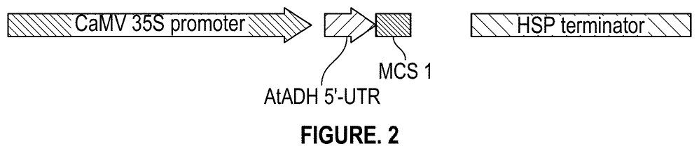

The present inventors generated a Cannabis plant based production systems for the bioconversion of terpenes into glycosylated terpene glycosides. As showing in FIG. 2, for plant expression of transgenes, the present inventors used Agrobacterium Ti-plasmid mediated transformation with the plant expression vector pRI201-AN, a binary vector for high-level expression of a foreign gene in dicotyledonous plants carrying the constitutive 35S promoter and an Arabidopsis Alcohol dehydrogenase (AtAdh) as a translational enhancer. All genes of interest were cloned in the multiple cloning site.

Overnight cultures of Agrobacterium AGL1 expressing UGT94P1, UGT85K11, and NtGT4 were transferred to a 250 mL flask with 50 mL LB medium supplemented with 50 mg/L of Kanamycin, 50 mg/L of Gentamycin and 10 mg/L of Rifampicin and grown for 4-8 hours until the optical density at 600 nm (OD600) reached approximately between 0.75 and 1. The cells were pelleted in a centrifuge at room temperature and resuspended 45 mL of infiltration medium containing 5 g/L D-glucose, 10 mM MES, 10 mM MgCl2 and 100 μM acetosyringone. Hemp plants (Youngsim 10 genotype), 4-weeks old, were vacuum-infiltrated with Agrobacterium tumefaciens AGL1 cultures expressing UGT94P1, UGT85K11, and NtGT4.

Expression of the transgenes was confirmed 2-4 days after infiltration by RT-PCR. For RT-PCR analysis, 100 mg of leaf tissue were frozen in liquid nitrogen and ground in a TissueLyser (QIAGEN Inc, USA). RNA was extracted following the EZNA plant RNA extraction kit (Omega Bio-tek Inc, USA). Up to a microgram of total RNA was used to synthesize cDNA using the superscript III cDNA synthesis kit (Thermo Fisher Scientific, USA). The cDNA was used to check for the expression of transgenes by RT-PCR.

### Example 4: In Planta Production of Terpene Glycosides in a Cannabis-Based Bioconversion System

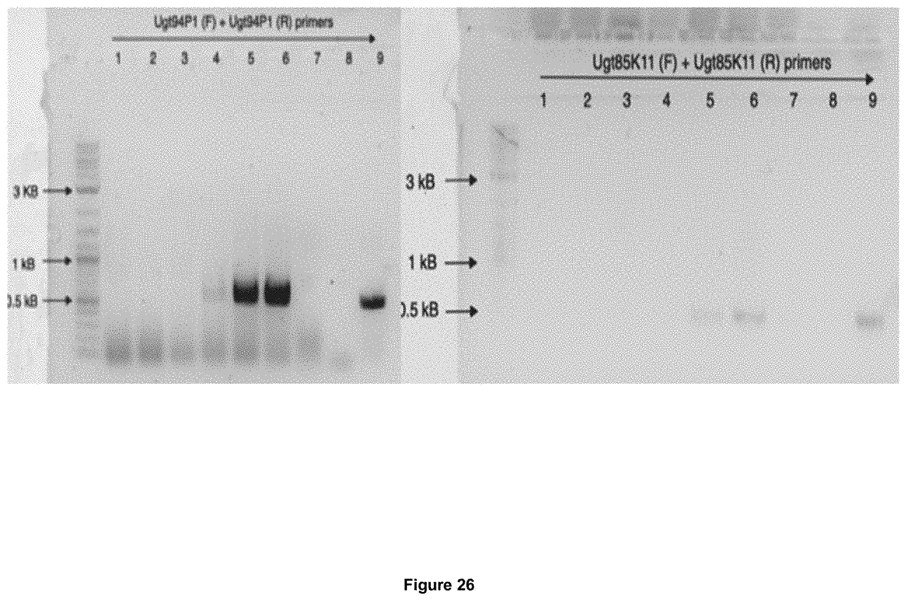

In one preferred embodiment, the present inventors demonstrated in in vivo production of terpene glycosides in a Cannabis plant. In this embodiment, four weeks old, Cannabis (Hemp) plants (Youngsim 10 genotype), were vacuum-infiltrated with Agrobacterium tumefaciens AGL1 cultures expressing UGT94P1 and UGT85K11 together, or NtGT4 alone. The wild-type concentration of free terpenes present in these samples prior to infiltration was previously determined. Samples were harvested 2-10 days post infiltration, weighted, and extracts analyzed for the formation of terpene glycosides. As shown in FIG. 26, RT-PCR analysis was conducted 2 DPI and confirmed expression of transgenes.

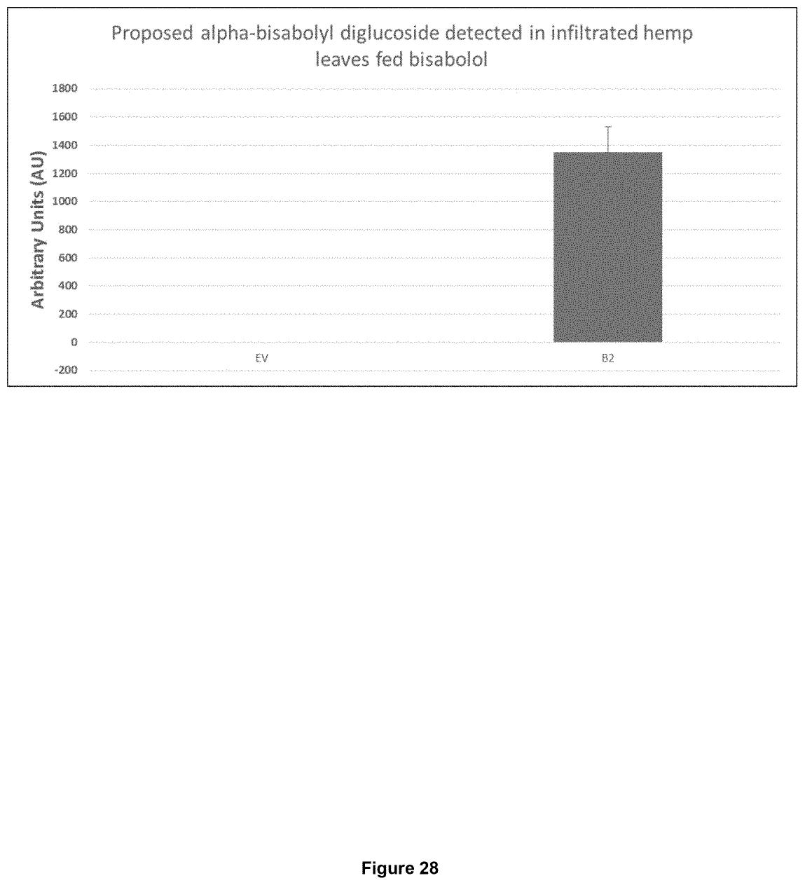

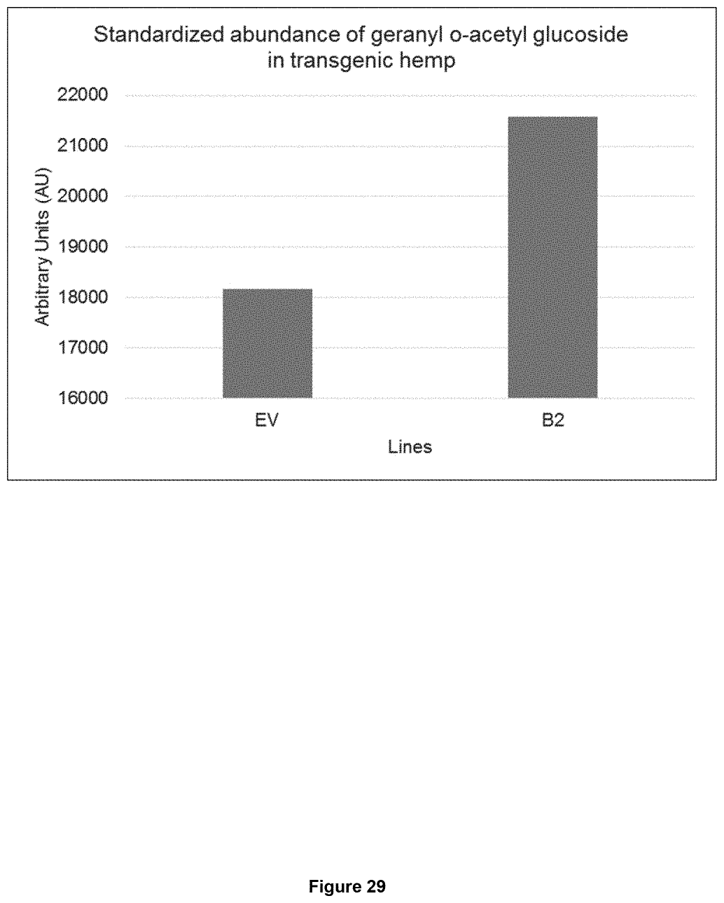

Agrobacterium-infiltrated samples relying on endogenous terpene pools identified above were harvested 5 days post infiltration and analyzed for possible terpene conjugate formation. In one embodiment, While alpha-bisabolyl diglucoside was detected in samples co-expressing UGT85K11 and UGT94P1, this product was not detected in the empty vector (prI201-AN) control samples (FIG. 28). Another conjugated product observed was geranyl o-acetyl glucoside, in which detection was increased in UGT85K11/UGT94P11-expressing lines, compared to control (FIG. 29).

### Example 5: Production of Terpene Glycosides in a Tobacco Cell Suspension Culture Based Bioconversion System

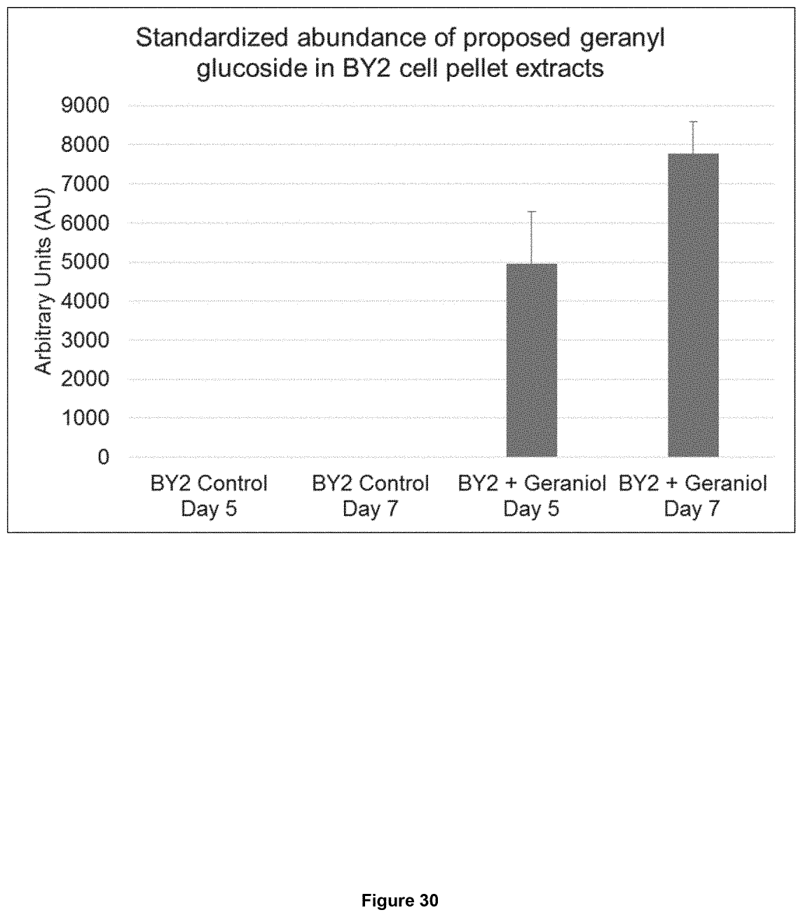

The present in inventors demonstrated a novel non-GM method for the bioconversion of terpenes using wild type tobacco (BY2) cell suspension cultures for in vivo glycosylation of terpenes. In this embodiment, healthy BY2 cell suspension cultures (15 mL) were fed in triplicates with 200 ug/mL of geraniol (1.290 uM). Cultures used as control were fed the equivalent methanol volume used to deliver the terpenes to treated cultures. The cultures were harvested 5 and 7 days-post treatment. As demonstrated in FIG. 30 and Table 7, analysis of cell pellet extracts indicates that geraniol was glycosylated by the tobacco BY2 endogenous glycosyltransferases. Moreover, geraniol monoglucoside was not detected in control samples (FIG. 30). Interestingly, the relative amount of glycosylated geraniol increases from 5 to 7 days of incubation, further demonstrating that the bioconversion in BY2 cell suspension cultures occurs slower than in yeast, in which most bioconversion occurs within 48 h.

### Example 6: Materials and Methods

Detection of Terpene Glycosides

In a preferred embedment, free volatile terpene and terpenyl glycoside content may be analyzed by LC-MS/MS. Endogenous free and bound terpene content may further be screened in both wild type yeast and/or plant cell suspension cultures to establish an expected background profile. Additionally, Cannabis-specific terpenes may be screened by feeding a known concentration of Cannabis terpene standard mixtures to yeast and/or plant cell suspension cultures. Control cultures may be incubated with the delivery solvent (isopropanol). Cultures can then be incubated for 16 hr, followed by extraction for downstream analysis by LC-MS/MS

Free and bound terpenes may be extracted from 0.2-1.0 g starting material or equivalent, in the case of cell suspension cultures, and this material can be homogenized into 10 volumes methanol. For free terpenes, an incubation period of 1 hr may be followed by centrifugation (10,000×g, 5 min) to clear cellular debris. The filtered supernatant can then be spiked with either 0.3% toluene or 5 μM 4-methyl-2-pentanol as an internal standard (ISTD). For terpenyl glycosides, the filtered supernatant can be applied to a pre-conditioned Supleco© XAD-2 solid-phase extraction (SPE) cartridge (Sigma, US), which may then be washed with dichloromethane to remove phenolics and free terpenoids. Bound terpenes may then be eluted with methanol, and concentrated to dryness under nitrogen.

Extracts may then be analyzed by LC-MS/MS using an Acquity M-Series UPLC system coupled with a Synapt G2-Si mass spectrometer (Waters, US). Briefly, a 2 μL aliquot may be injected onto a HALO C18 2.7 μm fused-particle column 150 mm×300 □m (Sciex, US). Mobile phases (A) water with 0.1% formic acid and (B) methanol with 0.1% formic can be used for a gradient separation over 20 min at a flow rate of 0.1 mL min⁻¹, and atmospheric pressure chemical ionization (APCI) may be used to ionize free terpene species. MS and MS/MS data can be collected using multiple reaction monitoring (MRM), with the proposed retention times, collision energies and MRM transitions for free terpenes are listed in table 9 directly below. Gradient separation (retention time) and collision energy may differ between LC-MS/MS systems.

The dominant free terpenoids present in C. sativa are listed above; however, other free terpenoids have been observed within C. sativa, H. lupulus and N. tabacum, and additional MRM parameters may be determined during screening. Additionally, MRM parameters can be established for terpenyl glycosides. Previous reports have also indicated a high degree of promiscuity for certain glycosyltransferases; therefore, the method can be expanded to screen for additional non-target products, such as flavonoids, steroids, phenolic compounds, alcohols, and aldehydes and the like.

Sample Preparation for the Analysis of Water-Soluble Terpenes

From yeast and tobacco cell culture: Yeast and BY2 cell suspension cultures were centrifuged at 4000 rpm, 10 min, and the supernatant were collected in fresh 15 mL falcon tubes. Samples were processed following generally known protocols by those of ordinary skill in the art. Cell pellets were lyophilized overnight, and dry cell weights were collected. Dried cells were extracted with 5 mL methanol containing 0.1 ppm 7-hydroxycoumarin as an internal standard, and homogenized in an ultrasonic bath for 30 min. Supernatant and dried cell extracts were cleaned up by solid-phase extraction (SPE) using 6 mL Resprep bonded reversed-phase (C18) SPE cartridges (Restek). Briefly, cartridges were pre-conditioned with three bed volumes of water, samples were applied, washed with one bed volume of water and one bed volume of 30% methanol, and terpene glycosides were eluted in 5 mL of 100% methanol and free terpenes were eluted in 1 mL of 100% hexane.

From leaf tissue: Hemp leaves were processed following generally known protocols by those of ordinary skill in the art. with the following modifications. Hemp leaves were weighed and then ground in liquid nitrogen with a micropestle in a 2 mL centrifuge tube. Free and glycosylated terpenes were extracted with 1 mL acetone and incubated for 10 min. Extracts were centrifuged (15,000 rpm×2 min×RT) to clear cell debris and then split for analysis by LC-MS and GD-FID. Acetone extracts were dried under a nitrogen stream, and extracts for LC-MS analysis were dissolved in 70% methanol and extracts were dissolved in hexane.

Liquid Chromatography-Mass Spectrometry (LC-MS)

Dried extracts containing terpene glycosides were resolubilized in 1 mL of 70% methanol and 1 μL injections were made onto an HSS T3 C18 column (300 μm×150 mm, particle size 1.8 μm) maintained at 40° C. and equipped to an ACQUITY M-Class UPLC System and a Synapt G2-Si HDMS (Waters). A flow rate of 5.0 μL/min is used with mobile solvents (A) acetonitrile with 0.1% formic acid and (B) water with 0.1% formic acid following a linear gradient: initial conditions 85:15% (A:B %) for 2 minutes, linear ramp to 15:85% in 12 min, hold at 15:85% for 4.5 min, then equilibrate back to initial conditions 85:15% for 5.5 min and a total run time of 22 min. A LockMass solution of 200 μg/mL leucine enkephalin (554.2615 m/z) is infused through an auxiliary pump at a flow rate of 5.0 μL/min to maintain mass accuracy.

Data are acquired in negative ionization mode (ES-) using a data-independent acquisition (MSe) method in continuum mode. Sample and lockspray capillary voltages are set to 2.0 and 3.2 kV, respectively, and sample cone and cone offset are set to 30 and 40 V, respectively. Cone and desolvation gases are set to 20 and 500 L/hr and nebulizer gas is maintained at 6.5 bar. MS acquisition is performed from 0.0-22.0 minutes over a mass range of 100-600 m/z with a 0.486 s scan time with 0.014 s interscan delay. A high energy collision ramp of 5-30 V is applied, and LockSpray measurements are acquired every 30 s.

Data Processing

LC-MS data were processed in UNIFI v 9.3 (Waters) using an MSe analysis method developed to screen for both predicted and known terpene glycosides. Free terpenes were not detected by LC-ESI-MS. A terpene glycoside library was generated in-house using known and predicted structures drawn and exported as (.mol) files in ChemDoodle 2D Sketcher (ChemDoodle). Fragmentation ions produced by cleavage of the glyosidic bond [C₆H₁₀O₆]⁻ (179.0561 m/z) and [C₆H₉O₅]⁻ (161.0456 m/z), and [C₂H₄O₂]⁻ (59.0142 m/z) produced by fragmentation of glucose, were used to identify terpene glycosides. In addition, transformed products resulting from both phase I and II metabolism were also screened, including acetylation, hydration, dehydration, reduction and desaturation. All LC-MS data are presented as signal intensity—counts per second (CPS)—standardized to dry cell weight.

Mass Spectral Analysis of Water-Soluble Terpenes: Identification of Modified Terpenes by Mass Spectrometry

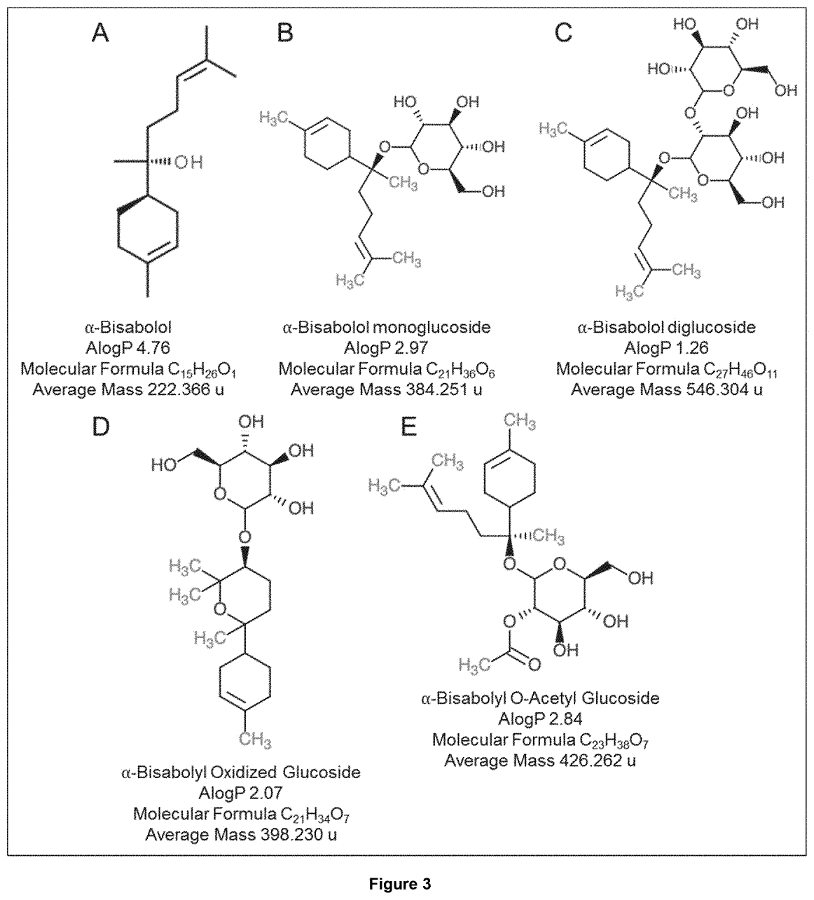

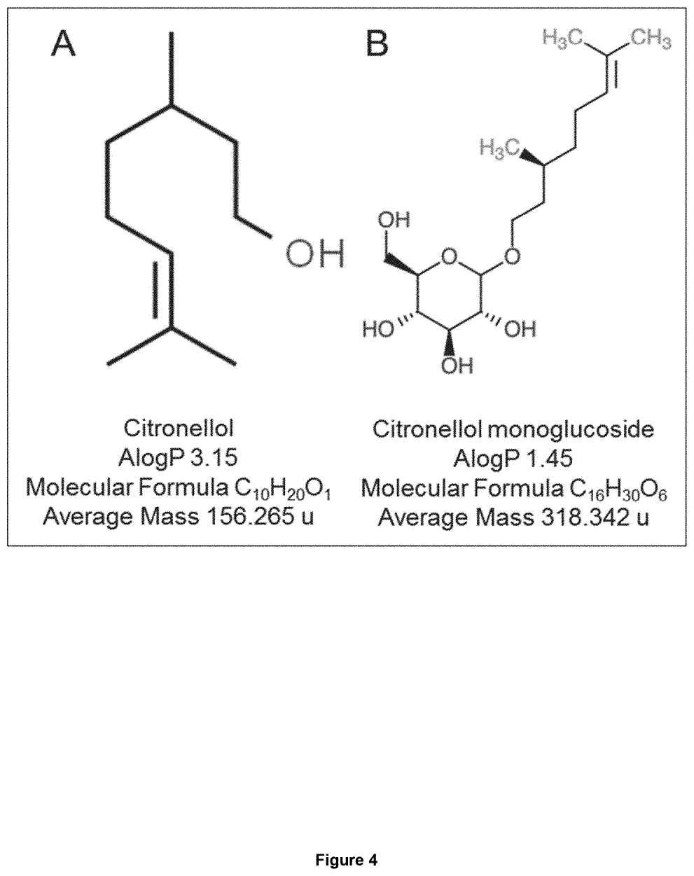

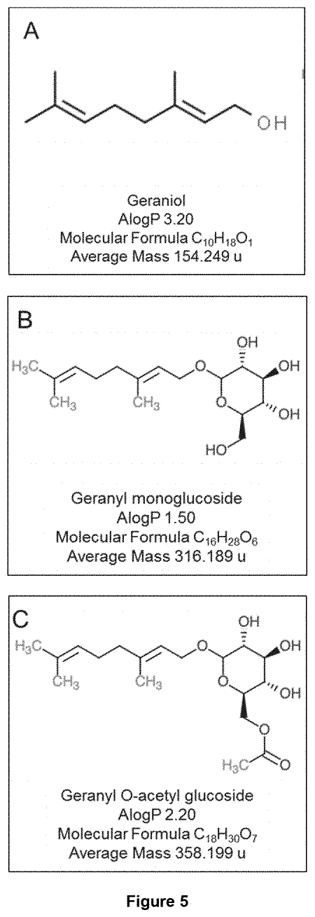

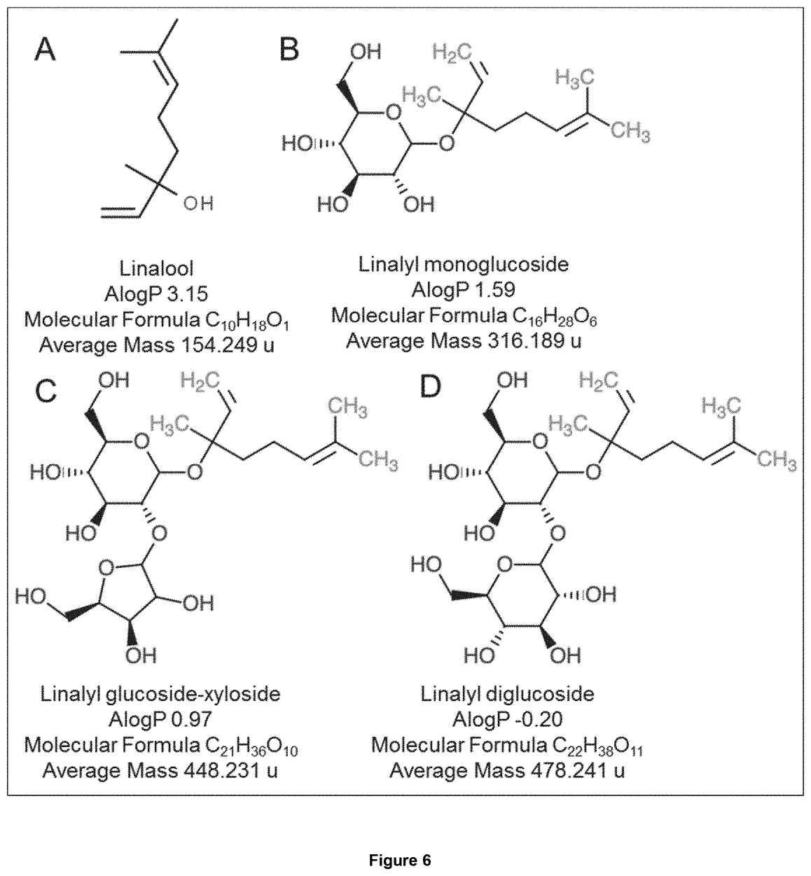

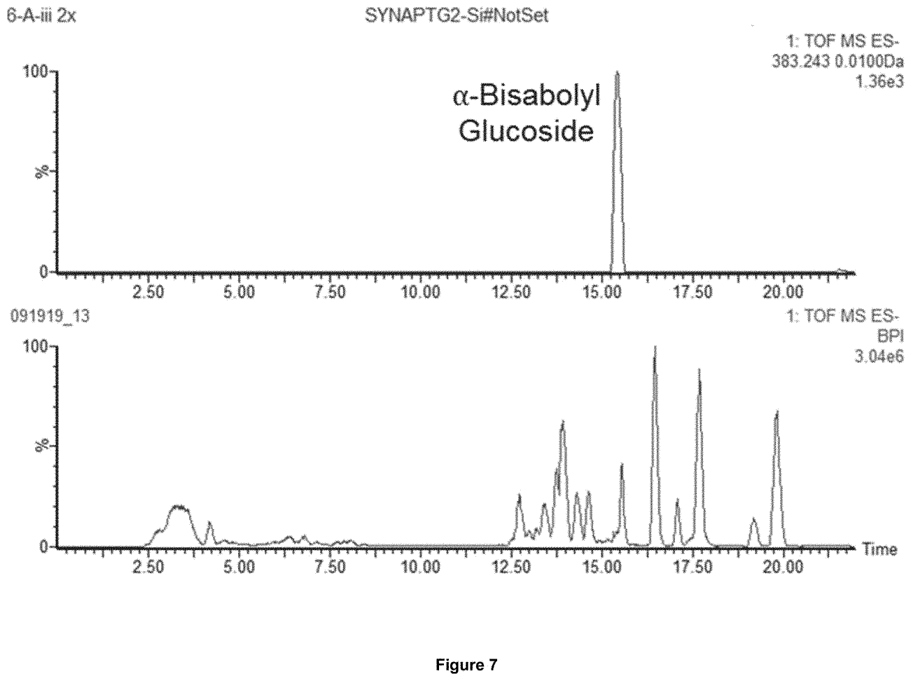

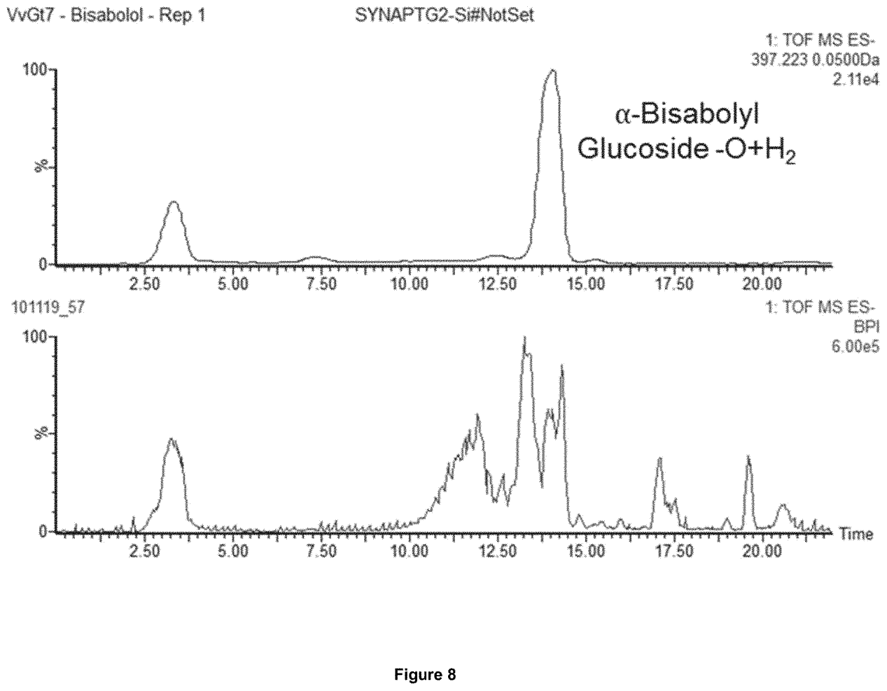

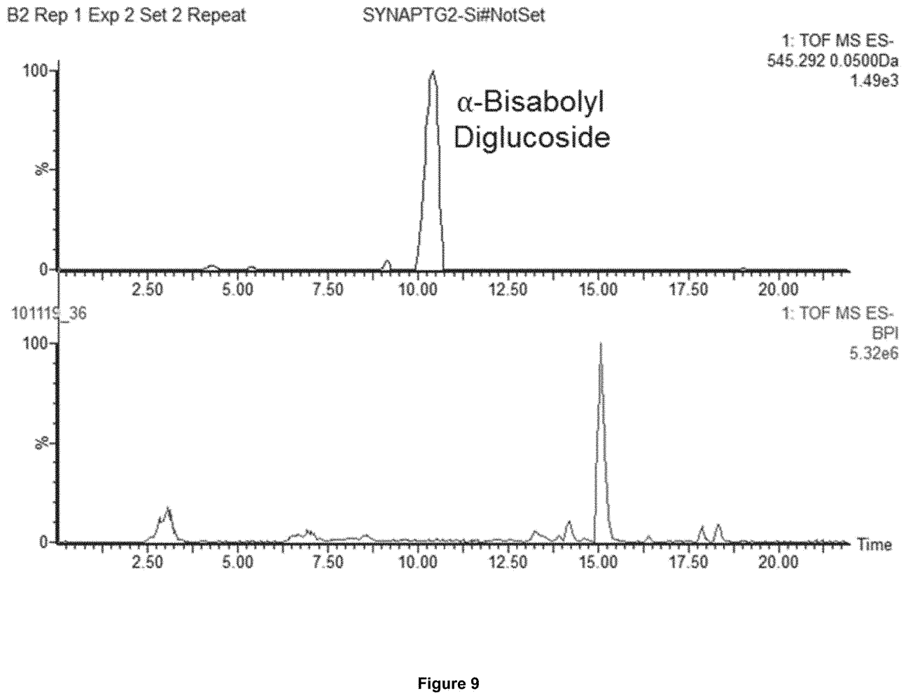

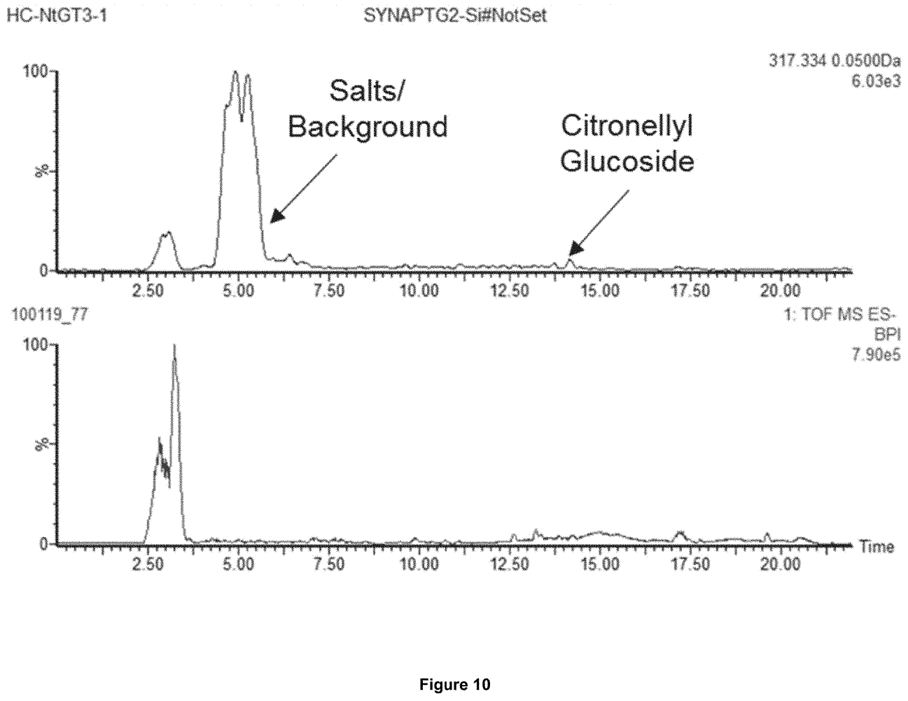

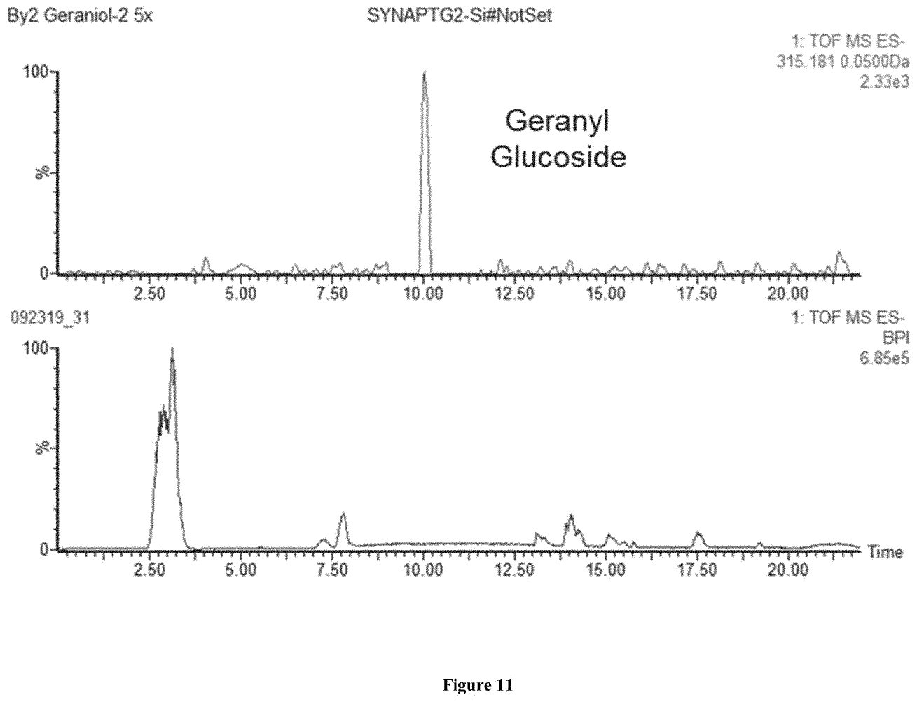

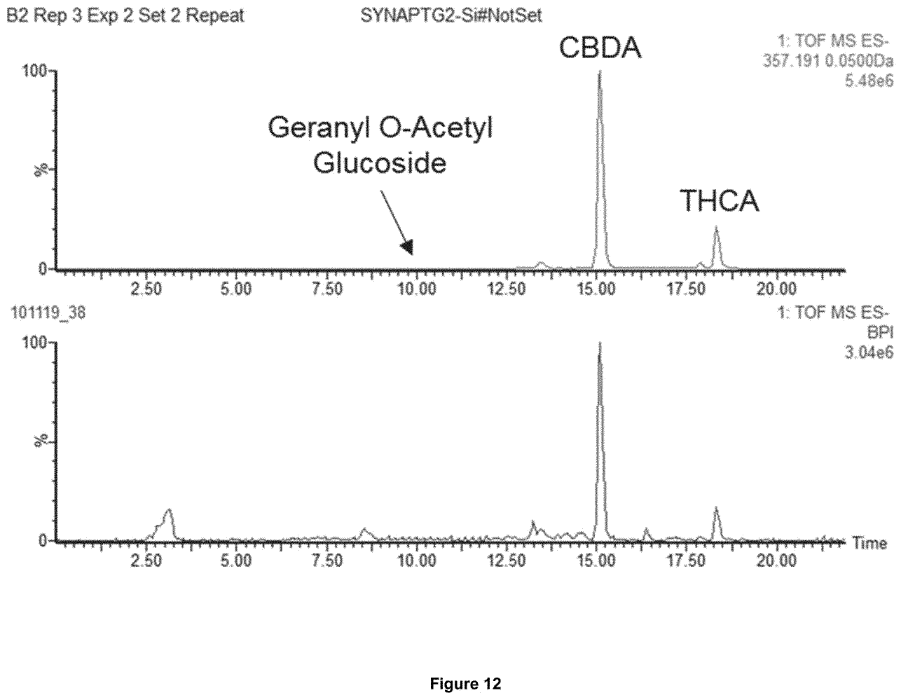

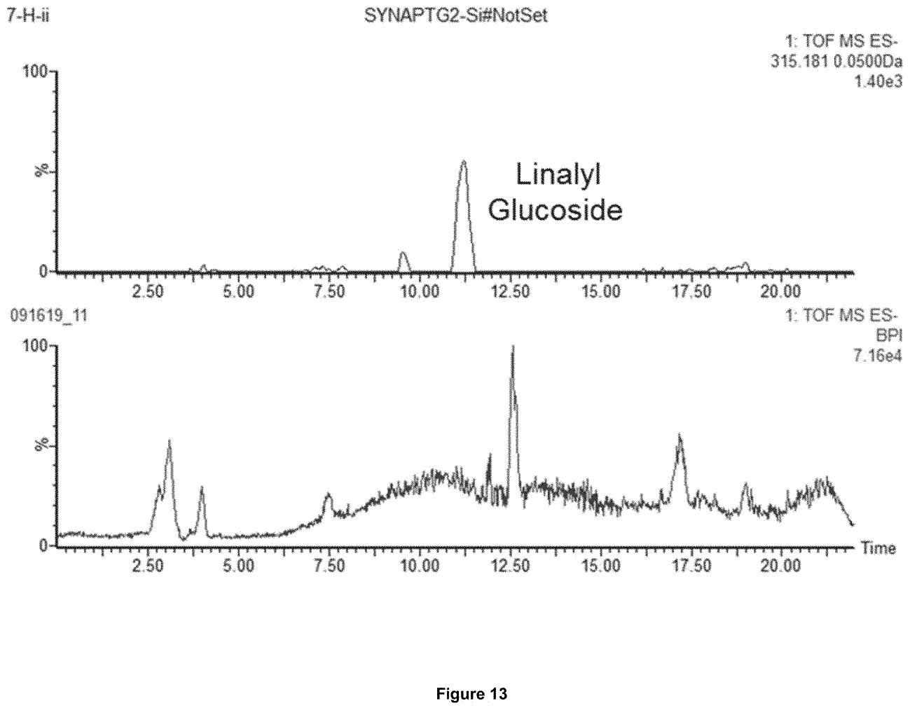

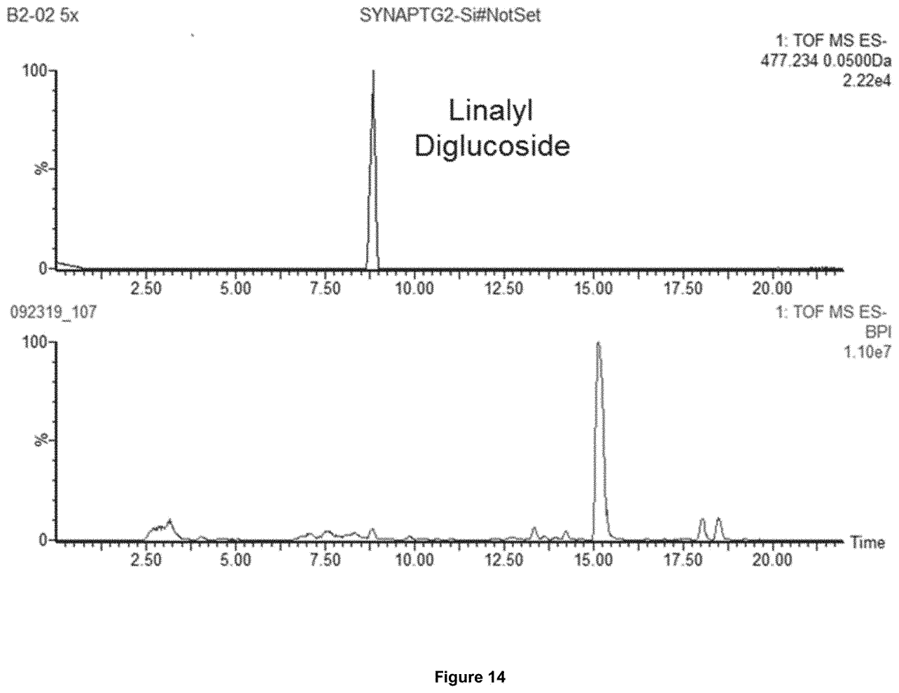

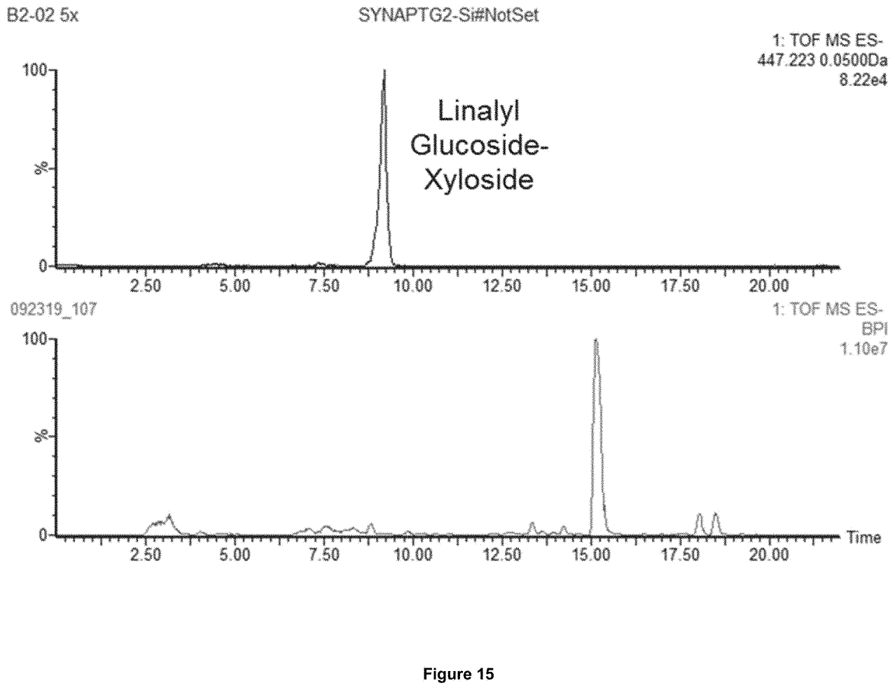

The present inventors used mass spectroscopy analysis to detect bioconversion products generated from UGTs in transgenic yeast, BY2 cells and hemp. Known and predicted glycosylation reactions for individual terpene glycosides, along with empirical data from chromatographic results, were used to generate both known and predicted structures and physiochemical properties indicating increased water solubility for observed terpene glycosides (FIG. 3-6). Chromatographic analysis produced extracted ion chromatograms indicating predicted species (FIG. 7-15). Terpene glycosides were identified based on accurate mass measurements using known and predicted molecular formulae. All measurements were within 5.0 ppm of expected mass-to-charge ratio values. Additionally, MS2 product ions (179.0561, 161.0456 and 59.0142 m/z) were used for species identification. Peak area standardized to dry cell weight was used to compare relative abundance between treatments. In this study, eight terpene glycoside species were identified across three platforms.

Each of the below embodiments is specifically incorporated into the specification of the current application. Each of the below embodiments may be amended and presented as a formal claim and further represents an independent invention. Also, it should be specifically noted that for each preserved embodiment and supporting description in the specification, the term “glycosylated terpene” encompasses glycosylated-xylosylated terpene, and/or xylosylated terpenes.

1. A composition comprising:
an aqueous solution;
water-soluble terpene dissolved in said aqueous solution wherein said water-soluble terpene comprises a glycosylated terpene, and/or an acetylated terpene glycoside, and/or a mixture of both;
wherein said composition may be introduced to a food or beverage.
   2. The composition of embodiment 1, wherein said glycosylated terpene, and/or said acetylated terpene glycoside were generated in vivo.
   3. The composition of embodiment 1, wherein said glycosylated terpene, and/or said acetylated terpene glycoside were generated in vitro.
   4. The composition of embodiment 1, wherein said water-soluble terpene is non-psychoactive.
   5. The composition of embodiment 1, wherein said aqueous solution comprises an aqueous solution selected from the group consisting of: saline, purified water, ethanol.
   6. The composition of embodiment 1, wherein said aqueous solution comprises propylene glycol, deionized water, an alcohol.
   7. The composition of embodiment 1, wherein said alcohol comprises ethanol.
   8. The composition of embodiment 7, further comprising a buffer.
   9. The composition of embodiment 8, wherein said buffer maintains said aqueous solution at a pH below 7.4.
   10. The composition of embodiment 7, further comprising formic acid, or ammonium hydroxide.
   11. A consumable food additive comprising at least one water-soluble glycosylated terpene.
   12. A consumable food additive as described in embodiment 11 and further comprising a food additive polysaccharide.
   13. A consumable food additive as described in embodiment 12 wherein said food additive polysaccharide comprises dextrin and/or maltodextrin.
   14. A consumable food additive as described in embodiment 11 and further comprising a emulsifier.
   15. A consumable food additive as described in embodiment 14 wherein said emulsifier is selected from the group consisting of: gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.
   16. A consumable food additive as described in embodiment 11, wherein said water-soluble glycosylated terpene is a non-psychoactive terpene.
   17. A consumable food additive as described in embodiment 11, wherein said water-soluble glycosylated terpene is generated in vivo.
   18. A consumable food additive as described in embodiment 11, wherein said water-soluble glycosylated terpene is generated in vitro.
   19. A consumable food additive as described in embodiment 13, wherein said consumable food additive is a homogenous composition.
   20. A consumable food additive as described in embodiment 11, and further comprising a flavoring agent.
   21. A consumable food additive as described in embodiment 20 wherein said flavoring agent comprises a flavoring agent selected from the group consisting of: Sucrose (sugar), glucose, fructose, sorbitol, mannitol, corn syrup, high fructose corn syrup, saccharin, aspartame, sucralose, acesulfame potassium (acesulfame-K), neotame.
   22. A consumable food additive as described in embodiment 11, and further comprising a coloring agent.
   23. A consumable food additive as described in embodiment 22 wherein said coloring agent comprises a coloring agent selected from the group consisting of: FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, saffron, Monosodium glutamate (MSG), hydrolyzed soy protein, autolyzed yeast extract, disodium guanylate or inosinate
   24. A consumable food additive as described in embodiment 11, and further comprising a surfactant.
   25. A consumable food additive as described in embodiment 24 wherein said surfactant comprises a surfactant selected from the group consisting of glycerol monostearate and polysorbate 80.
   26. A consumable food additive as described in embodiment 11, and further comprising a preservative.
   27. A consumable food additive as described in embodiment 26, wherein said preservative comprises a preservative selected from the group consisting of: ascorbic acid, citric acid, sodium benzoate, calcium propionate, sodium erythorbate, sodium nitrite, calcium sorbate, potassium sorbate, BHA, BHT, EDTA, tocopherols.
   28. A consumable food additive as described in embodiment 11 and further comprising a nutrient supplement.
   29. A consumable food additive as described in embodiment 28, wherein said nutrient supplement comprises a nutrient supplement selected from the group consisting of: thiamine hydrochloride, riboflavin, niacin, niacinamide, folate or folic acid, beta carotene, potassium iodide, iron or ferrous sulfate, alpha tocopherols, ascorbic acid, Vitamin D, amino acids, multi-vitamin, fish oil, co-enzyme Q-10, and calcium.
   30. A consumable food additive as described in embodiment 11 and further comprising at least one water-soluble acetylated terpene glycoside.
   31. A consumable food additive comprising at least one water-soluble acetylated terpene glycoside.
   32. A consumable food additive as described in embodiment 31 and further comprising a food additive polysaccharide.
   33. A consumable food additive as described in embodiment 32 wherein said food additive polysaccharide comprises dextrin and/or maltodextrin.
   34. A consumable food additive as described in embodiment 32 and further comprising a emulsifier.
   35. A consumable food additive as described in embodiment 34 wherein said emulsifier is selected from the group consisting of: gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.
   36. A consumable food additive as described in embodiment 31, wherein said water-soluble acetylated terpene glycoside is a non-psychoactive terpene.
   37. A consumable food additive as described in embodiment 31, wherein said water-soluble acetylated terpene glycoside is generated in vivo.
   38. A consumable food additive as described in embodiment 31, wherein said water-soluble acetylated terpene glycoside is generated in vitro.
   39. A consumable food additive as described in embodiment 31, wherein said consumable food additive is a homogenous composition.
   40. A consumable food additive as described in embodiment 31, and further comprising a flavoring agent.
   41. A consumable food additive as described in embodiment 40 wherein said flavoring agent comprises a flavoring agent selected from the group consisting of: Sucrose (sugar), glucose, fructose, sorbitol, mannitol, corn syrup, high fructose corn syrup, saccharin, aspartame, sucralose, acesulfame potassium (acesulfame-K), neotame.
   42. A consumable food additive as described in embodiment 31, and further comprising a coloring agent.
   43. A consumable food additive as described in embodiment 42 wherein said coloring agent comprises a coloring agent selected from the group consisting of: FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, saffron, Monosodium glutamate (MSG), hydrolyzed soy protein, autolyzed yeast extract, disodium guanylate or inosinate
   44. A consumable food additive as described in embodiment 31, and further comprising a surfactant.
   45. A consumable food additive as described in embodiment 44 wherein said surfactant comprises a surfactant selected from the group consisting of glycerol monostearate and polysorbate 80.
   46. A consumable food additive as described in embodiment 31, and further comprising a preservative.
   47. A consumable food additive as described in embodiment 46, wherein said preservative comprises a preservative selected from the group consisting of: ascorbic acid, citric acid, sodium benzoate, calcium propionate, sodium erythorbate, sodium nitrite, calcium sorbate, potassium sorbate, BHA, BHT, EDTA, tocopherols
   48. A consumable food additive as described in embodiment 31 and further comprising a nutrient supplement.
   49. A consumable food additive as described in embodiment 48, wherein said nutrient supplement comprises a nutrient supplement selected from the group consisting of: thiamine hydrochloride, riboflavin, niacin, niacinamide, folate or folic acid, beta carotene, potassium iodide, iron or ferrous sulfate, alpha tocopherols, ascorbic acid, Vitamin D, amino acids, multi-vitamin, fish oil, co-enzyme Q-10, and calcium.
   50. A consumable food additive as described in embodiment 31 and further comprising at least one water-soluble glycosylated terpene.
   51. A consumable food additive comprising a mixture of at least one water-soluble glycosylated terpene and at least one water-soluble acetylated terpene glycoside.
   52. A consumable food additive as described in embodiment 51 and further comprising a food additive polysaccharide.
   53. A consumable food additive as described in embodiment 52 wherein said food additive polysaccharide comprises dextrin and/or maltodextrin.
   54. A consumable food additive as described in embodiment 51 and further comprising a emulsifier.
   55. A consumable food additive as described in embodiment 54 wherein said emulsifier is selected from the group consisting of: gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.
   56. A consumable food additive as described in embodiment 51, wherein said water-soluble acetylated terpene glycoside and said water-soluble glycosylated terpene are non-psychoactive terpenes.
   57. A consumable food additive as described in embodiment 51, wherein said water-soluble acetylated terpene glycoside and said water-soluble glycosylated terpene are generated in vivo.
   58. A consumable food additive as described in embodiment 51, wherein said water-soluble acetylated terpene glycoside and said water-soluble glycosylated terpene are generated in vitro.
   59. A consumable food additive as described in embodiment 51, wherein said consumable food additive is a homogenous composition.
   60. A consumable food additive as described in embodiment 51, and further comprising a flavoring agent.
   61. A consumable food additive as described in embodiment 60 wherein said flavoring agent comprises a flavoring agent selected from the group consisting of: Sucrose (sugar), glucose, fructose, sorbitol, mannitol, corn syrup, high fructose corn syrup, saccharin, aspartame, sucralose, acesulfame potassium (acesulfame-K), neotame.
   62. A consumable food additive as described in embodiment 51, and further comprising a coloring agent.
   63. A consumable food additive as described in embodiment 62 wherein said coloring agent comprises a coloring agent selected from the group consisting of: FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, saffron, Monosodium glutamate (MSG), hydrolyzed soy protein, autolyzed yeast extract, disodium guanylate or inosinate
   64. A consumable food additive as described in embodiment 51, and further comprising a surfactant.
   65. A consumable food additive as described in embodiment 64 wherein said surfactant comprises a surfactant selected from the group consisting of glycerol monostearate and polysorbate 80.
   66. A consumable food additive as described in embodiment 51, and further comprising a preservative.
   67. A consumable food additive as described in embodiment 66, wherein said preservative comprises a preservative selected from the group consisting of: ascorbic acid, citric acid, sodium benzoate, calcium propionate, sodium erythorbate, sodium nitrite, calcium sorbate, potassium sorbate, BHA, BHT, EDTA, tocopherols
   68. A consumable food additive as described in embodiment 51 and further comprising a nutrient supplement.
   69. A consumable food additive as described in embodiment 68, wherein said nutrient supplement comprises a nutrient supplement selected from the group consisting of: thiamine hydrochloride, riboflavin, niacin, niacinamide, folate or folic acid, beta carotene, potassium iodide, iron or ferrous sulfate, alpha tocopherols, ascorbic acid, Vitamin D, amino acids, multi-vitamin, fish oil, co-enzyme Q-10, and calcium.
   70. A consumable fluid comprising at least one water-soluble glycosylated terpene.
   71. A consumable fluid as described in embodiment 70, further comprising a food additive polysaccharide.
   72. A consumable fluid as described in embodiment 70, wherein said food additive polysaccharide comprises maltodextrin and/or dextrin.
   73. A consumable fluid as described in embodiment 73, wherein said maltodextrin is an aqueous maltodextrin solution.
   74. A consumable fluid as described in embodiment 73, wherein said aqueous maltodextrin solution further comprises sorbic acid and an acidifying agent to provide a food grade aqueous solution of maltodextrin having a pH of 2-4 and a sorbic acid content of 0.02-0.1% by weight.
   75. A consumable fluid as described in embodiment 70, wherein said consumable fluid is water.
   76. A consumable fluid as described in embodiment 75, wherein said consumable fluid is selected from the group consisting of: an alcoholic beverage; a non-alcoholic beverage, a noncarbonated beverage, a carbonated beverage, a cola, a root beer, a fruit-flavored beverage, a citrus-flavored beverage, a fruit juice, a fruit-containing beverage, a vegetable juice, a vegetable containing beverage, a tea, a coffee, a dairy beverage, a protein containing beverage, a shake, a sports drink, an energy drink, and a flavored water.
   77. A consumable fluid as described in embodiment 70, wherein said water-soluble glycosylated terpene is a non-psychoactive terpene.
   78. A consumable fluid as described in embodiment 70, wherein said water-soluble glycosylated terpene is generated in vivo.
   79. A consumable fluid as described in embodiment 70, wherein said water-soluble glycosylated terpene is generated in vitro.
   80. A consumable fluid as described in embodiment 70 further comprising at least one of: xanthan gum, cellulose gum, whey protein hydrolysate, ascorbic acid, citric acid, malic acid, sodium benzoate, sodium citrate, sugar, phosphoric acid, and water.
   81. A consumable fluid comprising at least one water-soluble acetylated terpene glycoside.
   82. A consumable fluid as described in embodiment 81 further comprising a food additive polysaccharide.
   83. A consumable fluid as described in embodiment 81 wherein said food additive polysaccharide comprises maltodextrin and/or dextrin.
   84. A consumable fluid as described in embodiment 83, wherein said maltodextrin is an aqueous maltodextrin solution.
   85. A consumable fluid as described in embodiment 84, wherein said aqueous maltodextrin solution further comprises sorbic acid and an acidifying agent to provide a food grade aqueous solution of maltodextrin having a pH of 2-4 and a sorbic acid content of 0.02-0.1% by weight.
   86. A consumable fluid as described in embodiment 81, wherein said consumable fluid is water.
   87. A consumable fluid as described in embodiment 81, wherein said consumable fluid is selected from the group consisting of: an alcoholic beverage; a non-alcoholic beverage, a noncarbonated beverage, a carbonated beverage, a cola, a root beer, a fruit-flavored beverage, a citrus-flavored beverage, a fruit juice, a fruit-containing beverage, a vegetable juice, a vegetable containing beverage, a tea, a coffee, a dairy beverage, a protein containing beverage, a shake, a sports drink, an energy drink, and a flavored water.
   88. A consumable fluid as described in embodiment 81, wherein said water-soluble acetylated terpene glycoside is a non-psychoactive terpene.
   89. A consumable fluid as described in embodiment 81, wherein said water-soluble acetylated terpene glycoside is generated in vivo.
   90. A consumable fluid as described in embodiment 81, wherein said water-soluble acetylated terpene glycoside is generated in vitro.
   91. A consumable fluid as described in embodiment 81 further comprising at least one of: xanthan gum, cellulose gum, whey protein hydrolysate, ascorbic acid, citric acid, malic acid, sodium benzoate, sodium citrate, sugar, phosphoric acid, and water.
   92. A consumable gel comprising at least one water-soluble glycosylated terpene and gelatin in an aqueous solution.
   93. A consumable gel as described in embodiment 92 wherein said water-soluble glycosylated terpene is generated in vivo.
   94. A consumable gel as described in embodiment 92 wherein said water-soluble glycosylated terpene is generated in vitro.
   95. A consumable gel comprising at least one water-soluble acetylated terpene glycoside and gelatin in an aqueous solution.
   96. A consumable gel as described in embodiment 95 wherein said water-soluble acetylated terpene glycoside is generated in vivo.
   97. A consumable gel as described in embodiment 95 wherein said water-soluble acetylated terpene glycoside is generated in vitro.
   98. A consumable gel comprising at least one water-soluble acetylated terpene glycoside, at least one water-soluble glycosylated terpene and gelatin in an aqueous solution.
   99. A consumable gel as described in embodiment 98 wherein said water-soluble acetylated terpene glycoside and said water-soluble acetylated terpene glycoside are generated in vivo.
   100. A consumable gel as described in embodiment 99 wherein said water-soluble acetylated terpene glycoside and said water-soluble acetylated terpene glycoside are generated in vitro.
   101. A method of making a consumable fluid additive comprising the steps:
solubilizing a water-soluble glycosylated terpene with a food additive polysaccharide to provide an aqueous solution containing said water-soluble glycosylated terpene and said food additive polysaccharide; and
adding said water-soluble glycosylated terpene and food additive polysaccharide aqueous solution to a consumable fluid.
   102. The method of embodiment 101, wherein said food additive polysaccharide is selected from the group consisting of: maltodextrin and/or dextrin.
   103. The method of embodiment 102, wherein said food additive polysaccharide is maltodextrin.
   104. The method of embodiment 103, wherein said maltodextrin is an aqueous maltodextrin solution.
   105. The method of embodiment 104, wherein said aqueous maltodextrin solution further comprises sorbic acid and an acidifying agent to provide a food grade aqueous solution of maltodextrin having a pH of 2-4 and a sorbic acid content of 0.02-0.1% by weight.
   106. The method of embodiment 104, wherein said consumable fluid is water.
   107. The method of embodiment 106, wherein said consumable fluid is selected from the group consisting of: an alcoholic beverage; a non-alcoholic beverage, a noncarbonated beverage, a carbonated beverage, a cola, a root beer, a fruit-flavored beverage, a citrus-flavored beverage, a fruit juice, a fruit-containing beverage, a vegetable juice, a vegetable containing beverage, a tea, a coffee, a dairy beverage, a protein containing beverage, a shake, a sports drink, an energy drink, and a flavored water.
   108. The method of embodiment 101, wherein said water-soluble glycosylated terpene is a non-psychoactive terpene.
   109. The method of embodiment 101, wherein said water-soluble glycosylated terpene is generated in vivo.
   110. The method of embodiment 101, wherein said water-soluble glycosylated terpene is generated in vitro.
   110. The method of embodiment 101, and further comprising the step of adding a flavor to said consumable fluid.
   111. The method of embodiment 101, further comprising the step of adding at least one of: xanthan gum, cellulose gum, whey protein hydrolysate, ascorbic acid, citric acid, malic acid, sodium benzoate, sodium citrate, sugar, phosphoric acid, and water.
   112. A composition comprising:
a first quantity of water;
a water-soluble terpene solubilized in said first quantity of water; and
at least one of: xanthan gum, cellulose gum, whey protein hydrolysate, ascorbic acid, citric acid, malic acid, sodium benzoate, sodium citrate, sugar, phosphoric acid, and/or a sugar alcohol.
   113. The composition of embodiment 112, wherein said water-soluble terpene comprises a glycosylated water-soluble terpene, an acetylated water-soluble terpene or a mixture of both.
   114. The composition of embodiment 113, wherein said water-soluble terpene is non-psychoactive.
   115. The composition of embodiment 112, and further comprising ethanol.
   116. The composition of embodiment 112, comprising less than 10 mass % water.
   117. The composition of embodiment 112, comprising more than 95 mass % water.
   118. The composition of embodiment 113, comprising about 0.1 mg to about 1000 mg of the water-soluble terpene.
   119. The composition of embodiment 113, comprising about 0.1 mg to about 500 mg of the water-soluble terpene.
   120. The composition of embodiment 113, comprising about 0.1 mg to about 200 mg of the water-soluble terpene.
   121. The composition of embodiment 113, comprising about 0.1 mg to about 100 mg of the water-soluble terpene.
   122. The composition of embodiment 113, comprising about 0.1 mg to about 100 mg of the water-soluble terpene.
   123. The composition of embodiment 113, comprising about 0.1 mg to about 10 mg of the water-soluble terpene.
   124. The composition of embodiment 113, comprising about 0.5 mg to about 5 mg of the water-soluble terpene.
   125. The composition of embodiment 113, comprising about 1 mg/kg to 5 mg/kg (body weight) in a human of the water-soluble terpene.
   126. The composition of embodiment 113, comprising water-soluble terpene in the range of 50 mg/L to 300 mg/L.
   127. The composition of embodiment 113, comprising water-soluble terpene in the range of 50 mg/L to 100 mg/L.
   128. The composition of embodiment 113, comprising water-soluble terpene in the range of 50 mg/L to 500 mg/L.
   129. The composition of embodiment 113, comprising water-soluble terpene over 500 mg/L.
   130. The composition of embodiment 113, comprising water-soluble terpene under 50 mg/L.
   131. The composition of embodiment 112, wherein the composition is homogeneous.
   132. The composition of embodiment 112, comprising a flavoring agent.
   133. The composition of embodiment 112, comprising a coloring agent.
   134. The composition of embodiment 112, comprising caffeine.
   135. The composition of embodiment 112, comprising a coloring agent.
   136. A composition comprising:
a first quantity of water;
a water-soluble terpene solubilized in said first quantity of water; and
a first quantity of ethanol in a liquid state.
   137. A composition according to embodiment 136 wherein said water-soluble terpene is a glycosylated terpene.
   138. A composition according to embodiment 136 wherein said water-soluble terpene is an acetylated terpene glycoside.
   139. A composition according to embodiment 136 wherein said water-soluble terpene is a mixture of glycosylated terpenes and acetylated terpene glycoside.
   140. A composition according to embodiment 137 wherein said glycosylated terpene is glycosylated in vivo.
   141. A composition according to embodiment 137 wherein said glycosylated terpene is glycosylated in vitro.
   142. A composition according to embodiment 138 wherein said acetylated terpene glycoside is acetylated in vivo.
   143. A composition according to embodiment 138 wherein said acetylated terpene glycoside is acetylated in vitro.
   144. A composition according to embodiment 139 wherein said acetylated terpene glycoside is acetylated in vivo and glycosylated terpene is glycosylated in vivo.
   145. A composition according to embodiment 139 wherein said acetylated terpene glycoside is acetylated in vitro and glycosylated terpene is glycosylated in vitro.
   146. A composition according to embodiment 136 wherein said ethanol can be up to about ninety-nine point nine-five percent (99.95%) by weight and said water-soluble terpene about zero point zero five percent (0.05%) by weight.
   147. A composition according to embodiment 136, wherein said water-soluble terpene is non-psychoactive.
   148. A composition according to embodiment 136, wherein said ethanol is an ethyl alcohol.
   149. A terpene enriched alcohol composition according to embodiment 148, wherein said ethyl alcohol has a proof greater than 100.
   150. A composition according to embodiment 148, wherein said ethyl alcohol has a proof less than 100.
   151. A composition according to embodiment 148, wherein said ethyl alcohol is a spirit.
   152. A composition according to embodiment 148, wherein said ethyl alcohol is beer, and/or wine.
   153. A terpene enriched alcohol composition for human consumption, said composition comprising by weight about:
a first quantity of water;
a water-soluble terpene solubilized in said first quantity of water; and
a first quantity of ethanol in a liquid state wherein said first quantity of ethanol is between 1% to 20% weight by volume.
   154. A terpene enriched alcohol composition according to embodiment 153 wherein said water-soluble terpene is a glycosylated terpene.
   155. A terpene enriched alcohol composition according to embodiment 153 wherein said water-soluble terpene is an acetylated terpene glycoside.
   156. A terpene enriched alcohol composition according to embodiment 153 wherein said water-soluble terpene is a mixture of glycosylated terpenes and acetylated terpene glycoside.
   157. A terpene enriched alcohol composition according to embodiment 154 wherein said glycosylated terpene is glycosylated in vivo.
   158. A terpene enriched alcohol composition according to embodiment 154 wherein said glycosylated terpene is glycosylated in vitro.
   159. A terpene enriched alcohol composition according to embodiment 155 wherein said acetylated terpene glycoside is acetylated in vivo.
   160. A terpene enriched alcohol composition according to embodiment 155 wherein said acetylated terpene glycoside is acetylated in vitro.
   161. A terpene enriched alcohol composition according to embodiment 156 wherein said acetylated terpene glycoside is acetylated in vivo and glycosylated terpene is glycosylated in vivo.
   162. A terpene enriched alcohol composition according to embodiment 156 wherein said acetylated terpene glycoside is acetylated in vitro and glycosylated terpene is glycosylated in vitro.
   163. A terpene enriched alcohol composition according to embodiment 153, wherein said water-soluble terpene is non-psychoactive.
   164. A terpene enriched alcohol composition according to embodiment 153, wherein said ethanol is an ethyl alcohol.
   165. A terpene enriched alcohol composition according to embodiment 164, wherein said ethyl alcohol has a proof greater than 100.
   166. A terpene enriched alcohol composition according to embodiment 164, wherein said ethyl alcohol is beer.
   167. A terpene enriched alcohol composition according to embodiment 164, wherein said ethyl alcohol is wine.
   168. A terpene enriched alcohol composition according to embodiment 164, wherein said ethyl alcohol is a distilled spirit.
   169. A chewing gum composition comprising:
a first quantity of at least one water-soluble terpene;
a gum base comprising a buffering agent selected from the group consisting of acetates, glycinates, phosphates, carbonates, glycerophosphates, citrates, borates, and mixtures thereof;
at least one sweetening agent; and
at least one flavoring agent.
   170. The chewing gum composition of embodiment 169, wherein said water-soluble terpene comprises at least one water-soluble glycosylated terpene.
   171. The chewing gum composition of embodiment 169, wherein said water-soluble terpene comprises at least one water-soluble acetylated terpene glycoside.
   172. The chewing gum composition of embodiment 169, wherein said water-soluble terpene comprises at least one water-soluble acetylated terpene glycoside, and at least one water-soluble glycosylated terpene.
   173. The chewing gum composition of embodiment 172, wherein said water soluble glycosylated terpene, and/or said acetylated terpene glycoside were glycosylated and acetylated in vivo respectively
   174. The chewing gum composition of embodiment 169, comprising
0.01 to 1% by weight of said water-soluble terpene;
25 to 85% by weight of said gum base;
10 to 35% by weight of said at least one sweetening agent; and
1 to 10% by weight of said flavoring agent.
   175. The chewing gum composition of embodiment 174, wherein said flavoring agents comprise a flavoring agent selected from the group consisting of menthol flavor, Eucalyptus, mint flavor and/or L-menthol.
   176. The chewing gum composition of embodiment 174, wherein said sweetening agent comprises a sweetening agent selected from the group consisting of xylitol, sorbitol, isomalt, aspartame, sucralose, acesulfame potassium, and saccharin.
   177. The chewing gum composition according to embodiment 169, wherein the chewing gum composition comprises an antioxidant.
   178. The chewing gum composition according to embodiment 169, wherein the chewing gum composition comprises a pharmaceutically acceptable excipient selected from the group consisting of fillers, disintegrants, binders, lubricants, and antioxidants.
   179. The chewing gum composition according to embodiment 169, wherein the chewing gum composition is non-disintegrating.
   180. The chewing gum composition according to embodiment 169, wherein the chewing gum comprises natural flavors.
   181. The chewing gum composition according to embodiment 169, and further comprising a coloring agent.
   182. The chewing gum composition according to embodiment 169, and further comprising a flavoring agent.
   183. The chewing gum composition according to embodiment 169, wherein said water-soluble terpene is non-psychoactive.
   184. A composition for a water-soluble terpene infused solution comprising:
purified water;
at least one water-soluble terpene;
at least one flavoring agent.
   185. The composition of embodiment 1, and further comprising a sweetener selected from the group consisting of: glucose, sucrose, invert sugar, corn syrup, Stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same.
   186. The composition of embodiment 184, and further comprising sodium chloride.
   187. The composition of embodiment 184, and further comprising glycerin.
   188. The composition of embodiment 184, and further comprising a coloring agent.
   189. The composition of embodiment 184, and further comprising a first quantity of a demulcent.
   190. The composition of embodiment 184, wherein said demulcent is selected from the group consisting of: pectin, glycerin, honey, methylcellulose, and propylene glycol.
   191. The composition of embodiment 184, wherein said water-soluble terpene is selected from the group consisting of: a water soluble glycosylated terpene, a water soluble acetylated terpene glycoside, or a mixture of both.
   192. The composition of embodiment 191, wherein said water soluble glycosylated terpene, and/or said acetylated terpene glycoside were glycosylated and acetylated in vivo respectively.
   193. The composition of embodiment 184, wherein said water-soluble terpene is non-psychoactive.
   194. A composition for a water-soluble terpene infused anesthetic solution comprising:
purified water;
at least one water-soluble terpene;
at least one oral anesthetic.
   195. The composition of embodiment 194, and further comprising a sweetener selected from the group consisting of: glucose, sucrose, invert sugar, corn syrup, Stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same.
   196. The composition of embodiment 194, and further comprising sodium chloride.
   197. The composition of embodiment 194, and further comprising glycerin.
   198. The composition of embodiment 194, and further comprising a coloring agent.
   199. The composition of embodiment 194, wherein said anesthetic is selected from the group consisting of: benzocaine, and phenol.
   200. The composition of embodiment 199, wherein said first quantity of anesthetic is between 0.1% to 15% volume by weight.
   201. The composition of embodiment 194, and further comprising a first quantity of a demulcent.
   202. The composition of embodiment 201, wherein said demulcent is selected from the group consisting of: pectin, glycerin, honey, methylcellulose, and propylene glycol.
   203. The composition of embodiment 194, wherein said water-soluble terpene is selected from the group consisting of: a water soluble glycosylated terpene, a water soluble acetylated terpene glycoside, or a mixture of both.
   204. The composition of embodiment 203, wherein said water soluble glycosylated terpene, and/or said acetylated terpene glycoside were glycosylated and acetylated in vivo respectively.
   205. The composition of embodiment 203, wherein said water soluble glycosylated terpene, and/or said acetylated terpene glycoside were glycosylated and acetylated in vitro respectively.
   206. The composition of embodiment 194, wherein said water-soluble terpene is non-psychoactive.
   207. A composition for a hard lozenge for rapid delivery of water-soluble terpenes through the oral mucosa, the lozenge comprising:
a crystalized sugar base;
at least one water-soluble terpene;
wherein said hard lozenge has a moisture content between 0.1 to 2%.
   208. The composition of embodiment 207, wherein said crystalized sugar base comprises a crystalized sugar base selected from the group consisting of: sucrose, invert sugar, corn syrup, and isomalt or a combination of the same.
   209. The composition of embodiment 207, and further comprising at least one acidulant.
   210. The composition of embodiment 209, wherein said acidulant is selected from the group consisting of: citric acid, tartaric acid, fumaric acid, and malic acid.
   211. The composition of embodiment 209, and further comprising at least one pH adjustor.
   212. The composition of embodiment 211, wherein said pH adjustor is selected from the group consisting of: calcium carbonate, sodium bicarbonate, and magnesium trisilicate.
   213. The composition of embodiment 207, and further comprising at least one anesthetic.
   214. The composition of embodiment 213, wherein said anesthetic is selected from the group consisting of: benzocaine, and phenol.
   215. The composition of embodiment 213, wherein said first quantity of anesthetic is between 1 mg to 15 mg.
   216. The composition of embodiment 1207, and further comprising a first quantity of menthol.
   217. The composition of embodiment 216, wherein said first quantity of menthol is between 1 mg to 20 mg.
   218. The composition of embodiment 207, and further comprising a first quantity of a demulcent.
   219. The composition of embodiment 218, wherein said demulcent is selected from the group consisting of: pectin, glycerin, honey, methylcellulose, propylene glycol, and glycerine.
   220. The composition of embodiment 218, wherein said first quantity of demulcent is between 1 mg to 10 mg.
   221. The composition of embodiment 207, wherein said water-soluble terpene is selected from the group consisting of: a water soluble glycosylated terpene, an acetylated terpene glycoside, or a mixture of both.
   222. The composition of embodiment 221, wherein said water soluble glycosylated terpene, and/or said acetylated terpene glycoside were glycosylated and acetylated in vivo respectively
   223. The composition of embodiment 221, wherein said water soluble glycosylated terpene, and/or said acetylated terpene glycoside were glycosylated and acetylated in vitro respectively
   224. The composition of embodiment 221, wherein the water-soluble terpene is below 50 mg.
   225. The composition of embodiment 221, wherein the water-soluble terpene is above 50 mg.
   226. The composition of embodiment 221, wherein said water-soluble terpene is non-psychoactive.
   227. A chewable lozenge for rapid delivery of water-soluble terpenes through the oral mucosa, the lozenge comprising:
a glycerinated gelatin base;
at least one sweetener; and
at least one water-soluble terpene dissolved in a first quantity of water.
   228. The composition of embodiment 227, wherein said sweetener comprises a sweetener selected from the group consisting of: glucose, sucrose, invert sugar, corn syrup, Stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same.
   229. The composition of embodiment 227, and further comprising at least one acidulant.
   230. The composition of embodiment 229, wherein said acidulant is selected from the group consisting of: citric acid, tartaric acid, fumaric acid, and malic acid.
   231. The composition of embodiment 229, and further comprising at least one pH adjustor.
   232. The composition of embodiment 231, wherein said pH adjustor is selected from the group consisting of: calcium carbonate, sodium bicarbonate, and magnesium trisilicate.
   233. The composition of embodiment 227, and further comprising at least one anesthetic.
   234. The composition of embodiment 233, wherein said anesthetic is selected from the group consisting of: benzocaine, and phenol.
   235. The composition of embodiment 233, wherein said first quantity of anesthetic is between 1 mg to 15 mg.
   236. The composition of embodiment 227, and further comprising a first quantity of menthol.
   237. The composition of embodiment 236, wherein said first quantity of menthol is between 1 mg to 20 mg.
   238. The composition of embodiment 227, and further comprising a first quantity of a demulcent.
   239. The composition of embodiment 238, wherein said demulcent is selected from the group consisting of: pectin, glycerin, honey, methylcellulose, propylene glycol, and glycerine.
   240. The composition of embodiment 238, wherein said first quantity of demulcent is between 1 mg to 10 mg.
   241. The composition of embodiment 227, wherein said water-soluble terpene is selected from the group consisting of: a water soluble glycosylated terpene, an acetylated terpene glycoside, or a mixture of both.
   242. The composition of embodiment 241, wherein said water soluble glycosylated terpene, and/or said acetylated terpene glycoside were glycosylated and acetylated in vivo respectively
   243. The composition of embodiment 241, wherein the water-soluble terpene is below 50 mg.
   244. The composition of embodiment 241, wherein the water-soluble terpene is above 50 mg.
   245. The composition of embodiment 227, wherein said water-soluble terpene is non-psychoactive.
   246. A soft lozenge for rapid delivery of terpenes through the oral mucosa, the lozenge comprising:
a polyethylene glycol base;
at least one sweetener; and
at least one water-soluble terpene.
   247. The composition of embodiment 246, wherein said sweetener comprises a crystalized sugar base selected from the group consisting of: glucose, sucrose, invert sugar, corn syrup, Stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same.
   248. The composition of embodiment 246, and further comprising at least one acidulant.
   249. The composition of embodiment 248, wherein said acidulant is selected from the group consisting of: citric acid, tartaric acid, fumaric acid, and malic acid.
   250. The composition of embodiment 248, and further comprising at least one pH adjustor.
   251. The composition of embodiment 250, wherein said pH adjustor is selected from the group consisting of: calcium carbonate, sodium bicarbonate, and magnesium trisilicate.
   252. The composition of embodiment 247, and further comprising at least one anesthetic.
   253. The composition of embodiment 252, wherein said anesthetic is selected from the group consisting of: benzocaine, and phenol.
   254. The composition of embodiment 252, wherein said first quantity of anesthetic is between 1 mg to 15 mg.
   255. The composition of embodiment 246, and further comprising a first quantity of menthol.
   256. The composition of embodiment 255, wherein said first quantity of menthol is between 1 mg to 20 mg.
   257. The composition of embodiment 246, and further comprising a first quantity of a demulcent.
   258. The composition of embodiment 257, wherein said demulcent is selected from the group consisting of: pectin, glycerin, honey, methylcellulose, propylene glycol, and glycerine.
   259. The composition of embodiment 2258, wherein said first quantity of demulcent is between 1 mg to 10 mg.
   260. The composition of embodiment 246, wherein said water-soluble terpene is selected from the group consisting of: a water soluble glycosylated terpene, an acetylated terpene glycoside, or a mixture of both.
   261. The composition of embodiment 260, wherein said water soluble glycosylated terpene, and/or said acetylated terpene glycoside were glycosylated and acetylated in vivo respectively.
   262. The composition of embodiment 260, wherein the water-soluble terpene is below 50 mg.
   263. The composition of embodiment 260, wherein the water-soluble terpene is above 50 mg.
   264. The composition of embodiment 246, wherein said water-soluble terpene is non-psychoactive.
   265. A tablet or capsule consisting essentially of a water-soluble glycosylated terpene and maltodextrin.
   266. The tablet or capsule of embodiment 265, wherein said water-soluble glycosylated terpene comprises a water-soluble glycosylated terpene generated in vivo.
   267. The tablet or capsule of embodiment 265, wherein said water-soluble glycosylated terpene comprises a water-soluble glycosylated terpene generated in vitro.
   268. The tablet or capsule of embodiment 265, wherein said water-soluble glycosylated terpene comprises a non-psychoactive water-soluble glycosylated terpene.
   269. The tablet or capsule of embodiment 265, wherein the amount of water-soluble glycosylated terpene is 5 milligrams or less.
   270. The tablet or capsule of embodiment 265, wherein the amount of water-soluble glycosylated terpene 5 milligrams and 200 milligrams.
   271. The tablet or capsule of embodiment 265, wherein the wherein the amount of water-soluble glycosylated terpene is more than 200 milligrams.
   272. A tablet or capsule consisting essentially of a water-soluble glycosylated terpene and whey protein isolate.
   273. The tablet or capsule of embodiment 272, wherein said water-soluble glycosylated terpene comprises a water-soluble glycosylated terpene generated in vivo.
   274. The tablet or capsule of embodiment 272, wherein said water-soluble glycosylated terpene comprises a water-soluble glycosylated terpene generated in vitro.
   274. The tablet or capsule of embodiment 272, wherein said water-soluble glycosylated terpene comprises a non-psychoactive water-soluble glycosylated terpene.
   275. The tablet or capsule of embodiment 272, wherein the amount of water-soluble glycosylated terpene is 5 milligrams or less.
   276. The tablet or capsule of embodiment 272, wherein the amount of water-soluble glycosylated terpene 5 milligrams and 200 milligrams.
   277. The tablet or capsule of embodiment 272, wherein the wherein the amount of water-soluble glycosylated terpene is more than 200 milligrams.
   278. A tablet or capsule consisting essentially of a water-soluble glycosylated terpene and xanthan gum.
   279. The tablet or capsule of embodiment 278, wherein said water-soluble glycosylated terpene comprises a water-soluble glycosylated terpene generated in vivo.
   280. The tablet or capsule of embodiment 278, wherein said water-soluble glycosylated terpene comprises a water-soluble glycosylated terpene generated in vitro.
   281. The tablet or capsule of embodiment 278, wherein said water-soluble glycosylated terpene comprises a non-psychoactive water-soluble glycosylated terpene.
   282. The tablet or capsule of embodiment 278, wherein the amount of water-soluble glycosylated terpene is 5 milligrams or less.
   283. The tablet or capsule of embodiment 278, wherein the amount of water-soluble glycosylated terpene 5 milligrams and 200 milligrams.
   284. The tablet or capsule of embodiment 278, wherein the wherein the amount of water-soluble glycosylated terpene is more than 200 milligrams.
   285. A tablet or capsule consisting essentially of a water-soluble glycosylated terpene and guar gum.
   286. The tablet or capsule of embodiment 285, wherein said water-soluble glycosylated terpene comprises a water-soluble glycosylated terpene generated in vivo.
   287. The tablet or capsule of embodiment 285, wherein said water-soluble glycosylated terpene comprises a water-soluble glycosylated terpene generated in vitro.
   288. The tablet or capsule of embodiment 285, wherein said water-soluble glycosylated terpene comprises a non-psychoactive water-soluble glycosylated terpene.
   289. The tablet or capsule of embodiment 285, wherein the amount of water-soluble glycosylated terpene is 5 milligrams or less.
   290. The tablet or capsule of embodiment 285, wherein the amount of water-soluble glycosylated terpene 5 milligrams and 200 milligrams.
   291. The tablet or capsule of embodiment 285, wherein the wherein the amount of water-soluble glycosylated terpene is more than 200 milligrams.
   292. A tablet or capsule consisting essentially of water-soluble glycosylated terpene and diglycerides.
   293. The tablet or capsule of embodiment 292 wherein the diglycerides are in a mix with monoglycerides.
   294. The tablet or capsule of embodiment 292, wherein said water-soluble glycosylated terpene comprises a water-soluble glycosylated terpene generated in vivo.
   295. The tablet or capsule of embodiment 292, wherein said water-soluble glycosylated terpene comprises a water-soluble glycosylated terpene generated in vitro.
   296. The tablet or capsule of embodiment 292, wherein said water-soluble glycosylated terpene comprises a non-psychoactive water-soluble glycosylated terpene.
   297. The tablet or capsule of embodiment 292, wherein the amount of water-soluble glycosylated terpene is 5 milligrams or less.
   298. The tablet or capsule of embodiment 292, wherein the amount of water-soluble glycosylated terpene 5 milligrams and 200 milligrams.
   299. The tablet or capsule of embodiment 292, wherein the wherein the amount of water-soluble glycosylated terpene is more than 200 milligrams.
   300. A tablet or capsule consisting essentially of a water-soluble glycosylated terpene and guar gum.
   301. The tablet or capsule of embodiment 300, wherein said water-soluble glycosylated terpene comprises a water-soluble glycosylated terpene generated in vivo.
   302. The tablet or capsule of embodiment 300, wherein said water-soluble glycosylated terpene comprises a water-soluble glycosylated terpene generated in vitro.
   303. The tablet or capsule of embodiment 300, wherein said water-soluble glycosylated terpene comprises a non-psychoactive water-soluble glycosylated terpene.
   304. The tablet or capsule of embodiment 300, wherein the amount of water-soluble glycosylated terpene is 5 milligrams or less.
   305. The tablet or capsule of embodiment 300, wherein the amount of water-soluble glycosylated terpene 5 milligrams and 200 milligrams.
   306. The tablet or capsule of embodiment 300, wherein the wherein the amount of water-soluble glycosylated terpene is more than 200 milligrams.
   307. A tablet or capsule consisting essentially of water-soluble glycosylated terpene and carboxymethyl cellulose.
   308. The tablet or capsule of embodiment 307, wherein said water-soluble glycosylated terpene comprises a water-soluble glycosylated terpene generated in vivo.
   309. The tablet or capsule of embodiment 307, wherein said water-soluble glycosylated terpene comprises a water-soluble glycosylated terpene generated in vitro.
   310. The tablet or capsule of embodiment 307, wherein said water-soluble glycosylated terpene comprises a non-psychoactive water-soluble glycosylated terpene.
   311. The tablet or capsule of embodiment 307, wherein the amount of water-soluble glycosylated terpene is 5 milligrams or less.
   312. The tablet or capsule of embodiment 307, wherein the amount of water-soluble glycosylated terpene 5 milligrams and 200 milligrams.
   313. The tablet or capsule of embodiment 307, wherein the wherein the amount of water-soluble glycosylated terpene is more than 200 milligrams.
   314. A tablet or capsule consisting essentially a water-soluble glycosylated terpene and glycerin.
   315. The tablet or capsule of embodiment 314, wherein said water-soluble glycosylated terpene comprises a water-soluble glycosylated terpene generated in vivo.
   316. The tablet or capsule of embodiment 314, wherein said water-soluble glycosylated terpene comprises a water-soluble glycosylated terpene generated in vitro.
   317. The tablet or capsule of embodiment 314, wherein said water-soluble glycosylated terpene comprises a non-psychoactive water-soluble glycosylated terpene.
   318. The tablet or capsule of embodiment 314, wherein the amount of water-soluble glycosylated terpene is 5 milligrams or less.
   319. The tablet or capsule of embodiment 314, wherein the amount of water-soluble glycosylated terpene 5 milligrams and 200 milligrams.
   320. The tablet or capsule of embodiment 314, wherein the wherein the amount of water-soluble glycosylated terpene is more than 200 milligrams.
   321. A tablet or capsule consisting essentially of a water-soluble glycosylated terpene and gelatin.
   322. The tablet or capsule of embodiment 321, wherein said water-soluble glycosylated terpene comprises a water-soluble glycosylated terpene generated in vivo.
   323. The tablet or capsule of embodiment 321, wherein said water-soluble glycosylated terpene comprises a water-soluble glycosylated terpene generated in vitro.
   324. The tablet or capsule of embodiment 321, wherein said water-soluble glycosylated terpene comprises a non-psychoactive water-soluble glycosylated terpene.
   325. The tablet or capsule of embodiment 321, wherein the amount of water-soluble glycosylated terpene is 5 milligrams or less.
   326. The tablet or capsule of embodiment 321, wherein the amount of water-soluble glycosylated terpene 5 milligrams and 200 milligrams.
   327. The tablet or capsule of embodiment 321, wherein the wherein the amount of water-soluble glycosylated terpene is more than 200 milligrams.
   328. A tablet or capsule consisting essentially of water-soluble glycosylated terpene and polyethylene glycol.
   329. The tablet or capsule of embodiment 328, wherein said water-soluble glycosylated terpene comprises a water-soluble glycosylated terpene generated in vivo.
   330. The tablet or capsule of embodiment 328, wherein said water-soluble glycosylated terpene comprises a water-soluble glycosylated terpene generated in vitro.
   331. The tablet or capsule of embodiment 328, wherein said water-soluble glycosylated terpene comprises a non-psychoactive water-soluble glycosylated terpene.
   332. The tablet or capsule of embodiment 328, wherein the amount of water-soluble glycosylated terpene is 5 milligrams or less.
   333. The tablet or capsule of embodiment 328, wherein the amount of water-soluble glycosylated terpene 5 milligrams and 200 milligrams.
   334. The tablet or capsule of embodiment 328, wherein the wherein the amount of water-soluble glycosylated terpene is more than 200 milligrams.
   335. A tablet or capsule consisting essentially of a water-soluble acetylated terpene glycoside and maltodextrin.
   336. The tablet or capsule of embodiment 335, wherein said water-soluble acetylated terpene glycoside comprises a water-soluble acetylated terpene glycoside generated in vivo.
   337. The tablet or capsule of embodiment 335, wherein said water-soluble acetylated terpene glycoside comprises a water-soluble acetylated terpene glycoside generated in vitro.
   338. The tablet or capsule of embodiment 335, wherein said water-soluble acetylated terpene glycoside comprises a non-psychoactive water-soluble acetylated terpene glycoside.
   339. The tablet or capsule of embodiment 335, wherein the amount of water-soluble acetylated terpene glycoside is 5 milligrams or less.
   340. The tablet or capsule of embodiment 335, wherein the amount of water-soluble acetylated terpene glycoside 5 milligrams and 200 milligrams.
   340. The tablet or capsule of embodiment 335, wherein the wherein the amount of water-soluble acetylated terpene glycoside is more than 200 milligrams.
   341. A tablet or capsule consisting essentially of a water-soluble acetylated terpene glycoside and whey protein isolate.
   342. The tablet or capsule of embodiment 341, wherein said water-soluble acetylated terpene glycoside comprises a water-soluble acetylated terpene glycoside generated in vivo.
   343. The tablet or capsule of embodiment 341, wherein said water-soluble acetylated terpene glycoside comprises a water-soluble acetylated terpene glycoside generated in vitro.
   344. The tablet or capsule of embodiment 341, wherein said water-soluble acetylated terpene glycoside comprises a non-psychoactive water-soluble acetylated terpene glycoside.
   345. The tablet or capsule of embodiment 341, wherein the amount of water-soluble acetylated terpene glycoside is 5 milligrams or less.
   346. The tablet or capsule of embodiment 341, wherein the amount of water-soluble acetylated terpene glycoside 5 milligrams and 200 milligrams.
   347. The tablet or capsule of embodiment 341, wherein the wherein the amount of water-soluble acetylated terpene glycoside is more than 200 milligrams.
   348. A tablet or capsule consisting essentially of a water-soluble acetylated terpene glycoside and xanthan gum.
   349. The tablet or capsule of embodiment 348, wherein said water-soluble acetylated terpene glycoside comprises a water-soluble acetylated terpene glycoside generated in vivo.
   350. The tablet or capsule of embodiment 348, wherein said water-soluble acetylated terpene glycoside comprises a water-soluble acetylated terpene glycoside generated in vitro.
   351. The tablet or capsule of embodiment 348, wherein said water-soluble acetylated terpene glycoside comprises a non-psychoactive water-soluble acetylated terpene glycoside.
   352. The tablet or capsule of embodiment 348, wherein the amount of water-soluble acetylated terpene glycoside is 5 milligrams or less.
   353. The tablet or capsule of embodiment 348, wherein the amount of water-soluble acetylated terpene glycoside 5 milligrams and 200 milligrams.
   354. The tablet or capsule of embodiment 348, wherein the wherein the amount of water-soluble acetylated terpene glycoside is more than 200 milligrams.
   355. A tablet or capsule consisting essentially of a water-soluble acetylated terpene glycoside and guar gum.
   356. The tablet or capsule of embodiment 355, wherein said water-soluble acetylated terpene glycoside comprises a water-soluble acetylated terpene glycoside generated in vivo.
   357. The tablet or capsule of embodiment 355, wherein said water-soluble acetylated terpene glycoside comprises a water-soluble acetylated terpene glycoside generated in vitro.
   358. The tablet or capsule of embodiment 355, wherein said water-soluble acetylated terpene glycoside comprises a non-psychoactive water-soluble acetylated terpene glycoside.
   359. The tablet or capsule of embodiment 355, wherein the amount of water-soluble acetylated terpene glycoside is 5 milligrams or less.
   360. The tablet or capsule of embodiment 355, wherein the amount of water-soluble acetylated terpene glycoside 5 milligrams and 200 milligrams.
   361. The tablet or capsule of embodiment 355, wherein the wherein the amount of water-soluble acetylated terpene glycoside is more than 200 milligrams.
   362. A tablet or capsule consisting essentially of water-soluble acetylated terpene glycoside and diglycerides.
   363. The tablet or capsule of embodiment 362 wherein the diglycerides are in a mix with monoglycerides.
   364. The tablet or capsule of embodiment 362, wherein said water-soluble acetylated terpene glycoside comprises a water-soluble acetylated terpene glycoside generated in vivo.
   365. The tablet or capsule of embodiment 362, wherein said water-soluble acetylated terpene glycoside comprises a water-soluble acetylated terpene glycoside generated in vitro.
   366. The tablet or capsule of embodiment 362, wherein said water-soluble acetylated terpene glycoside comprises a non-psychoactive water-soluble acetylated terpene glycoside.
   367. The tablet or capsule of embodiment 362, wherein the amount of water-soluble acetylated terpene glycoside is 5 milligrams or less.
   368. The tablet or capsule of embodiment 362, wherein the amount of water-soluble acetylated terpene glycoside 5 milligrams and 200 milligrams.
   369. The tablet or capsule of embodiment 362, wherein the wherein the amount of water-soluble acetylated terpene glycoside is more than 200 milligrams.
   370. A tablet or capsule consisting essentially of a water-soluble acetylated terpene glycoside and guar gum.
   371. The tablet or capsule of embodiment 370, wherein said water-soluble acetylated terpene glycoside comprises a water-soluble acetylated terpene glycoside generated in vivo.
   372. The tablet or capsule of embodiment 370, wherein said water-soluble acetylated terpene glycoside comprises a water-soluble acetylated terpene glycoside generated in vitro.
   373. The tablet or capsule of embodiment 370, wherein said water-soluble acetylated terpene glycoside comprises a non-psychoactive water-soluble acetylated terpene glycoside.
   374. The tablet or capsule of embodiment 370, wherein the amount of water-soluble acetylated terpene glycoside is 5 milligrams or less.
   375. The tablet or capsule of embodiment 370, wherein the amount of water-soluble acetylated terpene glycoside 5 milligrams and 200 milligrams.
   376. The tablet or capsule of embodiment 370, wherein the wherein the amount of water-soluble acetylated terpene glycoside is more than 200 milligrams.
   377. A tablet or capsule consisting essentially of a water-soluble acetylated terpene glycoside and carboxymethyl cellulose.
   378. The tablet or capsule of embodiment 377, wherein said water-soluble acetylated terpene glycoside comprises a water-soluble acetylated terpene glycoside generated in vivo.
   379. The tablet or capsule of embodiment 377, wherein said water-soluble acetylated terpene glycoside comprises a water-soluble acetylated terpene glycoside generated in vitro.
   380. The tablet or capsule of embodiment 377, wherein said water-soluble acetylated terpene glycoside comprises a non-psychoactive water-soluble acetylated terpene glycoside.
   390. The tablet or capsule of embodiment 377, wherein the amount of water-soluble acetylated terpene glycoside is 5 milligrams or less.
   391. The tablet or capsule of embodiment 377, wherein the amount of water-soluble acetylated terpene glycoside 5 milligrams and 200 milligrams.
   392. The tablet or capsule of embodiment 377, wherein the wherein the amount of water-soluble acetylated terpene glycoside is more than 200 milligrams.
   393. A tablet or capsule consisting essentially a water-soluble acetylated terpene glycoside and glycerin.
   394. The tablet or capsule of embodiment 393, wherein said water-soluble acetylated terpene glycoside comprises a water-soluble acetylated terpene glycoside generated in vivo.
   395. The tablet or capsule of embodiment 393, wherein said water-soluble acetylated terpene glycoside comprises a water-soluble acetylated terpene glycoside generated in vitro.
   396. The tablet or capsule of embodiment 393, wherein said water-soluble acetylated terpene glycoside comprises a non-psychoactive water-soluble acetylated terpene glycoside.
   397. The tablet or capsule of embodiment 393, wherein the amount of water-soluble acetylated terpene glycoside is 5 milligrams or less.
   398. The tablet or capsule of embodiment 393, wherein the amount of water-soluble acetylated terpene glycoside 5 milligrams and 200 milligrams.
   399. The tablet or capsule of embodiment 393, wherein the wherein the amount of water-soluble acetylated terpene glycoside is more than 200 milligrams.
   400. A tablet or capsule consisting essentially of a water-soluble acetylated terpene glycoside and gelatin.
   401. The tablet or capsule of embodiment 400, wherein said water-soluble acetylated terpene glycoside comprises a water-soluble acetylated terpene glycoside generated in vivo.
   402. The tablet or capsule of embodiment 400, wherein said water-soluble acetylated terpene glycoside comprises a water-soluble acetylated terpene glycoside generated in vitro.
   403. The tablet or capsule of embodiment 400, wherein said water-soluble acetylated terpene glycoside comprises a non-psychoactive water-soluble acetylated terpene glycoside.
   404. The tablet or capsule of embodiment 400, wherein the amount of water-soluble acetylated terpene glycoside is 5 milligrams or less.
   405. The tablet or capsule of embodiment 400, wherein the amount of water-soluble acetylated terpene glycoside 5 milligrams and 200 milligrams.
   406. The tablet or capsule of embodiment 400, wherein the wherein the amount of water-soluble acetylated terpene glycoside is more than 200 milligrams.
   407. A tablet or capsule consisting essentially of a water-soluble acetylated terpene glycoside and polyethylene glycol.
   408. The tablet or capsule of embodiment 407, wherein said water-soluble acetylated terpene glycoside comprises a water-soluble acetylated terpene glycoside generated in vivo.
   409. The tablet or capsule of embodiment 407, wherein said water-soluble acetylated terpene glycoside comprises a water-soluble acetylated terpene glycoside generated in vitro.
   410. The tablet or capsule of embodiment 407, wherein said water-soluble acetylated terpene glycoside comprises a non-psychoactive water-soluble acetylated terpene glycoside.
   411. The tablet or capsule of embodiment 407, wherein the amount of water-soluble acetylated terpene glycoside is 5 milligrams or less.
   412. The tablet or capsule of embodiment 407, wherein the amount of water-soluble acetylated terpene glycoside is between 5 milligrams and 200 milligrams.
   413. The tablet or capsule of embodiment 407, wherein the wherein the amount of water-soluble acetylated terpene glycoside is more than 200 milligrams.
   414. A tablet or capsule consisting essentially of a water-soluble acetylated terpene glycoside and a water-soluble glycosylated terpene and a water-soluble glycosylated terpene and maltodextrin.
   415. The tablet or capsule of embodiment 414, wherein said water-soluble acetylated terpene glycoside comprises a water-soluble acetylated terpene glycoside and a water-soluble glycosylated terpene and a water-soluble glycosylated terpene generated in vivo.
   416. The tablet or capsule of embodiment 414, wherein said water-soluble acetylated terpene glycoside comprises a water-soluble acetylated terpene glycoside and a water-soluble glycosylated terpene and a water-soluble glycosylated terpene generated in vitro.
   417. The tablet or capsule of embodiment 414, wherein said water-soluble acetylated terpene glycoside comprises a non-psychoactive water-soluble acetylated terpene glycoside.
   418. The tablet or capsule of embodiment 414, wherein the amount of water-soluble acetylated terpene glycoside is 5 milligrams or less.
   419. The tablet or capsule of embodiment 414, wherein the amount of water-soluble acetylated terpene glycoside 5 milligrams and 200 milligrams.
   420. The tablet or capsule of embodiment 414, wherein the wherein the amount of water-soluble acetylated terpene glycoside is more than 200 milligrams.
   421. A tablet or capsule consisting essentially of a water-soluble acetylated terpene glycoside and a water-soluble glycosylated terpene and a water-soluble glycosylated terpene and whey protein isolate.
   422. The tablet or capsule of embodiment 421, wherein said water-soluble acetylated terpene glycoside comprises a water-soluble acetylated terpene glycoside and a water-soluble glycosylated terpene and a water-soluble glycosylated terpene generated in vivo.
   423. The tablet or capsule of embodiment 421, wherein said water-soluble acetylated terpene glycoside comprises a water-soluble acetylated terpene glycoside and a water-soluble glycosylated terpene and a water-soluble glycosylated terpene generated in vitro.
   424. The tablet or capsule of embodiment 421, wherein said water-soluble acetylated terpene glycoside comprises a non-psychoactive water-soluble acetylated terpene glycoside.
   425. The tablet or capsule of embodiment 421, wherein the amount of water-soluble acetylated terpene glycoside is 5 milligrams or less.
   426. The tablet or capsule of embodiment 421, wherein the amount of water-soluble acetylated terpene glycoside 5 milligrams and 200 milligrams.
   427. The tablet or capsule of embodiment 421, wherein the wherein the amount of water-soluble acetylated terpene glycoside is more than 200 milligrams.
   428. A tablet or capsule consisting essentially of a water-soluble acetylated terpene glycoside and a water-soluble glycosylated terpene and a water-soluble glycosylated terpene and xanthan gum.
   429. The tablet or capsule of embodiment 428, wherein said water-soluble acetylated terpene glycoside comprises a water-soluble acetylated terpene glycoside and a water-soluble glycosylated terpene and a water-soluble glycosylated terpene generated in vivo.
   430. The tablet or capsule of embodiment 428, wherein said water-soluble acetylated terpene glycoside comprises a water-soluble acetylated terpene glycoside and a water-soluble glycosylated terpene and a water-soluble glycosylated terpene generated in vitro.
   431. The tablet or capsule of embodiment 428, wherein said water-soluble acetylated terpene glycoside comprises a non-psychoactive water-soluble acetylated terpene glycoside.
   432. The tablet or capsule of embodiment 428, wherein the amount of water-soluble acetylated terpene glycoside is 5 milligrams or less.
   433. The tablet or capsule of embodiment 428, wherein the amount of water-soluble acetylated terpene glycoside 5 milligrams and 200 milligrams.
   432. The tablet or capsule of embodiment 428, wherein the wherein the amount of water-soluble acetylated terpene glycoside is more than 200 milligrams.
   433. A tablet or capsule consisting essentially of a water-soluble acetylated terpene glycoside and a water-soluble glycosylated terpene and a water-soluble glycosylated terpene and guar gum.
   434. The tablet or capsule of embodiment 433, wherein said water-soluble acetylated terpene glycoside comprises a water-soluble acetylated terpene glycoside and a water-soluble glycosylated terpene and a water-soluble glycosylated terpene generated in vivo.
   435. The tablet or capsule of embodiment 433, wherein said water-soluble acetylated terpene glycoside comprises a water-soluble acetylated terpene glycoside and a water-soluble glycosylated terpene and a water-soluble glycosylated terpene generated in vitro.
   436. The tablet or capsule of embodiment 433, wherein said water-soluble acetylated terpene glycoside comprises a non-psychoactive water-soluble acetylated terpene glycoside.
   437. The tablet or capsule of embodiment 433, wherein the amount of water-soluble acetylated terpene glycoside is 5 milligrams or less.
   438. The tablet or capsule of embodiment 433, wherein the amount of water-soluble acetylated terpene glycoside 5 milligrams and 200 milligrams.
   439. The tablet or capsule of embodiment 433, wherein the wherein the amount of water-soluble acetylated terpene glycoside is more than 200 milligrams.
   440. A tablet or capsule consisting essentially of a water-soluble acetylated terpene glycoside and a water-soluble glycosylated terpene and a water-soluble glycosylated terpene and diglycerides.
   441. The tablet or capsule of embodiment 440 wherein the diglycerides are in a mix with monoglycerides.
   442. The tablet or capsule of embodiment 440, wherein said water-soluble terpene comprises a water-soluble acetylated terpene glycoside and a water-soluble glycosylated terpene and a water-soluble glycosylated terpene generated in vivo.
   443. The tablet or capsule of embodiment 440, wherein said water-soluble terpene comprises a water-soluble acetylated terpene glycoside and a water-soluble glycosylated terpene and a water-soluble glycosylated terpene generated in vitro.
   444. The tablet or capsule of embodiment 440, wherein said water-soluble acetylated terpene glycoside comprises a non-psychoactive water-soluble acetylated terpene glycoside.
   445. The tablet or capsule of embodiment 440, wherein the amount of water-soluble acetylated terpene glycoside is 5 milligrams or less.
   446. The tablet or capsule of embodiment 440, wherein the amount of water-soluble acetylated terpene glycoside 5 milligrams and 200 milligrams.
   447. The tablet or capsule of embodiment 440, wherein the wherein the amount of a water-soluble acetylated terpene glycoside and a water-soluble glycosylated terpene is more than 200 milligrams.
   448. A tablet or capsule consisting essentially of a water-soluble acetylated terpene glycoside and a water-soluble glycosylated terpene and guar gum.
   449. The tablet or capsule of embodiment 448, wherein said a water-soluble acetylated terpene glycoside and a water-soluble glycosylated terpene comprises a water-soluble acetylated terpene glycoside and a water-soluble glycosylated terpene generated in vivo.
   450. The tablet or capsule of embodiment 448, wherein said a water-soluble acetylated terpene glycoside and a water-soluble glycosylated terpene comprises a water-soluble acetylated terpene glycoside and a water-soluble glycosylated terpene generated in vitro.
   451. The tablet or capsule of embodiment 448, wherein said a water-soluble acetylated terpene glycoside and a water-soluble glycosylated terpene comprises a non-psychoactive a water-soluble acetylated terpene glycoside and a water-soluble glycosylated terpene.
   452. The tablet or capsule of embodiment 448, wherein the amount of a water-soluble acetylated terpene glycoside and a water-soluble glycosylated terpene is 5 milligrams or less.
   453. The tablet or capsule of embodiment 448, wherein the amount of a water-soluble acetylated terpene glycoside and a water-soluble glycosylated terpene 5 milligrams and 200 milligrams.
   454. The tablet or capsule of embodiment 448, wherein the wherein the amount of a water-soluble acetylated terpene glycoside and a water-soluble glycosylated terpene is more than 200 milligrams.
   455. A tablet or capsule consisting essentially of comprises a water-soluble acetylated terpene glycoside and a water-soluble glycosylated terpene and a water-soluble glycosylated terpene and carboxymethyl cellulose.
   456. The tablet or capsule of embodiment 455, wherein said a water-soluble acetylated terpene glycoside and a water-soluble glycosylated terpene comprises a water-soluble acetylated terpene glycoside and a water-soluble glycosylated terpene generated in vivo.
   457. The tablet or capsule of embodiment 455, wherein said a water-soluble acetylated terpene glycoside and a water-soluble glycosylated terpene comprises a water-soluble acetylated terpene glycoside and a water-soluble glycosylated terpene generated in vitro.
   458. The tablet or capsule of embodiment 455, wherein said a water-soluble acetylated terpene glycoside and a water-soluble glycosylated terpene comprises a non-psychoactive a water-soluble acetylated terpene glycoside and a water-soluble glycosylated terpene.
   459. The tablet or capsule of embodiment 455, wherein the amount of a water-soluble acetylated terpene glycoside and a water-soluble glycosylated terpene is 5 milligrams or less.
   460. The tablet or capsule of embodiment 455, wherein the amount of a water-soluble acetylated terpene glycoside and a water-soluble glycosylated terpene 5 milligrams and 200 milligrams.
   461. The tablet or capsule of embodiment 455, wherein the wherein the amount of a water-soluble acetylated terpene glycoside and a water-soluble glycosylated terpene is more than 200 milligrams.
   462. A tablet or capsule consisting essentially a water-soluble acetylated terpene glycoside and a water-soluble glycosylated terpene and glycerin.
   463. The tablet or capsule of embodiment 462, wherein said a water-soluble acetylated terpene glycoside and a water-soluble glycosylated terpene comprises a water-soluble acetylated terpene glycoside and a water-soluble glycosylated terpene generated in vivo.
   464. The tablet or capsule of embodiment 462, wherein said a water-soluble acetylated terpene glycoside and a water-soluble glycosylated terpene comprises a water-soluble acetylated terpene glycoside and a water-soluble glycosylated terpene generated in vitro.
   465. The tablet or capsule of embodiment 462, wherein said a water-soluble acetylated terpene glycoside and a water-soluble glycosylated terpene comprises a non-psychoactive a water-soluble acetylated terpene glycoside and a water-soluble glycosylated terpene.
   466. The tablet or capsule of embodiment 462, wherein the amount of a water-soluble acetylated terpene glycoside and a water-soluble glycosylated terpene is 5 milligrams or less.
   467. The tablet or capsule of embodiment 462, wherein the amount of a water-soluble acetylated terpene glycoside and a water-soluble glycosylated terpene 5 milligrams and 200 milligrams.
   468. The tablet or capsule of embodiment 462, wherein the wherein the amount of a water-soluble acetylated terpene glycoside and a water-soluble glycosylated terpene is more than 200 milligrams.
   462. A tablet or capsule consisting essentially of a water-soluble acetylated terpene glycoside and a water-soluble glycosylated terpene and a water-soluble glycosylated terpene and gelatin.
   470. The tablet or capsule of embodiment 462, wherein said a water-soluble acetylated terpene glycoside and a water-soluble glycosylated terpene comprises a water-soluble acetylated terpene glycoside and a water-soluble glycosylated terpene generated in vivo.
   471. The tablet or capsule of embodiment 462, wherein said a water-soluble acetylated terpene glycoside and a water-soluble glycosylated terpene comprises a water-soluble acetylated terpene glycoside and a water-soluble glycosylated terpene generated in vitro.
   472. The tablet or capsule of embodiment 462, wherein said a water-soluble acetylated terpene glycoside and a water-soluble glycosylated terpene comprises a non-psychoactive a water-soluble acetylated terpene glycoside and a water-soluble glycosylated terpene.
   473. The tablet or capsule of embodiment 462, wherein the amount of a water-soluble acetylated terpene glycoside and a water-soluble glycosylated terpene is 5 milligrams or less.
   474. The tablet or capsule of embodiment 462, wherein the amount of a water-soluble acetylated terpene glycoside and a water-soluble glycosylated terpene 5 milligrams and 200 milligrams.
   475. The tablet or capsule of embodiment 462, wherein the wherein the amount of a water-soluble acetylated terpene glycoside and a water-soluble glycosylated terpene is more than 200 milligrams.
   476. A tablet or capsule consisting essentially of a water-soluble acetylated terpene glycoside and a water-soluble glycosylated terpene and a water-soluble glycosylated terpene and polyethylene glycol.
   477. The tablet or capsule of embodiment 476, wherein said a water-soluble acetylated terpene glycoside and a water-soluble glycosylated terpene comprises a water-soluble acetylated terpene glycoside and a water-soluble glycosylated terpene generated in vivo.
   478. The tablet or capsule of embodiment 476, wherein said a water-soluble acetylated terpene glycoside and a water-soluble glycosylated terpene comprises a water-soluble acetylated terpene glycoside and a water-soluble glycosylated terpene generated in vitro.
   479. The tablet or capsule of embodiment 476, wherein said a water-soluble acetylated terpene glycoside and a water-soluble glycosylated terpene comprises a non-psychoactive a water-soluble acetylated terpene glycoside and a water-soluble glycosylated terpene.
   480. The tablet or capsule of embodiment 476, wherein the amount of a water-soluble acetylated terpene glycoside and a water-soluble glycosylated terpene is 5 milligrams or less.
   481. The tablet or capsule of embodiment 476, wherein the amount of a water-soluble acetylated terpene glycoside and a water-soluble glycosylated terpene is between 5 milligrams and 200 milligrams.
   482. The tablet or capsule of embodiment 476, wherein the wherein the amount of a water-soluble acetylated terpene glycoside and a water-soluble glycosylated terpene is more than 200 milligrams.
   483. A method of manufacturing and packaging a terpene dosage, consisting of the following steps:
preparing a fill solution with a desired concentration of a water-soluble terpene in a liquid carrier wherein said terpene solubilized in said liquid carrier;
encapsulating said fill solution in capsules;
packaging said capsules in a closed packaging system; and
removing atmospheric air from the capsules,
   wherein the removing of atmospheric air consists solely of purging said packaging system with an inert gas, and wherein said packaging system provides a room temperature stable product.
   484. The method of embodiment 483, wherein the packaging system is a blister package.
   485. The method of embodiment 484 wherein the blister package is constructed of material that minimizes exposure to moisture and air.
   486. The method of embodiment 483, wherein the terpene is a glycosylated terpene, a acetylated terpene glycoside or a mixture of the two.
   487. The method of embodiment 486, wherein said glycosylated terpene and/or said acetylated terpene glycoside are generated in vivo.
   488. The method of embodiment 486, wherein said glycosylated terpene and/or said acetylated terpene glycoside are generated in vitro.
   489. The method of embodiment 483, wherein the liquid carrier is water-based carrier.
   490. The method of embodiment 487, wherein the water-based carrier is an aqueous sodium chloride solution.
   491. The method of embodiment 483, wherein the capsules are soft gelatin capsules.
   492. The method of embodiment 483, wherein the inert gas is nitrogen.
   493. The method of embodiment 483, wherein the desired terpene concentration is about 1-10% w/w.
   494. The method of embodiment 493 wherein the desired concentration is about 1.5-6.5% w/w.
   495. An oral pharmaceutical solution consisting essentially of a water-soluble terpene, 30-33% w/w water, about 50% w/w alcohol, 0.01% w/w butylated hydroxylanisole (BHA) or 0.1% w/w ethylenediaminetetraacetic acid (EDTA) and 5-21% w/w co-solvent, having a combined total of 100%, wherein said co-solvent is selected from the group consisting of propylene glycol, polyethylene glycol and combinations thereof, and wherein said water-soluble terpene is a glycosylated terpene, an acetylated terpene glycoside or a mixture of the two.
   496. The oral pharmaceutical solution of embodiment 495 consisting essentially of 0.1 to 5% w/w of said water-soluble terpene, about 50% w/w alcohol, 5.5% w/w propylene glycol, 12% w/w polyethylene glycol and 30-33% w/w water.
   497. The oral pharmaceutical solution of embodiment 496, wherein said alcohol is ethanol.
   498. An oral pharmaceutical solution consisting essentially of about 0.1% to 1% w/w water-soluble terpene, about 50% w/w alcohol, 5.5% w/w propylene glycol, 12% w/w polyethylene glycol, 30-33% w/w water, 0.01% w/w butylated hydroxyanisole, having a combined total of 100%, and wherein said water-soluble terpene is a glycosylated terpene, an acetylated terpene glycoside or a mixture of the two wherein that were generated in vivo.
   499. The oral pharmaceutical solution of embodiment 498 in sublingual spray form.
   500. An oral pharmaceutical solution comprising 0.54% w/w water-soluble terpene, 31.9% w/w water, 12% w/w polyethylene glycol 400, 5.5% w/w propylene glycol, 0.01% w/w butylated hydroxyanisole, 0.05% w/w sucralose, and 50% w/w alcohol.
   501. An solution for nasal and/or sublingual administration of a composition comprising:
an excipient of propylene glycol, ethanol anhydrous, or a mixture of both;
a water-soluble glycosylated terpene;
   502. The solution of embodiment 501, wherein said glycosylated terpene is generated in vivo.
   503. The solution of embodiment 501, wherein said glycosylated terpene is generated in vitro.
   504. The solution of embodiment 501, wherein said glycosylated terpene is non-psychoactive.
   505. The aqueous solution of embodiment 501, and further comprising a topical decongestant.
   506. The aqueous solution of embodiment 505, wherein said topical decongestant is selected from the group consisting of: phenylephrine hydrochloride, Oxymetazoline hydrochloride, and Xylometazoline.
   507. The aqueous solution of embodiment 501, and further comprising an antihistamine.
   508. The aqueous solution of embodiment 501, and further comprising a steroid.
   509. The aqueous solution of embodiment 509, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   510. The aqueous solution of embodiment 501, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   511. An solution for nasal and/or sublingual administration of a composition comprising:
an excipient of propylene glycol, ethanol anhydrous or a mixture of both; and
an water-soluble acetylated terpene glycoside.
   512. The solution of embodiment 511, wherein said acetylated terpene glycoside is generated in vivo.
   513. The solution of embodiment 511, wherein said acetylated terpene glycoside is generated in vitro.
   514. The solution of embodiment 511, wherein said acetylated terpene glycoside is non-psychoactive.
   515. The aqueous solution of embodiment 511, and further comprising a topical decongestant.
   516. The aqueous solution of embodiment 515, wherein said topical decongestant is selected from the group consisting of: phenylephrine hydrochloride, Oxymetazoline hydrochloride, and Xylometazoline.
   517. The aqueous solution of embodiment 511, and further comprising an antihistamine.
   518. The aqueous solution of embodiment 511, and further comprising a steroid.
   519. The aqueous solution of embodiment 518, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   520. The aqueous solution of embodiment 519, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   521. A solution for nasal and/or sublingual administration of a composition comprising:
an excipient of propylene glycol, ethanol anhydrous or a mixture of both; and
a water-soluble glycosylated terpene and an water-soluble acetylated terpene glycoside.
   522. The solution of embodiment 521, wherein said acetylated terpene glycoside and said glycosylated terpene is generated in vivo.
   523. The solution of embodiment 521, wherein said acetylated terpene glycoside and said glycosylated terpene is generated in vitro.
   524. The solution of embodiment 521, wherein said acetylated terpene glycoside and said glycosylated terpene are non-psychoactive.
   525. The aqueous solution of embodiment 521, and further comprising a topical decongestant.
   526. The aqueous solution of embodiment 525, wherein said topical decongestant is selected from the group consisting of: phenylephrine hydrochloride, Oxymetazoline hydrochloride, and Xylometazoline.
   527. The aqueous solution of embodiment 521, and further comprising an antihistamine.
   528. The aqueous solution of embodiment 521, and further comprising a steroid.
   529. The aqueous solution of embodiment 528, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   530. The aqueous solution of embodiment 529, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   531. An aqueous solution for nasal and/or sublingual administration of a compositions comprising:
a saline solution; and
a water-soluble glycosylated terpene.
   532. The aqueous solution of embodiment 531, wherein said glycosylated terpene is generated in vivo.
   533. The aqueous solution of embodiment 531, wherein said glycosylated terpene is generated in vitro.
   534. The aqueous solution of embodiment 531, wherein said glycosylated terpene is non-psychoactive.
   535. The aqueous solution of embodiment aqueous 531, and further comprising a topical decongestant.
   536. The aqueous solution of embodiment 535, wherein said topical decongestant is selected from the group consisting of: phenylephrine hydrochloride, Oxymetazoline hydrochloride, and Xylometazoline.
   537. The aqueous solution of embodiment 531, and further comprising an antihistamine.
   538. The aqueous solution of embodiment 531, and further comprising a steroid.
   539. The aqueous solution of embodiment 539, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   540. The aqueous solution of embodiment 531, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   541. An aqueous solution for nasal and/or sublingual administration of a composition comprising:
a saline solution; and
a water-soluble acetylated terpene glycoside.
   542. The aqueous solution of embodiment 541, wherein said acetylated terpene glycoside is generated in vivo.
   543. The aqueous solution of embodiment 541, wherein said acetylated terpene glycoside is generated in vitro.
   544. The aqueous solution of embodiment 541, wherein said acetylated terpene glycoside is non-psychoactive.
   545. The aqueous solution of embodiment 541, and further comprising a topical decongestant.
   546. The aqueous solution of embodiment 545, wherein said topical decongestant is selected from the group consisting of: phenylephrine hydrochloride, Oxymetazoline hydrochloride, and Xylometazoline.
   547. The aqueous solution of embodiment 546, and further comprising an antihistamine.
   548. The aqueous solution of embodiment 545, and further comprising a steroid.
   549. The aqueous solution of embodiment 548, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   550. The aqueous solution of embodiment 549, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   551. An aqueous solution for nasal and/or sublingual administration of a composition comprising:
a saline solution; and
a water-soluble acetylated terpene glycoside and a water-soluble glycosylated terpene.
   552. The aqueous solution of embodiment 551, wherein said acetylated terpene glycoside and said glycosylated terpene is generated in vivo.
   553. The aqueous solution of embodiment 551, wherein said acetylated terpene glycoside and said glycosylated terpene is generated in vitro.
   554. The aqueous solution of embodiment 551, wherein said acetylated terpene glycoside and said glycosylated terpene are non-psychoactive.
   555. The aqueous solution of embodiment 551, and further comprising a topical decongestant.
   556. The aqueous solution of embodiment 555, wherein said topical decongestant is selected from the group consisting of: phenylephrine hydrochloride, Oxymetazoline hydrochloride, and Xylometazoline.
   557. The aqueous solution of embodiment 551, and further comprising an antihistamine.
   558. The aqueous solution of embodiment 551, and further comprising a steroid.
   559. The aqueous solution of embodiment 557, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   560. The aqueous solution of embodiment 551, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   561. An aqueous solution for nasal and/or sublingual administration of a compositions comprising:
purified water; and
a water-soluble glycosylated terpene.
   562. The aqueous solution of embodiment 561, wherein said glycosylated terpene is generated in vivo.
   563. The aqueous solution of embodiment 561, wherein said glycosylated terpene is generated in vitro.
   564. The solution of embodiment 561, wherein said glycosylated terpene is non-psychoactive.
   565. The aqueous solution of embodiment 561, and further comprising a topical decongestant.
   566. The aqueous solution of embodiment 565, wherein said topical decongestant is selected from the group consisting of: phenylephrine hydrochloride, Oxymetazoline hydrochloride, and Xylometazoline.
   567. The aqueous solution of embodiment 561, and further comprising an antihistamine.
   568. The aqueous solution of embodiment 561, and further comprising a steroid.
   569. The aqueous solution of embodiment 568, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   570. The aqueous solution of embodiment 561, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   571. An aqueous solution for nasal and/or sublingual administration of a composition comprising:
purified water; and
a water-soluble acetylated terpene glycoside.
   572. The aqueous solution of embodiment 571, wherein said acetylated terpene glycoside is generated in vivo.
   573. The aqueous solution of embodiment 571, wherein said acetylated terpene glycoside is generated in vitro.
   574. The solution of embodiment 571, wherein said acetylated terpene glycoside is non-psychoactive.
   575. The aqueous solution of embodiment 571, and further comprising a topical decongestant.
   576. The aqueous solution of embodiment 575, wherein said topical decongestant is selected from the group consisting of: phenylephrine hydrochloride, Oxymetazoline hydrochloride, and Xylometazoline.
   577. The aqueous solution of embodiment 571, and further comprising an antihistamine.
   578. The aqueous solution of embodiment 571, and further comprising a steroid.
   579. The aqueous solution of embodiment 578, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   580. The aqueous solution of embodiment 579, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   581. An aqueous solution for nasal and/or sublingual administration of a composition comprising:
purified water; and
a water-soluble acetylated terpene glycoside and a water-soluble glycosylated terpene.
   582. The aqueous solution of embodiment 581, wherein said acetylated terpene glycoside and said glycosylated terpene is generated in vivo.
   583. The aqueous solution of embodiment 581, wherein said acetylated terpene glycoside and said glycosylated terpene is generated in vitro.
   584. The aqueous solution of embodiment 581, wherein said acetylated terpene glycoside and said glycosylated terpene are non-psychoactive.
   585. The aqueous solution of embodiment 581, and further comprising a topical decongestant.
   586. The aqueous solution of embodiment 585, wherein said topical decongestant is selected from the group consisting of: phenylephrine hydrochloride, Oxymetazoline hydrochloride, and Xylometazoline.
   587. The aqueous solution of embodiment 581, and further comprising an antihistamine.
   588. The aqueous solution of embodiment 581, and further comprising a steroid.
   589. The aqueous solution of embodiment 588, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   590. The aqueous solution of embodiment 581, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   591. A topical formulation consisting of a water-soluble glycosylated terpene, and/or water-soluble acetylated terpene glycoside, or a mixture of both, and a pharmaceutically acceptable excipient.
   592. The topical formulation according to embodiment 591, and further comprising a quantity of capsaicin.
   593. The topical formulation according to embodiment 591, and further comprising a quantity of benzocaine.
   594. The topical formulation according to embodiment 591, and further comprising a quantity of lidocaine.
   595. The topical formulation according to embodiment 591, and further comprising a quantity of camphor.
   596. The topical formulation according to embodiment 591, and further comprising a quantity of benzoin resin.
   597. The topical formulation according to embodiment 591, and further comprising a quantity of methylsalicilate.
   598. The topical formulation according to embodiment 591, and further comprising a quantity of triethanolamine salicylate.
   599. The topical formulation according to embodiment 591, and further comprising a quantity of hydrocortisone.
   600. The topical formulation according to embodiment 591, and further comprising a quantity of salicylic acid.
   601. The topical formulation according to embodiment 591, and further comprising a wherein the pharmaceutically acceptable excipient is selected from the group consisting of: gels, ointments, cataplasms, poultices, pastes, creams, lotions, plasters and jellies
   602. The topical formulation according to embodiment 591, and further comprising a polyethylene glycol.
   603. A gel for transdermal administration, the mixture preferably contains from 15% to about 90% ethanol, from about 10% to about 60% buffered aqueous solution or water, from about 0.1 to about 25% propylene glycol, from about 0.1 to about 20% of a gelling agent, from about 0.1 to about 20% of a base, from about 0.1 to about 20% of an absorption enhancer and from about 1% to about 25% polyethylene glycol and a water-soluble terpene.
   604. The gel of embodiment 603, wherein said water-soluble terpene comprises a water-soluble glycosylated terpene, and/or water-soluble acetylated terpene glycoside, or a mixture of both
   605. The gel of embodiment 604, wherein said glycosylated terpene, and/or said acetylated terpene glycoside were generated in vivo.
   606. The gel of embodiment 604, wherein said glycosylated terpene, and/or said acetylated terpene glycoside were generated in vitro.
   607. A formulation comprising the following volumetric amounts: (i) from about 15% to about 90% ethanol, (ii) a glycol selected from the group consisting of (a) propylene glycol from about 0.1% to about 25%, (b) polyethylene glycol from about 1 to about 30%, and (c) a combination of (a) and (b), (iii) from about 0.1 to about 20% of a gelling agent, (iv) from about 0.1 to about 20% of a base and (v) from about 0.1 to about 20% of an absorption enhancer, and a water-soluble terpene, said formulation being suitable for transdermal administration.
   608. The formulation of embodiment 607, wherein said water-soluble terpene comprises a water-soluble glycosylated terpene, and/or water-soluble acetylated terpene glycoside, or a mixture of both.
   609. The formulation of embodiment 608, wherein said glycosylated terpene, and/or said acetylated terpene glycoside were generated in vivo.
   610. The formulation of embodiment 608, wherein said glycosylated terpene, and/or said acetylated terpene glycoside were generated in vitro.
   611. A transdermal composition comprising a pharmaceutically effective amount of a water-soluble terpene for delivery of the terpene to the bloodstream of a user, said composition comprising:
a pharmaceutically acceptable excipient;
at least one water-soluble terpene;
wherein the terpene is capable of diffusing from the composition into the bloodstream of the user.
   612. The composition of embodiment 611, wherein the water-soluble terpene is selected from the group consisting of: a glycosylated terpene, an acetylated terpene glycoside, and a mixture of both.
   613. The composition of embodiment 612, wherein said glycosylated terpene, and/or said acetylated terpene glycoside were generated in vivo.
   614. The composition of embodiment 611, wherein the transdermal composition further comprises one or more pharmaceutically acceptable excipients to create a transdermal dosage form selected from the group consisting of: gels, ointments, cataplasms, poultices, pastes, creams, lotions, plasters and jellies.
   615. The composition of embodiment 611, and further comprising a surfactant.
   616. The composition of embodiment 611, wherein the surfactant is a surfactant-lecithin organogel.
   617. The composition of embodiment 611, wherein the surfactant-lecithin organogel is present in an amount of between about between about 95% and about 98% w/w.
   618. The composition of embodiment 611, wherein the surfactant-lecithin organogel comprises lecithin and PPG-2 myristyl ether propionate.
   619. The composition of embodiment 611, wherein the surfactant-lecithin organogel comprises a surfactant comprising high molecular weight polyacrylic acid polymers.
   622. The composition of embodiment 611, wherein the composition further comprises isopropyl myristate.
   623. The composition of embodiment 611, wherein the water-soluble terpene is non-psychoactive.
   624. The composition of embodiment 611, wherein the pharmaceutically acceptable excipients is selected from the group consisting of: gels, ointments, cataplasms, poultices, pastes, creams, lotions, plasters and jellies
   625. A transdermal composition comprising a pharmaceutically effective amount of a water-soluble terpene for delivery of the terpene to the bloodstream of a user, said composition comprising:
a permeation enhancer;
at least one water-soluble terpene;
wherein the terpene is capable of diffusing from the composition into the bloodstream of the user.
   626. The composition of embodiment 625, wherein the water-soluble terpene is selected from the group consisting of: a glycosylated terpene, an acetylated terpene glycoside, and a mixture of both.
   627. The composition of embodiment 626, wherein said glycosylated terpene, and/or said acetylated terpene glycoside were generated in vivo.
   628. The composition of embodiment 625, wherein the permeation enhancer is selected from the group consisting of: propylene glycol monolaurate, diethylene glycol monoethyl ether, an oleoyl macrogolglyceride, a caprylocaproyl macrogolglyceride, and an oleyl alcohol.
   629. The composition of embodiment 625, herein the transdermal composition further comprises one or more pharmaceutically acceptable excipients to create a transdermal dosage form selected from the group consisting of: gels, ointments, cataplasms, poultices, pastes, creams, lotions, plasters and jellies.
   630. A liquid terpene liniment composition consisting of water, isopropyl alcohol solution and a water-soluble terpene.
   631. The composition of embodiment 630, wherein said water-soluble terpene is selected from the group consisting of: a glycosylated terpene, an acetylated terpene glycoside, and a mixture of both.
   632. The composition of embodiment 632, wherein said glycosylated terpene, and/or said acetylated terpene glycoside were generated in vivo.
   633. The composition of embodiment 630, consisting of from about 97.5% to about 99.5% by weight of 70% isopropyl alcohol solution and from about 0.5% to about 2.5% by weight of a terpene mixture
   634. A commercially available topical creme composition infused with a glycosylated terpene, an acetylated terpene glycoside, and a mixture of both.
   635. The composition of embodiment 634, wherein said glycosylated terpene, and/or said acetylated terpene glycoside were generated in vivo.
   636. A commercially available lip balm composition supplemented with a water-soluble terpene wherein said comprises a glycosylated terpene, and/or an acetylated terpene glycoside, and/or a mixture of both.
   637. The composition of embodiment 636, wherein said glycosylated terpene, and/or said acetylated terpene glycoside were generated in vivo.
   638. A commercially available cosmetic composition supplemented with a water-soluble terpene wherein said comprises a glycosylated terpene, and/or an acetylated terpene glycoside, and/or a mixture of both.
   639. The composition of embodiment 638, wherein said glycosylated terpene, and/or said acetylated terpene glycoside were generated in vivo.
   640. A tobacco plant containing at least one water-soluble terpenes.
   641. The tobacco plant in embodiment 640, wherein said water-soluble terpene comprises a glycosylated terpene, and/or an acetylated terpene glycoside, and/or a mixture of both.
   642. The tobacco plant of embodiment 641, wherein said glycosylated terpene, and/or said acetylated terpene glycoside were generated in vivo.
   643. The tobacco plant of embodiment 641, wherein said glycosylated terpene, and/or said acetylated terpene glycoside were generated in vitro.
   644. The tobacco plant of embodiment 640, wherein said water-soluble terpene is non-psychoactive.
   645. The tobacco plant of embodiment 640, wherein said tobacco plant is used to generate a water-soluble terpene infused tobacco product.
   646. The tobacco plant of embodiment 645, wherein said terpene infused tobacco product is a cigarette, pipe tobacco, chewing tobacco, cigar, smokeless tobacco.
   646. A composition comprising:
an aqueous solution;
water-soluble terpene dissolved in said aqueous solution wherein said water-soluble terpene comprises a glycosylated terpene, and/or an acetylated terpene glycoside, and/or a mixture of both;
wherein said composition may be introduced to a cigarette and/or a tobacco leaf such that said aqueous solution may evaporate generating a cigarette and/or a tobacco leaf that contains said water-soluble terpene.
   647. The composition of embodiment 646, wherein said glycosylated terpene, and/or said acetylated terpene glycoside were generated in vivo.
   648. The composition of embodiment 646, wherein said glycosylated terpene, and/or said acetylated terpene glycoside were generated in vivo.
   649. The composition of embodiment 646, wherein said water-soluble terpene is non-psychoactive.
   650. The composition of embodiment 646, wherein said aqueous solution comprises purified water.
   651. A method of treating a medical condition in a mammal comprising the step of administering a therapeutically effective amount of a glycosylated terpene, and/or an acetylated terpene glycoside, and/or a mixture of both or a pharmaceutically acceptable salt thereof, wherein the medical condition is selected from the group consisting of: obesity, post-traumatic stress syndrome, anorexia, nausea, emesis, pain, wasting syndrome, HIV-wasting, chemotherapy induced nausea and vomiting, alcohol use disorders, anti-tumor, amyotrophic lateral sclerosis, glioblastoma multiforme, glioma, increased intraocular pressure, glaucoma, Cannabis use disorders, Tourette's syndrome, dystonia, multiple sclerosis, inflammatory bowel disorders, arthritis, dermatitis, Rheumatoid arthritis, systemic lupus erythematosus, anti-inflammatory, anti-convulsant, anti-psychotic, anti-oxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, synovitis, and juvenile rheumatoid arthritis.
   652. The method of embodiment 651 wherein the compound is administered by a route selected from the group consisting of: transdermal, topical, oral, buccal, sublingual, intra-venous, intra-muscular, vaginal, rectal, ocular, nasal and follicular.
   653. The method of embodiment 652, wherein said glycosylated terpene, and/or an acetylated terpene glycoside, and/or a mixture of both are glycosylated terpene, and/or acetylated in vivo.

TABLE 1
Proposed terpene glycoside species. Molecular formula and weight, expected and observed mass-to-charge ratio (m/z),
mass error (ppm) and observed retention time (RT, min) are provided for each observed terpene glycoside species

| | Molecular | MW | Expected | Observed | Mass | Observed |
|---|---|---|---|---|---|---|
| Species | Formula | (g/mol) | m/z | m/z | error (ppm) | RT (min) |
| alpha-Bisabolol | C15H26O1 | 222.36634 | — | — | — | — |
| alpha-Bisabolol monoglucoside | C21H36O6 | 384.2512 | 383.2432 | 383.2458 | 4.8 | 15.50 |
| alpha-Bisabolol O-acetyl glucoside | C23H38O7 | 426.2618 | 425.2538 | 425.2558 | 3.1 | 15.45 |
| alpha-Bisabolol oxidized glucoside | C21H34O7 | 398.2305 | 397.2225 | 397.22295 | −0.4 | 13.74 |
| alpha-Bisabolol diglucoside | C27H46O11 | 546.3040 | 545.2961 | 545.2977 | 1.8 | 10.58 |
| Citronellyl monoglucoside | C16H30O6 | 318.3418 | 317.3339 | 317.3336 | −3.0 | 14.18 |
| Geranyl O-acetyl glucoside | C18H38O7 | 358.1992 | 357.1912 | 357.1918 | −0.3 | 9.99 |
| Geranyl monoglucoside | C16H28O6 | 316.1886 | 315.1806 | 315.1798 | −5.0 | 10.07 |
| Linalyl monoglucoside | C16H28O6 | 316.1886 | 315.1806 | 315.1803 | −3.0 | 11.21 |
| Linalyl glucoside-xyloside | C21H36O10 | 448.2308 | 447.2229 | 447.2227 | −1.9 | 9.16 |
| Linalyl glucoside | C22H38O11 | 478.2414 | 477.2335 | 477.2330 | −2.5 | 8.84 |
| Neryl monoglucoside | C16H28O6 | 316.1886 | 315.1806 | 315.1807 | −1.9 | 11.15 |
| Neryl diglucoside | C22H38O11 | 478.2414 | 477.2335 | 477.2341 | −0.2 | 7.77 |
| Terpineol diglucoside | C22H38O11 | 478.2414 | 477.2335 | 477.2352 | 2.3 | 7.66 |

TABLE 2
Yeast transgenic lines screened for terpene glycosylation.
Yeast transformant lines
HC-empty vector
UGT58A1
MhGt1
VvGt7
UGT85K11
UGT94P1
UGT85K11 + UGT94P1 (B2)
NtGt3
NtGt4

TABLE 3
Summary of the proposed terpene glycoside species observed in
yeast cell pellet extracts from a 96-well plate screening experiment

| | | | | | | Ob- |
|---|---|---|---|---|---|---|
| | | | | Ob- | Mass | served |
| | Molecular | MW | Expected | served | error | RT |
| Species | Formula | (g/mol) | m/z | m/z | (ppm) | (min) |
| Linalyl | C16H28O6 | 316.1886 | 315.1806 | 315.1803 | −3.0 | 11.214 |
| mono- | | | | | | |
| glucoside | | | | | | |
| Alpha- | C21H36O6 | 384.2512 | 383.2432 | 383.2458 | 4.8 | 15.495 |
| bisabolyl | | | | | | |
| glycoside | | | | | | |

TABLE 4
Summary of the proposed terpene glycoside species observed in yeast cell pellet extracts from
10 mL flask incubation experiments. Molecular formula and weight, expected and observed
mass-to-charge ratio (m/z), mass error (ppm) and observed retention time (RT, min)
are provided for each observed terpene glycoside species

| | Molecular | MW | Expected | Observed | Mass | Observed |
|---|---|---|---|---|---|---|
| Species | Formula | (g/mol) | m/z | m/z | error (ppm) | RT (min) |
| alpha-Bisabolol | C21H34O7 | 398.2305 | 397.2225 | 397.22295 | −0.35 | 13.74 |
| oxidized glucoside | | | | | | |
| Citronellyl | C16H30O6 | 318.3418 | 317.3339 | 317.3336 | −3.0 | 14.18 |
| monoglucoside | | | | | | |
| Neryl monoglucoside | C16H28O6 | 316.1886 | 315.1806 | 315.1807 | −1.9 | 11.15 |
| Neryl diglucoside | C22H38O11 | 478.2414 | 477.2335 | 477.2341 | −0.2 | 7.77 |
| Terpineol diglucoside | C22H38O11 | 478.2414 | 477.2335 | 477.2352 | 2.3 | 7.66 |

TABLE 6
Common Terpenoids and Synergistic Cannabinoids

| | | | Synergistic |
|---|---|---|---|
| Terpenoid | Structure | Pharmacological activity (Reference) | cannabinoid |
| Limonene | 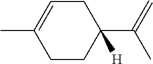 | Potent AD/immunostimulant via inhalation (Komori et al., 1995) Anxielytic (Carvalho-Freitas and Costa, 2002; Pultrini Ade et al., 2006) via 5-HT_{EA} (Komiya | CBD CBD |
| | | et al., 2006) | |
| | | Apoptosis of breast cancer cells (Vigushin | CBD, CBG |
| | | et al., 1998) | |
| | | Active against acne bacteria (Kim et al., 2008) | CBD |
| | | Dermatophytes (Sanguinetti et al., 2007; | CBG |
| | | Singh et al., 2010) | |
| | | Gastro-aesophageal reflux (Harris, 2010) | THC |
| α-Pinene | 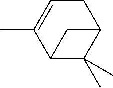 | Anti-inflammatory via PGE-3 (Ce et al., 1989) Bronchodilatory in humans (Falk et al., 1990) Acetylcholinesterase inhibitor, aiding memory (Perry et al., 2000) | CBD THC THC?, CBD |
| β-Myrcene | 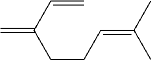 | Blocks inflammation via PGE-2 (Lorenzetti et al., 1991) Analgesic, antagonized by naloxone (Rao et al., 1990) | CBD CBD, THC |
| | | Sedating, muscle relaxant, hypnotic (da Vale | THC |
| | | et al., 2002) | |
| | | Blocks hepatic carcinogenesis by aflatoxin | CBD, CBG |
| | | (de Oliveira et al., 1997) | |
| Linalool | 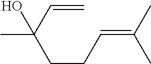 | Anti-anxiety (Passso, 2001) Sedative on inhalation in mice (Buchbauer et al., 1993) Local anesthetic (Re et al., 2000) | CBD, CBG? THC THC |
| | | Analgesic via adenosine Aza (Peana et al., | CBD |
| | | 2006) | |
| | | Anticonvulsant/anti-glutamate (Elisabetsky | CBD, THCV, |
| | | et al., 1995) | CBDV |
| | | Potent anti-leshmarial (do Socorro et al., | ? |
| | | 2003) | |
| β-Caryophyllene | 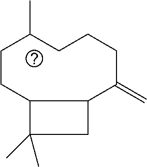 | A1 via PGE-1 comparable phenylbutazone (Basile et al., 1988) Gastric cytoprotective (Tambe et al., 1996) Anti-malarial (Campbell et al., 1997) Selective CB₂ agonist (100 nM) Gertsch et al., 2008) Treatment of peruritust (Karsek et al., 2007) Treatment of addiction? (Xi et al., 2010) | CBD THC ? THC THC CBD |
| Caryophyllene Oxide | 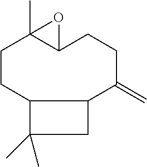 [SMILES: C=C1CCC2OC2(C)CCC2C1CC2(C)C] | Decreases platelet aggregation (Lin et al., 2003) Antifungal in onychomycosis comparable to ciclopiroxolamine and sulcanazole (Yang et al., 1999) Insecticidal/anti-fredant (Bettarini et al., 1993) | THC CBC, CBG THCA, CBGA |
| Nerolidol | 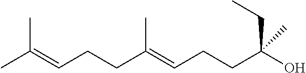 [SMILES: CC[C@@](C)(O)CC/C=C(\C)CCC=C(C)C] | Sedative (Binet et al., 1972) Skin penetrant (Cornwell and Barry, 1994) Potent antimalarial (Lopes et al., 1999, Rodrigues Gouiart et al., 2004) Anti-leishmarial activity (Arruda et al., 2005) | THC, CBN — ? ? |
| Phytol | 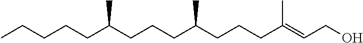 [SMILES: CCCCC[C@@H](C)CCC[C@@H](C)CCC/C(C)=C/CO] | Breakdown product of chlorophyll Prevents Vitamin A teratogenesis (Arnhold et al., 2002) | — — |
| | | ↑GABA via SSADH inhibition (Bang et al., | CBG |
| | | 2002) | |

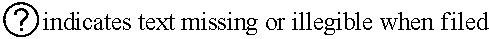

TABLE 7
Geranyl glycoside species observed in BY2 cell suspension
cultures fed with geraniol

| | | | | | | Ob- |
|---|---|---|---|---|---|---|
| | | | | Ob- | Mass | served |
| | Molecular | MW | Expected | served | error | RT |
| Species | Formula | (g/mol) | m/z | m/z | (ppm) | (min) |
| Geranyl | C16H28O6 | 316.1886 | 315.1806 | 315.1798 | −5.0 | 10.07 |
| mono- | | | | | | |
| glucoside | | | | | | |

TABLE8
RT-PCR primers used in this application.

| Target | | |
|---|---|---|
| gene | Forward primer | Reverse primer |
| UGT58A1 | CCACCACCACATGATCAAAGAAC(SEQ | GTCATTTGTCTAGCAGTACCCCA |
| | IDNO. 15) | (SEQ ID NO. 16) |
| MhGT1 | GATTGATATGGCTTTGTGGGCTG(SEQ | CTCTCAAACCAGCAGCAGTAGTA |
| | IDNO. 17) | (SEQ ID NO. 18) |
| NtGT3 | ATGAAAGAGACTAAAAAAATTGAGT | CATCACGCAGATTTTGAATATGG |
| | (SEQ ID NO. 19) | (SEQ ID NO. 20) |
| NtGT4 | ATGGCTACTCAGGTGCATAAATTGC | GGCCTTAGTTAGCTCGACACGG |
| | (SEQ ID NO. 21) | (SEQ ID NO. 22) |
| VvGT7 | TACAACACTGGTTCTACTGCTCC(SEQ | GCCCAAGACTTAACCATCAAACC |
| | IDNO. 23) | (SEQ ID NO. 24) |
| UGT85K11 | CCAGAGTGACGGTTCATTAGACA (SEQ | TCAACGAAGTGTTCGGGTAAGAT |
| | IDNO. 25) | (SEQ ID NO. 26) |
| UGT94P1 | CTCCACCATCCTCAACACACTAA (SEQ | CCATGCTAAGCTCTAACCCATGA |
| | IDNO. 27) | (SEQ ID NO. 28) |

TABLE 9
dominant free terpenoids present in C. sativa.

| | Retention | Collision | |
|---|---|---|---|
| Compound | time (mins) | energy (V) | Q1 > Q3 |
| Beta-Myrcene | 7.47 | 17, 16 | 137 > 95, 81 |
| Limonene | 8.16 | 17, 16 | 137 > 95, 81 |
| Alpha-Pinene | 8.37 | 17, 16 | 137 > 95, 81 |
| Linalool | 3.52 | 17, 16 | 137, 95, 81 |
| Linalyl glucoside | 11.8 | −12, −20* | 315 > 119, 161 |
| Beta-Caryophyllene | 12.07 | 17, 27 | 205 > 149, 93 |
| Caryophyllene Oxide | 6.10 | TBD | 203 > 147, 105 |
| Nerolidol | 6.68 | TBD | 205 > 149, 93 |
| Phytol | TBD | TBD | 297 > TBD |

### REFERENCES

[1] Bachhawat, Preeti, Saroj Mishra, Yukti Bhatia, and V. S. Bisaria. 2004. “Enzymatic Synthesis of Oligosaccharides, Alkyl and Terpenyl Glucosides, by Recombinant Escherichia coli Expressed Pichia Etchellsiiβ-Glucosidase II.” Applied Biochemistry and Biotechnology 118 (1-3): 269-82. https://doi.org/10.1385/ABAB:118:1-3:269.
[2] Bonisch, F., J. Frotscher, S. Stanitzek, E. Ruhl, M. Wust, O. Bitz, and W. Schwab. 2014. “A UDP-Glucose:Monoterpenol Glucosyltransferase Adds to the Chemical Diversity of the Grapevine Metabolome.” PLANT PHYSIOLOGY 165 (2): 561-81. https://doi.org/10.1104/pp. 113.232470.
[3] Bönisch, Friedericke, Johanna Frotscher, Sarah Stanitzek, Ernst Rühl, Matthias Wüst, Oliver Bitz, and Wilfried Schwab. 2014. “Activity-Based Profiling of a Physiologic Aglycone Library Reveals Sugar Acceptor Promiscuity of Family 1 UDP-Glucosyltransferases from Grape.” Plant Physiology 166 (1): 23-39. https://doi.org/10.1104/pp. 114.242578.
[4] Caputi, Lorenzo, Eng-Kiat Lim, and Dianna J. Bowles. 2008. “Discovery of New Biocatalysts for the Glycosylation of Terpenoid Scaffolds.” Chemistry—A European Journal 14 (22): 6656-62. https://doi.org/10.1002/chem.200800548.
[5] Eiichiro Ono, Nobuo Tsuruoka. n.d. “MONOTERPENEGLYCOSYLTRANSFERASE ORIGINATING FROM HOPAND METHOD FOR USING SAME.” U.S. Pat. No. 9,574,182
[6] Feng, Jin, Peng Zhang, Yinglu Cui, Kai Li, Xue Qiao, Ying-Tao Zhang, Shu-Ming Li, et al. 2017. “Regio- and Stereospecific o-Glycosylation of Phenolic Compounds Catalyzed by a Fungal Glycosyltransferase from Mucor hiemalis.” Advanced Synthesis & Catalysis 359 (6): 995-1006. https://doi.org/10.1002/adsc.201601317.
[7] Gebrehiwot, Hadush, R. Bachetti, and Aman Dekebo. 2015. “Chemical Composition and Antimicrobial Activities of Leaves of Sweet Basil (Ocimum basilicum L.) Herb.” International Journal of Basic and Clinical Pharmacology, 869-75. https://doi.org/10.18203/2319-2003.ijbcp20150858.
[8] Hyland, K. C., Christopher Borton, Paul Winkler, Simon Roberts, and Mathew Noestheden. 2017. “Quantitation of Terpenes in Cannabis Products Using the Triple Quad™ 3500 LC-MS/MS System.” SCIEX, Redwood City, Calif., 2 SCIEX Concord, Ontario (Canada).
[9] Kollmannsberger, Hubert, M. Biendl, and S. Nitz. 2006. “Occurrence of Glycosidically Bound Flavour Compounds in Hops, Hop Products and Beer.” Monatsschrift Fur Brauwissenschaft. 59: 83-89.
[10] Kren, V., and L. Martinková. 2001. “Glycosides in Medicine: ‘The Role of Glycosidic Residue in Biological Activity.’” Current Medicinal Chemistry 8 (11): 1303-28.
[11] Kwon, S., K. Shimoda, H. Hamada, K. Ishihara, N. Masuoka, and H. Hamada. 2008. “High Production of β-Thujaplicin Glycosides by Immobilized Plant Cells of Nicotiana tabacum.” Acta Biologica Hungarica 59 (3): 347-55. https://doi.org/10.1556/ABiol.59.2008.3.8.
[12] Li, Xiang-Yi, Ya-Qin Wen, Nan Meng, Xu Qian, and Qiu-Hong Pan. 2017. “Monoterpenyl Glycosyltransferases Differentially Contribute to Production of Monoterpenyl Glycosides in Two Aromatic Vitis vinifera Varieties.” Frontiers in Plant Science 8 (July). https://doi.org/10.3389/fpls.2017.01226.
[13] Magnard, J.-L., A. Roccia, J.-C. Caissard, P. Vergne, P. Sun, R. Hecquet, A. Dubois, et al. 2015. “Biosynthesis of Monoterpene Scent Compounds in Roses.” Science 349 (6243): 81-83. https://doi.org/10.1126/science.aab0696.
[14] McHale, Kevin J., and Rory Doyle. n.d. “Measurement of Terpenes in Plant Extracts via LC-MS/MS.” Thermo Fisher Scientific, Somerset, N.J. 08873.
[15] Park, Si-Hyung, and Soo-Un Kim. 1998. “Modified Monoterpenes from Biotransformation of (−)-Isopiperitenone by Suspension Cell Culture of Mentha piperita.” Journal of Natural Products 61 (3): 354-57. https://doi.org/10.1021/np970388k.
[16] Rivas, Francisco, Andres Parra, Antonio Martinez, and Andres Garcia-Granados. 2013. “Enzymatic Glycosylation of Terpenoids.” Phytochemistry Reviews 12 (2): 327-39. https://doi.org/10.1007/s11101-013-9301-9.
[17] Roode, B. M. de, L. Oliehoek, A. van der Padt, M. C. R. Franssen, and R. M. Boom. 2001. “Downstream Processing of Enzymatically Produced Geranyl Glucoside.” Biotechnology Progress 17 (5): 881-86. https://doi.org/10.1021/bp010081i.
[18] Russo, Ethan B. 2011. “Taming THC: Potential Cannabis Synergy and Phytocannabinoid-Terpenoid Entourage Effects: Phytocannabinoid-Terpenoid Entourage Effects.” British Journal of Pharmacology 163 (7): 1344-64. https://doi.org/10.1111/j.1476-5381.2011.01238.x.
[19] Schwab, Wilfried, Thilo Fischer, and Matthias Wüst. 2015. “Terpene Glucoside Production: Improved Biocatalytic Processes Using Glycosyltransferases.” Engineering in Life Sciences 15 (4): 376-86. https://doi.org/10.1002/elsc.201400156.
[20] Sethi, Benu, Monika Jain, Manish Chowdhary, Yogesh Soni, Yukti Bhatia, Vikram Sahai, and Saroj Mishra. 2002. “Cloning, Characterization Of Pichia etchellsii β-Glucosidase II and Effect of Media Composition and Feeding Strategy on Its Production in a Bioreactor.” Biotechnology and Bioprocess Engineering 7 (1): 43-51. https://doi.org/10.1007/BF02935879.
[21] Shimoda, Kei, Yoko Kondo, Tomohisa Nishida, Hatsuyuki Hamada, Nobuyoshi Nakajima, and Hiroki Hamada. 2006. “Biotransformation of Thymol, Carvacrol, and Eugenol by Cultured Cells of Eucalyptus perriniana.” Phytochemistry 67 (20): 2256-61. https://doi.org/10.1016/j.phytochem.2006.07.006.
[22] Wang, G., L. Tian, N. Aziz, P. Broun, X. Dai, J. He, A. King, P. X. Zhao, and R. A. Dixon. 2008. “Terpene Biosynthesis in Glandular Trichomes of Hop.” PLANT PHYSIOLOGY 148 (3): 1254-66. https://doi.org/10.1104/pp. 108.125187.
[23] Wen, Ya-Qin, Gan-Yuan Zhong, Yuan Gao, Yi-Bin Lan, Chang-Qing Duan, and Qiu-Hong Pan. 2015. “Using the Combined Analysis of Transcripts and Metabolites to Propose Key Genes for Differential Terpene Accumulation across Two Regions.” BMC Plant Biology 15 (1). https://doi.org/10.1186/s12870-015-0631-1.
[24] Xanthakis, Epameinondas, Sophia Magkouta, Heleni Loutrari, Haralambos Stamatis, Charis Roussos, and Fragiskos N. Kolisis. 2009. “Enzymatic Synthesis of Perillyl Alcohol Derivatives and Investigation of Their Antiproliferative Activity.” Biocatalysis and Biotransformation 27 (3): 170-78. https://doi.org/10.1080/10242420902811089.
[25] Yang, Li, Jianhua Zhu, Liyan Song, Xiaojian Shi, Xingyi Li, and Rongmin Yu. 2012. “Three Sesquiterpene Compounds Biosynthesised from Artemisinic Acid Using Suspension-Cultured Cells of Averrhoa carambola (Oxalidaceae).” Natural Product Research 26 (15): 1388-94. https://doi.org/10.1080/14786419.2011.589055.
[26] Yauk, Yar-Khing, Claire Ged, Mindy Y. Wang, Adam J. Matich, Lydie Tessarotto, Janine M. Cooney, Christian Chervin, and Ross G. Atkinson. 2014. “Manipulation of Flavour and Aroma Compound Sequestration and Release Using a Glycosyltransferase with Specificity for Terpene Alcohols.” The Plant Journal: For Cell and Molecular Biology 80 (2): 317-30. https://doi.org/10.1111/tpj.12634.
[27] Iijima, Y. 2004. “Characterization of Geraniol Synthase from the Peltate Glands of Sweet Basil.” PLANT PHYSIOLOGY 134 (1): 370-79. https://doi.org/10.1104/pp. 103.032946.
[28] PCT/US2014/046694
[29] Marks, M. D., Tian, L., Wenger, J. P., Omburo, S. N., Soto-Fuentes, W., He, J., . . . Dixon.
   R. A. (2009). Identification of candidate genes affecting Δ⁹-tetrahydrocannabinol biosynthesis in Cannabis sativa. Journal of Experimental Botany, 60(13), 3715-3726. http://doi.org/10.1093/jxb/erp210
[30] Matías-Hernández, et. at., (2017), AaMYB1 and its orthologue AtMYB61 affect terpene metabolism and trichome development in Artemisia annua and Arabidopsis thaliana. The Plant Journal, 90: 520-534. doi: 10.1111/tpj.13509
[31] Taura, Futoshi, et al., (2007) Cannabidiolic-acid synthase, the chemotype-determining enzyme in the fiber-type Cannabis sativa. Feb Letters Volume 581, Issue 16: 2929-2934
[32] Booth J K, Page J E, Bohlmann J (2017) Terpene synthases from Cannabis sativa. PLOS ONE 12(3): e0173911
[33] Use of a Promiscuous Glycosyltransferase from Bacillus subtilis 168 for the Enzymatic Synthesis of Novel Protopanaxatriol-Type Ginsenosides. Longhai Dai, Jiao Li, Jiangang Yang, Yueming Zhu, Yan Men, Yan Zeng, Yi Cai, Caixia Dong, Zhubo Dai, Xueli Zhang, and Yuanxia Sun Journal of Agricultural and Food Chemistry 2018 66 (4), 943-949

### SEQUENCE LISTINGS

All nucleotide and amino acid sequences listed in U.S. Provisional Patent Application No. 62/746,053, filed Oct. 16, 2018 are specifically incorporated herein by reference. The following sequences are further provided herewith and are hereby incorporated into the specification in their entirety:

SEQ ID NO. 1
DNA
UGT58A1 (codon-optimized)
Absidia coerulea
ATGGGTATTTTTTTGATGAAGTTGTTGTTTTTGTTGGTTTCTTCTATTATTACTTTGGTTTCTTGTTTGC
AATTGGAACCAACTTTTAGATCTACTCCAAAGAGAATTGTTTTTTGTTCTTTGTTGCCAGGTTCTTCTCA
TGTTAACTGGGTTTTGCCATGGTTGGATGAATTGGCTTTGAGAGGTCATGATGTTACTTTTATTACTATG
GATATGAACGTTAAGTTTGCTGTTCCATTTCCAAGAATTGAAACTATTCCAGTTTCTCATGCTAAGATTG
ATGTTAAGTCTATTTTTAGAGATTTGGGTTCTCATCCAGATCCATTGAAGATGATTTCTAAGATTTTTAC
TATTCATAAGTCTGGTTGGAGAACTGGTTTTGATTCTGTTTCTAACTACATTGAATCTAACCAAGTTGAT
GTTGTTATTTGTGATCATATGTCTGATCCATGTATTGATGCTGTTTTGGCTCATGATTTGCCATTGGTTG
TTACTTCTACTTTGGCTTTTTTTCCAGATGCTAAGACTGGTTACATTAACAACGATCCATTGACTATGAC
TGAATTTACTACTCAACATCAATCTATTTCTAGAAGATTTTACAACAAGTACATTTTGCCATTGATTTTG
TTGGCTAAGATTAAGCCACAATTGAACCAATTGGATCAATTGAAGAGACAAGCTGGTTTTATTCCACCAC
CACATGATCAAAGAACTTCTAACGCTTTGAAGATTGTTTCTTCTTTGTACGGTTTGGAACCAGCTAGACC
AATGGGTCCATTGGTTGAATTGGTTGGTCCAGTTTTGTCTCCAAACTACGCTGATTTGGATTTGACTTTT
AAGTCTTACTTGGATACTCATCATAAGGTTGCTTACGTTGCTTTTGGTCAACATGCTGCTCCAGATAGAC
AAGATATTGCTTTGATTTTGACTTCTTTGTTGGCTCAAGTTGAAAAGGGTCATTTGGATGGTATTATTTG
GTCTTCTAGATCTGCTTCTGTTGATACTTTTCCACCATCTATTGTTTCTTCTTACTCTAGAAAGACTTGG
GATTTGACTGGTGTTTTTCATCAAGAACAAAAGGGTCCATTGTTTATTACTTCTTGGTCTCCACAATTGG
CTGTTTTGTTGCATCCATCTGTTTCTGTTTTTTTGTCTCATGGTGGTTCTAACTCTTTGGTTGAATCTTT
GTACGCTGGTAAGAGATTGTTGTTTTACCCATTTTTTGGTGATCAATGGGGTACTGCTAGACAAATGACT
TTGTGTGGTTTGGGTGAATACTTTGGTCCAACTACTCCACAAGCTCAAGTTGATGAAAGAGTTGAAGCTG
TTGTTGTTGATCAACAAGGTAGATACCAAGCTAACGTTGGTAGATACCAAGCTATGGTTCAAATTCATTC
TCAAACTGCTCCAAGAAGAGCTGCTGATTTGGTTGAAGAAGTTGCTTTTGCTTCTACTCAATCTACTTTG
CCACATAGACATGATGTTGGTAGATCTATGTCTATGATTAAGAGAAACAACTGGGATATTCATACTGCTG
CTGTTTTGATTGTTGTTACTTTGATTGGTTTGGTTTACAGAGGTGTTTCTTCTAGAAGAATTAAGCAAAA
GATTTTGTAA
SEQ ID NO. 2
Amino Acid
UGT58A1
Absidia coerulea
MGIFLMKLLFLLVSSIITLVSCLQLEPTFRSTPKRIVFCSLLPGSSHVNWVLPWLDELALRGHDVTFITM
DMNVKFAVPFPRIETIPVSHAKIDVKSIFRDLGSHPDPLKMISKIFTIHKSGWRTGFDSVSNYIESNQVD
VVICDHMSDPCIDAVLAHDLPLVVTSTLAFFPDAKTGYINNDPLTMTEFTTQHQSISRRFYNKYILPLIL
LAKIKPQLNQLDQLKRQAGFIPPPHDQRTSNALKIVSSLYGLEPARPMGPLVELVGPVLSPNYADLDLTF
KSYLDTHHKVAYVAFGQHAAPDRQDIALILTSLLAQVEKGHLDGIIWSSRSASVDTFPPSIVSSYSRKTW
DLTGVFHQEQKGPLFITSWSPQLAVLLHPSVSVFLSHGGSNSLVESLYAGKRLLFYPFFGDQWGTARQMT
LCGLGEYFGPTTPQAQVDERVEAVVVDQQGRYQANVGRYQAMVQIHSQTAPRRAADLVEEVAFASTQSTL
PHRHDVGRSMSMIKRNNWDIHTAAVLIVVTLIGLVYRGVSSRRIKQKIL
SEQ ID NO. 3
DNA
MhGT1 codon-optimized:
Mucor hiemalis
ATGGTTGGTGTTCATATGGCTGAAAAGTTGCAAGTTCCATACTTTAGATCTATGCCATTTCCATTTTCTA
GAACTACTAAGTTTCCACATCCATTTGCTATTGCTCAAACTTCTGCTGGTGGTAGAATTTACAACGATAT
GACTTACGTTATGATTGATATGGCTTTGTGGGCTGGTACTTCTAAGTACGTTAACAGATTTAGAAGAAAC
GTTTTGAACTTGCCATCTACTACTTTGGATAGATTGCATTTGTGGAAGGTTCCACATATTTACTCTTTTT
CTCCAACTGTTGTTCATCCACCAAAGGATTGGCCAGATTACATTCATTGTACTGGTTACTGGTTTTTGGA
TAACCCAGATAGAGTTTGGCAACCAGATCCAGCTTTGGTTTCTTTTTTGGAAAACAAGGAAAAGCCAATT
GTTTACATTGGTTTTGGTTCTATTATTGTTCCAGATGCTGAAGAAACTACTAGAACTATTGTTCAAGCTG
TTTTGCAATCTGGTGTTAAGGCTATTATTTGTAAGGGTTGGTCTACTAGAGGTACTGAAAAGGATAAGAA
GGAAGATGGTGAATTGTTGGATCAATACCCAGGTATTATTCATCATTTGGATTCTGTTCCACATGATTGG
TTGTTTCCACAAATTCAAGGTGTTGTTCATCATGGTGGTGCTGGTACTACTGCTGCTGGTTTGAGAGCTG
GTTTGCCAACTATTATTAAGCCATTTTTTGGTGATCAAAGATTTTGGGGTCAAAGAGTTGAAGAATTGCA
AGTTGGTATTTGTTTGAACAAGTTGACTAAGTCTTCTTTGGCTGAAGCTTTGATTGCTATTACTCAAAAC
AAGACTATGATTGCTAAGGCTAACATTATTGGTGAAACTATTAGAAAGGAAAACGGTCCAAGACAAGCTG
TTGAAAACATTTACAGAGAATTTGATTACGCTAGAGAACAAAGAACTAACCAAGGTTCTTAA
SEQ ID NO. 4
Amino Acid
MhGT1
Mucor hiemalis
MVGVHMAEKLQVPYFRSMPFPFSRTTKFPHPFAIAQTSAGGRIYNDMTYVMIDMALWAGTSKYVNRFRRN
VLNLPSTTLDRLHLWKVPHIYSFSPTVVHPPKDWPDYIHCTGYWFLDNPDRVWQPDPALVSFLENKEKPI
VYIGFGSIIVPDAEETTRTIVQAVLQSGVKAIICKGWSTRGTEKDKKEDGELLDQYPGIIHHLDSVPHDW
LFPQIQGVVHHGGAGTTAAGLRAGLPTIIKPFFGDQRFWGQRVEELQVGICLNKLTKSSLAEALIAITQN
KTMIAKANIIGETIRKENGPRQAVENIYREFDYAREQRTNQGS
SEQ ID NO. 5
DNA
UGT85K11 codon-optimized
Camellia sinensis
ATGGGATCCAGAAAACAGCCTCACGCCGTTTGTGTACCTTTTCCTGCTCAGGGACACATTAACCCTATGA
TGCAGCTTGCCAAGCTACTGCATTCAAGAGGCTTCTACATAACGTTCGTCAACACCGAGTTTAACCATAG
GAGACTGCTGCAATCCAAGGGTCCCGAATTTTTGAAAGGTTGTGCAGACTTCCAGTTTGAGTCCATTCCA
GATGGTTTACCTCCCTCAGACAGAGACGCCACGCAAGACCCACCTACTCTGTGTATTGCAATGAGGGATA
ATTGTCTTGACCCATTCAGGGTGTTATTGAAGAAGTTAAATAATAACAACAACTCCATTGCTTCCAGGCA
AGTCCCAGGAGTGACATGTGTCGTTAGTGACGGAGCTATGAATTTTGCCATGAAAGCCGCTGAAGAAGCT
GGAATACCCGAGGTCCAGTTCTGGACCGCTTCCGCCTGCGGTTTTATGGGCTATTTGCATTATCCACAGC
TAGTGCAGAGGGGCATCTTCCCCTTCAAGGATGAAAGTTTCCAGAGTGACGGTTCATTAGACACTACTAT
CGATTGGATCCCTGGAATGCGTAACATTAGACTAAAGGACATGCCTTCCTTCATCCGTACCACAGATCCA
AACGATATACTGTTTAATTACCTTAGTGAGGAAGTTCAGAACTGTTTAAAAGCCAGTGCAATCATATTTA
ACACATTCGATACACTTGAGCACCAGGTCCTGCAGGCTATCGCCTCCAAATTTCACAATATATACACGAT
AGGTCCTTTGAGTCTGCTATCTAAACAGGTTATAGATGGCGAGTTTAAGTCTCTGAACAGTTCTTTATGG
AAAGAAGATACTAAATGCCTACAGTGGCTAGACACGAAGGAACCCAACTCTGTTGTATACGTTAATTATG
GCTCAATAACAGTAATGACAGACCAGCATCTTAAGGAGTTCGCCTGGGGTCTTGCCAATTCTAAACACCC
CTTCCTTTGGATCGTGAGACCCGACATTGTCATGGGAGATAGTGCTATCTTACCCGAACACTTCGTTGAG
GAAACCAAAGACAGGGGACTATTAGTATCATGGTGTCCTCAAGAACAAGTCTTATCACATCCTTCTATTG
GTGTTTTTCTAACCCATTGCGGTTGGAACTCCACCTTGGAGTCCATATGTGGTGGTGTGCCCATTATATG
CTGGCCATTCTTCGCCGAACAACAGACCAACTGCAGGTACGCCTGCACGGAATGGGGTATAGGCATGGAA
GTGAACCATGACGTAAAGCGTAACGAAATTGTGGCCTTAATAAATGAGATGTTAGAGGGCGACAAGGGAA
AACAGATGAGAAAGAAGGCTTTGAAATTAAAAAAGGAGGCTGAGGAAGCCACTGACGTCGGAGGCCTATC
CTACAACAATTTTGATAGATTGATTAAGGAGGCACTACATTACTGCGAGCAATACTAA
SEQ ID NO. 6
Amino Acid
UGT85K11
Camellia sinensis
MGSRKQPHAVCVPFPAQGHINPMMQLAKLLHSRGFYITFVNTEFNHRRLLQSKGPEFLKGCADFQFESIP
DGLPPSDRDATQDPPTLCIAMRDNCLDPFRVLLKKLNNNNNSIASRQVPGVTCVVSDGAMNFAMKAAEEA
GIPEVQFWTASACGFMGYLHYPQLVQRGIFPFKDESFQSDGSLDTTIDWIPGMRNIRLKDMPSFIRTTDP
NDILFNYLSEEVQNCLKASAIIFNTFDTLEHQVLQAIASKFHNIYTIGPLSLLSKQVIDGEFKSLNSSLW
KEDTKCLQWLDTKEPNSVVYVNYGSITVMTDQHLKEFAWGLANSKHPFLWIVRPDIVMGDSAILPEHFVE
ETKDRGLLVSWCPQEQVLSHPSIGVFLTHCGWNSTLESICGGVPIICWPFFAEQQTNCRYACTEWGIGME
VNHDVKRNEIVALINEMLEGDKGKQMRKKALKLKKEAEEATDVGGLSYNNFDRLIKEALHYCEQY
SEQ ID NO. 7
DNA
UGT94P1 codon-optimized
Camellia sinensis
ATGGATTCAAAAAAGAGCAAAATGAATGTCCTAATGCTACCATGGTTAGCCCAAGGTCACATCACTCCCT
TCCTAGAGCTTGCCAAGAAACTCACAAACAAAAACTTCCACATATATTTCTGTTCTACACCAATCAATCT
CATATCCATCAAGAAAAGAATCACAGACAAGTACTCTCTCTCCATTGAACTTGTAGAAATCCACCTTCCA
TCCTTGCCTGAGCTCCCTCCTCACTACCACACCACCAATGGCCTCCCCATCCATCTCAATTCCACTCTCA
AAACAGCCTTTGAGATGGCAAGCACAAGCTTCTCCACCATCCTCAACACACTAAGCCCAGATCTTGTAAT
TTATGACGTCAGCCCCTCATGGGCACAATCCACTGCCTTGTCGTTTGATATTCCCGCAGTCCAGCTCATG
ATTACCGGAGCCACGGTGGTTTCTTTTGGCCAACATATGATTAAGCACTGTGGCAGTGTTGAATTCCCAT
TTCCGGCAATTAAGCTGCAGGGATTCCATGAAACGCAGTTTCGCCACTTTGTTGAGACTGTTGTAAAAGA
ATACAATGACAAACAAGTTGCCTCTGTAAATGATCAACCATCTTGCAACTTTATGTTGTACAACACTTTT
AGAGAGCTTGAAGGTAAATACATTGATTATCTACCGGTTATAGGTGAGAAGAAGGTTGTCCCTGTTGGTC
CACTTGTTCAAGGCATTGATGATGATGAAAATGAGCACTCTGAGATCATACAATGGCTTGACAACAAGGG
TGAGTACTCCACTTTGTTTGTTTCTTTTGGGAGTGAGTATTTTATGTCCAAGGAAGAGATTGAAGAGATA
GCTCATGGGTTAGAGCTTAGCATGGTTAATTTCATATGGGTTGTTAGGTTTCCTGAGGTAGAGAAAGTTG
AGCTTGAAGAAGCACTTCCTAAGGGGTTTATTGACAGGGTAGGAGAGAGAGGACTGGTTGTGGAGGGTTG
GGCTCCACAGGCAAGAATTCTAACCCATTCAAGCACTGGTGGATTTGTGAGTCACTGTGAATGGAATTCA
GTATTGGAAAGCCTAAAGTTTGGGGTTCCAATGGTAGCCATACCCATGCAATATGAGCAGCCGTTGAATG
CTAAACTAGTGGAGGAGGTTGGTGTGGCTGCGGAGGTCAACAGAGACATCAATGGAAGACTCAACAGAGA
AGAGATAGCACAGGTAATAAGGAAGGTTGTGGTGGAGAAAAGTGGAGAGGACATAAGGATTAAAGCGAGA
ATTTTTGGGGATAAAATAAGAATGAAAGGAGATGAAGAGATAGACGAGGCAGTAGAAGTGTTGCTGCAGC
TTTGTAAGGATGTGAAGTTGCTCAAAAATTAG
SEQ ID NO. 8
Amino Acid
UGT94P1
Camellia sinensis
MDSKKSKMNVLMLPWLAQGHITPFLELAKKLTNKNFHIYFCSTPINLISIKKRITDKYSLSIELVEIHLP
SLPELPPHYHTTNGLPIHLNSTLKTAFEMASTSFSTILNTLSPDLVIYDVSPSWAQSTALSFDIPAVQLM
ITGATVVSFGQHMIKHCGSVEFPFPAIKLQGFHETQFRHFVETVVKEYNDKQVASVNDQPSCNFMLYNTF
RELEGKYIDYLPVIGEKKVVPVGPLVQGIDDDENEHSEIIQWLDNKGEYSTLFVSFGSEYFMSKEEIEEI
AHGLELSMVNFIWVVRFPEVEKVELEEALPKGFIDRVGERGLVVEGWAPQARILTHSSTGGFVSHCEWNS
VLESLKFGVPMVAIPMQYEQPLNAKLVEEVGVAAEVNRDINGRLNREEIAQVIRKVVVEKSGEDIRIKAR
IFGDKIRMKGDEEIDEAVEVLLQLCKDVKLLKN
SEQ ID NO. 9
DNA
VvGT7 codon-optimized
Vitis vinifera
ATGGAATCTGTTGTTTTGTACCCATCTCCAGGTATGGGTCATTTGATTTCTATGGTTGAATTGGGTAAGT
TGATTTTGAAGCATCATCCATCTTTTTCTATTACTATTTTTATTGTTACTCCACCATACAACACTGGTTC
TACTGCTCCATACTTGGCTAGAGTTTCTTCTACTATTCCATCTATTACTTTTCATCATTTGCCAACTATT
TCTTTGCCATTGGATTCTTTTTCTTCTCCAAACCATGAAACTTTGGCTTTTGAATTGTTGAGATTGAACA
ACCCAAACATTCATCAAGCTTTGGTTTCTATTTCTAACAACTCTTCTGTTAGAGCTTTGATTGTTGATTG
TTTTTGTACTGCTGCTTTGTCTGTTGCTGCTCAATTGAACATTCCATTTTACTACTTTTTTACTTCTGGT
GCTTGTTGTTTGGCTTCTTTTTTGTACTTGCCATTTATTCATCAACAAACTACTAAGTCTTTTAAGGATT
TGAACACTCATTTGCATATTCCAGGTTTGCCACCAGTTCCAGCTTCTGATATGGCTAAGCCAATTTTGGA
TAGAGAAGATAAGGCTTACGAATTGTTTGTTAACATGTCTATTCATTTGCCAAGATCTGCTGGTATTATT
GTTAACACTTTTGAAGCTTTGGAACCAAGAGCTGTTAAGACTATTTTGGATGGTTTGTGTGTTTTGGATG
GTCCAACTTCTCCAATTTTTTGTATTGGTCCATTGATTGCTGCTGATGATAGATCTGGTGGTGGTGGTGG
TGGTGGTGGTGGTTCTGGTATTCCAGAATGTTTGACTTGGTTGGAATCTCAACCAAAGAGATCTGTTTTG
TTTTTGTGTTTTGGTTCTTTGGGTTTGTTTTCTGAAGAACAATTGAAGGAAATTGCTGTTGGTTTGGAAA
GATCTGGTCAAAGATTTTTGTGGGTTGTTAGATCTCCACCATCTAAGGATCCATCTAGAAGATTTTTGGC
TCCACCAGAACCAGATTTGAACTCTTTGTTGCCAGATGGTTTTTTGGATAGAACTAAGGAAAGAGGTTTG
ATGGTTAAGTCTTGGGCTCCACAAGTTGCTGTTTTGAACCATGCTTCTGTTGGTGGTTTTGTTACTCATT
GTGGTTGGAACTCTGTTTTGGAAGCTGTTTGTGCTGGTGTTCCAATGGTTGCTTGGCCATTGTACGCTGA
ACAAAGATTTAACAGAGTTGTTTTGGTTGAAGAAATGAAGTTGGCTTTTCCAATGGAAGAATCTGAAGAA
GGTTTTGTTACTGCTACTGAAGTTGAAAAGAGAGTTAGAGAATTGATGGAATCTGAAGAAGGTAACACTT
TGAGATTGAGAATTATGGCTATGAAGGAAGCTGCTGAAACTGCTATGTCTGATGGTGGTTCTTCTAGAAC
TGCTTTGACTAAGTTGGTTAAGTCTTGGAGACCAGGTTAA
SEQ ID NO. 10
Amino Acid
VvGT7
Vitis vinifera
MESVVLYPSPGMGHLISMVELGKLILKHHPSFSITIFIVTPPYNTGSTAPYLARVSSTIPSITFHHLPTI
SLPLDSFSSPNHETLAFELLRLNNPNIHQALVSISNNSSVRALIVDCFCTAALSVAAQLNIPFYYFFTSG
ACCLASFLYLPFIHQQTTKSFKDLNTHLHIPGLPPVPASDMAKPILDREDKAYELFVNMSIHLPRSAGII
VNTFEALEPRAVKTILDGLCVLDGPTSPIFCIGPLIAADDRSGGGGGGGGGSGIPECLTWLESQPKRSVL
FLCFGSLGLFSEEQLKEIAVGLERSGQRFLWVVRSPPSKDPSRRFLAPPEPDLNSLLPDGFLDRTKERGL
MVKSWAPQVAVLNHASVGGFVTHCGWNSVLEAVCAGVPMVAWPLYAEQRFNRVVLVEEMKLAFPMEESEE
GFVTATEVEKRVRELMESEEGNTLRLRIMAMKEAAETAMSDGGSSRTALTKLVKSWRPG
SEQ ID NO. 11
DNA
NtGT3 codon-optimized
Nicotiana tabacum
ATGAAAGAGACTAAAAAAATTGAGTTAGTTTTTATCCCCAGTCCTGGTATAGGACACTTAGTCTCAACTG
TGGAGATGGCCAAACTGTTGATAGCCCGTGAAGAGCAACTTTCTATTACTGTCCTGATTATACAATGGCC
TAATGATAAAAAGCTAGACAGTTATATCCAGTCCGTCGCAAACTTTAGTTCTAGACTGAAGTTTATACGT
CTGCCCCAAGATGACTCAATCATGCAACTTTTGAAATCAAACATTTTCACGACATTCATCGCCTCTCACA
AGCCAGCTGTAAGAGACGCCGTTGCTGACATACTAAAGAGTGAAAGTAATAACACATTGGCAGGCATTGT
AATCGATCTTTTCTGCACATCCATGATCGATGTAGCCAATGAGTTTGAGCTGCCTACTTATGTGTTTTAC
ACTAGTGGCGCAGCCACGTTGGGTCTGCACTACCATATTCAAAATCTGCGTGATGAGTTTAATAAAGACA
TTACCAAATATAAGGATGAGCCAGAAGAAAAATTAAGTATAGCCACGTACCTTAACCCATTCCCTGCTAA
GTGTCTACCCTCCGTGGCATTGGATAAGGAAGGAGGATCAACGATGTTCCTAGACTTAGCTAAGAGGTTC
AGGGAGACCAAAGGCATAATGATTAACACTTTTCTTGAGCTGGAATCATACGCTCTAAACTCATTGTCTA
GAGATAAAAACTTGCCCCCTATATACCCTGTAGGCCCTGTTTTGAACTTGAACAACGTTGAGGGTGATAA
CTTGGGCTCTAGTGATCAAAATACCATGAAATGGCTGGACGACCAGCCAGCTTCTTCCGTTGTGTTCCTA
TGTTTTGGCTCAGGAGGAAGTTTCGAAAAACACCAAGTCAAAGAAATAGCTTATGCCTTAGAATCTTCCG
GATGCAGGTTCTTGTGGAGTTTGCGTAGACCCCCCACGGAAGATGCTAGGTTCCCTTCTAATTACGAAAA
CTTAGAGGAAATTTTACCAGAGGGATTTCTGGAAAGAACGAAAGGCATTGGTAAGGTCATTGGATGGGCC
CCACAGTTAGCAATCTTGTCTCACAAGTCCACAGGAGGATTCGTGTCTCATTGCGGATGGAACTCTACCC
TTGAAAGTACCTATTTCGGCGTTCCTATTGCTACTTGGCCAATGTATGCTGAACAACAGGCCAACGCTTT
TCAACTTGTTAAAGATTTGAGGATGGGTGTTGAGATCAAAATGGATTATAGGAAGGATATGAAGGTAATG
GGCAAGGAGGTTATCGTTAAGGCAGAAGAAATTGAAAAGGCCATAAGGGAAATCATGGACTCAGAATCAG
AAATCAGGGTCAAGGTCAAAGAGATGAAGGAGAAAAGTCGTGCAGCCCAAATGGAAGGAGGATCATCATA
TACCTCTATCGGCGGCTTCATTCAAATAATCATGGAGAACTCACAGTAA
SEQ ID NO. 12
Amino Acid
NtGT3
Nicotiana tabacum
MKETKKIELVFIPSPGIGHLVSTVEMAKLLIAREEQLSITVLIIQWPNDKKLDSYIQSVANFSSRLKFIR
LPQDDSIMQLLKSNIFTTFIASHKPAVRDAVADILKSESNNTLAGIVIDLFCTSMIDVANEFELPTYVFY
TSGAATLGLHYHIQNLRDEFNKDITKYKDEPEEKLSIATYLNPFPAKCLPSVALDKEGGSTMFLDLAKRF
RETKGIMINTFLELESYALNSLSRDKNLPPIYPVGPVLNLNNVEGDNLGSSDQNTMKWLDDQPASSVVFL
CFGSGGSFEKHQVKEIAYALESSGCRFLWSLRRPPTEDARFPSNYENLEEILPEGFLERTKGIGKVIGWA
PQLAILSHKSTGGFVSHCGWNSTLESTYFGVPIATWPMYAEQQANAFQLVKDLRMGVEIKMDYRKDMKVM
GKEVIVKAEEIEKAIREIMDSESEIRVKVKEMKEKSRAAQMEGGSSYTSIGGFIQIIMENSQ
SEQ ID NO. 13
DNA
NtGT4 codon-optimized
Nicotiana tabacum
ATGGCTACTCAGGTGCATAAATTGCATTTCATTCTGTTCCCACTGATGGCTCCCGGTCACATGATCCCTA
TGATAGACATCGCAAAACTATTGGCTAACCGTGGCGTGATAACTACCATAATAACTACGCCCGTTAACGC
CAATCGTTTTTCCTCTACGATCACTAGGGCCATTAAATCAGGCCTAAGAATCCAGATTTTAACCTTAAAA
TTCCCATCAGTTGAGGTAGGCCTGCCTGAAGGATGTGAAAACATCGACATGTTGCCATCTTTGGACTTAG
CCTCTAAATTCTTTGCTGCTATTTCTATGCTTAAACAACAAGTGGAGAACTTGCTAGAGGGTATTAACCC
TAGTCCCTCATGCGTTATTTCTGACATGGGCTTCCCATGGACGACACAGATCGCTCAAAATTTCAATATT
CCTCGTATCGTATTTCATGGCACGTGTTGCTTTTCTCTTCTTTGTTCTTACAAAATCCTGTCATCCAATA
TCTTAGAGAACATTACTAGTGACTCAGAGTATTTTGTCGTGCCAGATCTGCCAGACCGTGTCGAGCTAAC
TAAGGCCCAAGTCTCTGGATCTACAAAGAATACTACATCAGTAAGTAGTTCAGTACTGAAGGAGGTTACA
GAGCAGATCAGGCTTGCAGAGGAATCATCCTACGGTGTGATAGTTAATTCCTTCGAAGAACTGGAACAGG
TGTATGAAAAAGAGTACAGAAAAGCCAGGGGCAAAAAGGTCTGGTGCGTGGGTCCTGTCTCTTTGTGCAA
CAAGGAGATTGAAGATCTTGTTACTAGAGGAAACAAAACCGCTATAGACAATCAGGATTGTCTTAAGTGG
TTAGACAACTTCGAGACTGAATCCGTCGTCTATGCAAGTTTAGGCTCACTAAGTAGGCTTACGTTACTGC
AAATGGTTGAGCTGGGATTGGGACTGGAGGAGAGTAATAGGCCATTTGTATGGGTTCTGGGAGGAGGAGA
CAAACTAAATGATCTTGAGAAATGGATATTGGAGAATGGCTTTGAACAGCGTATAAAGGAGAGAGGTGTC
CTGATACGTGGCTGGGCACCTCAAGTATTGATTTTAAGTCACCCCGCAATTGGAGGAGTTTTAACGCATT
GTGGATGGAACTCTACATTAGAGGGCATTTCAGCCGGACTACCCATGGTCACCTGGCCACTATTTGCCGA
ACAGTTCTGTAACGAAAAATTAGTAGTGCAGGTTCTTAAAATCGGTGTCTCACTTGGAGTGAAGGTCCCT
GTTAAGTGGGGTGACGAAGAGAACGTAGGTGTCTTAGTGAAAAAGGATGACGTTAAAAAAGCACTGGATA
AGCTAATGGATGAGGGTGAGGAGGGCCAGGTTAGGAGGACCAAAGCCAAAGAGCTTGGTGAGTTAGCTAA
AAAAGCCTTTGGAGAGGGCGGATCATCCTACGTGAACCTAACGTCCCTAATTGAAGATATAATCGAGCAG
CAGAACCATAAGGAGAAGTAG
SEQ ID NO. 14
Amino Acid
NtGT4
Nicotiana tabacum
MATQVHKLHFILFPLMAPGHMIPMIDIAKLLANRGVITTIITTPVNANRFSSTITRAIKSGLRIQILTLK
FPSVEVGLPEGCENIDMLPSLDLASKFFAAISMLKQQVENLLEGINPSPSCVISDMGFPWTTQIAQNFNI
PRIVFHGTCCFSLLCSYKILSSNILENITSDSEYFVVPDLPDRVELTKAQVSGSTKNTTSVSSSVLKEVT
EQIRLAEESSYGVIVNSFEELEQVYEKEYRKARGKKVWCVGPVSLCNKEIEDLVTRGNKTAIDNQDCLKW
LDNFETESVVYASLGSLSRLTLLQMVELGLGLEESNRPFVWVLGGGDKLNDLEKWILENGFEQRIKERGV
LIRGWAPQVLILSHPAIGGVLTHCGWNSTLEGISAGLPMVTWPLFAEQFCNEKLVVQVLKIGVSLGVKVP
VKWGDEENVGVLVKKDDVKKALDKLMDEGEEGQVRRTKAKELGELAKKAFGEGGSSYVNLTSLIEDIIEQ
QNHKEK
[DSK INSERT]
SEQ ID NO. 29
DNA
Bs-YjiC codon-optimized
Bacillus subtilis
ATGAAAAAGTACCATATTTCTATGATCAATATCCCAGCTTACGGACATGTCAATCCTACGCTTGCTTTAG
TAGAGAAGCTTTGTGAGAAAGGGCACCGTGTCACGTACGCTACGACTGAGGAGTTTGCTCCCGCTGTTCA
GCAAGCCGGTGGAGAAGCATTGATCTATCATACATCCTTGAATATTGATCCTAAGCAAATCAGGGAGATG
ATGGAAAAGAATGACGCTCCCTTGTCTCTTTTGAAAGAATCATTGTCTATTCTGCCACAGCTTGAGGAGT
TATATAAGGATGATCAGCCTGATCTGATCATCTATGACTTTGTTGCTCTGGCTGGTAAATTGTTTGCTGA
AAAGCTTAATGTTCCAGTCATTAAGTTGTGTTCTTCATATGCCCAAAATGAATCCTTTCAGTTAGGAAAT
GAAGACATGCTGAAAAAAATAAGAGAAGCAGAGGCTGAATTTAAAGCCTACTTGGAGCAAGAGAAGTTGC
CAGCTGTTTCATTTGAACAGTTAGCTGTGCCAGAAGCATTAAATATTGTCTTTATGCCAAAGTCTTTTCA
GATTCAGCATGAGACGTTCGATGACCGTTTCTGTTTTGTCGGCCCCTCTTTGGGAGAAAGAAAGGAAAAA
GAATCTCTGTTGATTGACAAGGATGATAGACCACTTATGCTGATTTCTTTGGGTACGGCTTTTAACGCAT
GGCCAGAATTTTACAAGATGTGCATCAAGGCATTTAGAGATTCTTCATGGCAAGTGATCATGTCTGTTGG
GAAAACGATTGATCCAGAATCTTTGGAGGATATTCCTGCTAACTTTACCATTAGACAAAGTGTGCCACAG
CTTGAGGTGTTAGAGAAAGCTGATTTGTTCATCTCTCATGGCGGGATGAACAGTACGATGGAAGCTATGA
ACGCAGGTGTGCCACTTGTCGTCATTCCACAAATGTATGAGCAGGAGTTGACTGCAAATAGAGTTGATGA
ATTAGGCCTTGGCGTTTATTTGCCAAAAGAGGAAGTGACTGTTTCCTCTCTGCAGGAAGCTGTTCAGGCT
GTATCCAGTGATCAAGAGCTGTTGTCTAGAGTCAAGAATATGCAAAAGGATGTAAAAGAAGCTGGCGGAG
CTGAGCGTGCTGCAGCTGAGATTGAAGCTTTTATGAAAAAATCCGCTGTCCCACAGTAA
SEQ ID NO. 30
Amino Acid
Bs-YjiC
Bacillus subtilis
MKKYHISMINIPAYGHVNPTLALVEKLCEKGHRVTYATTEEFAPAVQQAGGEALIYHTSLNIDPKQIREM
MEKNDAPLSLLKESLSILPQLEELYKDDQPDLIIYDFVALAGKLFAEKLNVPVIKLCSSYAQNESFQLGN
EDMLKKIREAEAEFKAYLEQEKLPAVSFEQLAVPEALNIVFMPKSFQIQHETFDDRFCFVGPSLGERKEK
ESLLIDKDDRPLMLISLGTAFNAWPEFYKMCIKAFRDSSWQVIMSVGKTIDPESLEDIPANFTIRQSVPQ
LEVLEKADLFISHGGMNSTMEAMNAGVPLVVIPQMYEQELTANRVDELGLGVYLPKEEVTVSSLQEAVQA
VSSDQELLSRVKNMQKDVKEAGGAERAAAEIEAFMKKSAVPQ
SEQ ID NO. 31
DNA
VvGT14 codon-optimized
Vitis vinifera
ATGGGTTCTATGGAAAAGCCACATGCTGTTTGTATTCCATACCCAGCTCAAGGTCATATTAACCCAATGT
TGAAGGTTGCTAAGTTGTTGCATTTTAGAGGTTTTAGAATTACTTTTGTTAACACTGAATTTAACCATAC
TAGATTGTTGAAGGCTCAAGGTCCAAACTCTTTGAACGGTTTGCCAACTTTTCAATTTGAAACTATTCCA
GATGGTTTGCCACCATCTAACGTTGATGCTACTCAAGATATTCCATCTTTGTGTGCTTCTACTAAGAAGA
ACTGTTTGGCTCCATTTAGAAGATTGTTGGCTAAGTTGAACGATAGAGGTCCACCAGTTACTTGTATTTT
TTCTGATGCTGTTATGTCTTTTACTTTGGATGCTGCTCAAGAATTGGGTATTCCAGATTTGTTGTTGTGG
ACTGCTTCTGCTTGTGGTTTTATGGCTTACGTTCAATACAGATCTTTGATTGATAAGGGTTTTACTCCAT
TGAAGGATGAATCTTACTTGACTAACGGTTACTTGGATACTGTTGTTGATTGGATTCCAGGTATGAAGGG
TATTAGATTGAAGGATTTGCCATCTTTTATTAGAACTACTGATCCAGATGATATTATGTTGGATTTTGCT
ATGGGTGAATTGGAAAGAGCTAGAAAGGCTTCTGCTATTATTTTTAACACTTTTGATGCTTTGGAACAAG
AAGTTTTGGATGCTATTGCTCCAATGTACCCACCAATTTACACTATTGGTCCATTGCAATTGTTGCCAGA
TCAAATTCATGATTCTGAATTGAAGTTGATTGGTTCTAACTTGTGGAAGGAAGAACCAGAATGTTTGAAG
TGGTTGGATTCTAAGGAACCAAACTCTGTTGTTTACGTTAACTACGGTTCTATTACTGTTATGACTCCAC
AACAATTGATTGAATTTGCTTGGGGTTTGGCTAACTCTAACCAATCTTTTTTGTGGATTTTGAGACCAGA
TTTGGTTTCTGGTGAATCTGCTATTTTGCCACCAGAATTTGTTGCTGAAACTGAAGATAGAGGTTTGTTG
GCTGGTTGGTGTCCACAAGAACAAGTTTTGACTCATCAAGCTATTGGTGGTTTTTTGACTCATAACGGTT
GGAACTCTACTATTGAAGGTTTGTGTGCTGGTGTTCCAATGATTTGTTGGCCATTTTTTGCTGAACAACA
AACTAACTGTAGATACTGTTGTACTGAATGGGGTGTTGGTATGGAAATTGATTCTGATGTTAAGAGAGAT
GAAGTTGCTAAGTTGGTTAGAGAATTGATGGTTGGTGAAAAGGGTAAGGTTATGAAGAAGAAGACTATGG
AATGGAAGCATAGAGCTGAAGTTGCTACTACTGGTCCAGATGGTTCTTCTTACTTGAACTTGGAAAAGAT
TTTTGAACAAGTTTTGTTGTAA
SEQ ID NO. 32
Amino Acid
VvGT14
Vitis vinifera
MGSMEKPHAVCIPYPAQGHINPMLKVAKLLHFRGFRITFVNTEFNHTRLLKAQGPNSLNGLPTFQFETIP
DGLPPSNVDATQDIPSLCASTKKNCLAPFRRLLAKLNDRGPPVTCIFSDAVMSFTLDAAQELGIPDLLLW
TASACGFMAYVQYRSLIDKGFTPLKDESYLTNGYLDTVVDWIPGMKGIRLKDLPSFIRTTDPDDIMLDFA
MGELERARKASAIIFNTFDALEQEVLDAIAPMYPPIYTIGPLQLLPDQIHDSELKLIGSNLWKEEPECLK
WLDSKEPNSVVYVNYGSITVMTPQQLIEFAWGLANSNQSFLWILRPDLVSGESAILPPEFVAETEDRGLL
AGWCPQEQVLTHQAIGGFLTHNGWNSTIEGLCAGVPMICWPFFAEQQTNCRYCCTEWGVGMEIDSDVKRD
EVAKLVRELMVGEKGKVMKKKTMEWKHRAEVATTGPDGSSYLNLEKIFEQVLL

1. An in vivo method for producing water-soluble terpenes comprising the steps:
genetically modified a yeast cell to express a nucleotide sequence encoding at least one heterologous glycosyltransferase, operably linked to a promotor, having glycosylation activity towards one or more terpenes;
establishing a suspension cell culture of genetically modified yeast cells;
introducing one or more terpenes to said suspension cell culture of genetically modified yeast cells; and
converting said one or more terpenes into one or more water-soluble glycosylated terpenes through the activity of said at least one heterologous glycosyltransferase.

2. The method of claim 1, wherein said genetically modified yeast cells comprise genetically modified yeast cells selected from the group consisting of: genetically modified Pichia pastoris cells, genetically modified Saccharomyces cerevisiae cells, and/or genetically modified Kluyveromyces marxianus cells.

3. The method of claim 1, wherein said nucleotide sequence encoding a heterologous glycosyltransferase having glycosylation activity towards one or more terpenes comprises a nucleotide sequence selected from the group consisting of: SEQ ID NO. 1; SEQ ID NO. 3; SEQ ID NO. 9; SEQ ID NO. 5; SEQ ID NO. 7; SEQ ID NO. 11; SEQ ID NO. 13; SEQ ID NO. 29; and SEQ ID NO. 31.

4. The method of claim 1, wherein said heterologous glycosyltransferase having glycosylation activity towards one or more terpenes comprises a heterologous glycosyltransferase selected from the group consisting of: UGT58A1; MhGt1; VvGt7; UGT94P1; Bs-YjiC; VvGT14; and a homolog protein having glycosylation activity towards one or more terpenes.

5. The method of claim 1, wherein said heterologous glycosyltransferase having glycosylation activity towards one or more terpenes comprises a heterologous glycosyltransferase selected from the group consisting of: SEQ ID NO. 2; SEQ ID NO. 4; SEQ ID NO. 10; SEQ ID NO. 6; SEQ ID NO. 8; (SEQ ID NO. 30; SEQ ID NO. 32; and a heterologous homolog protein having glycosylation activity towards one or more terpenes.

6. The method of claim 1, wherein said one or more water-soluble glycosylated terpenes comprises at least one water-soluble glycosylated terpene selected from the group consisting of: a terpene glycoside, a glycosylated-xylosylated terpene, and an acetylated glycoside terpene.

7. The method of claim 1, wherein said one or more water-soluble glycosylated terpenes comprises at least one water-soluble glycosylated terpene selected from the group consisting of: alpha-Bisabolyl monoglucoside, alpha-Bisabolyl O-acetyl glucoside, alpha-Bisabolyl oxidized glucoside, alpha-Bisabolyl diglucoside, Citronellyl monoglucoside, Geranyl O-acetyl glucoside, Geranyl monoglucoside, Linalyl monoglucoside, Linalyl glucoside-xyloside, Linalyl diglucoside, Neryl monoglucoside, Neryl diglucoside and Terpineol diglucoside.

8. The method of claim 1, wherein said step of introducing one or more terpenes to said suspension cell culture of genetically modified yeast cells comprises the step of introducing one or more isolated terpenes to the yeast suspension cell culture.

9. The method of claim 1, wherein said step of introducing one or more terpenes to said suspension cell culture of genetically modified yeast cells comprises the step of introducing a Cannabis extract having one or more terpenes to the yeast suspension cell culture.

10. The method of claim 1, wherein said step of introducing one or more terpenes to said suspension cell culture of genetically modified yeast cells comprises the step of introducing a full spectrum Cannabis extract having one or more terpenes and one or more cannabinoid to the yeast suspension cell culture.

11. The method of claim 10, wherein said heterologous glycosyltransferase having glycosylation activity towards one or more terpenes comprises a heterologous glycosyltransferase protein having glycosylation activity towards one or more terpenes and one or more cannabinoids.

12. (canceled)

13. The method of claim 11, wherein said heterologous glycosyltransferase protein having glycosylation activity towards one or more terpenes and one or more cannabinoids comprises a glycosyltransferase protein selected from the group consisting of: SEQ ID NO. 12, and SEQ ID NO. 14, or an amino acid sequence having at least 90% sequence identity with SEQ ID NO. 12, and SEQ ID NO. 14, and that encodes a protein having glycosylation activity towards one or more terpenes and one or more cannabinoids.

14-84. (canceled)

85. A method for producing glycosylated terpenes comprising the steps:
introducing one or more terpene compounds to a glycosyltransferase enzyme, wherein the glycosyltransferase enzyme is selected from the group of amino acid sequences consisting of: SEQ ID NO. 2, 4, 6, 8, 10, 12, 14, 30, and 32.
glycosylating the one or more terpene compounds; and
isolating the glycosylated terpene compounds.

86. The method of claim 85, wherein said glycosylated terpene comprises at least one glycosylated terpene selected from the group consisting of: alpha-Bisabolyl monoglucoside, alpha-Bisabolyl O-acetyl glucoside, alpha-Bisabolyl oxidized glucoside, alpha-Bisabolyl diglucoside, Citronellyl monoglucoside, Geranyl O-acetyl glucoside, Geranyl monoglucoside, Linalyl monoglucoside, Linalyl glucoside-xyloside, Linalyl diglucoside, Neryl monoglucoside, Neryl diglucoside and Terpineol diglucoside.

87. The method of claim 85, wherein said step of introducing comprises the step of introducing the one or more terpene compounds with a glycosyltransferase enzyme in vitro, or with a glycosyltransferase enzyme expressed in vivo in a yeast cell, a bacterial cell, or a plant cell.

88. A method comprising the steps:
providing a first quantity of a terpene compound and a first quantity of a cannabinoid compound;
introducing the terpene compound to a first glycosyltransferase, wherein the glycosyltransferase enzyme is selected from the group of amino acid sequences consisting of: SEQ ID NO. 2, 4, 6, 8, 10, 12, 14, 30, and 32; and
introducing the cannabinoid compound to a glycosyltransferase, wherein the glycosyltransferase enzyme is selected from the group of amino acid sequences consisting of: SEQ ID NO. 12, and SEQ ID NO. 14.

89. The method of claim 88, wherein said step of introducing comprises the step of introducing the one or more terpene compounds with a glycosyltransferase enzyme in vitro, or with a glycosyltransferase enzyme expressed in vivo in a yeast cell, a bacterial cell, or a plant cell.

90. The method of claim 88, wherein said glycosylated terpene comprises at least one glycosylated terpene selected from the group consisting of: alpha-Bisabolyl monoglucoside, alpha-Bisabolyl O-acetyl glucoside, alpha-Bisabolyl oxidized glucoside, alpha-Bisabolyl diglucoside, Citronellyl monoglucoside, Geranyl O-acetyl glucoside, Geranyl monoglucoside, Linalyl monoglucoside, Linalyl glucoside-xyloside, Linalyl diglucoside, Neryl monoglucoside, Neryl diglucoside and Terpineol diglucoside.

91. The method of claim 88, wherein said cannabinoid compound is selected from the group consisting of: cannabidiol (CBD), and tetrahydrocannabinol (THC).

92. The method of claim 11, wherein said cannabinoid compound is selected from the group consisting of: cannabidiol (CBD), and tetrahydrocannabinol (THC).
